(19) United States
(12) Patent Application Publication
(10) Pub. No.: US 2026/0014207 A1
(43) Pub. Date: Jan. 15, 2026

(54) METHODS AND COMPOSITIONS FOR TREATING PERINATAL HYPOXIC-ISCHEMIC BRAIN INJURY

(71) Applicant: Sanford Burnham Prebys Medical Discovery Institute, La Jolla, CA (US)

(72) Inventor: Evan Y. SNYDER, La Jolla, CA (US)

(21) Appl. No.: 19/278,323

(22) Filed: Jul. 23, 2025

Related U.S. Application Data
(63) Continuation of application No. PCT/US24/12581, filed on Jan. 23, 2024.
(60) Provisional application No. 63/481,149, filed on Jan. 23, 2023.

(51) Int. Cl. A61K 35/30

(52) U.S. Cl. CPC A61K 35/30

(57) ABSTRACT

Various aspects of the pharmaceutical compositions, kits, and associated methods for treating perinatal hypoxic-ischemic injuries (HII) are provided herein, including methods of preparing and administering hNSCs (e.g., HFB 2050 hNSCs) or progenitors thereof.

### CROSS REFERENCE

This application is a continuation of International Application No. PCT/US2024/012581, filed on Jan. 23, 2024, and claims the benefit of U.S. Provisional Application Ser. No. 63/481,149, filed on Jan. 23, 2023, all which are incorporated herein by reference in their entirety.

### BACKGROUND

Neonates affected by perinatal/neonatal cerebral hypoxic-ischemic injury (HII; also termed “perinatal asphyxia”) can develop neurological signs despite the use of standard of care treatment (e.g., therapeutic hypothermia; or HT). 10%-20% of affected babies become significantly impaired for life with neurological sequelae (e.g., cerebral palsy, mental and motor disability, and epilepsy). Currently there is no impactful treatment for perinatal HII based on its pathophysiology. HT must be administered within 6 hours after birth and offers only marginal neuroprotection in about 10% of Sarnat Stage 2 (clinically “moderate”) HII, but not Sarnat Stage 3 (clinically “severe”) HII. Hence, there is a great need for treatments with flexible timelines and are directed against pathophysiological mechanisms that cannot be addressed by HT.

### SUMMARY

One aspect of the present disclosure provides a method of treating perinatal hypoxic-ischemic injuries (HII) in a human subject, comprising administering to the human subject an effective amount of human neural stem cells (hNSCs) or progenitors thereof comprising an HFB2050 cell or a progenitor thereof, thereby treating the perinatal hypoxic-ischemic injuries in a human subject.

Aspects disclosed in methods include a method of treating perinatal hypoxic-ischemic injuries (HII) in a human subject, comprising administering to the human subject an effective amount of human neural stem cells (hNSCs) or progenitors thereof comprising an HFB2050 cell or a progenitor thereof, thereby treating the perinatal hypoxic-ischemic injuries in a human subject. In some embodiments, the method further comprises providing a hypothermia therapy to the human subject.

Aspects disclosed in methods include a method of treating perinatal hypoxic-ischemic injuries (HII) in a human subject comprising: providing a hypothermia therapy to the human subject, and administering to the human subject an effective amount of human neural stem cells (hNSCs) or progenitors thereof, thereby treating the perinatal hypoxic-ischemic injuries in a human subject.

Aspects disclosed in methods include a method of treating perinatal hypoxic-ischemic injuries (HII) in a human subject comprising administering to the human subject an effective amount of human neural stem cells (hNSCs) or progenitors thereof, wherein the human subject has received a hypothermia therapy.

Aspects disclosed in methods include a method of treating perinatal hypoxic-ischemic injuries (HII) in a human subject comprising administering to the human subject an effective amount of human neural stem cells (hNSCs) or progenitors thereof, wherein the human subject has not received a hypothermia therapy.

Aspects disclosed in methods include a method of treating perinatal hypoxic-ischemic injuries (HII) in a human subject comprising identifying the human subject (i) did not receive a hypothermia therapy, (ii) is not qualified for the hypothermia therapy, or (iii) did not respond to the hypothermia therapy, and administering to the human subject an effective amount of human neural stem cells (hNSCs) or progenitors thereof. In some embodiments, the hNSCs or progenitors thereof comprise an HFB2050 cell. In some embodiments, the hNSCs or progenitors thereof are genetically modified. In some embodiments, the hNSCs or progenitors thereof comprises a transgene. In some embodiments, the transgene comprises SOX2 or Nestin. In some embodiments, the hNSCs or progenitors thereof comprise an HSC-derived stable cell line. In some embodiments, the method does not comprise immunosuppression. In some embodiments, the hNSCs or progenitors thereof comprise a characteristic of able to differentiate into three cardinal neural cell types in a stable ratio after prolonged passaging, self-renewal, possess normal growth kinetics, able to be cryopreserved and retain normal characteristics upon thaw and return to culture, or any combination thereof. In some embodiments, the hNSCs or progenitors thereof are able to integrate into periventricular germinal zone in the human subject. In some embodiments, the human subject is a neonate. In some embodiments, the neonate is about 3.5 kg. In some embodiments, the neonate is full-term. In some embodiments, the method further comprises administering the hNSCs or progenitors thereof to the human subject no more than 6 hours or no more than three (3) days after birth or post injury of the human subject. In some embodiments, the method further comprises administering the hNSCs or progenitors thereof to the human subject 6 hours after birth of the human subject. In some embodiments, the method further comprises administering the hNSCs or progenitors thereof to the human subject at about 3 days after birth or post injury of the human subject. In some embodiments, the method further comprises administering the hNSCs or progenitors thereof to the human subject at about 2 days after birth or post injury of the human subject. In some embodiments, the administration of the hNSCs or progenitors thereof and the hypothermia therapy occur concurrently or sequentially. In some embodiments, the administration of the hNSCs or progenitors thereof occurs prior to the hypothermia therapy. In some embodiments, the administration of the hNSCs or progenitors thereof occurs subsequent to the hypothermia therapy. In some embodiments, the hNSCs or progenitors thereof are administered to the human subject upon completion of the hypothermia therapy. In some embodiments, the perinatal HII comprises Sarnat clinical Stage 2 (moderate) perinatal HII. In some embodiments, the perinatal HII comprises Sarnat clinical Stage 3 (severe) perinatal HII. In some embodiments, the human subject has substantially no other medically diagnosed disorders. In some embodiments, the perinatal HII comprises acute or sub-acute HII. In some embodiments, the method further comprises a supportive therapy or an investigational therapy. In some embodiments, the supportive therapy comprises seizure medications, inhaled nitric oxide, high frequency ventilation, extracorporeal membrane oxygenation therapies, volume replacement, hemodialysis, inotropic agents, and vitamins. In some embodiments, the investigational therapy comprises erythropoietin. In some embodiments, the hypothermia therapy comprises a whole-body hypothermia therapy, optionally the whole-body hypothermia therapy begins within or about 6 hours after birth. In some embodiments, the hypothermia therapy is completed in 3 days. In some embodiments, the method further comprises administering a magnetic resonance imaging (MRI) diagnostic test, wherein the results of the MRI inform treatment parameters. In some embodiments, the hNSCs or progenitors thereof are administered to the human subject about 7 days, about 8 days, about 9 days, or about 10 days post injury, or wherein the hNSCs or progenitors thereof are administered to the human subject within 10 days post injury. In some embodiments, the hNSCs or progenitors thereof are administered to the human subject about 3 days, about 4 days, about 5 days, about 6 days, about 7 days, about 8 days, about 9 days, or about 10 days post injury. In some embodiments, the hNSCs or progenitors thereof are administered to the human subject about 3 days post injury. In some embodiments, the hNSCs or progenitors thereof are administered via intracerebral instillation through a fontanelle. In some embodiments, the hNSCs or progenitors thereof are administered via intracerebral instillation by a 30-34-gauge needle (e.g., 30 or 34 gauge needle). In some embodiments, the hNSCs or progenitors thereof are administered via intracerebral instillation through the open non-bony anterior fontanelle (AF) into each of the lateral ventricles (LVs) of the human subject. In some embodiments, the hNSCs or progenitors thereof are administered by (i) inserting a needle or a catheter percutaneously into a cerebral lateral ventricle through an anterior fontanelle (AF) via an AF tap; (ii) aspirating a volume of cerebrospinal fluid (CSF) equivalent to that of the hNSCs to be administered from the catheter; (iii) instilling the hNSCs into the AF; (iv) flushing the catheter with a buffer; (v) removing the catheter; (vi) compressing the area of puncture and applying an aseptic dressing; and (vii) repeating steps (i) to (iv) on ipsilateral side. In some embodiments, the hNSCs or progenitors thereof are administered to the human subject by a 30-34-gauge needle (e.g., 30 or 34 gauge needle) for minimally-invasive, ultrasound guided insertion into the ventricles via a gentle puncture through the skin overlying the open AF. In some embodiments, the hNSCs or progenitors thereof are administered contralateral to a lesion in brain of the human subject. In some embodiments, the hNSCs or progenitors thereof are administered ipsilateral to a lesion in brain of the human subject. In some embodiments, the hNSCs or progenitors thereof are administered without general anesthesia. In some embodiments, the hNSCs or progenitors thereof are administered using a needle or catheter inserted into an anterior fontanelle (AF) to a depth, wherein the depth is about 2.5 cm, 2.4 cm, 2.3 cm, 2.2 cm, 2.1 cm, 2.0 cm, 1.9 cm, 1.8 cm, 1.7 cm, 1.6 cm, 1.5 cm, or a depth within a range defined by any of the preceding values. In some embodiments, the administration of the hNSCs or progenitors thereof is monitored by a cranial ultrasound. In some embodiments, the hNSCs or progenitors thereof are administered in one or more doses. In some embodiments, the hNSCs or progenitors thereof are administered in a single dose. In some embodiments, the administration is completed within 30, 25, 20, 15 minutes, or a period of time within a range defined by any of the proceeding values. In some embodiments, the hNSCs or progenitors thereof are instilled into each LV over about 60 seconds. In some embodiments, the hNSCs or progenitors thereof are administered to the human subject from about 0.5×10⁷ to about 10×10⁷ cells/kg/ventricle. In some embodiments, the hNSCs or progenitors thereof are administered to the human subject about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, or about 7×10⁷ cells/kg/ventricle. In some embodiments, the hNSCs or progenitors thereof are administered to the human subject from about 0.5×10⁷ to about 5×10⁷ cells/kg, from about 1×10⁷ to about 4×10⁷ cells/kg, or from about 1×10⁷ to about 3×10⁷ cells/kg. In some embodiments, the hNSCs or progenitors thereof are administered to the human subject at about 1×10⁷ cells/kg, 2×10⁷ cells/kg, or 3×10⁷ cells/kg. In some embodiments, the hNSCs or progenitors thereof are administered to the human subject at 1×10⁷ cells/kg. In some embodiments, the hNSCs or progenitors thereof are administered to the human subject at 2×10⁷ cells/kg. In some embodiments, the hNSCs or progenitors thereof are administered to the human subject at 3×10⁷ cells/kg. In some embodiments, the hNSCs or progenitors thereof are administered from about 0.1 to about 1 mL suspension per ventricle. In some embodiments, the hNSCs or progenitors thereof are administered from about 0.5 to about 1 mL suspension per ventricle. In some embodiments, the hNSCs or progenitors thereof are administered at about 0.1, about 0.2, about 0.3, about 0.4, about 0.5, about 0.6, about 0.7, about 0.8, about 0.9, or about 1 mL suspension per ventricle. In some embodiments, the hNSCs or progenitors thereof are administered at a concentration of about 2×10⁴ to about 10×10⁴ cells/μL in a buffer. In some embodiments, the hNSCs or progenitors thereof are administered at a concentration of about 4×10⁴ or about 5×10⁴ cells/μL in the buffer. In some embodiments, the hNSCs or progenitors thereof are administered at a concentration of about 2×10⁷ to about 10×10⁷ cells/mL in a buffer. In some embodiments, the hNSCs or progenitors thereof are administered at a concentration of about 4×10⁷ or about 5×10⁷ cells/mL in the buffer. In some embodiments, the buffer is normal saline (NS). In some embodiments, the hNSCs or progenitors thereof comprises hNSCs or progenitors thereof harvested when actively growing in late log-phase. In some embodiments, the hNSCs or progenitors thereof are not infected with a pathogen comprising CMV, EBV, HSV1, HSV2, Parvo B19, Human Herpes 6, 7, 8, HCMV, Hepatitis A, Hepatitis B, Hepatitis C, HIV1, HIV2 or Mycoplasma. In some embodiments, the hNSCs or progenitors thereof are configured to be stable and karyotypically normal subsequent to cryopreserving, thawing, and passaging. In some embodiments, the hNSCs or progenitors thereof are thawed, prepared, and sealed in a sterile container at a manufacturing facility. In some embodiments, the hNSCs or progenitors thereof are thawed from cryopreservation of cells of at most 20 passages. In some embodiments, the cells in cryopreservation was frozen when the cells are in late log-phase growth and/or has a less than 20, 19, or 18 passages. In some embodiments, the hNSCs or progenitors thereof are cultured from about 48 to about 72 hours after thawing. In some embodiments, the hNSCs or progenitors thereof are culture in a period sufficient to allow the hNSCs or progenitors thereof to recover from being thawed and re-enter S-phase, but not long enough to significantly affect cell count. In some embodiments, the hNSCs or progenitors thereof comprise a viability about 90% post-thawing. In some embodiments, the sterile container is a sterile syringe. In some embodiments, the hNSCs or progenitors thereof in the sterile container are transported in a temperature-controlled, continuously gently agitated storage container. In some embodiments, the temperature is controlled at 4° C. In some embodiments, the hNSCs or progenitors thereof in the sterile container is transported to a place of care (POC) for administration to the human subject on the same day. In some embodiments, the sterile container is unpackaged under a sterile condition after transported to the POC and after a single gentle cell trituration. In some embodiments, the method further comprises transporting the hNSCs or progenitors thereof at wet-ice temperature (e.g., about 0° C.), optionally the hNSCs are in a vial or syringe. In some embodiments, the human subject receiving the hNSCs or progenitors thereof and the hypothermia therapy has better prognosis or prevention of cerebral palsy (CP) as compared to a control receiving hypothermia therapy only. In some embodiments, the CP comprises a pathogenic mechanism of inflammation, apoptosis, excitotoxicity, oxidative damage, mitochondrial dysfunction, vascular disruption and rupture of the blood-brain barrier, demyelination, or any combination thereof. In some embodiments, the administration of the hNSCs or progenitors thereof achieves in the human subject matter (i) a decreasing size of the perinatal HII, (ii) rescuing salvageable penumbra, (iii) no increase in necrotic core, (iv) no parenchymal loss, (v) improvement in cognitive or motor tasks, or any combination thereof. In some embodiments, the hNSCs or progenitors thereof produce at least one cytokine selected from the group consisting of GDNF, BDNF, NGF, NT-3, VEGF, IL-6, NO, and PGE2. In some embodiments, the administration of the hNSCs or progenitors thereof and the hypothermia therapy achieve an additive or synergistic effect on treating the perinatal HII in the human subject. In some embodiments, the additive or synergistic effect comprises an additive or synergistic effect on (i) direct neuroprotection and trophic support, (ii) scavenging reactive oxygen species (ROS), (iii) reducing inflammation and scarring, (iv) repairing the blood-brain barrier, (v) mobilizing endogenous NSCs, (vi) promoting endogenous neurite outgrowth, (vii) replacing interneurons, (viii) providing glial support, (ix) providing extracellular matrix, (x) altering the niche, (xi) restoring normal metabolism to injured neural cells, (xii) inducing neural self-repair, or any combination thereof. In some embodiments, the method further comprises administering a magnetic resonance imaging (MRI) diagnostic test, wherein the results of the MRI inform treatment parameters. In some embodiments, the method further comprises analyzing the MRI, wherein analyzing the MRI comprises: configuring at least one processor to perform the functions of: 1) providing the MRI comprising a set of actual image values; 2) rescaling the actual image values to produce corresponding rescaled image values and to produce a rescaled image from the rescaled image values; 3) deriving a histogram of the rescaled image values; 4) using the histogram to derive an adaptive segmentation threshold that can be used to split the rescaled image into two sub-images, a first sub-image with intensities at or below the adaptive segmentation threshold and a second sub-image with intensities above the adaptive segmentation threshold, or a first sub-image with intensities below the adaptive segmentation threshold and a second sub-image with intensities at or above the adaptive segmentation threshold; 5) using the adaptive segmentation threshold to recursively split the rescaled image to generate a Hierarchical Region Splitting Tree of sub(sub) images based on consistency of the rescaled image values of the rescaled image; 6) terminating the recursive splitting of the sub(sub) images using one or more than one predetermined criteria thereby completing the Hierarchical Region Splitting Tree; and 7) identifying one sub(sub) image in the terminated Hierarchical Region Splitting Tree which comprises the region of interest. In some embodiments, the one sub(sub) image in the terminated Hierarchical Region Splitting Tree comprising the region of interest is two-dimensional. In some embodiments, the one sub(sub) image in the terminated Hierarchical Region Splitting Tree comprising the region of interest is three-dimensional. In some embodiments, the method further comprises analyzing the MRI, wherein analyzing the MRI comprises: measuring of water content and of movement (diffusion) of water molecules within brain tissues. In some embodiments, the measuring of water content and of movement (diffusion) of water molecules within brain tissues provides a data set. In some embodiments, the method further comprises calculating average diffusion coefficient (ADC) maps using the data set. In some embodiments, the method further comprises determining an injury state of the human subject. In some embodiments, the injury state informs a treatment protocol. In some embodiments, the hNSCs or progenitors thereof are thawed on the same day that the hypothermia therapy begins and the human subject is a full-term newborn where hNSCs are administered to the full-term newborn immediately and/or soon after the hypothermia therapy is completed. In some embodiments, the hypothermia therapy is completed in three (3) days. In some embodiments, the human subject is diagnosed as having Sarnat clinical Stage 2 (moderate) to Stage 3 (severe) perinatal hypoxic-ischemic cerebral injury.

Aspects disclosed in compositions include a patient-ready pharmaceutical composition for intracerebral administration to a subject, comprises an effective amount of human neural stem cells (hNSCs) or progenitors thereof, wherein the hNSCs or progenitors thereof are in a late log-phase growth condition. In some embodiments, the patient-ready pharmaceutical composition is capable of being formulated at any time point when a patient is ready for administration of the patient-ready pharmaceutical composition. In some embodiments, a growth profile of the hNSCs or progenitors thereof demonstrates a doubling time of about 6 days. In some embodiments, the hNSCs or progenitors thereof are freshly harvested from an actively growing flask of the hNSCs or progenitors thereof. In some embodiments, the patient-ready pharmaceutical composition is aseptic, and wherein the patient-ready pharmaceutical composition is prepared aseptically without a sterilizing procedure. In some embodiments, the sterilizing procedure is filtration, microfiltration, heating, autoclaving, or treating with an anti-microbial agent. In some embodiments, the hNSCs or progenitors thereof have at least 50%, at least 60%, at least 70%, or at least 80% viability. In some embodiments, the hNSCs or progenitors thereof are negative for endotoxin, Mycoplasma pulmonis, human cytomegalovirus (hCMV), Hepatitis A virus, Hepatitis B virus, Hepatitis C virus, human immunodeficiency virus 1 (HIV-1), human immunodeficiency virus 2 (HIV-2), CMV, EBV, HSV1, HSV2, Parvo B19, or Human Herpes 6, 7, 8. In some embodiments, the hNSCs or progenitors thereof are positive of Sox2 or Nestin. In some embodiments, the hNSCs or progenitors thereof are negative for a pluripotency marker. In some embodiments, the pluripotency marker is Oct4, NANOG, TRA1-81, TRA1-60, or SSEA4. In some embodiments, the patient-ready pharmaceutical composition comprises a buffer. In some embodiments, the buffer is normal saline (NS). In some embodiments, the hNSCs or progenitors thereof are suspended in the buffer at a concentration of, from about 2×10⁴ to about 10×10⁴ cells/μL, from about 3×10⁴ to about 9×10⁴ cells/μL, from about 4×10⁴ to about 8×10⁴ cells/μL, or from about 5×10⁴ to about 7×10⁴ cells/μL. In some embodiments, the hNSCs or progenitors thereof are suspended in the buffer at a concentration of about 4×10⁴ cells/μL, about 5×10⁴ cells/μL, or about 6×10⁴ cells/μL. In some embodiments, the hNSCs or progenitors thereof are suspended in the buffer at a concentration of, from about 2×10⁷ to about 10×10⁷ cells/mL, from about 3×10⁷ to about 9×10⁷ cells/mL, from about 4×10⁷ to about 8×10⁷ cells/mL, or from about 5×10⁷ to about 7×10⁷ cells/mL. In some embodiments, the hNSCs or progenitors thereof are suspended in the buffer at a concentration of about 4×10⁷ cells/mL, about 5×10⁷ cells/mL, or about 6×10⁷ cells/mL. In some embodiments, the hNSCs or progenitors thereof are suspended in the buffer at a concentration of about 5×10⁷ cells/mL. In some embodiments, the hNSCs or progenitors thereof are HFB 2050 hNSCs. In some embodiments, the hNSCs or progenitors thereof become contact-inhibited by 2-3 week post-seeding upon reaching confluency. In some embodiments, the hNSCs or progenitors thereof are thawed from cells of cryopreservation of at most 20 passages. In some embodiments, the cells in cryopreservation was frozen when the cells are in late log-phase growth and/or has a less than 20, 19, or 18 passages. In some embodiments, the hNSCs or progenitors thereof are cultured from about 48 to about 72 hours after thawing. In some embodiments, the hNSCs or progenitors thereof are culture in a period sufficient to allow the hNSCs or progenitors thereof to recover from being thawed and re-enter S-phase, but not long enough to significantly affect cell count. In some embodiments, the hNSCs or progenitors thereof comprise a viability about 90% post-thawing. In some embodiments, the hNSCs or progenitors thereof comprise multipotency or self-renewal. In some embodiments, the hNSCs or progenitors thereof are capable of differentiating into neurons, astrocytes, and oligodendrocytes. In some embodiments, the hNSCs or progenitors thereof are capable of forming gap junctions with host cells and producing extracellular matrix.

In some embodiments, the hNSCs or progenitors thereof are capable of producing new hNSCs or progenitors thereof. In some embodiments, the new hNSCs or progenitors thereof have a growth profile with less than 20% variability as compared to the hNSCs or progenitors thereof. In some embodiments, the hNSCs or progenitors thereof are genetically stable. In some embodiments, the hNSCs or progenitors thereof comprise normal female karyotype and maintain stable over at least 2, 3, 4, 5, 6, 7, or 8 passages. In some embodiments, the hNSCs or progenitors thereof are stored at wet-ice temperature (e.g., about 0° C.).

Aspects disclosed in methods include a method for preparing a patient-ready pharmaceutical composition for intracerebral administration to a subject, comprising (i) obtaining a first frozen stock of human neural stem cells (hNSCs) or progenitors thereof; (a) thawing the hNSCs or progenitors thereof; (b) culturing the hNSCs or progenitors thereof; (c) passaging the hNSCs or progenitors thereof; and optionally (ii) obtaining a second frozen stock of the hNSCs or progenitors thereof, and repeating steps (a)-(c) at a time point that is different from the first frozen stock; and (iii) at the time of preparing the patient-ready pharmaceutical composition, collecting the hNSCs or progenitors thereof that are in a late log-phase growth condition, wherein the first frozen stock is maintained at a first temperature. In some embodiments, the method further comprises obtaining a third, a fourth, a fifth, a sixth, a seventh, a ninth, a tenth, an eleventh, or a twelfth frozen stock of the hNSCs or progenitors thereof in step (ii), wherein each of the frozen stocks is cultured at a different time point from each other. In some embodiments, each of the frozen stocks is cultured at a time interval such that at any time point there is a culture of the hNSCs is in a late log-phase growth condition. In some embodiments, a growth profile of the hNSCs or progenitors thereof demonstrates a doubling time of about 6 days. In some embodiments, the hNSCs or progenitors thereof are cultured with an initial seeding number from about 300,000 to about 900,000 cells, from about 400,000 to about 800,000 cells, or from about 500,000 to about 700,000 cells. In some embodiments, the initial seeding number is about 350,000, about 400,000, about 450,000, about 500,000, about 550,000, about 600,000, about 650,000, about 700,000, about 750,000, about 800,000, or about 850,000 cells. In some embodiments, the initial seeding number is about 650,000 cells. In some embodiments, the patient-ready pharmaceutical composition is aseptic, and wherein the patient-ready pharmaceutical composition is prepared aseptically without a sterilizing procedure. In some embodiments, the sterilizing procedure is filtration, microfiltration, heating, autoclaving, or treating with an anti-microbial agent. In some embodiments, the collected hNSCs or progenitors thereof have at least 50%, at least 60%, at least 70%, or at least 80% viability. In some embodiments, the collected hNSCs or progenitors thereof have at least 80% viability. In some embodiments, the collected hNSCs or progenitors thereof are negative for endotoxin, Mycoplasma pulmonis, human cytomegalovirus (hCMV), Hepatitis A virus, Hepatitis B virus, Hepatitis C virus, human immunodeficiency virus 1 (HIV-1), or human immunodeficiency virus 2 (HIV-2). In some embodiments, the collected hNSCs or progenitors thereof are positive of Sox2 or Nestin. In some embodiments, at least 80% of the collected hNSCs or progenitors thereof are Nestin-positive. In some embodiments, at least 30% of the collected hNSCs or progenitors thereof are Ki67-positive. In some embodiments, the collected hNSCs or progenitors thereof are negative for a pluripotency marker. In some embodiments, the pluripotency marker is Oct4, NANOG, TRA1-81, TRA1-60, or SSEA4. In some embodiments, the method comprises suspending the collected hNSCs or progenitors thereof in a buffer at a concentration of, from about 2×10⁴ to about 10×10⁴ cells/μL, from about 3×10⁴ to about 9×10⁴ cells/μL, from about 4×10⁴ to about 8×10⁴ cells/μL, or from about 5×10⁴ to about 7×10⁴ cells/μL. In some embodiments, the method comprises comprising suspending the collected hNSCs or progenitors thereof in the buffer at a concentration of about 4×10⁴ cells/μL, about 5×10⁴ cells/μL, or about 6×10⁴ cells/μL. In some embodiments, the buffer is normal saline (NS). In some embodiments, the hNSCs or progenitors thereof are HFB 2050 hNSCs. In some embodiments, the hNSCs or progenitors thereof become contact-inhibited by 2-3 weeks post-seeding upon reaching confluency. In some embodiments, the hNSCs or progenitors thereof comprise multipotency or self-renewal. In some embodiments, the hNSCs or progenitors thereof are capable of differentiating into neurons, astrocytes, or oligodendrocytes. In some embodiments, the hNSCs or progenitors thereof are capable of differentiating into neurons, astrocytes, and oligodendrocytes. In some embodiments, the hNSCs or progenitors thereof are capable of forming gap junctions with host cells and producing extracellular matrix. In some embodiments, the hNSCs or progenitors thereof are capable of producing new hNSCs or progenitors thereof. In some embodiments, the new hNSCs or progenitors thereof have a growth profile with less than 20% variability as compared to the hNSCs or progenitors thereof. In some embodiments, the hNSCs or progenitors thereof are genetically stable. In some embodiments, the hNSCs or progenitors thereof comprise normal female karyotype and maintain stable over at least 2, 3, 4, 5, 6, 7, or 8 passages. In some embodiments, the method comprises storing the collected hNSCs or progenitors thereof in a 1 mL volume container. In some embodiments, the first frozen stock is maintained at the first temperature in liquid nitrogen. In some embodiments, the first frozen stock is allowed to warm up to a second temperature over a period of time. In some embodiments, the second temperature is wet-ice temperature (e.g., about 0° C.) or room temperature. In some embodiments, the period of time is one day, 2 days, 2.5 days, 3 days, or 3.5 days, or the period of time is within a range defined by any of the preceding values.

Aspects disclosed in compositions include a composition for a patient-ready pharmaceutical composition for intracerebral administration to a prepared by methods disclosed herein.

Aspects disclosed in kits include a kit comprising a patient-ready pharmaceutical composition for intracerebral administration to a subject, comprises an effective amount of human neural stem cells (hNSCs) or progenitors thereof, wherein the hNSCs or progenitors thereof are in a late log-phase growth condition; and tubing for administering the hNSCs. In some embodiments, the tubing for administering the hNSCs or progenitors thereof comprises a hollow needle or a catheter. In some embodiments, the needle or catheter has a size from about 30 to about 34 gauges, from about 20 to about 30 gauges, from about 22 to about 28 gauges, or from about 24 to about 26 gauges. In some embodiments, the needle or catheter has a size of about 23, about 24, about 25, about 26, about 27, about 28, about 29, about 30, about 31, about 32, about 33, or about 34 gauges. In some embodiments, the needle or catheter has a size of 30 or 34 gauge. In some embodiments, the needle or catheter has a size of about 24 gauges. In some embodiments, the kit further comprises a user instruction, wherein the user instruction directs a user to deliver the hNSCs or progenitors thereof to a patient via anterior fontanelle tap by using the tubing. In some embodiments, the delivery is guided by cranial ultrasound. In some embodiments, the hollow needle is a butterfly needle.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a cascade of orchestrated activities leading to the administration of cells comprising HFB2050 on the 4^{th} to 5^{th} day of life (DOL 4-5) of a neonate diagnosed with HII.

FIG. 2 is a process flow diagram summarizes manufacturing and analytics for HFB2050.

FIGS. 3A-3B illustrate normal growth of HFB2050 hNSCs. FIG. 3A shows representative phase microscopic images showing healthy growth and proliferation of HFB2050 hNSCs in monolayers. The images show different confluency levels of HFB2050s, along with an example of the optimal density at which the cells are passaged. FIG. 3B is a growth curve of HFB2050 hNSCs. The doubling time for HFB2050 is about 6 days.

FIGS. 4A-4B illustrate extensive expression of hNSC marker (e.g., Sox2 and Nestin) by HFB2050 cells shortly after plating. FIG. 4A illustrates immunocytochemical staining of the HFB2050 hNSCs, which confirms an expected predominance of hNSC-related marker expression (estimated at ≥90%) Sox2+ and Nestin+ shortly after plating (DIV6). FIG. 4B illustrates flow cytometry confirming expression of the hNSC marker Sox2 in >90% of the cells shortly after passaging and prior to differentiation. Two different Sox2 antibodies were used, yielding the same results.

FIGS. 5A-5B further illustrate dual-expression of Nestin and Sox2 by HFB2050 hNSCs.

FIG. 5A illustrates immunocytochemical dual-staining of hNSC-related markers Sox2 and Nestin in HFB2050 cells shortly after plating. FIG. 5B illustrates the dual expression confirmed by flow cytometry. Developmentally, SOX2 is a slightly more immature marker than Nestin. It is thought that SOX2+ cells give rise to Nestin+ cells. At early stages, however, both markers are used to recognize an hNSC. SOX2 was determined as the most reliable hNSC marker. SOX2+ cells typically divide whereas Nestin+ cells can exit the cell cycle and remain as quiescent (or very slowly cycling) undifferentiated cells both in vitro and in vivo.

FIGS. 6A-6C illustrate confluent, contact-inhibited HFB2050 hNSCs have spontaneously differentiated into astrocyte. FIG. 6A illustrates staining of the astrocyte marker GFAP in hNSCs culture expressing Nestin and Sox2 multiple days after culturing. These figures capture that transition phase of small populations of astrocytes (GFAP+) (upper left) emerging from a well of hNSCs (Sox2+ and Nestin+ cells). FIG. 6B illustrates staining of the astrocyte marker GFAP on DIV35 after the hNCSs become confluent and contact-inhibited. FIG. 6C illustrates staining of the astrocyte marker EAAT1 on DIV35 and compares percentage of EAAT1-expressing cells from DIV6 and DIV35.

FIGS. 7A-7B illustrate HFB2050 hNSCs differentiated into neurons and oligodendrocytes. FIG. 7A illustrates HFB2050 cells expressing β-III-tubulin (Tuj1), a neuronal marker. All cells in the field are shown by the blue DAPI nuclear stain. Scale bar: 10 μm. FIG. 7B depicts confluent, contact-inhibited HFB2050 hNSCs have spontaneously differentiated into oligodendrocytes, the last cell type to emerge after becoming contact-inhibited, indicated by positive staining of Olig2. Oligodendrocytes constitute the smallest subpopulation of differentiated hNSCs—both in vitro and in vivo. Left panels show a low power view of a field of such cells, magnified on the right.

FIGS. 8A-8C illustrate the differentiation profile of HFB2050 hNSCs, which confirms multipotency and self-renewal. FIG. 8A depicts all cells start out as SOX2+ and Nestin+. By DIV 35, the following distribution of differentiated neural cell types are present, with only slight variations as one expects in a biological system. Basically, the hNSCs differentiate into the 3 cardinal neural lineages: neurons (Tuj1+), astrocytes (GFAP+), and oligodendrocytes (Olig2+)—indicating multipotency—as well as into new and/or residual hNSCs (Nestin+)—the basis of self-renewal. FIG. 8B illustrates that multipotency and self-renewal, as indicated by immunomarkers, appears to be essentially unaffected by the initial seeding density of the hNSCs, i.e., high vs. low confluence. FIG. 8C illustrates that early passages and later passages retain the same differentiation profile, including the generation of new hNSCs to insure persistence of the line.

FIGS. 9A-9C illustrate that large populations of HFB2050 hNSCs incorporate BrdU and stain for Ki67 at early timepoints post-plating (i.e., dividing), but many fewer as they become contact-inhibited. As depicted in FIG. 9A, on DIV6, shortly after passaging, most cells (99%) are undifferentiated early hNSCs, i.e., Sox2, and, of those many are proliferative (Ki67+). After 35 days in vitro, as depicted by FIG. 9B, when the cultures become confluent, the % of BrdU+ cells fall from 65% at DIV6 to ˜40%. Equally significant, however, is the distribution of those BrdU+ cells at the 2 different time points: at DIV 6, BrdU+ cells are distributed uniformly throughout the colony (FIG. 9B upper) but, by DIV35 (FIG. 10), they are absent from the leading-edge (which is contact-inhibited and comprised of differentiated cells), but rather in the central core where undifferentiated hNSCs (some slowly cycling) reside. (BrdU was placed on the culture for 36 hrs.; longer exposure can be toxic). As depicted in FIG. 9C, the % of Ki67+ cells (analyzed by flow cytometry) fall from 35% to just 10%. In summary, the analysis above indicates that HFB2050 hNSC proliferation becomes contact-inhibited. After 35 days in vitro, the hNSC culture contains dramatically fewer BrdU+ and/or Ki67+ cells than cultures analyzed 6 days after post-seeding. Furthermore, the cultures now contain the 3 cardinal neural cell types as well as new (or residual) hNSCs, the basis for self-renewal following the next passage.

FIGS. 10A-10B illustrate that proliferation of HFB2050 hNSCs decreases as the cultures become confluent (e.g., 35 days post-passaging) as determined by BrdU incorporation (FIG. 10A) and Ki67-expression (FIG. 10B). In contrast to the distribution of proliferative cells shortly after passaging (present throughout the colony in FIGS. 9B-C), the few residual proliferative cells at confluence are located in the center of the colony where the immature residual hNSCs reside. All hNSCs at the edges which make contact with the flask are contact-inhibited and begin to differentiate.

FIGS. 11A-11D illustrate normal neural stem cell dynamics of both contact inhibition and self-renewal confirmed for the HFB2050 cells. FIGS. 11A-11B demonstrate that Sox2+ hNSCs give rise to Nestin+ hNSCs. Both are abundant and proliferative after passaging (DIV6).

FIGS. 11C-11D illustrate that as the culture becomes contact-inhibited, the number of proliferative (BrdU+/Ki67+) hNSCs falls, as does the number of Sox2+ early hNSCs. But, the subpopulation of quiescent (non- or minimally-dividing/slowly cycling) Nestin+ late hNSCs in the center of the culture (which derived from the Sox2+ hNSCs) remains constant as a reservoir for self-renewal, poised for the next split/passage; the original Nestin+ cells in the passage gave rise, with time in culture, to differentiated progeny, but one does not want the hNSC pool depleted; Sox2 replenished that pool (i.e., kept it constant) and the Sox2+ subpopulation consequently diminished since proliferation had largely been suppressed by contact inhibition. Upon the next passage, however, Sox2+ numbers will rebound with the re-onset of proliferation (as shown by evidence for self-renewal in FIG. 10B wherein the Sox2+ subpopulation re-attains 99% prevalence immediately upon passaging remains the same in that regard from passage-to-passage, whether an early passage or a later passage.

FIG. 12 illustrate that HFB2050 hNSCs are negative for all pluripotency markers (e.g., Oct-4, NANOG, TRA1-81, TRA1-61, and SSEA4).

FIG. 13 demonstrates that HFB2050 hNSCs have a normal human female karyotype. G-banded karyotyping confirms that HFB2050 hNSCs contain a normal human female karyotype, even after a series of passages.

FIGS. 14A-14B illustrate that viability of HFB2050 hNSCs is preserved following cryopreservation and re-thawing. A threshold of ≥70% viability has been deemed adequate for product development. FIG. 14A shows cell counts from 14 independent samples ranging from 1 million to 3 million cells-per-vial were frozen; upon thawing, ≥80% viable cells were recovered and could be further maintained and passaged, achieving the same growth kinetics as in FIG. 3, and the same differentiation profile as baseline (see FIG. 14B). The viability counts from the histograms on the left and presented as a violin plot on the right. FIG. 14B illustrates that the expression of multipotency and self-renewal proteins is not affected by cryopreservation (freeze/thaw). Immunocytochemical profiles were compared between actively growing hNSCs (“fresh”) vs. those that were frozen, thawed, and then maintained and passaged (“frozen”). There are no changes in the markers for multipotency and self-renewal previously established as the HFB2050 hNSC profile.

FIGS. 15A-15D show stability studies of the SOP for sending hNSCs (at patient-ready concentrations) from point-of-production/preparation to point-of-care (POC) without loss of key attributes and release criteria (viability, nestin+, sterility, proliferative capacity). FIG. 15A depicts flow cytometry for nestin using Alexa Fluor 488. FIG. 15B depicts the peaks for nestin with 488-A. FIG. 15C depicts flow cytometry for nestin using fluorescein isothiocyanate (FITC). FIG. 15D shows the acceptable and actual values for viability, nestin+, Ki67+, and sterility following the septic SOP for thawing hNSCs, preparing them for a given patient at the desired concentration in the cell production/preparation facility, and transporting them to POC for delivery by neurosurgeon to recipient newborn in OR or procedure room adjoining NICU and that it does not alter release criteria which ensured a sterile, viable, proliferative undifferentiated donor hNSC population.

FIGS. 16A-16B shows procedures or experimental perinatal hypoxic-ischemic cerebral injury (HII) in Postnatal Day 10 (P10) Wistar rat pups via the Rice-Vannucci Model (RVM).

FIG. 16A depicts the first step of RVM which entails ligation of the left common carotid artery (CCA). FIG. 16B depicts the second step of RVM of exposing the animal to hypoxia (8% FiO₂) for 90 minutes.

FIGS. 17A-17E shows endogenous neural stem cells (NSCs), located in their germinal zone, the subventricular zone (SVZ) which lines the cerebral ventricles, respond to hypoxic-ischemic injury (HII) by migrating to the lesion (to which they appear to be attracted) and“attempting” to restore homeostasis by repopulating it all neural lineages. Implantation of exogenous NSCs into the ventricles in juxtaposition to the SVZ, augments this natural constitutive response of the mammalian brain. FIG. 17A shows results from mice that were subjected to right HI at PND7 via the Rice-Vannucci Model (RVM) as shown in FIG. 16A-B; the area delineated by the red box is magnified. Two hours after HI, the pups were pulsed with BrdU 4 times (every 4 hours for 12 hours). Coronal brain sections from the mice [A-D]were analyzed 1 week after the final BrdU injection with BrdU immunofluorescence [green]. The marked area (*) in the lesion in [C] is magnified in [C′]. HI brain injury induced a significantly increased proliferation of the SVZ progenitor population ipsilateral to the lesion [C, D](represented by increased BrdU immunoreactivity [green]) compared to the homologous grossly intact contralateral side [A, B] (quantification not shown here but supporting that observation). Expansion of BrdU+ cells was most pronounced in the dorsolateral wall of the lateral ventricles adjacent to the infarction cavity. The density of newborn cells entering the injured neocortex increased [C, D, C′]. Indeed, the BrdU+ cells were observed streaming towards the infarct and penumbra (HI) [C′, yellow arrow]. FIG. 17B demonstrates that endogenous NSCs give rise to replacement neurons, oligodendrocytes, astrocytes, and even new progenitors in appropriate normal ratios in this classically “non-neurogenic” region where the penumbra is located, interpreted as an “attempt” by the system to constitutively repopulate the region and restore homeostasis. Robust Nestin-IR (red) was induced within 24 hours of HI in experimental brains in the medial septum, striatum, corpus callosum, external capsule, and cerebral cortex of the ipsilateral hemisphere, particularly surrounding the cortical HI injury site (* in [I.C]). A day after unilateral HI and the final BrdU pulse, many cells around the SVZ and in the injured cortex showed co-localization of both anti-Nestin [A, D] (red) and anti-BrdU [B, E] (green) immunoreactivity (arrows), appreciated in merged images [C, F, respectively]. 1-3 weeks after HI, many of the BrdU+ cells (arrows in [H, K, N, Q, T]) had differentiated into “new” neurons [G-O], oligodendrocytes [P-R] and astrocytes [S-U] in the injured hemisphere, supplementing non-BrdU+(arrowheads in [I, L, O, R, U]) of the respective cell type with which they seamlessly intermixed and were often directly juxtaposed. The “new” neurons, recognized here as neurofilament-immunopositive (red) cells (arrows in [G, J, M] and under the dual-wavelength filter in [I, L, O]), were present in the neocortex and hippocampal CA1 area ipsilateral to the lesion [arrows in M-O—all classically regarded as “non-neurogenic” regions). Note: neurons and oligodendrocytes are the neural cell types most prominently eliminated by HI. The mature oligodendrocyte marker CNPase and the mature astrocyte marker GFAP were employed in [P](and under the dual filter in [R]) and [S] (and under the dual filter in [U]), respectively. This cell type analysis is confirmed under confocal microscopy (serial optical sections co-staining BrdU+ cells for the mature neuronal marker NeuN [A′-N′]) as well as by using an independent NSC marking technique, replication incompetent retroviral vectors encoding lacZ (which incorporate only into dividing cells) [III]. FIG. 17C shows immunostaining of retrovirally-infected endogenous mitotic periventricular NSCs: migration and differentiation of “new” neurons in “non-neurogenic” regions following cerebral injury. Mitotic periventricular NSCs were identified by their incorporation of a provirus encoding lacZ following injection of retroviral vector particles into both lateral ventricles of mice being subjected to unilateral HII. (Such retroviral vectors will incorporate their provirus—in this case lacZ which encodes the reporter protein E. coli β-galactosidase [βgal]—only into dividing cells). Animals here were sacrificed 1 week later. In response to HII, lacZ-expressing βgal+ cells (red) migrated widely, including into the striatum [A], the cortex ipsilateral to the lesion [D], and the cortical penumbra of the infarct [J]—all classically “non-neurogenic” regions in the intact “post-fetal” mammalian brain. Some of these lacZ+ cells [A, D, J] (red; arrows) expressed the mature neuronal marker, NeuN [B, E, K, respectively] (green via an FITC secondary antibody; arrows) with co-localization of both antibodies visualized under a dual filter [C, F, L, respectively] (arrows)—suggesting de novo neurogenesis. Arrowheads in [B, C] indicate neurons that were not the progeny of retrovirus-infected NSCs. In the grossly “intact” contralateral hemisphere (likely the residence of some transcallosally projecting neurons that lost synaptic targets on the infarcted side), there was also evidence for the migration and differentiation into NeuN+ neurons of some lacZ+ cells in the neocortex [G-I] (arrows) and hippocampal CA1 region [M-O] (arrows)—also all non-neurogenic under normal circumstances—in response to severe HII. These phenomena are further illustrated by a low magnification view of the cerebral cortex and hippocampus in which a significant number of lacZ+ periventricular NPCs that migrated to these regions [P] (red) differentiated into NeuN+ neurons [Q] (yellow/orange under a dual filter when NeuN is tagged with an FITC secondary antibody). Insets in [P] and [Q] show the representative lacZ+ cell indicated by the arrow in [P] and [Q], respectively, further enlarged to illustrate the typical morphology of a newly-born neuron derived from a labeled periventricular cell that had migrated to the neocortex following injury. [R-W] That such “new” lacZ+ NeuN+ neurons in, for example, damaged right cortex were active, integrated and responsive was suggested by their being decorated by synapsin [Syn] (indicative of synaptic input) [R-T] and displaying immunocytochemically detectable upregulation and expression of c-fos in the living animal (a surrogate for electrophysiological activity) [U-W]. The representative lacZ+ cells in those regions (red cells with arrows in [R]) are immunoreactive to an anti-c-fos nuclear antibody (same green immunopositive cells with arrows in [V]), visualized under a dual filter (cells with arrows in [W]). This constitutive process of “attempted self-repair” by endogenous NSCs in the SVZ involves multiple genes and factors. FIG. 17D illustrates an example of one of those: the spontaneous change in a fundamental developmental program (NSC migration) induced by HII in an “attempt” to compensate for cerebral HII. The Slit family of secreted guidance proteins are located in the choroid plexus, septum, neocortex, and SVZ of the developing mammalian brain and act as chemorepulsive factors for neuronal migration, “pushing” neurons from the ganglionic eminence to the cortex to become inhibitory interneurons and neurons from SVZ into the rostral migratory stream and towards the olfactory bulbs to become olfactory interneurons. Slit-2 expression (green) was determined by immunocytochemistry 2 days post-HII. Its distribution is normal on the intact side (*): surrounding the ventricles within the SVZ (arrowhead). However, on the injured side, Slit has re-distributed itself such that it will now be “pushing” SVZ neurons not towards the olfactory bulbs but rather dorsally towards the infarct. In addition to repulsive molecules that push endogenous NSCs to the site of HII, there are also, elaborated within the infarcted regions, chemoattractants secreted by the reactive astrocytes and vascular endothelial cells (e.g., SDF1) and from the microglia. Scale Bar: 100 μm. FIG. 17E provide further evidence that SVZ attempts to “provide” new neurons to the HI-injured neocortex: Doublecortin (DCX) (green)—a microtubule-associated protein associated with migratory neural progenitor cells or neuroblasts destined to become young migratory neurons—is much more extensive on the HI-injured size [B] (green arrow) compared to its restricted distribution to lateral SVZ in the intact SVZ [A] from which new neurons destined for the olfactory bulb will emerge.

FIGS. 18A-18B shows the transplantation of human neural stem cells (hNSCs) in a manner and timing that enables them to integrate into the subventricular zone (SVZ) following a minimally-invasive intracerebral ventricular injection. FIG. 18A shows migration to and altered differentiation of transplanted murine NSCs within the ischemic area of a mouse brain subjected to unilateral HI injury via the Rice-Vanucci Model (RVM). [Inset, coronal H and E, arrowhead]. [A-I] Robust migration by lacZ-expressing NSCs from their injection site in the left contralateral ventricle (as per Paradigm I.) to and throughout regions of ischemic damage (arrows) experimentally imposed 3 days prior at P7. [A-H] are semi-serial sections throughout the cerebrum, rostral to caudal. Xgal+ donor-derived cells are seen migrating through the corpus callosum and commissures (arrowhead) and into and throughout the ischemic right hemisphere, drawn by tropic signals. A representative Xgal+ cell in the corpus callosum is magnified in [I], revealing a classic migratory leading process (arrowhead). When NSCs are injected directly into the ischemic region as per Paradigm II, they never migrate towards the intact side but rather integrate throughout the infarcted cerebrum [J-P]; a representative stably engrafted region is magnified in [Q]2 mos. post-transplant. Ultrastructural [R] and immunocytochemical [S-J′] analysis shows the NSCs to yield neurons and oligodendrocytes, the 2 cells types most severely damaged by HI. [R] The Xgal precipitate forms electron dense precipitates “p” localized typically to the nuclear membrane, cytoplasmic organelles). 3 donor-derived cells are seen. The 2 small cells are Xgal-labeled oligodendroglia (LO). These are situated next to a large labeled pyramidal neuron (LPN) (outlined by small arrowheads). At the top of the apical dendrite (blocked area with * shown at higher power in inset), one can see the donor-derive PN receiving synaptic input from the host. (p indicating precipitate confirms that the post-synaptic region is donor-derived). [S-J′]. Donor-derived, lacZ-expressing cells are identified by anti-β-gal immunofluorescence (Texas red, top row) and neural cell type-specific markers are visualized by FITC (middle row). Double labeling, seen as a yellow/orange fluorescence, is observed by dual bypass filter microscopy (bottom row). Donor derived cells within the infarct express the mature neuronal markers NeuN and neurofilament (NF) (donor-derived neurons indicated by arrowheads, non-neuronal engrafted cell indicated by arrow); the oligodendroglial marker CNPase (donor-derived oligodendrocytes indicated by arrowheads); and the mature astrocytic intermediate filament GFAP. [H′-J′] Even donor-derived cells as much as 1 mm from the infarct on the ipsilateral side can be influenced to differentiate into neurons (lacZ/MAP-2+ cells (arrowheads).

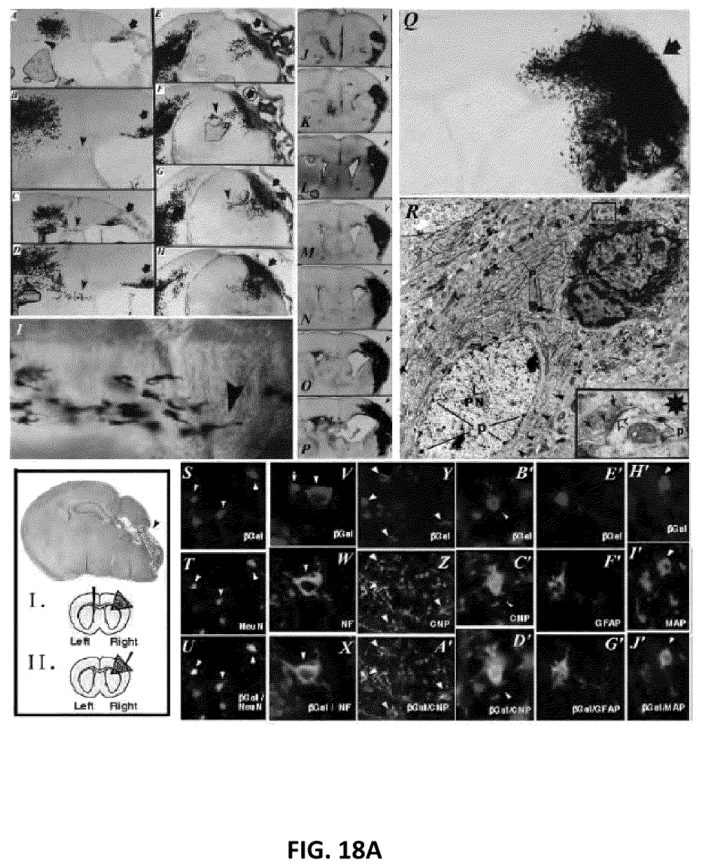

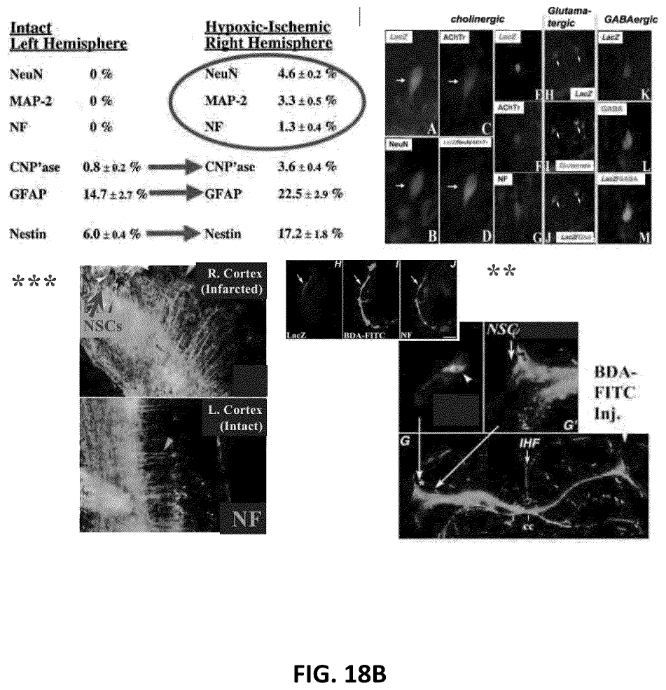

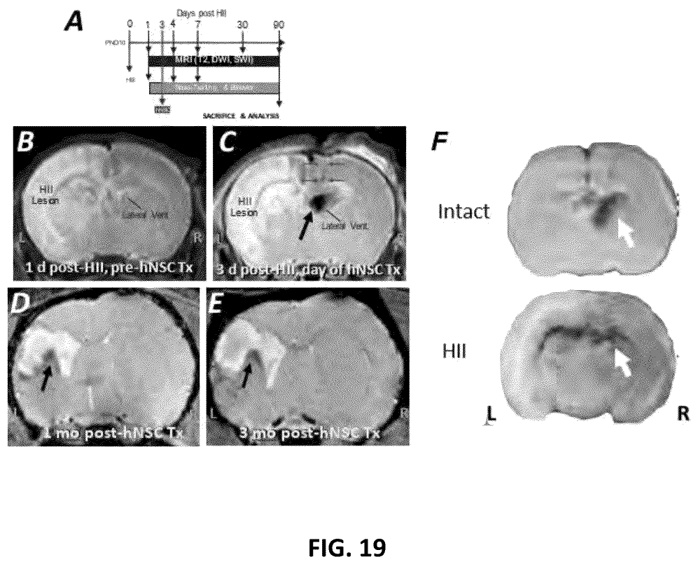

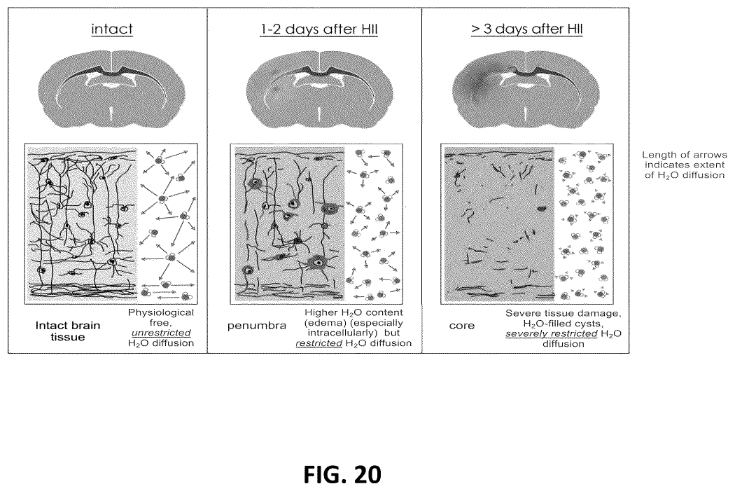

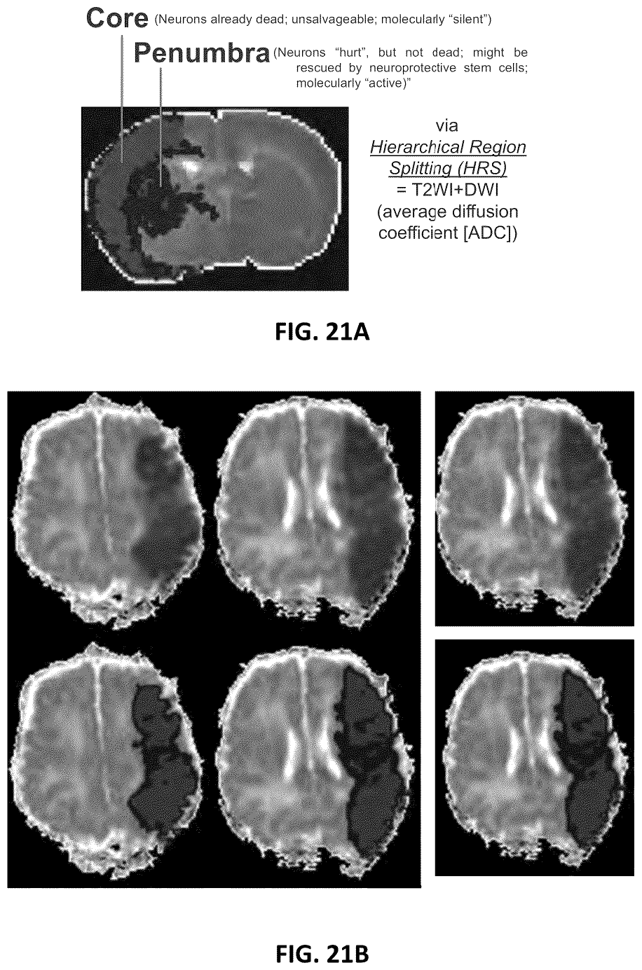

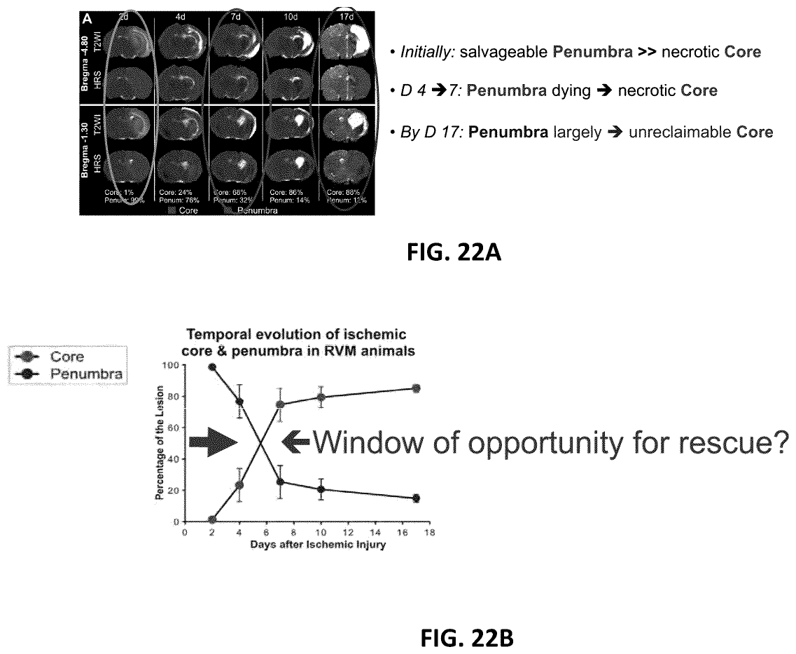

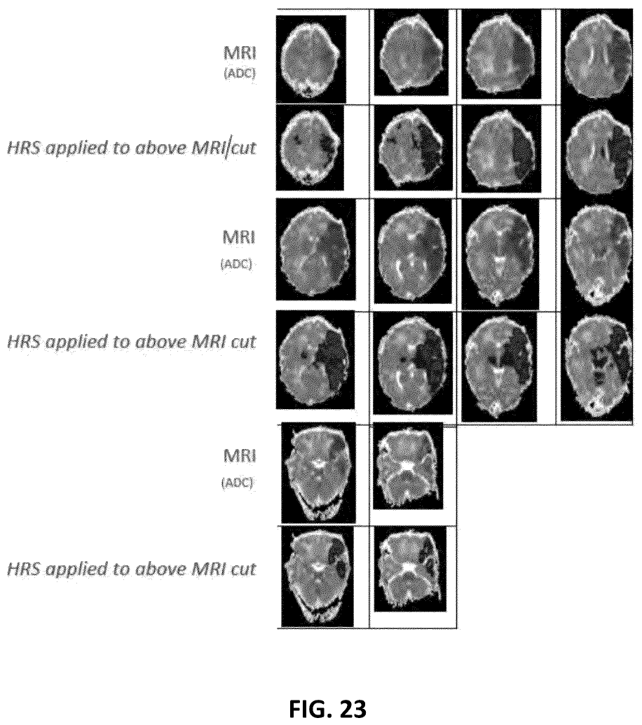

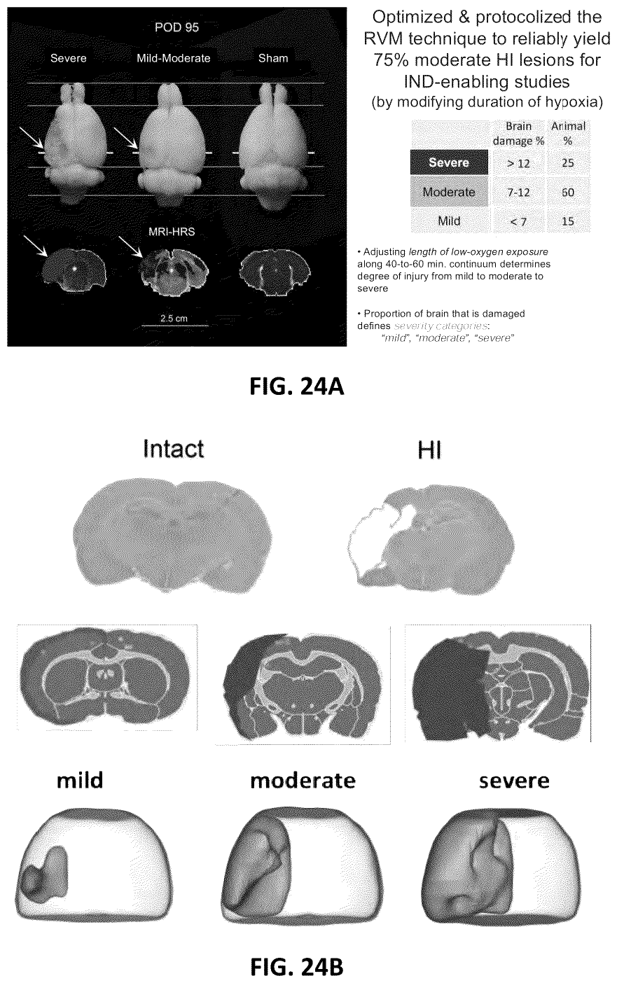

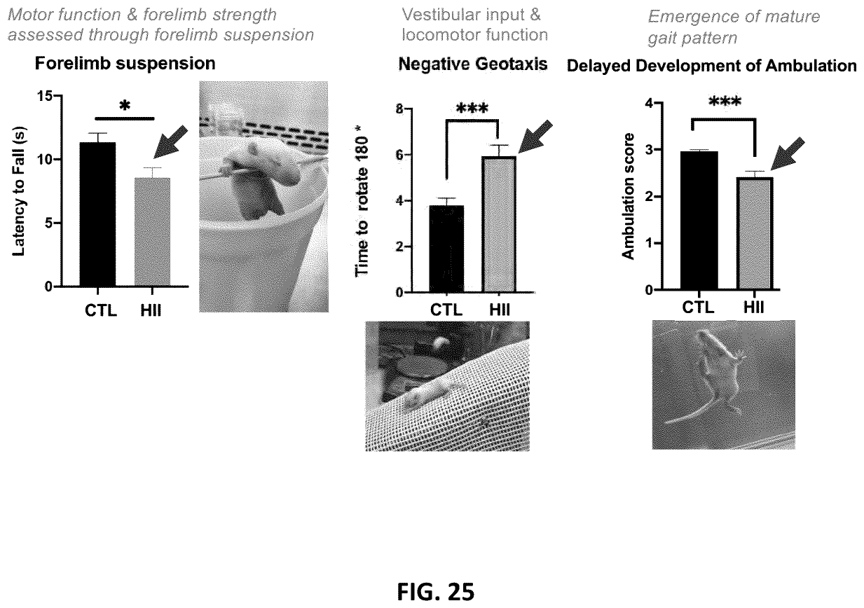

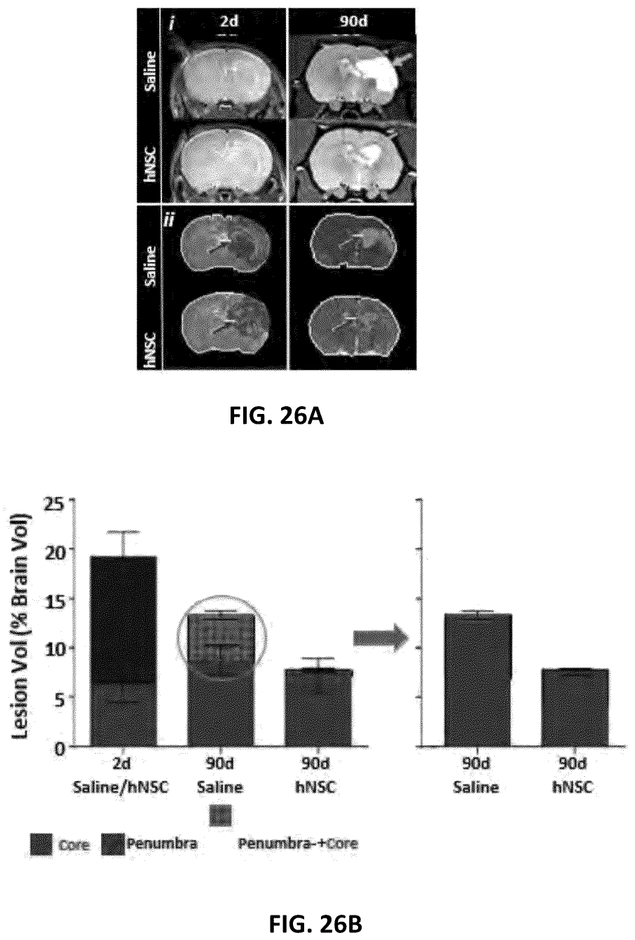

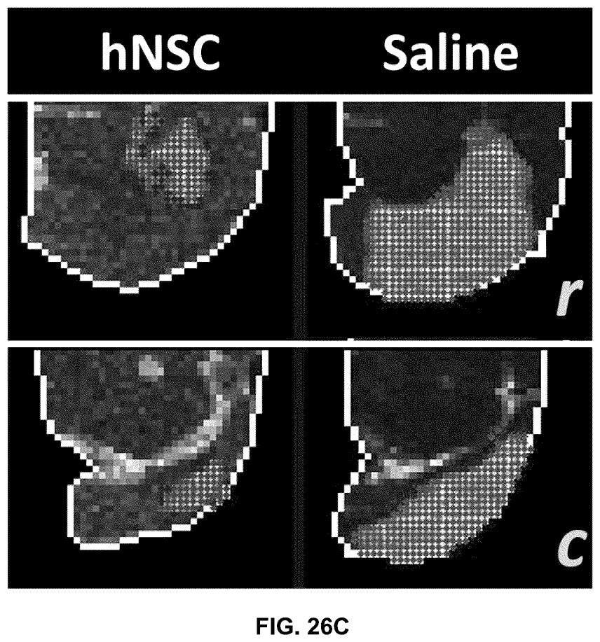

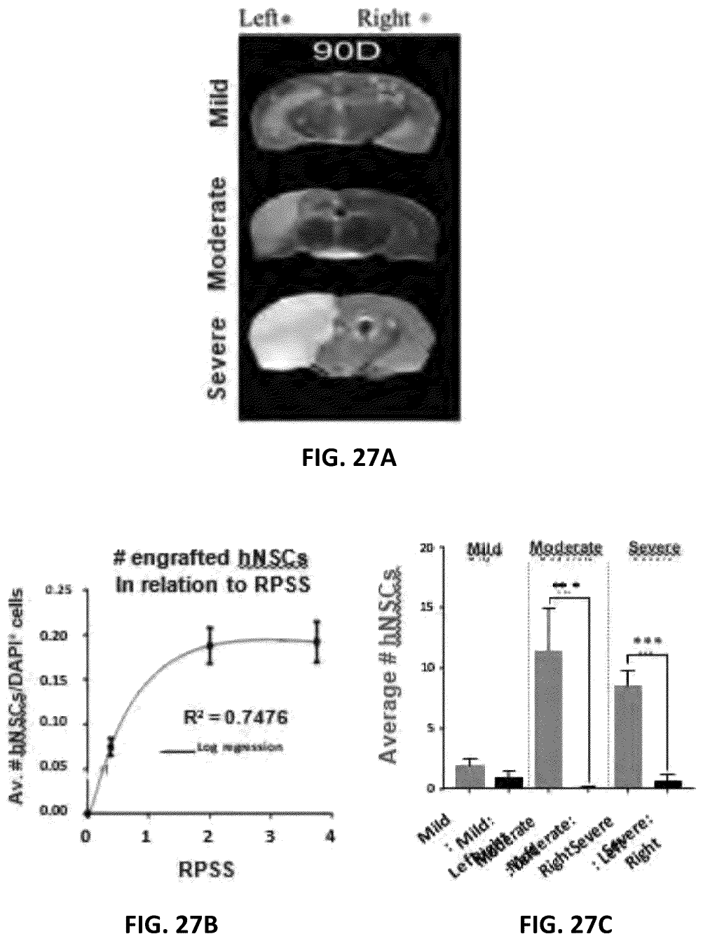

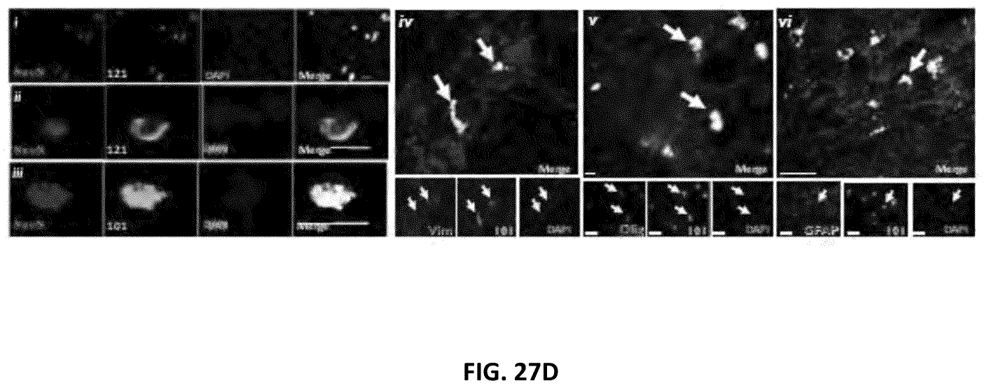

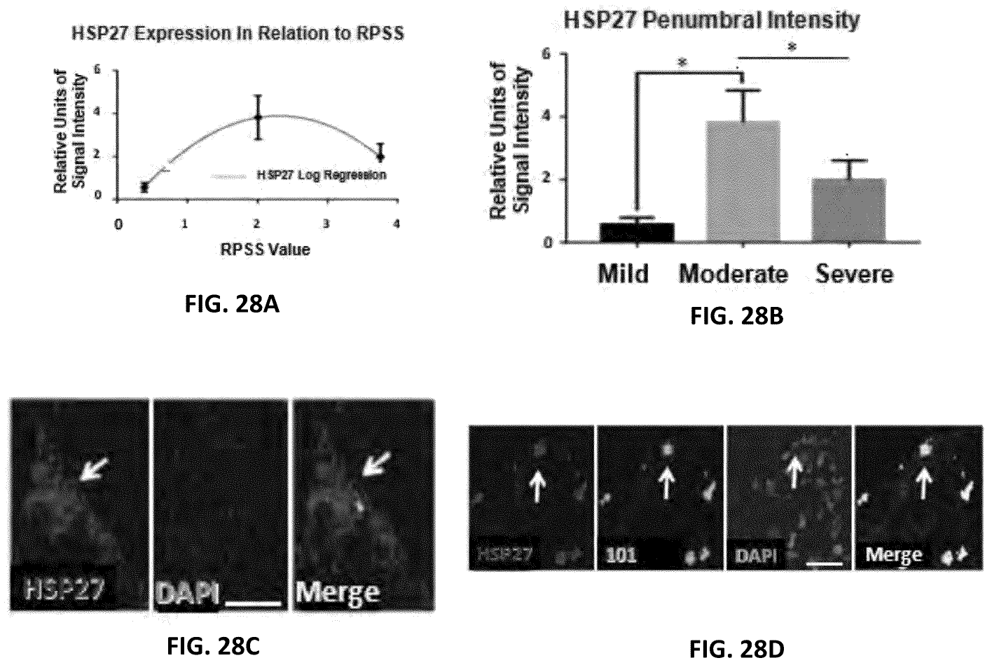

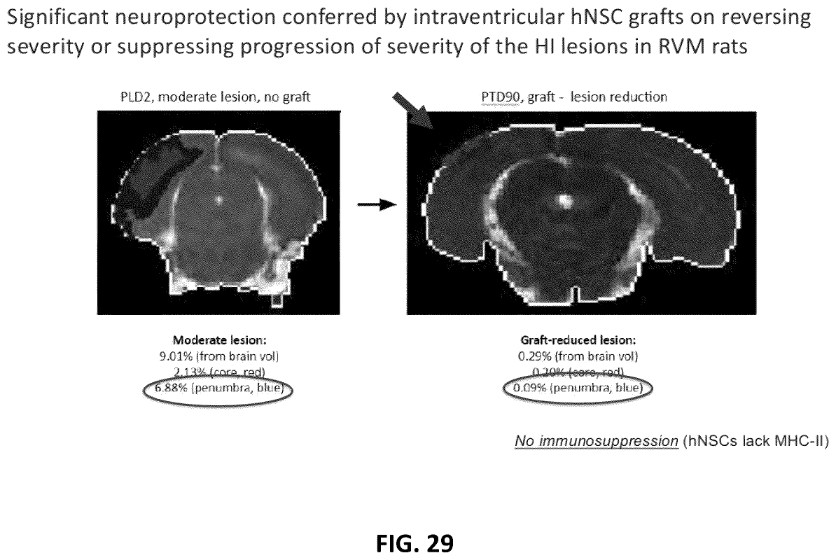

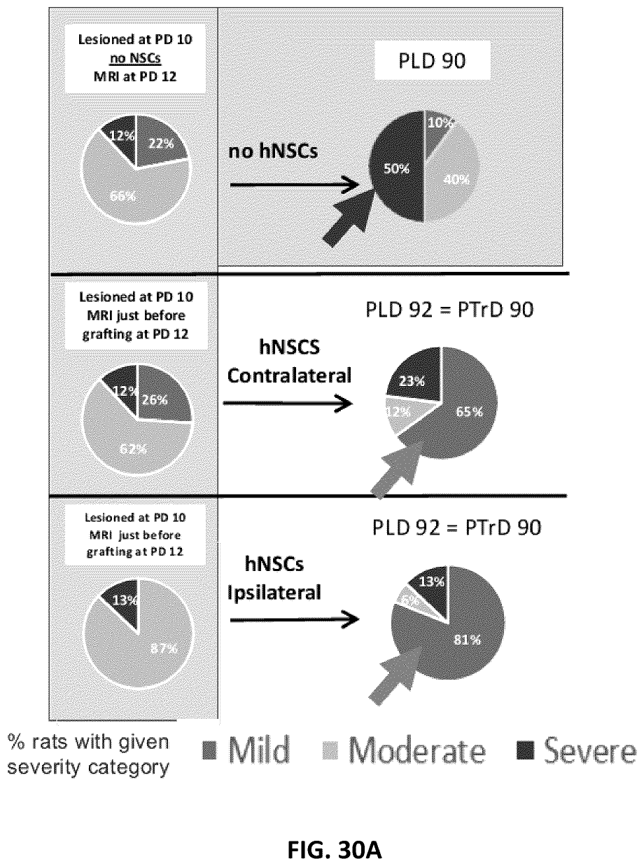

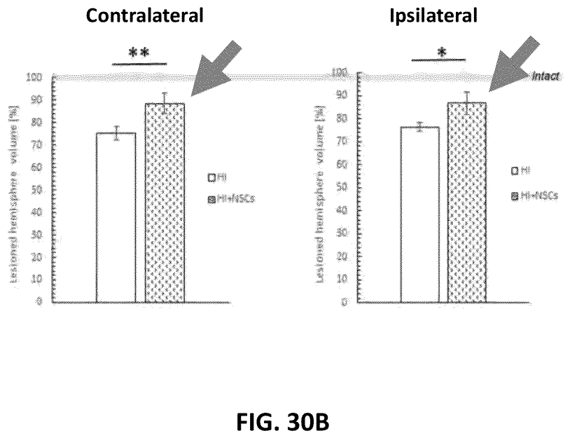

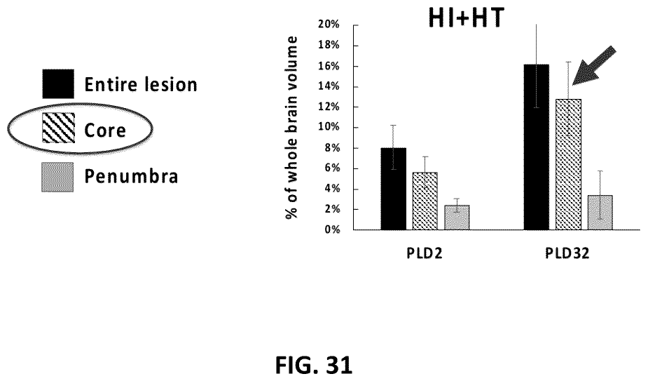

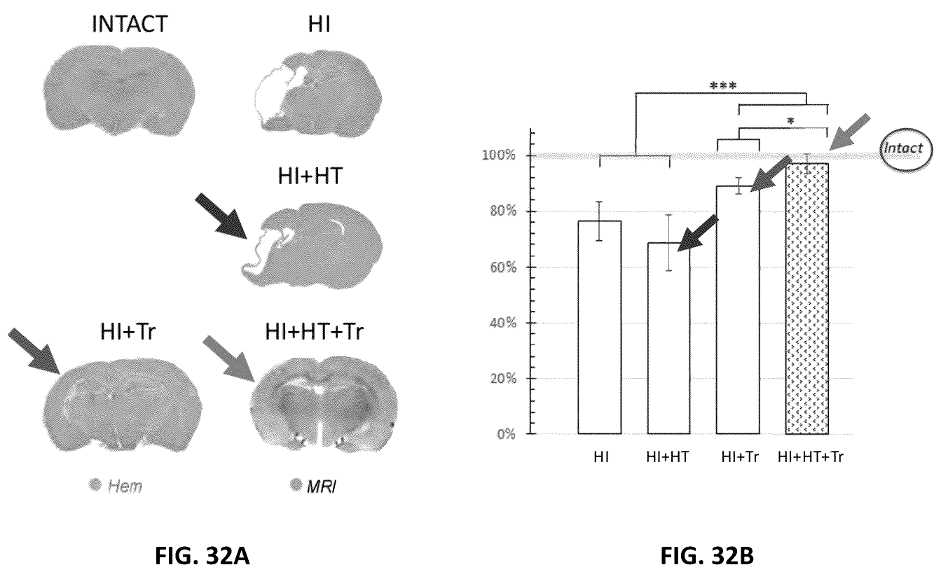

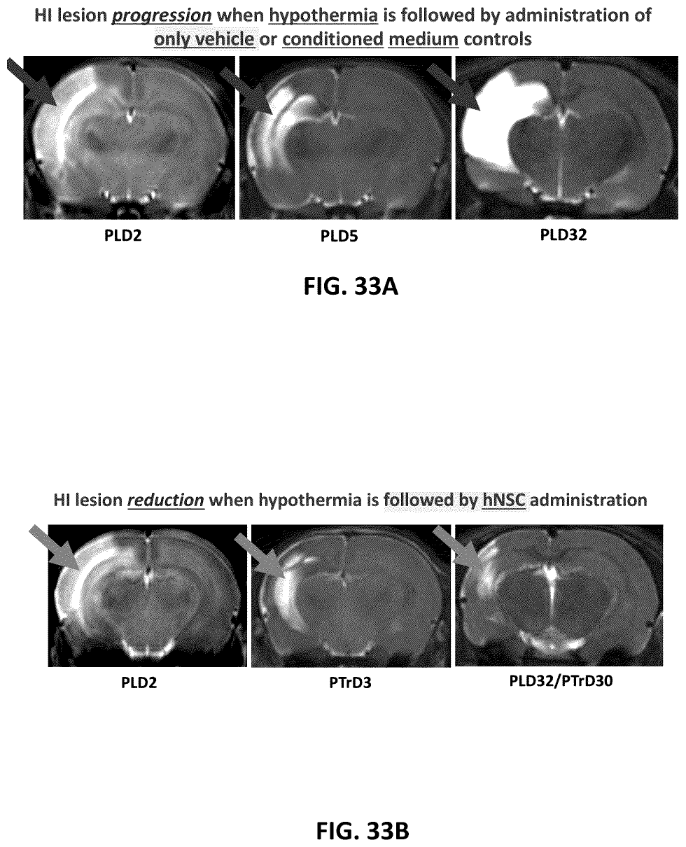

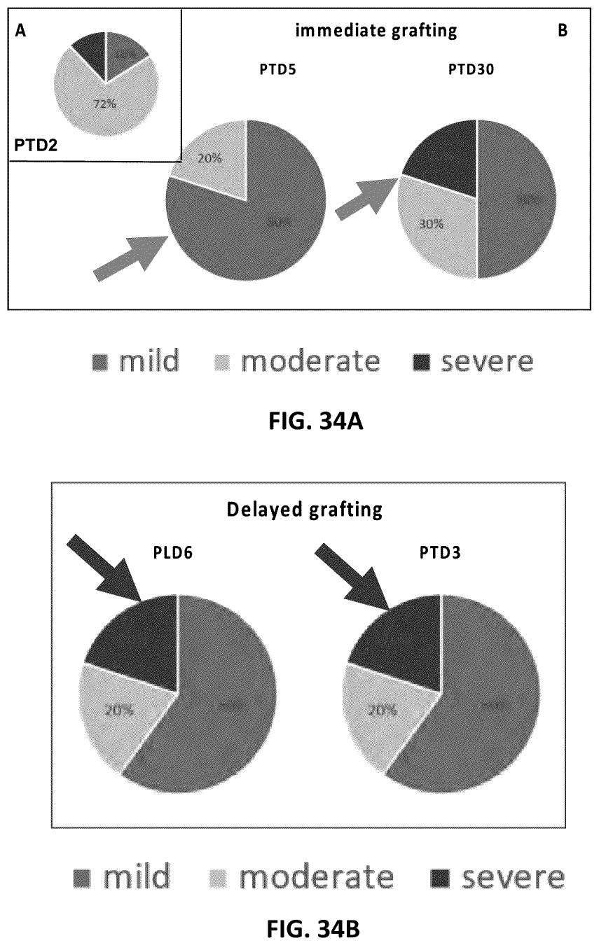

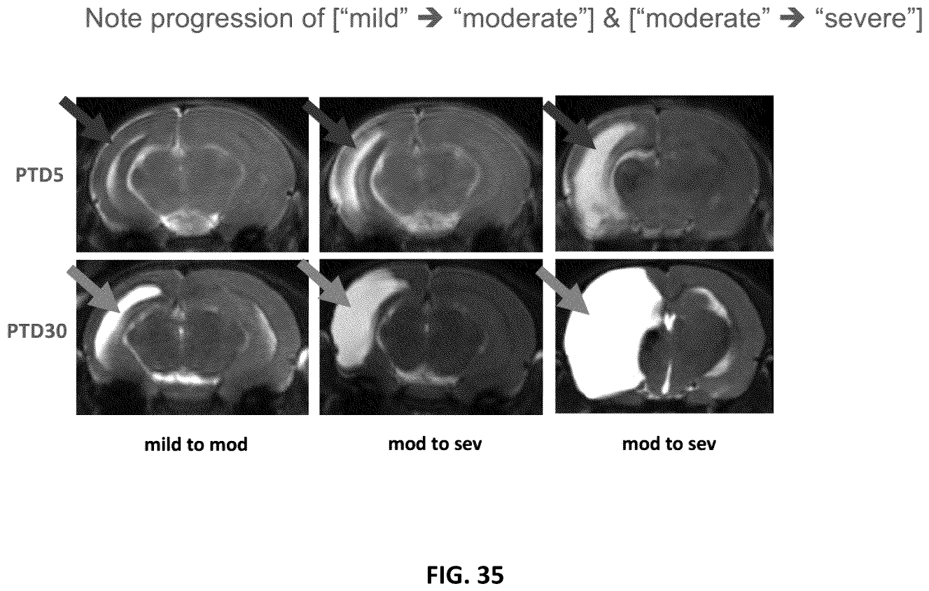

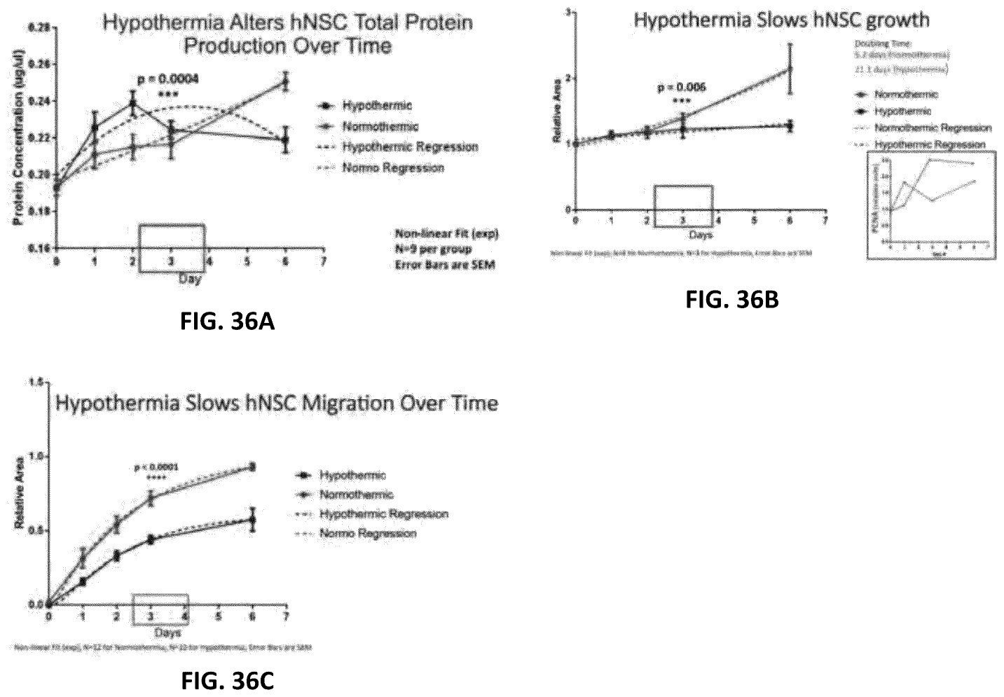

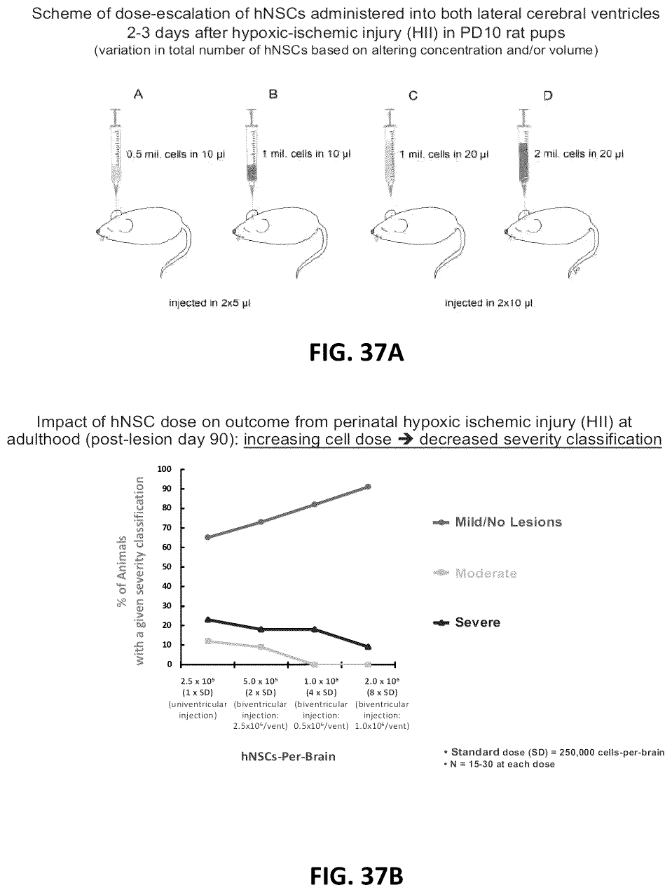

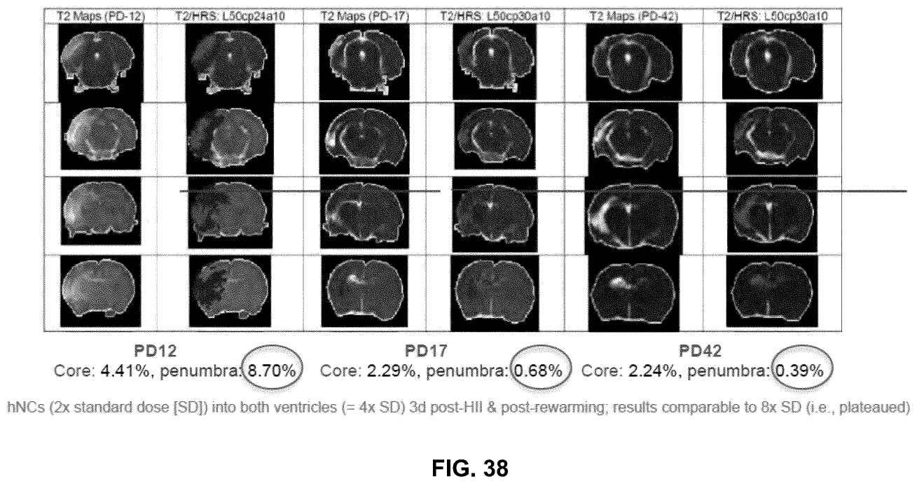

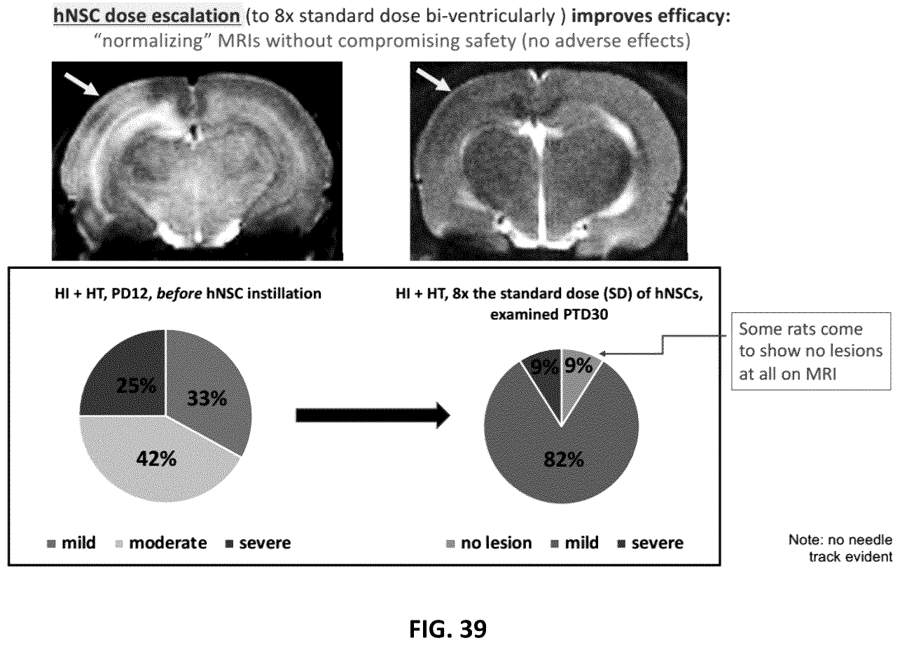

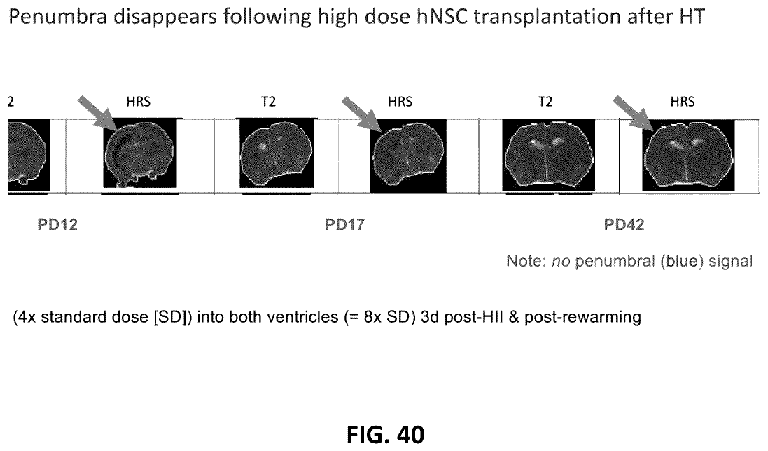

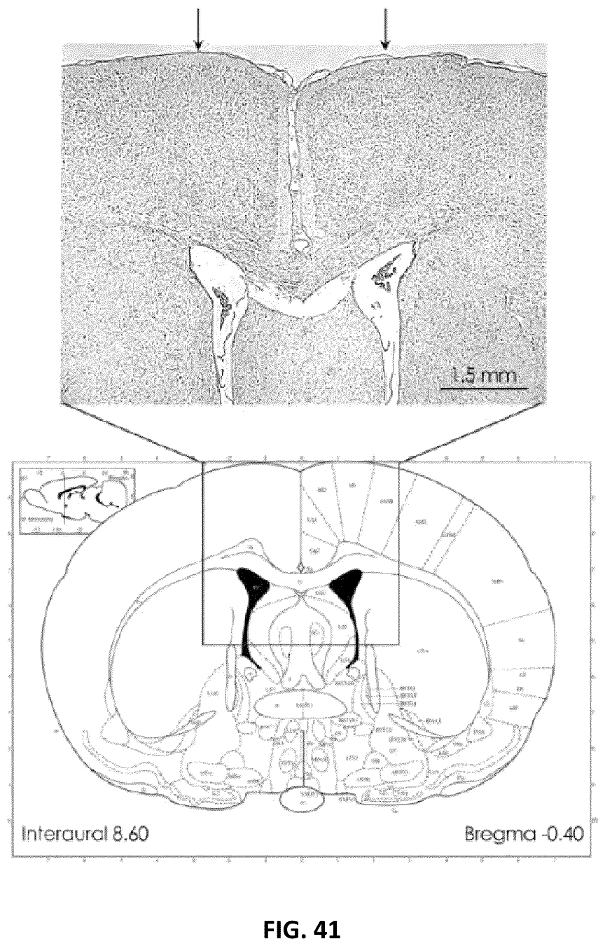

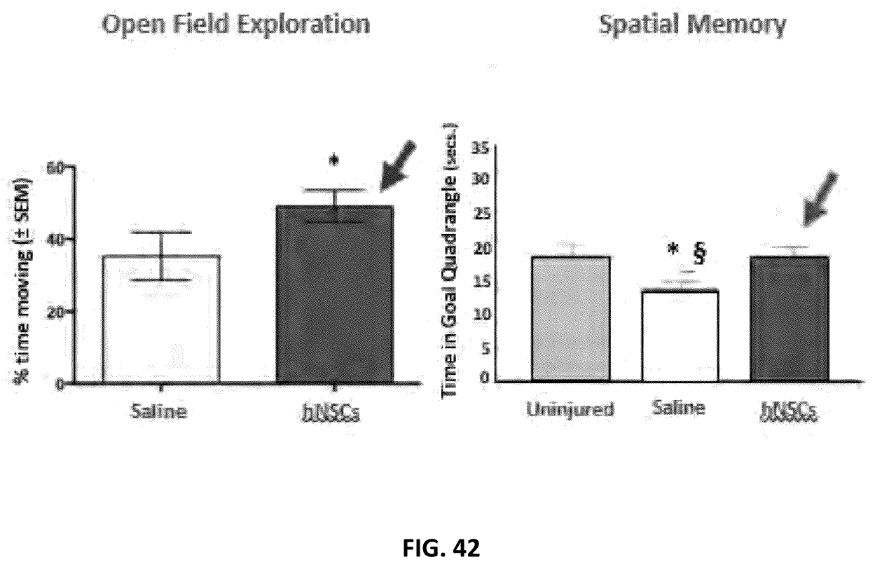

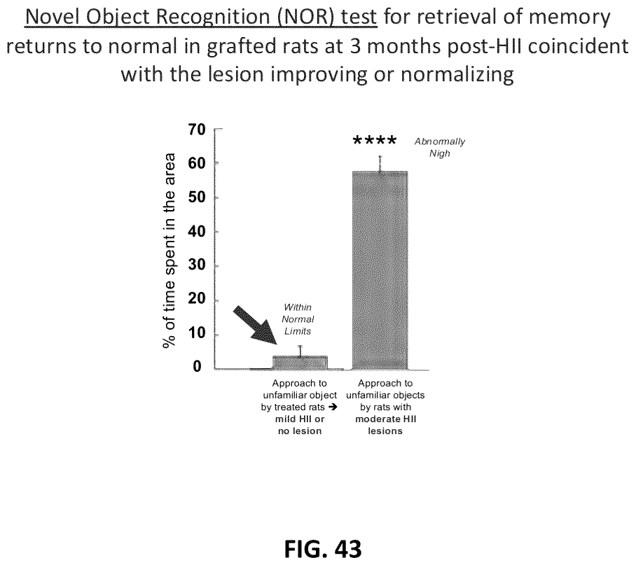

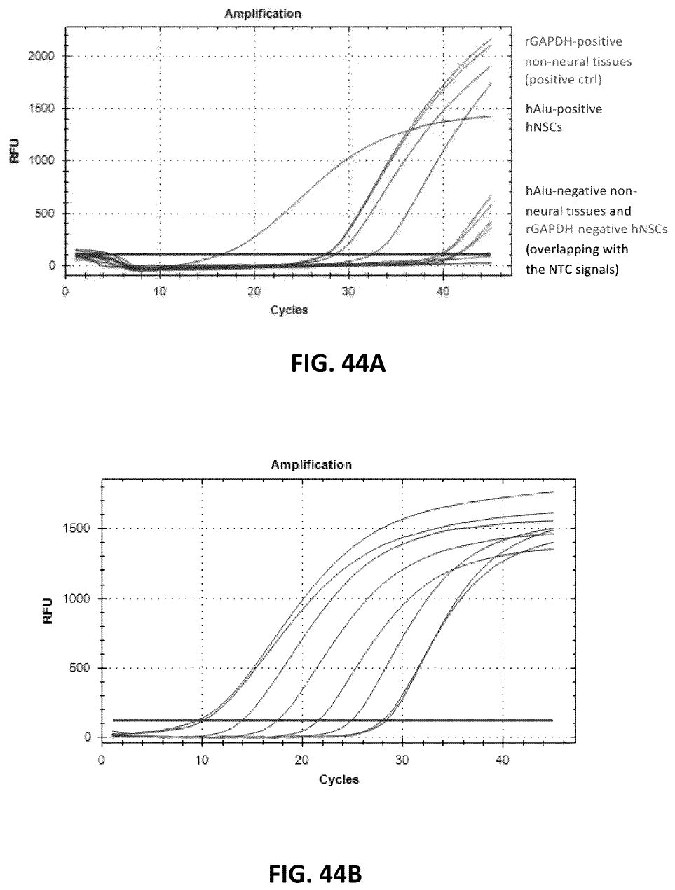

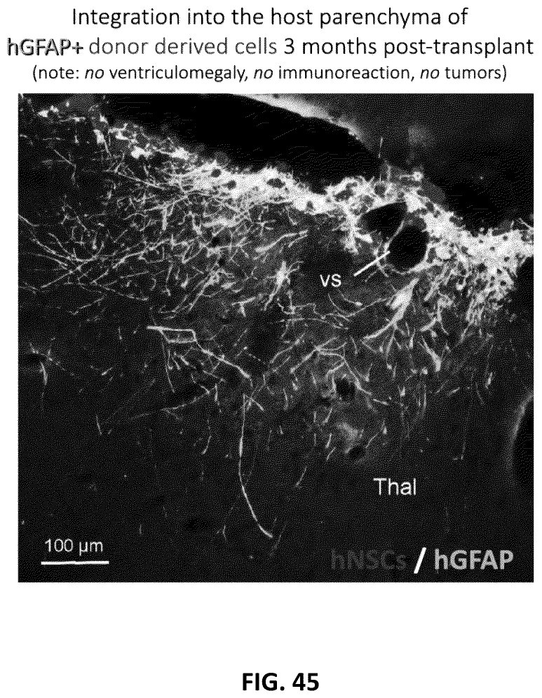

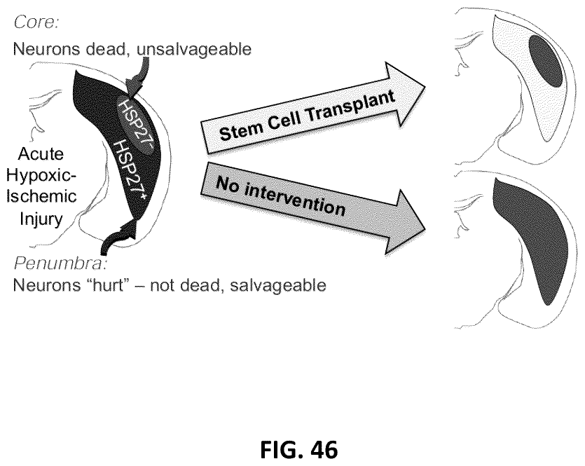

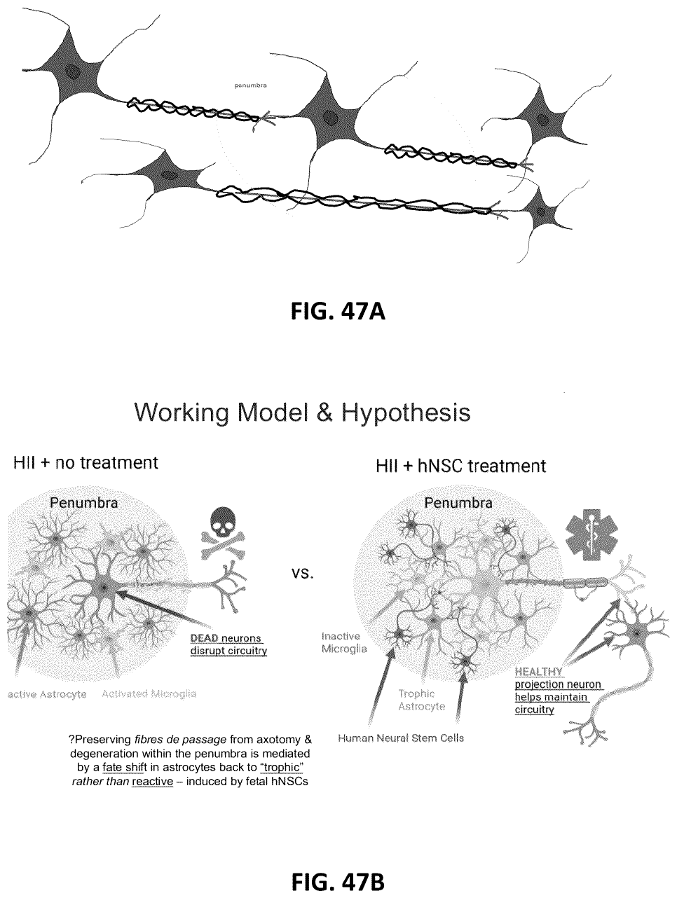

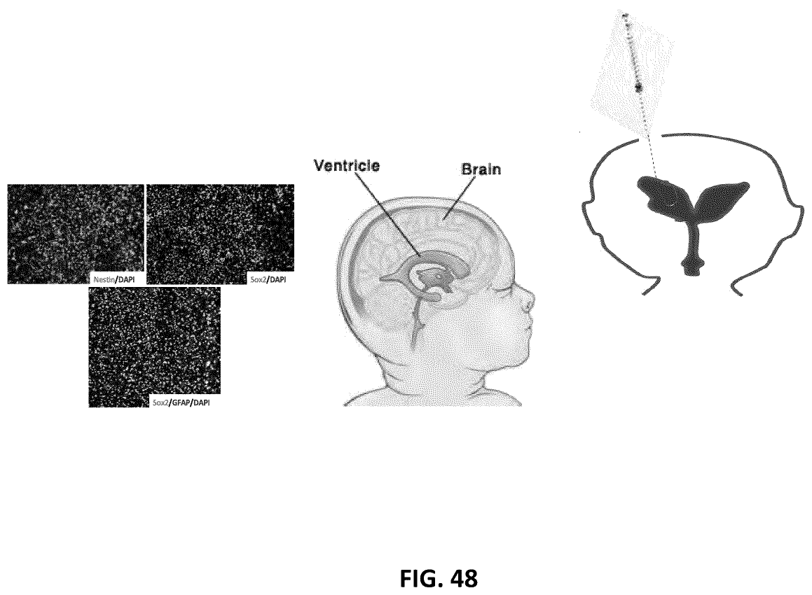

The table in FIG. 18B indicates that the same NSC clone yields no neurons in an intact cortex and a <1% oligodendrocytes, the normal developmental profile of the post-fetal mammalian cortex. However, in the “post-developmental” infarcted cortex, new neurogenesis occurs. Therefore, HI must create an altered milieu to which the NSCs respond by shifting their differentiation fate toward one of cell type compensation. These data have been replicated using human NSCs. The table also shows that “new neurons” are not the only cell type that the multipotent NSCs yield in the injured cortex as suggested by FIG. 18A where the immunostaining in Panels Y-D′ and the EMs in Panel Q, they quadruple the number of oligodendrocytes, double the number of astrocytes [GFAP+ cells, E′-G′], and triple the number of undifferentiated neural progenitors [Nestin+ cells], suggesting that the NSC is “attempting” to reconstitute the entire milieu of the damaged cortex, including the often non-neuronal “chaperone” cells that play an important role in functional recovery. The series of panels in the top right of FIG. 18B indicate that donor-derived neurons have the ability to differentiate into cells with the expected range of cortical neurotransmitter types—cholinergic [A-D] and [E-G], glutamatergic [H-J], and GABAergic [K-M]. The series of panels in the bottom right of FIG. 18B show that NSC-derived neurons are capable of sending long-distance transcollosal connections from the infarcted cortex to appropriate targets regions in the intact contralateral hemisphere. To confirm that long-distance processes projected from the injured cortex into host parenchyma, a series of antegrade and retrograde tract tracing studies were performed on the donor-derived βgal+ neurons, one of which is illustrated in [G-G″ ]. To confirm that such long axons were derived from the injured, transplanted regions, BDA-FITC was implanted into the opposite (left) intact hemisphere within the target region of such transcallosal axons (at 8 weeks following implantation into the infarction cavity) (n=10), in order to monitor retrograde transport of the tracer back to their source in the right hemisphere. Axonal projections (labeled green with fluorescein under an FITC filter) are visualized (via the retrograde transport of BDA) leading back to (across the interhemispheric fissure [“IHF” ] via the corpus callosum [“cc” ]) and emanating from cells within the damaged engrafted cortex and penumbra (seen at progressively higher magnification in [G′] (region indicated by arrow to [G]) and [G″ ] (region indicated by arrow and asterisk in [G]). In [G″ ], the retrogradely BDA-FITC-labeled perikaryon of a representative neuron is well-visualized. That such cells are neurons of donor-derivation is supported by their triple-labeling [H-J] for lacZ (βgal) [H], BDA-FITC [I], and the neuronal marker NF [J]; arrow in [H-J], indicates same cell in all 3 panels). Scale bars: [G], 500 μm; [G″ ], 20 μm; [H-J], 30 μm. The bottom left of FIG. 18B shows that not only are donor NSC-derived neural cells present in the infarct, but also fibers and projections from host-derived cells enter the NSC-infiltrated ischemic milieu, representing another mechanism by which the penumbra of an infarct likely remains alive and active following NSC engraftment. (Note: This particular set of experiments was performed by supporting the NSCs with biodegradable, biosynthetic polyglycolic acid [PGA] scaffold because the lesion—generated by the RVM—was so large. However, the same phenomenon occurs with NSCs alone if they are supported by the penumbra.) The host fibers, induced by the donor NSCs, participate in parenchymal preservation becomes startling when one compares the Upper Panel (“R. Cortex Infarcted”) with the Lower Panel (“L. Cortex Intact”). While in the intact contralateral left cortex, host NF+ processes (green, indicated by blue arrowhead) are largely non-branching and perpendicular, running straight from subcortical regions to the pial surface, those in the injured right cortex (blue arrowhead) (particularly in cortex adjacent to the transplantation site) are elongated with extensive delicate arborizations, no longer strictly perpendicular to the pial surface but rather arcing towards the infarcted region, entering the infarct/NSC complex (arrowheads) within the infarct cavity (arrow) (n=15). These host fibers, intermingling with donor-derived neurons process combine to create an intricate network of multiple long, branching NF+ (green) processes within the infarct and its adjoining parenchyma. In other words, NF+ processes were of both host- and donor-derivation. Scale bars: 100 μm.

FIG. 19 shows a preclinical protocol and representative MRI of rodent brains at various key points. The top panel shows the experimental timeline. The HII lesion is further subdivided into necrotic core vs. penumbra when these MRI images are subjected to the HRS algorithm. [B] depicts a representative T2WI (using an 11.7T MRI) of the brain of a postnatal day 11 pup, 1 day after inducing left HII and prior to hNSC transplantation and is beginning to show an increasingly intense “water signal” (white) on the left [“HII lesion” ]. [C] depicts a representative T2WI (using an 11.7T MRI) 3 days post-HII, shortly after implantation of SPIO pre-labeled hNSCs into the contralateral cerebral ventricle [“Lateral Vent” ]. The “HII lesion” on the left is becoming hyperintense (white) and the black signal void of the SPIO-labeled hNSCs in the lateral ventricle (black arrow). Red arrows denote the needle track. In contrast to what occurs in the intact brain, in a brain subjected to left HII, the implanted SPIO-labeled hNSCs (black signal void) (black arrow), migrate from the right (“R”) to the left (“L”) hemisphere to enter the lesion. Shown here (using a 4.7T MRI) are SPIO-labeled hNSCs (black signal void) (black arrow) at 1-month post-implantation into the contralateral ventricle [D] and, in the same representative animal, at 3 months post-implantation [E]—stably integrated and surrounding a much-reduced residual lesion, with no interval enlargement of the graft or ventricles. [F] illustrates the early and relatively rapid migration of SPIO-labeled NSCs to the contralateral hemisphere—if there is an HII lesion—can be visualized under MRI. In the intact brain (F, top image), SPIO-labeled NSCs (black, signal void), 1 day after implantation into the right cerebral ventricle, began to migrate into the ipsilateral periventricular parenchyma but remained largely restricted to the transplanted hemisphere [white arrow]. However, in a brain subjected to left HII [F, bottom image], the implanted NSCs began to migrate from the right to the left hemisphere to enter the lesion (“pathotropic migration”) (white arrow). Such SPIO-labeled cells, which migrate at ˜100 μm/day for ˜3-5 days post-implantation and then plateau when the NSCs enter the penumbra of the lesion, can be detected for at least 58 weeks.

FIG. 20 depicts the biophysical basis of the ability of the hierarchical region splitting (HRS) algorithm applied (post-hoc) to MRI of the brain to segment an ischemic lesion into salvageable penumbra versus unsalvageable necrotic core. HRS analysis from MRI data based on water diffusion dynamics. Diffusion-Weighted Imaging (DWI) is a form of magnetic resonance imaging (MRI) based upon measuring of water content and of movement (diffusion) of water molecules within tissues. Brain lesions resulting from hypoxic ischemic injury (HII) are often characterized by a higher water content with more restricted diffusion (i.e., with a lower diffusion coefficient). This is visualized in the figure in schematics of coronal brain sections (top row) and, in more detail, of neocortical brain parenchyma (bottom row). Shown are intact tissue (left panel), an early HII (after 1-2 days) with damaged neuropil and intracellular edema (middle panel, blue areas), and a more advanced HII (older than 3 days) characterized by severe tissue damage and extracellular edema with water-filled cysts (right panel, red areas). To unequivocally distinguish these 2 types of tissue, hierarchical region splitting (HRS) uses the DWI data to calculate the corresponding average diffusion coefficient (ADC) maps from each of them, represented here in the right part of the drawings in the lower row as water molecules with varying lengths of movement arrows. These maps help to precisely define penumbra (blue, restricted diffusion) and core (red, large water content and extremely restricted diffusion) within the injury, the state of which evolves in time (compare early and later injury regions in the brain sections in the middle and right panels). While the penumbra still contains salvageable cells, core regions are irreversibly damaged and are characterized by water-filled cavities containing cell debris.

FIGS. 21A-21B depicts an example of applying the HRS algorithm to a representative MRI of the brain, segmenting an ischemic lesion into salvageable penumbra (blue) vs. unsalvageable necrotic core (red). FIG. 21A shows how an HI lesion in a rat pup detected by MRI can be segmented into “penumbra” (pseudo-colored blue) vs. “necrotic core” (pseudo-colored red) using the biophysical principles. FIG. 21B shows that these same principles can be applied to an HI lesion in a human newborn visualized under MRI: the HI lesion can be segmented into “penumbral” vs. “necrotic core” regions/volumes by HRS just as in the rat pup. The top row shows raw ADC images from a term newborn with arterial ischemic stroke at 3-5 days post-injury (with the typical focal diffusion restriction encompassing the middle cerebral artery territory), while the bottom row shows the same images with the HRS algorithm applied showing how that lesion can be subdivided into core (red) and penumbra (blue), and the volumes quantified. Note that cores can be interdigitated by penumbra.

FIGS. 22A-22B shows spatiotemporal evolution of the core and penumbra in the RVM of hypoxic-ischemic cerebral injury (HII) in rat pups, showing that penumbra (blue) will transition into core (red) as the cells in the penumbra die unless there is intervention. The window of opportunity for rescue of the significant expanse of the penumbra is approximately 2-3 days post-HII. FIG. 22A shows serial imaging over 17 days of a 10 day old rat pup subjected to an RVM lesion (unilateral carotid occlusion followed by 90 min. of hypoxia [8% ambient 02]). Shown is the evolution of core-penumbra at 2 Bregma levels from the same RVM rat pup. The top row for each Bregma level shows T2 maps and the bottom row at each Bregma level shows the Hierarchical Region Splitting (HRS) determined core (red) and penumbra (blue). FIG. 22B shows quantification of the temporal evolutions of core and penumbral volumes. The core and penumbral volumes are expressed as a percentage of the total ischemic injury volume. In this rat pup, the penumbral region initially was much greater than the core, but subsequently, there was rapid transformation of penumbra to core; the total lesion volume remained essentially unchanged, but it gradually became charactered by irreclaimable core (which increased) and less so by salvageable penumbra as it contributed to core and, hence, occupied a smaller percentage of the lesion volume.

FIG. 23 the ability of the HRS algorithm to segment the infarct into penumbra (blue) versus necrotic core (red) in human neonatal brains. Rows 1, 3, and 5 illustrate ADC MRI maps (at serially deeper brain depths from top to bottom) computed from the diffusion MRI sequences captured from a representative full-term human neonate with a perinatal ischemic injury. On an ADC map, an acute lesion of that type is hypointense (dark). When the HRS algorithm is applied to each ADC map (Rows 2, 4, and 6, respectively), the infarct can be computationally segmented into regions of necrotic core (red) vs. penumbra (blue). Each HRS pseudo-colored image is superimposed on its respective ADC image just above it. These data suggest that such core vs. penumbra designation, segmentation, localization, and quantification by HRS can be used clinically for human neonates with perinatal asphyxia as it has been used preclinically in the rat pup model of perinatal asphyxia.

FIGS. 24A-24B shows an RVM protocol that was optimized to yield consistently mold-to moderate HI lesions in 70-80% of rat pups for IND-enabling studies. MRI classification of severity correlates with histological classification of severity with both based on percent of brain damage based on volume. HRS can be used to classify the severity of the HII lesion based on the amount of volume infarcted in both rodents and humans and assess the evolution of brain injury. For each severity category, an example of whole rat brains, of hematoxylin and eosin (H and E) stained coronal rat brain sections, representative corresponding camera lucida drawings (at the same anatomical position), and 3-dimensional renderings of the HI injury volumes (extracted from axial MRI images) are shown in FIG. 24B reach corresponding nicely to MRI images of the same areas (FIG. 24A). (In the camera lucidas, ischemic areas are delineated in red). Mild injuries were often only cortical and unilateral, whereas moderate injury involved bilateral portions of the cortex. Severe injuries included virtually the entire cortex and also subcortical regions (basal ganglia, thalamus, and, at times, hippocampus). The percentage-lesion volumes based on 3D volumetric results and the HRS values are remarkably similar.

FIG. 25 shows neonatal behavioral tests suggest developmental delay in HI-injured pups. These 3 tasks test various aspects of motor function. The forelimb suspension task tests forelimb strength. HII impairs the ability of a pup to hang onto a horizontal rod compared to uninjured age-matched littermates. The negative geotaxis task tests the ability of a rat pup to sense, via vestibular input, that its head is facing downward, in response to which a normal pup will rotate 180° so that it is now facing upward; HII delays that response. In the third task, ambulation is scored on a 1-4 scale based on weight support and ability to have coordinated alternating stepping of both hindlimbs and forelimbs. Normal mouse pups achieve a 3 (4 being reserved only for adult gaits); HII delays the maturation of that mature gait.

FIGS. 26A-26C depicts MRI and quantification by HRS of the “Necrotic Core” and the “Salvaged Penumbra” of an HII lesion following minimally-invasive hNSC transplantation into the subventricular zone via an intracerebral ventricular injection. The top panel of FIG. 26A depicts coronal T2WI at 2 days after HII (just prior to hNSC implantation) where the hyperintense (white) regions indicate injured brain tissues. At 90 days post-HII (after hNSC implantation), the remaining injured tissue has become predominately cystic (very bright signal) (green arrows). The bottom panel of FIG. 26A depicts the relative “core” (pseudocolored red) and “penumbral” (pseudocolored blue) tissues after HRS assessment of those coronal images. Note the transition from “penumbra” (blue) (blue arrows) at 2 days to primarily “core” (red) (red arrows) at 90 days post-HII in saline-treated brains. (The 2 representative animals from the saline and hNSC groups started out with an equal penumbra:core ratio at 2 days post-HII prior to any intervention). FIG. 26B depicts a quantitative assessment of total lesion volume (core+penumbra) demonstrating a significant (p<0.04) decrease in total lesion volume in the hNSC-implanted pups due to “rescue”—and hence diminution in the size—of the penumbra (blue); the core (red) is not reduced in size. Penumbra that is not “rescued” progresses to become “core” (red and blue speckling). It is largely this speckled area (green circle) of parenchyma that is salvaged in the hNSC group, i.e., prevented from progressing to the core (solid red) ultimately seen to the right of the green arrow in saline-treated animals. (n=9 pups receiving hNSCs, 8 receiving saline). FIG. 26C shows coronal MRI slices subjected to HRS as in [the bottom panel of FIG. 26A] from most rostral (“r”) to most caudal (“c”) poles of representative injured cerebral hemispheres of saline-treated vs. hNSC-implanted rat pups at 90 days post-HII, showing that hNSC treatment was associated with a decreased lesion volume throughout the brain based on diminution of the penumbra (blue) which then approximated normal tissue (based on MRI) and did not progress to necrotic core (red) (as in the saline-treated groups).

FIGS. 27A-27D depicts the relationship of HII severity to interhemispheric migration and engraftment of donor human derived neural stem cells. FIG. 27A shows distribution of hNSCs (as identified by immunoreactivity to the human-specific antibody Stem-101) superimposed upon a T2WI of transplanted brains at 90-day post-HII Day. The identities of these cells (which are principally located in the penumbra, particularly in the MRI-classified “moderate” animals) are shown by immunohistochemical analysis in FIG. 27D. FIG. 27B shows a positive correlation between the density of engrafted hNSCs in relation to the RPSS11, plateauing at “moderate”. RPSS uses a 1-4 scale to classify HII lesion severity based on total lesion volume as measured under T2WI): mild (0.25-1.49), moderate (1.5-2.49), or severe (≥2.5). hNSC density was calculated as the ratio of Stem 101+ cells to total (DAPI+) cells in each field. The blue line delineates the log regression (R2=0.7476) and illustrates that the positive correlation between RPSS and stem cell density reaches a maximum engraftment saturation point at a moderate RPSS score and plateaus at higher RPSS values. Correlation by linear regression between RPSS and the average number of hNSCs is even higher at R2=0.994, corroborating a positive correlation between RPSS and the presence of stably engrafted hNSCs that peaks at a point where the penumbra is of maximal size. Error bars=SEM; statistical significance calculated by One-Way ANOVA (α=0.05). FIG. 27C compares distribution of hNSCs between the left (injured) and right (intact) hemisphere in each HII severity condition at 90 days post-HII (87 days post-transplant). Note a significant difference in the location of hNSCs in the moderate and severe brains between the injured left hemisphere compared to the intact right hemisphere. As in FIG. 27B, more donor hNSCs engrafted in the moderately and severely injured brains compared to the mildly injured brain. Histograms represent cell counts of Stem-101+ cells with statistical significance calculated via a One-Way Holm-Sidak ANOVA (α=0.05); error bars=SEM.). FIG. 27D shows the fate of stably engrafted hNSCs in the penumbra after 3 months in vivo, the positions of which are shown schematically at low power in FIG. 27A. The hNSCs (recognized by human-specific markers) differentiate into the 3 cardinal neural cell types as well as residual self-renewing hNSCs. Representative confocal images at low [i] and high [ii, iii] magnification of immunoreactivity to the mature neuronal marker NeuN (red) co-localizing with the cytoplasmic human-specific marker Stem-121 (green, “121”) [ii] and the nuclear human-specific marker Stem-101 (green, “101”). DAPI (blue) staining shows all cells in the field. The majority of the donor-derived cells remained as [iv] undifferentiated hNSCs (vimentin+[red]); [v] young oligodendrocytes (Olig2+[red] but MBP—[not shown]); and [vi] astrocytes (GFAP+[red]). White arrows identify the non-neuronal progeny of the engrafted hNSCs in the merged image of the respective low magnification, individually-stained insets. Scale bars=20 μm.

FIGS. 28A-28D shows that human derived neural stem cells preferentially migrate to areas of moderate and severe HII (though not mild), congruent with upregulation of the repair-associated gene HSP27 which is most robust in regions where host cells are still salvageable (penumbra) and becomes upregulated in donor human derived neural stem cells. FIG. 28A depicts RPSS and HSP27 immunoreactivity in ischemic areas, particularly the penumbra, have a moderately strong positive correlation (based on a log regression; R2=0.403) with a peak at an RPSS of 2.5, consistent with the presence of a salvageable penumbra and identical to the point of peak hNSC engraftment density. FIG. 28B shows HSP27 expression by endogenous host cells is greatest in the penumbra of the moderately severe HII animals. Comparisons by ANOVA; error bars=SEM; α=0.05). FIG. 28C shows representative photomicrographs of a field of HSP27+(red) endogenous (human marker-negative) cells in the penumbra of a representative moderate HII rat. Nuclear DAPI (blue) recognizes all cells in the field. Counting such cells yielded the histograms in FIG. 28B. FIG. 28D shows HSP27 (red) and donor hNSCs (Stem-101+, nuclear green) co-localize consistently in the penumbra. In the same way HII appears to induce upregulation of HSP27 in host cells (as seen in FIG. 28C), so too does it upregulate HSP27 in the engrafted hNSCs. Scale bar=100 μm. HSP27 upregulates when the salvageable penumbra is most prominent, which correlates also with where hNSCs are found to have migrated most robustly and with the greatest degree of parenchymal rescue and functional recovery.

FIG. 29 shows beneficial effects of an hNSC graft contralateral to HII as soon as 30 days post-transplant. Two days after an ischemic insult (postlesion day 2), the hNSCs were instilled into the cerebral ventricle contralateral to the lesion which, because its “penumbra” (in blue) predominates over its “core” (in red) in this HRS-processed MRI, would be categorized as of “moderate severity”. No hypothermia was applied. Shown is one representative cut through the animal's MRI. The HRS volume data for each component of the lesion are shown as percentages of the total lesion. Other than some residual unreclaimable necrotic core, almost all of the penumbra was returned to normal tissue based on MRI criteria, dramatically diminishing the overall volume of the lesion.

FIGS. 30A-30B shows that significant neuroprotection is conferred by intraventricular hNSC grafts on reversing severity or suppressing progression of severity of the HI lesions in RMV rats. Administration of hNSCs contralateral vs. ipsilateral to the lesion appears essentially to be equally efficacious and safe, boding well for biventricular administration of hNSCs in the actual clinical situation where the perinatal lesions are typically bilateral, asymmetric, or even global. FIG. 30A shows assessment based on improving degree of lesion severity as quantified by MRI/HRS. PD10 rat pups were imaged by MRI 2 days after lesioning (left gray column) to quantify their degree of injury severity. On the same day, a suspension of 2.5×10⁵ hNSCs was gently injected into one of the pup's lateral cerebral ventricles—either contralateral (middle row, n=17) or ipsilateral (row C, n=8) to the lesion. The animals were then monitored by MRI on PTrDs 3, 30, and 90 to follow evolution/progression/recession of their HI injuries compared to non-grafted or vehicle-grafted controls (gray, top row). While in untransplanted controls, the lesions progress principally to a severe phenotype (red sectors), (and mild lesions [blue sectors] evolve into moderate lesions [orange sectors]), the neuroprotective effect of the grafted hNSCs leads to a robust amelioration, reflected in the reversion of moderate lesions (orange sectors) into mild lesions (blue sectors), presumably by rescuing the penumbra, the predominance of which defines a moderate lesion by the MRI/HRS criteria. hNSCs do not reduce the volume of the necrotic core (the predominance of which defines a severe lesion [red sectors]) because those host cells are already dead; hence the severe lesions do not disappear, but relatively few moderate lesions (orange sectors) progress into severe lesions (red sectors), as they do dramatically in the absence of hNSC implantation [gray, top row]. Although this analysis based on HRS suggests that ipsilateral administration perhaps salvages a bit more of the “moderate” regions (orange sectors) to improve to “mild” (blue sectors) and prevents a bit more of those moderate regions from progressing to “severe” (red sectors), on balance there seems to be little difference between the 2 ROA. Also noteworthy is that the terrain appears to stabilize by 1-month post-transplant: the rescue at 30 days post-transplant persists until at least 3 months post-transplant, and clearly does not progress, as seen in untreated brains. FIG. 30B shows assessment based on preservation of brain volumes measured by MRI in unilaterally HI-lesioned rat brains measured at adulthood (PD90). Quantitative comparison of total hemispheric volumes in brains of animals with a unilateral, moderate HII in their left hemisphere, with or without subsequent contralateral vs. ipsilateral intraventricular transplantation of hNSCs measured at adulthood (PD90) (no HT was applied). Tissue volume was measured under MRI. White bars show the tissue reduction in the left hemisphere in non-grafted rats. Stippled bars show the same in transplanted animals. The size of the hemispheres is given as a percent relative to intact controls (100%) (horizontal gray threshold). The lesioned untreated hemisphere is dramatically smaller than the intact control (p<0.0005). After neonatal hNSC transplantation, this loss of tissue volume is significantly reduced, suggesting significant neuroprotection. In this study in which tissue volume alone was measured but not the type of lesion encountered (i.e., core vs. penumbra, as in I), it appears that contralateral grafts give a slightly better outcome (p<0.005) than ipsilateral ones (p<0.05). In FIG. 30A, where the improvement in lesion severity was calculated, an ipsilateral ROA appeared to be slightly more efficacious. Taking the 2 data sets (FIG. 30A and FIG. 30B) together, on balance it appears that comparisons of hNSC injections contralateral vs. ipsilateral to the infarct shows both ROA to be essentially identical in efficacy and safety—the slightly greater reduction in severity for an ipsilateral ROA compared to a slight advantage in volume preserved for contralateral ROA not only balances each other, but also reinforces the intent to administer hNSCs biventricularly in preclinical rat pups going forward and in actual babies given that true clinical perinatal HII is less restricted to one side and/or more global than can be emulated in the RVM.

FIG. 31 shows that hypothermia alone does not prevent evolution of penumbra to core and that the total HI lesion volume increases because the core grows in volume. PD 10 RVM rat pups were subject to HT (4 hours of HT in these rat pups are regarded in the field as equivalent to 3 days of HT in full-term human neonates). Shown are the outcomes on PLD 2 and PLD 32 as obtained from MRI/HRS data. Illustrated is the contribution of core (hashed histograms) and penumbra (gray histograms) volumes to the entire lesion (black histograms) after HT in moderate lesions on PLD 2 and PLD 32. The volumetric data were obtained from the MRI-based HRS analyses and expressed as a percentage of the entire brain's volume. The lesion volume grows over the course of the month because more brain volume transitions to core volume. The evolution of the lesion resembles that of an HI lesion with no intervention at all. Even using a colder temperature (29-30° C.) than is typically used clinically (33-34° C.) did not improve outcome. Lesion reduction only becomes evident when HT is followed by hNSC transplantation.

FIGS. 32A-32B shows the impact of hypothermia (HT) (as clinically performed)+hNSC transplantation (Tr) on brain damage in adult rats (PD 90) treated following hypoxic-ischemic (HI) cerebral injury as rat pups (PD10). FIG. 32A depicts coronal hematoxylin-stained histological brain sections from “INTACT” and “HI” animals, and animals additionally treated with HT (emulating present SOC) (HI+HT) (red arrow) or injected with hNSCs without HT (HI+Tr) or after HT (HI+HT+Tr). Groups designated (randomly) to be transplanted (Tr) 2 days post-HII and post-HT received 2.5×10⁵ hNSCs slowly infused as a cell suspension (in 5 μl) into the cerebral ventricle contralateral to the lesioned hemisphere. The sections were selected from comparable rostro-caudal levels. Damage was observed principally in the neocortex and hippocampus. In brains injected with hNSCs, (as seen in representative sections from the HI+Tr [blue arrow] and HI+HT+Tr [green arrow] groups) such extensive tissue damage at PD90 was never observed. FIG. 32B depicts brain area calculations of histological sections at comparable coronal levels as seen in FIG. 32A. The measurements of brain tissue are expressed as percentage of brain volume in relation to intact controls (100%) (gray horizontal threshold and purple circle). The white bars represent section areas from lesioned (HI), lesioned+HT (HI+HT), and transplanted (HI+Tr) rats. Stippled bars show the values from rats receiving HT+hNSCs (HI+HT+Tr). The brains with the largest lesions (i.e., HI and HI+HT), show a very significant brain size reduction (an ominous sign) compared to the brains of intact normal controls as well as compared to brains from HI+Tr and HI+HT+Tr animals (***p<0.0005) (n=8-14 group). In lesioned animals receiving a contralateral hNSCs alone even without HT (HI+Tr), brain tissue is significantly preserved (due to the hNSC's neuroprotective) but is slightly yet significantly improved when HT is added (*p<0.05), suggesting some synergy (not just additive benefit) between HT and hNSCs. HII animals who were cooled on PLD 0 and subsequently received hNSCs on PLD 2 (stippled bar HI+HT+Tr) had brain volumes statistically indistinguishable from those of intact controls (purple circle). These results using grafts contralateral to the lesion were also confirmed using ipsilateral hNSC grafts (also using volumetric measurements of the lesioned hemisphere).

FIGS. 33A-33B shows graft-dependent lesion reduction after hypothermia (HT), not achieved by HT alone. Animals were subject to left common carotid occlusion and exposure to 8% 02 at PD10 (the RVM). After 4 hours at 36.5° C., they were cooled for 4 hours. At PLD 2, after checking the lesion status by MRIs (T2WI; first column), the rat pups were grafted into the cerebral ventricle contralateral to the injury with hNSCs (2.5×10⁵ cells). Shown are coronal MRI images from corresponding brain levels of FIG. 33A a representative grafted (cell deposits are more rostral to the brain slice shown and, therefore, not visible) or FIG. 33B a representative “hNSC-conditioned medium”-grafted control animal on PLD 2, 5, and 32, and on post-transplantation-day 3 and 30 (PTrD 3 and PTrD 30, respectively). While at PLD 2, ˜5 hours before hNSC transplantation, the MRIs (T2WI) in both rats show comparably “moderate” HII, by as soon as 1 week after hNSC transfer, the grafted pup shows lesion improvement (i.e., resolution of T2 signal intensity [white]) compared to the control in FIG. 33B. This progressive amelioration continues until one month after grafting; the lesion resolves into a very mild form, while the hNSC-conditioned-medium-alone-grafted control continues to develop a severe lesion (excessive T2 signal intensity [white]) with large porencephalic cysts taking up large portions of the cortex and hippocampus of the lesioned hemisphere. Of note, because medium conditioned by the hNSCs alone had no therapeutic effect, this experiment rules out a role being played by simply growth factors or exosomes being acutely secreted by the hNSCs as the therapeutic agent; the hNSCs themselves are the mediators of therapeutic action (by likely multi-modal mechanisms).

FIGS. 34A-34B shows that the optimal time for grafting hNSCs in relation to occurrence of hypoxic-ischemic injury (HII) and administration of hypothermia (HT) is 3 days-post-lesion which would coincide with the cessation of HT. Additionally, administering the hNSCs contemporaneously with the initiation of HT is too soon, while waiting 1 week post-HII is too long. FIG. 34A shows that grafting hNSCs contemporaneously with initiation of HT (termed “immediate grafting”) is less effective than waiting for the rat pup to be rewarmed. PD10 rat pups (experimentally asphyxiated via the RVM) received an implantation of hNSCs into both lateral cerebral ventricles (5 μL/ventricle; 50,000 cells/μL HBSS) at the same time as instituting HT (32.5° C.) 4 hours post-insult. After the period of HT, the pup was rewarmed for 15 minutes, and returned to the dam. On PTD 5 (B, “PTD 5”) and 30 (B, “PTD 30”), MRI evaluation was performed to characterize the HII lesion—including applying HRS post-hoc analysis to distinguish “penumbra” from “core” within the infarct and, hence, to be able to grade severity of the injury based on MRI. Although the logistics of doing immediate grafting precluded being able to perform an immediate pre-grafting MRI, RVM protocols are so consistent and reproducible that they could reliably know what the spectrum of lesion types and their prevalence would be [A]: mild (blue) (16%), moderate (orange) (72%), and severe (red) (12%). MRI on PTD 5 (B, Left Pie Chart), initially suggested an impressive beneficial effect from immediate grafting: a disappearance of severe lesions and reduction of moderate lesions to 20%, with the majority being minor lesions (80%). The improvement could also be due to a doubling of the cell dose in that the hNSCs were administered into both cerebral ventricles simultaneously. The efficacy of HT alone was minor. However, the beneficial effect of immediate grafting was not stable or sustainable, as seen one month later (PTD 30) [B, Right Pie Chart]: “mild” lesions reverted back to “moderate” ones, and “moderate” lesions progressed to becoming “severe” ones. This was never observed in rats grafted during the “optimal window” of 3 days after rewarming (which coincided with 3 days post-HII), where lesion reduction remained stable for up to 3 months or longer. The findings above in vivo are consistent with other experiments performed that indicated that hNSC behavior is compromised by prolonged exposure the temperatures used for HT. FIG. 34B shows that grafting hNSCs 1 week following a course of HT is ineffective in altering the fate of an HII lesion—in stark contrast to the efficacy when implantation was performed 3 days post-rewarming/post-HII. Asphyxiated PD 10 rat pups (via RVM) received HT starting 4 hours post-HII (emulating the ≤6-hour window used clinically). After being rewarmed, the pups were returned to the dam. One week later (post-HT Day 7 and post-lesion Day 7, “PLD 7”), the lesions were evaluated by MRI with HRS, and the animals were grafted with hNSCs into both lateral cerebral ventricles (5 μL/ventricle; 50,000 cells/μL HBSS). In this “delayed grafting” paradigm, MRI could be performed on PLD 6, just 12 hours prior to grafting, enabling us to know exactly the spectrum of lesion severity in the experimental animals pre-intervention (Left Pie Chart; “PLD 6”). Here, all the lesion severity types: 20% severe (red), 20% moderate (orange), 60% mild (blue). By this time, 6 days after the insult, the lesions have already started to adopt their final appearance. Delayed bilateral hNSC grafting of these animals (on PLD 7-1-week post-rewarming and post-lesion) does NOT appear to have any beneficial effects on the resolution of lesion severity or forestalling disease severity progression. On PTD 3, a time point where, in rats grafted 3 days post-HII, impact from hNSCs reflected in lesion reduction could already be routinely seen, the “delayed grafting” animals showed exactly the same lesion spectrum as before transplantation. In other words, to reiterate, delaying the transplantation of hNSCs to 1-week post-HII and post-HT compromised their neuroprotective actions.

FIG. 35 shows hNSC transplantation does not provide lasting rescue if started at same time as hypothermia rather than waiting 3 days post-HII (post-rewarming). Transplantation of hNSCs is most efficacious when waiting 3 days post-HII, but less than 1 week post-HII. Shown are 3 examples of HI-lesioned rat pups with hNSCs grafted contemporaneously with the initiation of HT. Bilateral deposits of 250,000 hNSCs in 5 μl of vehicle were injected into the lesioned PD10 rats after HI and just as the pups were being readied for the initiation of HT (4 hours post-HII—modeling SOC HT in human babies). While 5 days after transplantation (PTD5, upper row) the lesions initially appeared reduced (red arrows), 1 month later, the severity and size of the core region increased significantly (mild lesion progressed into moderate and moderate into severe) (green arrows). This progression and worsening never occurred with successful grafts 2-3 days post-HII (into PD12 animals), i.e., 2-3 days after SOC HT, where a reduction in the lesion persisted into adulthood (until at least 3 months of age, 90 days post-HII).

FIGS. 36A-36C shows that HT decreases the metabolism, proliferation, and migration of hNSCs earlier than 3 days of exposure to HT and while keeping HNSCs hypothermic for 3 days can negatively impact their function, if they are transplanted after the 3-day hypothermia regime when babies have been rewarmed, then problems are circumvented. FIG. 36A shows metabolism, as gauged by non-specific protein synthesis via a BCA assay, after an initial bump on Day 2 (stress proteins), is suppressed in a protracted manner in HT hNSCs compared to NT hNSCs (p=0.0004); by Day 6, protein in NT hNSCs remains higher than in HT hNSCs (p=0.0016). FIG. 36B shows growth HT (blue) vs. NT (red) hNSCs. Colonies of the former grew slower than of the latter (p=0.006) with a longer doubling time (21.1 day vs. 5.2 day). PCNA (a cell division marker) was also diminished [inset]. Again, the divergence becomes apparent a bit prior to 3 day of HT. FIG. 36C shows migration of HT hNSCs (blue) is slower compared to NT cells (red) (p<0.0001). Differences start emerging early, and become statistically significant just prior to 3 day of HT. Therefore, this study of the hNSCs under hypothermic conditions in vitro, when taken together with the in vivo data of suboptimal unsustained neuroprotection by the hNSCs when administered during the HT therapy, reinforces the conclusion that hNSCs are most effective when administered at the completion of rewarming from HT, which also, in human neonates, corresponds with 3 days post-HII.

FIGS. 37A-37B depicts the cell dose-response that increasing hNSC dose leads to decrease in severity of HII lesions. FIG. 37A shows a scheme of dose-escalation of hNSCs administered into both lateral cerebral ventricles 2-3 days after hypoxic-ischemic injury (HII) in PD10 rat pups. FIG. 37B shows the impact of hNSC dose on outcome from perinatal hypoxic ischemic injury (HII) at adulthood (post-lesion day 90).

FIG. 38 shows that penumbral tissue is normalized when an optimized hNSC dose, route-of-administration, and timing following cerebral hypoxic-ischemic injury (HII) and therapeutic hypothermia (HT) is used in rat pups subjected to the RVM. PD10 Wistar rat pups received a cerebral HI lesion according to the optimized RVM and were cooled for 4 hrs. at 33° C. (the rodent equivalent of 3 days in a human baby). Two days later (PD12), the animals received a bilateral injection of a 4× standard dose (SD) of hNSCs (i.e., 2×5 μl with 10⁵ cells/μl) into both lateral ventricles and the evolution of the lesion was monitored in vivo by MRI and HRS analyses. Shown here is the example of a moderate lesion resolving into a mild one under the neuroprotective synergistic action of HT+hNSC transplantation. While the small red core areas remain (perhaps decreased a bit based on quantification), the initially large blue penumbra is rescued and has virtually disappeared by PD42 (and remained unchanged throughout the remainder of the 3-month monitoring period into adulthood). Here, the progression has been reversed because of the interposition of neuroprotection via the hNSCs: penumbra reverts to normal rather than into core.

FIG. 39 depicts imaging- and population-based evidence that an escalation of the number of grafted hNSCs to 8× standard dose (SD) (2×10⁶ cells/rat pup brain) results in pronounced improvement on MRI lesion volume to the point of entirely eradicating lesions in some animals. An escalation in the number of grafted hNSCs to 8× standard dose (SD) (2×10⁶ cells/rat pup brain) results in even more pronounced improvement on MRI lesion volume by 30 days post-transplant (PTD30) than does SD. SD has been 250,000 cells/5 μl/ventricle, where only 1 ventricle had been implanted (implantation into the ventricle ipsilateral vs. contralateral to the lesion is equally efficacious and safe. Increased cell dose leads to a slightly but significantly improved outcome. The top left image shows an MRI of a representative animal with an initial high-moderate HII lesion on PD12, as defined by MRI. The top right image shows an MRI of the same animal on PTD30, appearing virtually lesion-free by MRI criteria. The pie graphs show the outcome from whole cohort of 12 animals who received 4-8×SD. While, after lesioning and HT of PD10 pups, and just prior to grafting on PD12, the entire spectrum of lesion severities was present (left pie chart), one month later (right pie chart), there appeared already to be complete disappearance of “moderate” lesions and even a significant percentage of “lesion-free” animals (n=12). After injections of hNSCs at SD (both unilateral or bilaterally) following HT, even though outcomes at PTD90 were very much improved, there were always some residual “moderate” lesions and, while reduced in number, they did not completely disappear but remained of a “mild type” (based on MRI grading). However, now with increased hNSC dose, those lesions seem to disappear (based on MRI) in a substantial proportion of the animals. Importantly, No adverse effects have been observed with higher donor cell numbers: no hydrocephalus, CSF obstruction, cell overgrowth, increased intracranial pressure, tumors/masses, displacement of normal cytoarchitectural structures, edema.

FIG. 40 shows the penumbra disappearing following high dose (8× standard dose) hNSC transplantation bi-ventricularly with no adverse effects.

FIG. 41 shows a single, one time intraventricular injection by an ultrathin needle (e.g., 30-34 gauge Hamilton syringe) causes no mechanical or inflammatory injuries in the overlaying cortex which the needle will pass through. Intact, normal PD12 rats received stereotactic-guided bilateral intraventricular injections of cell culture medium (10 μl per site) into the intact brain and then were sacrificed on Post-natal 90. The coordinates (in mm) were as follows: from Bregma −0.4, medio-lateral 1.5, dorso-ventral 3.5). The top image shows a coronal, hematoxylin-stained histological section from the PD90 rat brain with the injection sites marked by arrows. The brains of such animals (n=5) were examined for needle-induced damage to the cortical parenchyma. Neither mechanical nor inflammatory injuries were detected.

FIG. 42 shows motor and cognitive improvement following hNSC implantation into HII rat pups with salvageable penumbra. Three months post-transplant, rats with HII and an hNSC-associated rescue and diminution of the penumbra (red arrows) were more active and showed greater exploratory behavior in an open field test during over a 30-minute trial period (*p<0.006) compared to saline-treated HII age-matched control rats [left], and spent more time in the proper quadrant with the hidden platform in the Morris Water Maze test of working spatial memory, similar to uninjured rodents and in contrast to saline-treated control HII animals that displayed memory deficits (95% confidence intervals) [right]. (uninjured rats, n=18; saline-treated rats, n=9; hNSC-treated rats, n=16) (*p<0.05 vs. control; § p<0.05 vs. hNSC transplanted).

FIG. 43 shows implanting hNSC following HT to reduce HII cerebral lesion also normalizes adult rat function on a cognitive test (the novel object recognition test) to normal. HII rats, in which hNSC grafts lead to reduction of moderate lesions to mild ones or to a lesion-free state, behave like intact Wistar rats, by avoiding unfamiliar objects (n=12) and being able to retrieve memory engrams of what they have already encountered and what differs from that. In contrast, rats with moderate lesions do not avoid unfamiliar strange objects as Wistar rats normally do (consistent with the blocking anxiolytic effect of the HI lesion) (n=8). Results show means +/−SEM. Orange columns: unfamiliar object.

FIGS. 44A-44B shows that hNSC implanted into the cerebral ventricles were not detected in any tissues outside the cerebrum. FIG. 44A shows qPCR analysis for the presence of human Alu DNA in non-neural tissues of rats 3 months after intraventricular grafting of hNSCs. While the DNA from the hNSCs used for grafting showed a clear signal from amplified human Alu sequences, all non-neural tissues were, as expected, negative (red lines; the weak signal starting beyond 40 cycles coincided with the background signal of the NTC). On the other hand, amplification of the control rat-specific GAPDH sequence was clearly visible in the non-neural tissues while no signal was detectable in reactions using the DNA of hNSCs (blue lines). Non-neural tissues tested: thymus, lung, liver, spleen. FIG. 44B shows that the test for examining biodistribution of hNSCs outside the human brain is sensitive enough to pick up a single human cell in rat organ. These data from the titration of the human DNA used in FIG. 44A demonstrate that the PCR reactions are sensitive enough to amplify Alu sequences from even 0.1 pg of template. Since a mammalian cell contains on average 26 pg of DNA, the reactions are 260×more sensitive and would therefore easily pick up any isolated human cells in the rat tissues. From left to right: 100 ng, 50 ng, 10 ng, 1 ng, 100 pg, 10 pg, 1 pg, and 0.1 pg DNA template.

FIG. 45 shows integration within the host parenchyma of hGFAP+ donor-derived cells 3 months after grafting into the cerebral ventricle contralateral to the HII. hNSCs, injected 2-3 days after unilateral HII into the contralateral hemisphere, survived at least 3 months (time of sacrifice) and their hGFAP+ astrocytic progeny integrated within the host parenchyma from the subependymal region. The extension of the GFAP+ processes from the donor-derived cells through the ventricular walls suggests that these cells might also exert beneficial influence locally, in the affected parenchyma of the penumbra, in addition to the humoral actions of the undifferentiated donor hNSCs. The donor-derived cells were intermingled with ependymal cells of the host and in close contact with the host's blood vessels where an exchange of nutrients and neuroprotective molecules might have occurred.

FIG. 46 shows a schematic wherein neuroprotective NCSs are used to rescue the cells and the fibres de passage within the penumbra of an ischemic lesion returning the region to close to normal in contrast to no intervention where the penumbra will transition to core as the cells within the penumbra die.

FIGS. 47A-47B shows schematics of the processes as disclosed herein. FIG. 47A depicts the importance of rescuing the penumbra. Preservation of components of neural networks passing through that region would otherwise die and disrupt global functioning of the brain. FIG. 47B depicts the cellular and molecular basis of the rescue as mediated by hNSCs: the NSCs (a) inhibit the endogenous astrocytes from becoming reactive and toxic and rather return to being fetal-like and trophic, and (b) the hNSCs themselves differentiate into such astrocytes.

FIG. 48 shows a schematic of minimally invasive “One-and-Done” hNSC administration using a 30-34 gauge Hamilton syringe placed percutaneously under ultrasonic guidance into the cerebral lateral ventricles through the open anterior fontanelle over each ventricle.

### DETAILED DESCRIPTION

Certain specific details of this description are set forth in order to provide a thorough under-standing of various embodiments. However, one skilled in the art will understand that the pre-sent disclosure can be practiced without these details. In other instances, well-known structures have not been shown or described in detail to avoid unnecessarily obscuring descriptions of the embodiments.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below. Further, headings provided herein are for convenience only and do not interpret the scope or meaning of the claimed disclosure. It is contemplated that any embodiment discussed in this specification can be implemented with respect to any method or composition of the present dis-closure, and vice versa. Furthermore, compositions of the present disclosure can be used to achieve methods of the present disclosure. There is a need for more effective and late-stage treatments (e.g., for neonates who miss the 6 hour window for HT) and more broadly-applicable adjunctive treatments against perinatal HII that more elegantly target its multiple injurious processes, most of which are not addressed by HT, e.g., inflammatory, apoptotic, necrotic, excitotoxic, oxidative vascular and demyelinative causes.

As such, disclosed herein is a novel cell therapy for the treatment of moderate to severe perinatal hypoxic-ischemic brain injury in full-term neonates before, or preferably following, the completion of therapeutic hypothermia. The cell line that comprises the cell therapy can differentiate into all three cardinal neural cell types in a stable ratio even after prolonged passaging and can be cryopreserved while retaining these normal characteristics upon thaw and return to culture. In some aspects, the cell therapy comprises HFB2050-human derived neural stem cells (hNSC) isolated in 2000 from the forebrain ventricular zone (VZ) of a single anonymous human female fetus cadaver aged 13 weeks gestation. In some aspects, the cell therapy is administered in Sarnat clinical Stage 2 (moderate) to Stage 3 (severe) perinatal hypoxic-ischemic cerebral injury (HII) in full term neonates who qualify for hypothermia (HT). The cell therapy can be administered to full-term newborns before, during, or after the completion of three (3) days of HT treatment. In some aspects, the eligible neonates will receive an MRI before, during, or after HT treatment. The MRI can help determine if cell therapy is appropriate. In some aspects, the cells for use in the cell therapy are thawed on the same day that the HT treatment begins and the cell therapy is administered to the full-term newborn immediately and/or soon after the three (3) days of HT treatment is completed. In some aspects, the cell therapy is administered via minimally invasive aseptic intracerebral instillation through the anterior fontanelle into each of the lateral ventricles.

### Definitions

To facilitate an understanding of the present disclosure, a number of terms and phrases are de-fined below.

As used in this specification and the appended claims, the singular forms “a,” “an,” and “the” include plural referents unless the content clearly dictates otherwise.

As used herein, “about” or “approximately” means ±20% of the stated value, and includes more specifically values of 10%, ±5%, ±2% and 10% of the stated value.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “some embodiments,” “an embodiment,” “one embodiment,” “embodiments” or “other embodiments” means that a particular feature, structure, or characteristic described in connection with the embodiments is included in at least some embodiments, but not necessarily all embodiments, of the present disclosures.

As used herein, the term “neural stem cells (NSCs)” and “neural progenitor cells” both refer to cells that can generate progeny that are either neuronal cells (such as neuronal progenitors or mature neurons) or glial cells (such as astrocytes and oligodendrocytes). While neural stem cells are self-renewable (i.e., able to proliferate indefinitely), neural progenitor cells can be, but are not necessarily, capable of self renewal. In some embodiments, the NSCs originate from human (e.g., hNSCs). As used herein, the HFB2050 hNSCs cell line (e.g., HFB2050) refers to hNSCs originally isolated in 2000 from the ventricular zone of a single anonymous 13-week gestational female fetal cadaver.

A “committed progenitor cell” is a progenitor cell that is committed, or destined, to become a specific type of mature cell. This is in contrast to a multipotent or a pluripotent progenitor cell, which has the potential to become one of two or more types of mature cells.

The term “glia” and “glial cells” can be used interchangeably. As used herein, they refer to non-neuronal cells of the central nervous system and comprise mature oligodendrocytes, astrocytes, and committed progenitor cells for either or both of these cell types. An “oligodendrocyte” is a type of glial cell whose main function is to insulate nerve cell axons in the central nervous system of some vertebrates.

As used herein, “pluripotency” refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Neural stem cells are multipotent and not pluripotent. Embryonic stem cells are pluripotent and not multipotent.

As used herein, “cell-specific marker”, or a “marker” refer to a biological macromolecule (e.g., gene, RNA, protein, glycan) that is expressed more in and/or on some cells than others. A cell-specific marker can distinguish a cell from surrounding cells via common experimental techniques (e.g., immunofluorescence, FISH, flow cytometry, lectins). Neuron-specific markers comprise proteins expressed primarily by neurons, comprising neuron-specific class III beta-tubulin (TuJ1). Astrocyte markers comprise glial fibrillary acidic protein (GFAP) and Excitatory amino acid transporter 1 (EAAT1). Oligodendrocyte markers comprise oligodendrocyte transcription factor 1 (Olig 1) and oligodendrocyte transcription factor 2 (Olig 2). Ki67 is a nuclear, non-histone protein that is expressed only during active phase of the cell cycle, hence a marker for proliferating cell.

As used herein, “gene” refers to a polynucleotide containing at least one open reading frame, while the open reading frame encodes a particular protein. Gene can be a group of genes, a cDNA, or a synthetic nucleic acid (e.g., synthetic DNA or RNA).

As used herein, “transgene” refers to a gene that is partly or entirely heterologous (i.e., foreign) to an organism or a cell into which it is introduced. The transgene can be transferred naturally or by any of a number of genetic engineering techniques. A transgene can comprise one or more transcriptional regulatory sequences and any other nucleic acid sequence (e.g., intron).

As used herein, the term “vector” refers to any genetic element, such as a plasmid, phage, transposon, cosmid, chromosome, virus, virion, etc., which is capable of replication when associated with the proper regulatory elements and/or which can transfer nucleic acid sequences be-tween cells. Thus, the term can include cloning and expression vectors, as well as viral vectors.

Vectors can include transcription sequences such as polyadenylation sites, selectable markers or reporter genes, enhancer sequences, and other regulatory elements which allow for the induction of transcription.

As used herein, the terms “host” can refer to organisms and/or cells which harbor an exogenous DNA sequence (e.g., via transfection), vehicle, as well as organisms and/or cells that are suitable for use in expressing a recombinant gene or protein.

As used herein, neonate,” refers to a human in the first 28 days after birth.

A “subject” in need thereof, can refer to an individual who has a disease, a symptom of the dis-ease, or a predisposition toward the disease, with the purpose to cure, heal, alleviate, relieve, alter, remedy, ameliorate, improve, or affect the disease, the symptom of the disease, or the predisposition toward the disease.

The terms “treat,” “treating,” or “treatment,” and its grammatical equivalents as used herein, can include alleviating, abating, or ameliorating at least one symptom of a disease or a condition, preventing additional symptoms, inhibiting the disease or the condition, e.g., delaying, decreasing, suppressing, attenuating, diminishing, arresting, or stabilizing the development or progression of a disease or the condition, relieving the disease or the condition, causing regression of the disease or the condition, relieving a condition caused by the disease or the condition, reducing disease severity, or stopping the symptoms of the disease or the condition either prophylactically and/or therapeutically. “Treating” can also include lessening the frequency of occurrence or recurrence, or the severity, of any symptoms or other ill effects related to a disease or condition and/or the side effects associated with the disease or condition. “Treating” does not necessarily require curative results. It is appreciated that, although not precluded, treating a disorder or condition also does not require that the disorder, condition, or symptoms associated therewith be completely eliminated. The term “treating” encompasses the concept of “managing” which refers to reducing the severity of a particular disease or disorder in a patient or delaying its recurrence, e.g., lengthening the period of remission in a patient who had suffered from the disease.

The term “treating” further encompasses the concept of “prevent,” “preventing,” and “prevention.” The terms “prevent,” “preventing,” and “prevention,” as used herein, refer to a decrease in the occurrence of pathology of a condition in a subject, who does not have, but is at risk of or susceptible to developing a disease or condition. The prevention can be complete, e.g., the total absence of pathology of a condition in a subject. The prevention can also be partial, such that the occurrence of pathology of a condition in a subject is less than that which would have occurred without the present disclosure.

“Administering” and its grammatical equivalents as used herein refer to providing pharmaceutical compositions described herein to a subject or a patient.

The terms “pharmaceutical composition” and its grammatical equivalents as used herein refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein refer to an attribute of a material which is useful in preparing a pharmaceutical composition that is generally safe, non-toxic, and neither biologically nor otherwise undesirable and is acceptable for human pharmaceutical use.

As used herein “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

As used herein, “therapeutic agent” refers to any agent that, when administered to a subject, has a therapeutic, diagnostic, and/or prophylactic effect and/or elicits a desired biological and/or pharmacological effect.

A “therapeutically effective amount” can vary, as recognized by those skilled in the art, depending on the particular condition being treated, the severity of the condition, the individual subject parameters including age, physical condition, size, gender and weight, the duration of the treatment, the specific route of administration and like factors within the knowledge and expertise of the health practitioner. These factors are well known to those of ordinary skill in the art and can be addressed with no more than routine experimentation. Empirical considerations, such as the half-life, generally can contribute to the determination of the dosage. A “therapeutically effective amount” can be of any of the compositions of the disclosure used alone or in conjunction with one or more agents used to treat a condition. A therapeutically effective amount can be administered in one or more administrations.

As used herein “patient-ready” indicates the pharmaceutical composition is in a condition to be administered to a patient. The patient-ready aspects may include being at the correct dose, volume, temperature, sterility, or any combination thereof. In some embodiments, being patient-ready also means being on demand, such as available when needed.

### Perinatal Hypoxic-Ischemic Injuries (HII/HIE)

Disclosed herein are methods, compositions, and kits related to treating perinatal hypoxic-ischemic injuries (HII; also referred to as perinatal hypoxic-ischemic encephalopathy; HIE). Hypoxic-ischemic encephalopathy (HIE) is a type of neonatal encephalopathy caused by systemic hypoxemia and/or reduced cerebral blood flow resulting from an acute peripartum or intrapartum event. HIE can be a clinical consequence of perinatal, birth and/or neonatal asphyxia, and can cause significant mortality and long-term morbidity. Of affected newborns, 15%-20% of affected newborns will die in the postnatal period, and an additional 25% will develop severe and permanent neuropsychological sequelae, including mental retardation, visual motor or visual perceptive dysfunction, increased hyperactivity, cerebral palsy, and epilepsy. The outcomes of HII are devastating and permanent, making it a major burden for the patient, the family, and society. Therapeutic strategies to reduce brain injury in newborns with HII are lacking. The underlying pathophysiology of perinatal HIE is difficult to study in the human, thus the neonatal animal models for hypoxic-ischemic (HI) brain injury has been developed to model this human condition.

There is no ‘gold standard’ test or early biomarker for the diagnosis of HIE. HIE is suspected in a neonate who is depressed at birth and who, in the earliest hours of life, presents with disturbed neurological function. Diagnosis can be made based on evidence of a hypoxic and/or ischemic injury during the perinatal and/or intrapartum period. Clinical features of HIE can include abnormal state of consciousness, reduced spontaneous movements, respiratory difficulties, altered tone, abnormal primitive reflexes, and seizure activities. Other aspects of consideration in diagnosis can include blood gas values, Apgar score, physical examination, onset of multisystem organ failure, evaluation of the placenta and umbilical cord, neuroimaging, and electroencephalogram (EEG). Assessment of HIE stage should initiate as soon as possible after the neonate is resuscitated and stabilized and should continue for at least 6 hours.

The Sarnat scoring system is often used to assess the severity of encephalopathy, upon which HIE staging can be based. Sarnat staging includes evaluation of neuromuscular control, complex reflexes, autonomic functions, seizures, and EEG findings. HIE is classified in 3 Sarnat stages: Stage 1 (mild), Stage 2 (moderate), and Stage 3 (severe). These stages are not absolute and can change as a neonate's condition progresses or recovers. In Stage 2 and 3 (moderate and severe), the baby will be significantly unwell, and the level of support required is dependent on the degree of organ compromise.

Clinical management of HII primarily involves intensive monitoring and supportive care. Typically, neonates with HII require respiratory support, resuscitation, and monitoring of temperature, blood gas, respiration, and oxygen saturation. Supportive therapy for HII comprises seizure medications, inhaled nitric oxide, high frequency ventilation, extracorporeal membrane oxygenation therapies, volume replacement, hemodialysis, inotropic agents, and vitamins. Serial assessments for encephalopathy (Sarnat score) are performed and if criteria for moderate or severe encephalopathy (Sarnat stage 2 or 3) are met within 6 hours of birth, a hypothermia therapy (HT; or therapeutic hypothermia, TH) can be administered. HT is currently the only proven treatment for moderate and severe HIE that improves mortality and long-term outcomes for affected neonates. In some embodiments, an HT comprises a whole-body HT.

Inclusion criteria for a hypothermia therapy (HT) can comprise: gestational age greater than or equal to 35 weeks, birth weight greater than or equal to 1.8 kg, evidence of perinatal/intrapartum hypoxia, evidence of moderate or severe encephalopathy, no contraindications to HT, and cooling can begin before 6 hours of age. More specifically, to qualify for HT, and neonate can have an Apgar score of equal or less than 5 at 10 min after birth and continue to need resuscitation at 10 minutes. In some embodiments, the neonate has a blood pH lower than 7.00 or a base deficit of greater or equal to 16.0 mmol/L, indicated by an umbilical arterial or venous blood sample prenatally, or by a venous, arterial, or capillary blood sample within 60 minutes of birth. In some embodiments, the neonate presents encephalopathy signs comprising lethargy, stupor, or coma, and/or hypotonia, abnormal reflexes, absent/week sucking, and seizures.

Exclusion criteria for HT can comprise: critical bleeding, major congenital abnormalities, severe head trauma or intracranial bleeding, and/or moribundus. HT and rewarming can be administered stepwise with supportive care and intensive monitoring known to those who are skilled in the art. Cooling can be commenced within 6 hours of birth to limit secondary reperfusion injury and can be continued for 72 hours at target temperature, at about 33 to about 34 degrees Celsius, unless complications develop.

The standard of care (SOC) treatment for perinatal HII (e.g., HT) has the following limitations: 1) HT is ineffective in clinically “severe” cases (Sarnat Stage 3) and only marginally effective in ˜10% of clinically “moderate” cases (Sarnat Stage 2); 2) HT is believed to be neuroprotective by slowing metabolic demand and preventing re-perfusion injury, but does not address the multiple other pathogenic mechanisms that contribute to development of CP (e.g., inflammation, apoptosis, necrosis, excitotoxicity, oxidative damage, mitochondrial dysfunction, vascular disruption and/or rupture of the blood-brain barrier, demyelination, etc.); 3) There is no therapy at all for asphyxiated infants who are unable to receive HT within 6 hours of birth; and 4) There is no therapy for asphyxiated infants who do not respond to HT.

Neurological outcomes of concern of HII can include spasticity, choreoathetosis, dystonia and/or ataxia that are often grouped together as cerebral palsy (CP). The development of CP can originate from adverse effects of hypoxia, such as inflammation, apoptosis, necrosis, excitotoxicity, oxidative damage, mitochondrial dysfunction, vascular disruption and/or rupture of the blood-brain barrier, demyelination, seizures, neurosensory deficits (deafness or blindness), or cognitive deficits. Thus, an urgent need existing for an alternative and effective treatment for HII, particularly, perinatal HII. The present disclosure provides compositions and methods for treating perinatal HII with human neural stem cells (hNSCs) or progenitors thereof.

Human Neural Stem Cells (hNSCs)

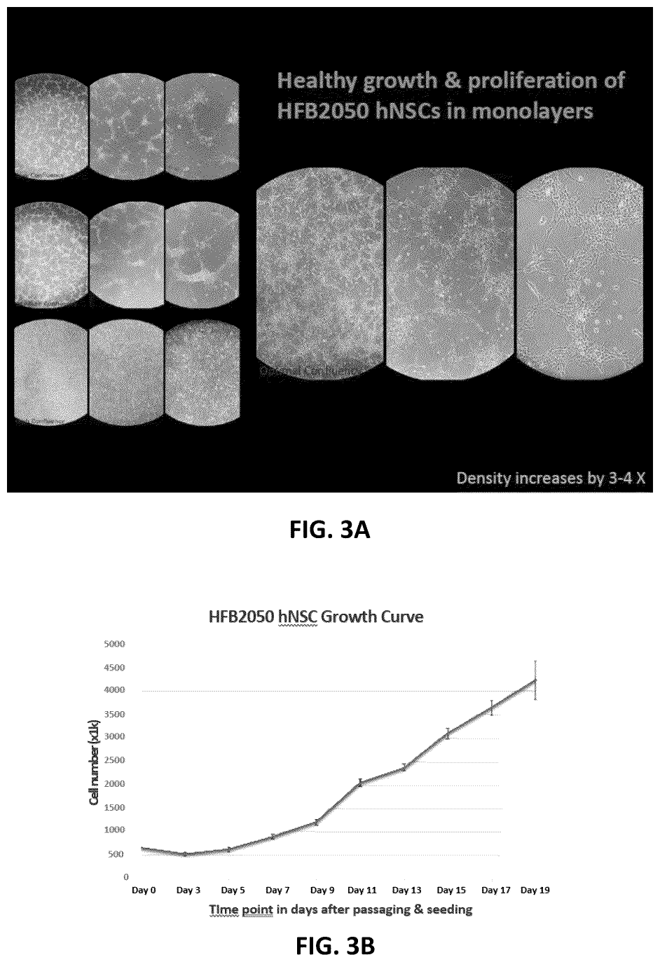

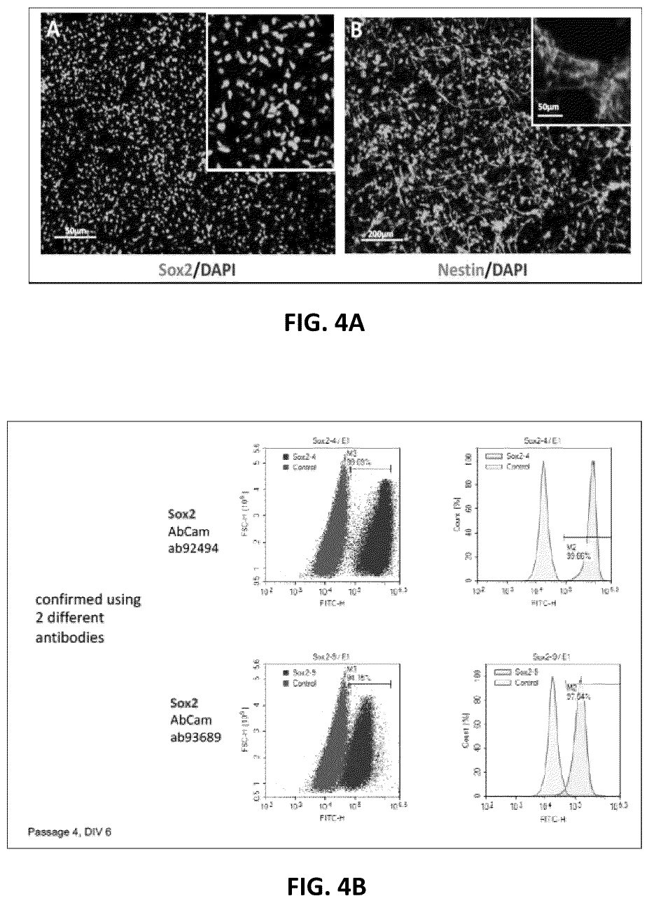

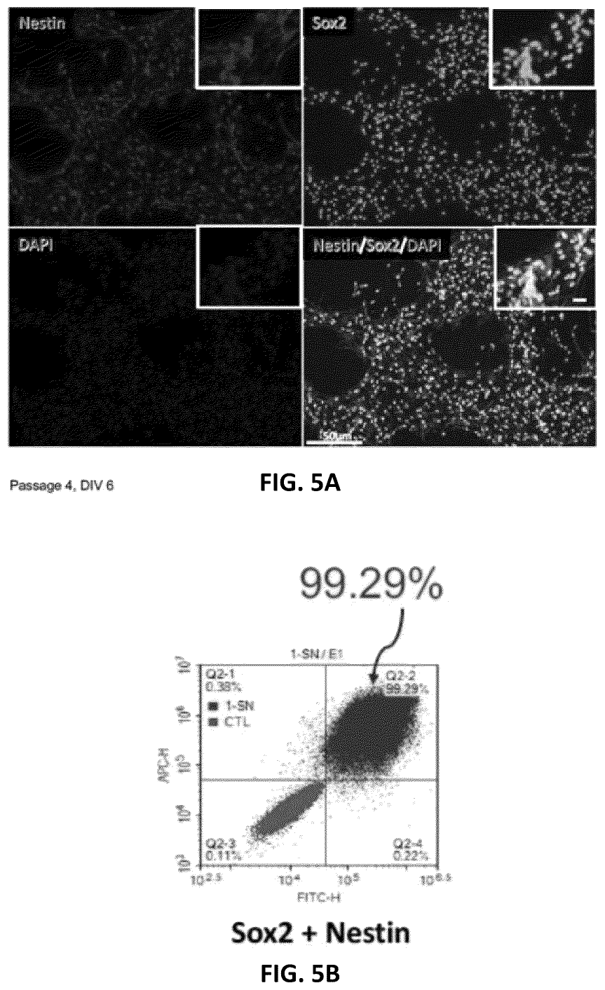

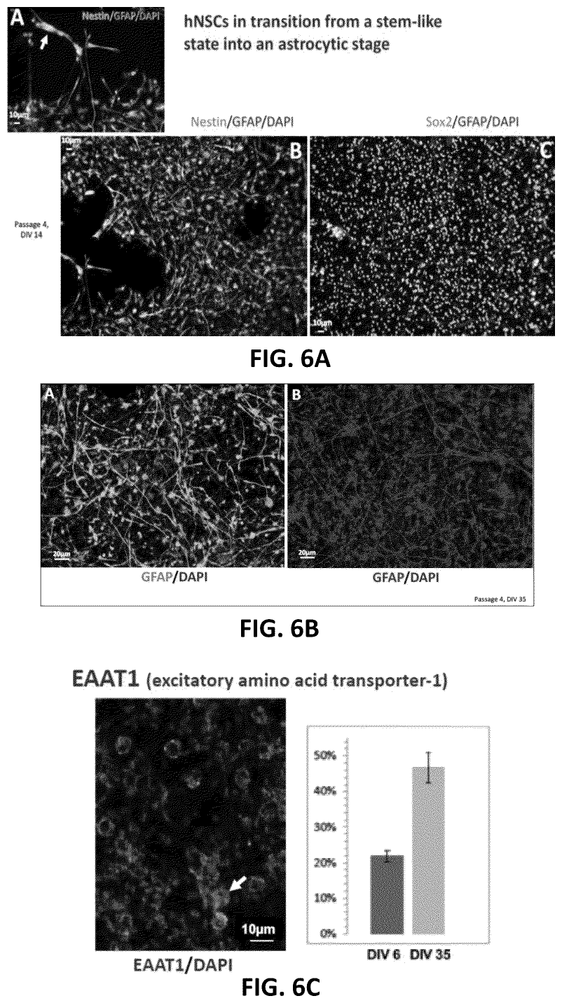

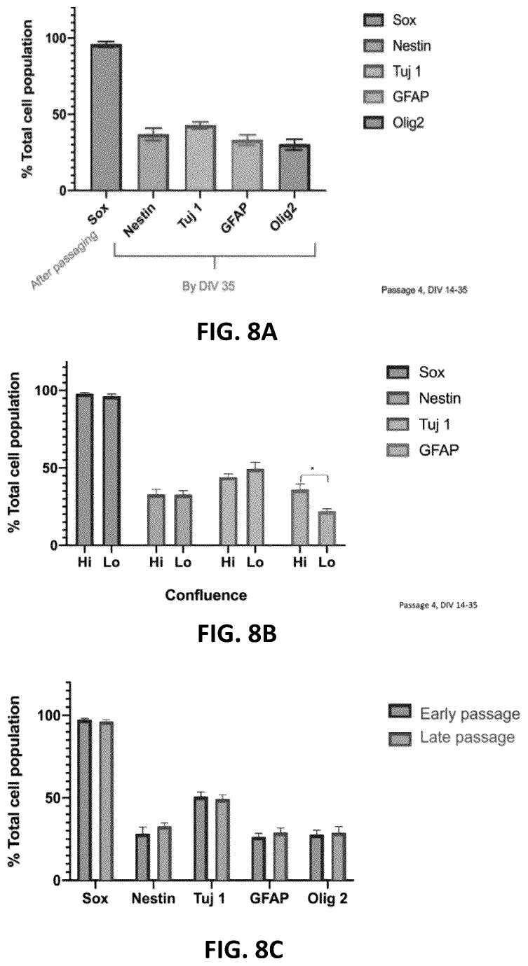

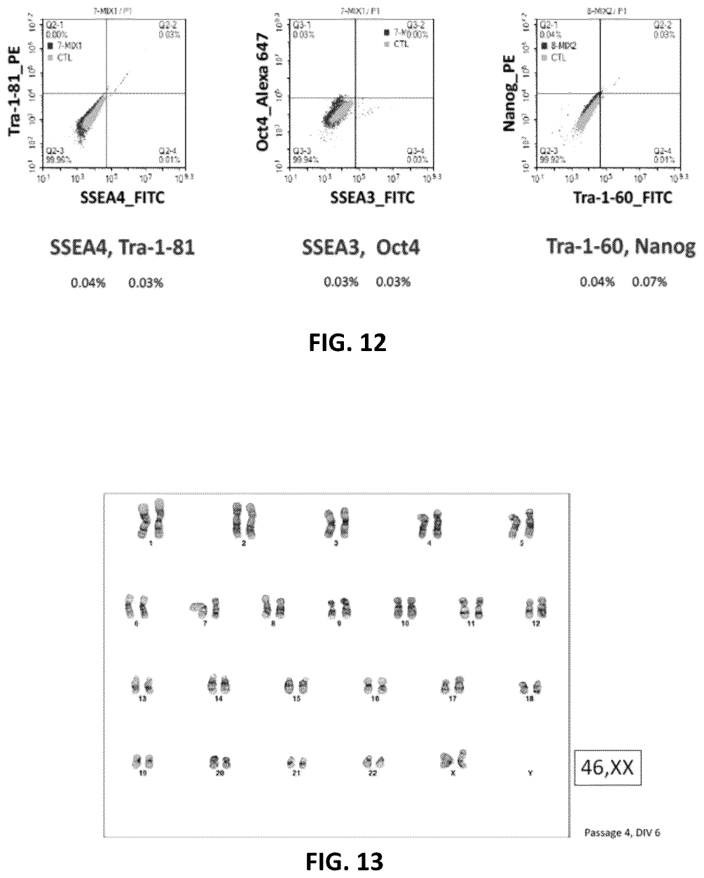

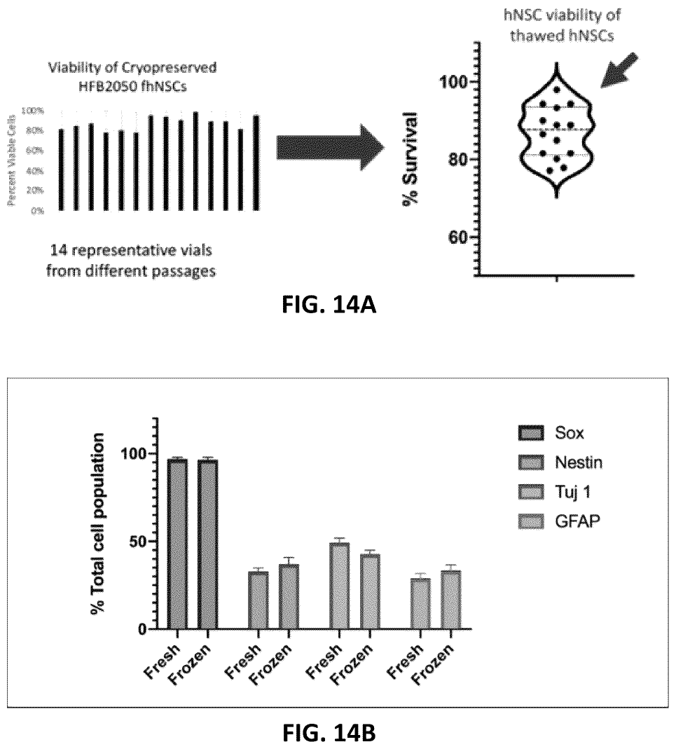

Neural stem cells (NSCs) are stem cells in the nervous system that can self-renew and give rise to differentiated progenitor cells to generate lineages of neurons as well as glia, such as astrocytes and oligodendrocytes. The isolation and generation of NSCs can be achieved by various methods known in the art, including isolating from primary tissues, differentiation of pluripotent stem cells into NSCs, and transdifferentiation of somatic cells. Provided herein are methods and compositions related to human NSCs (hNSCs). In some embodiments, the hNSCs are isolated from the ventricular zone of a single 13-week human female fetal cadaver brain. In some embodiments, the hNSCs comprise or consist of a stable hNSC cell line. In some embodiments, the hNSCs are the HFB2050 hNSCs cell line (“HFB2050 hNSCs”). In some embodiments, the HFB2050 hNSCs demonstrate stable characteristics in vitro. As depicted in FIG. 3, the HFB2050 hNSCs demonstrate stable characteristics including healthy growth and proliferation in monolayers. In some embodiments, the HFB2050 hNSCs possess normal growth kinetics and has a doubling time of about 6 days. In some embodiments, the HFB2050 demonstrate stable expression of hNSC markers (e.g., Sox2 and Nestin) and ability to proliferate and self-renew. As depicted in FIGS. 4-5, the HFB2050 express hNSC markers (e.g., Sox2 and Nestin) shortly after plating (DIV6), shortly after passaging, and prior to differentiation. As depicted in FIGS. 8-11, the HFB2050 cell line (Sox-2+ and nestin+) can proliferate and persist after many passages, demonstrating self-renewal. In some embodiments, the HFB2050 can differentiate into three cardinal neural cell types in a stable ratio after prolonged passaging. As illustrated in FIGS. 6-8, confluent, contact-inhibited HFB2050 hNSCs have spontaneously differentiated into astrocyte (e.g., GFAP+ and/or EAAT1+), neurons (Tuj1+), and oligodendrocytes (oligo2+). In some embodiments, the hNSCs become contact-inhibited by about 2-3 weeks after seeding upon reaching confluency. In some embodiments, the HFB2050 hNSCs are negative for all pluripotency markers (e.g., Oct-4, NANOG, TRA1-81, TRA1-61, and SSEA4), as illustrated by FIG. 12. In some embodiments, the HFB2050 hNSCs demonstrate stable karyotype after many passages. As illustrated by FIG. 13, HFB2050 hNSCs have a normal human female karyotype. In some embodiments, the HFB2050 can be cryopreserved and retain characteristics upon thaw and return to culture. As illustrated by FIG. 14, after freeze-thaw ≥80% viable cells were recovered and could be further maintained and passaged, achieving the same growth kinetics and the differentiation profile, and express the same multipotency and self-renewal protein markers.

In some embodiments, the NSCs are genetically stable and unmodified. In some embodiments, the NSCs can be genetically modified. In some embodiments, the NSCs can be genetically modified to include a transgene. In some embodiments, the transgene is Sox 2 or Nestin. In some embodiments, the NSCs are genetically modified by common gene editing tools that are known to those skilled in the art. A gene editing tool comprises vector, e.g., viral vector, for gene editing based on CRISPR-Cas9, TALEN, Zinc Finger, or other applicable technologies. In some embodiments, the NSCs are genetically modified to maintain and/or enhance certain characteristics of the NSCs, including the ability to integrate into the human subject, the ability to self-renew, and the ability to differentiate into other cell types. In some embodiments, the NSCs are genetically modified to maintain and/or enhance its effectiveness in treating perinatal HII.

### Treatment of Subjects

### Indication

Provided herein are methods, compositions, and kits related to treating perinatal hypoxic-ischemic injuries (HII). Clinical features of HII can include abnormal state of consciousness, reduced spontaneous movements, respiratory difficulties, altered tone, abnormal primitive reflexes, and/or seizure activities. Other aspects of consideration in diagnosis can include blood gas values, Apgar score, physical examination, onset of multisystem organ failure, evaluation of the placenta and umbilical cord, neuroimaging, and EEG. In some embodiments, the methods, compositions, and kits provided are used to treat HII after a formal diagnosis of HII is made by a medical professional skilled in the art. In some embodiments, the methods, compositions, and kits provided are used when a formal diagnosis of HII is suspected but not confirmed despite of presentation of clinical features of HII such as signs of brain injuries. Assessment of HII stage can initiate as soon as possible after the neonate is resuscitated and stabilized, and can continue for at least 6 hours.

The Sarnat scoring system can be used to assess the severity of encephalopathy, upon which HII staging can be based. Sarnat staging can include evaluation of neuromuscular control, complex reflexes, autonomic functions, seizures, and EEG findings. HII can be classified or staged using a Sarnat scoring scale. In some embodiments, HII stages are not absolute and can change as a neonate's condition progresses or recovers. In some embodiments, HII is not staged. In some embodiments, HII is staged as Sarnat 1 Stage 1 (mild), Stage 2 (moderate), or Stage 3 (severe). In some embodiments, HII is staged between two stages, such as Stage 2 (moderate) to Stage 3 (severe). HII can be diagnosed with other comorbidities comprising congenital abnormalities, metabolic disorders, organ failure, and/or meningitis. In some embodiments, the methods, compositions, and kits provided herein are used to treat HII without other comorbidities. In some embodiments, the methods, compositions, and kits provided herein are used to treat HII with other comorbidities.

Another aspect provides methods and compositions for treating perinatal HII in combination with a standard of care treatment (e.g., therapeutic hypothermia, HT). Another aspect provides methods and compositions for treating perinatal HII in combination with a supportive therapy. In some embodiments, the supportive therapy for HII comprises seizure medications, inhaled nitric oxide, high frequency ventilation, extracorporeal membrane oxygenation therapies, volume replacement, hemodialysis, inotropic agents, vitamins, and/or erythropoietin.

Effect of hNSCs on HII

hNSCs can have a different effect on HII treatment from a standard of care hypothermia therapy. In some embodiments, the hNSCs demonstrate cell engraftment, migration, and/or differentiation into region-appropriate electrophysiologically-active neurons, or cytokine- or myelin-elaborating glia. The hNSCs provided herein can present pathotropism. In some embodiments, the hNSCs can preferentially home to the ischemic region of a central nervous system (CNS) affected by ischemic injury. In some embodiments, the hNSCs can integrate into the periventricular germinal zone in a human subject. After integration, the hNSCs can self renew and/or differentiate into one of the three main cell types of the CNS (e.g., neurons, astrocytes, and oligodendrocytes). Without limited to any particular theory, the hNSC can affect HII treatment by a paracrine mechanism (e.g., the “Chaperone Effect”), rescuing the penumbra of the ischemic injury, circuit repair, neo-circuit creation, and/or remyelination. In some embodiments, the hNSCs and cells differentiated from hNSCs (e.g., neurons, oligodendrocytes, and astrocytes) produce a range of cytokines. In some embodiments, the cytokines produced comprise neuroprotective agents (e.g., glial cell line-derived neurotrophic factor (GDNF), brain-derived neurotrophic factor (BDNF), nerve growth factor (NGF), neurotrophin 3 (NT-3)), pro-angiogenic cytokines (e.g., vascular endothelial growth factor (VEGF)), and/or anti-inflammatory cytokines or agents (e.g., Interleukin 6 (IL-6), nitric oxide (NO), prostaglandin E2 (PGE2)). In some embodiments, the hNSCs form gap junctions with host cells in a subject receiving the treatment and compositions provided herein. In some embodiments, the hNSCs produce an extracellular matrix. The hNSCs can provide direct neuroprotection and/or trophic support via diffusible factors, gap junctions, exosomes; scavenging ROS; reducing inflammation and scarring; promoting angiogenesis; repairing the blood-brain barrier; mobilizing endogenous NSCs; promoting endogenous neurite outgrowth; replacing interneurons; providing glial support, including astrocytes and oligodendrocytes; providing extracellular matrix; altering the niche; and/or restoring normal metabolism to injured host neural cells.

In some embodiments, the methods and compositions provided herein are more effective than an HT in minimizing cerebral damage from HII. In some embodiments, the methods and compositions provided herein have additive or synergistic effects with HT in minimizing cerebral damage from HII. In some embodiments, the compositions to be administered have no to minimal side effects, including non-tumorigenic and non-immunogenic.

### Subjects

Any of the compositions provided herein can be administered to an individual. “Individual” can be used interchangeably with “subject” or “patient.” In some embodiments, the individual is a human neonate. In some embodiments, the human neonate is a pre-term, full-term, or post-term baby. In some embodiments, the human neonate is a full-term or post-term baby (e.g., greater, or equal to 37 weeks gestation). In some embodiments, the human neonate is diagnosed with perinatal hypoxic-ischemic injuries (HII). In some embodiments, the human neonate is diagnosed with Sarnat Stage 1 (mild) HII. In some embodiments, the human neonate is diagnosed with Sarnat Stage 2 (moderate) HII. In some embodiments, the human neonate is diagnosed with Sarnat Stage 3 (severe) HII. In some embodiments, the human neonate is diagnosed with Sarnat Stage 2 (moderate) to Sarnat Stage 3 (severe) HII. In some embodiments, the human neonate progresses or regresses in their HII Sarnat staging. In some embodiments, the human neonate can show signs and symptoms of asphyxia or hypoxic brain injuries without a formal diagnosis of HII. In some embodiments, the human neonate can have comorbidities, anomalies, or dysmorphologies with signs of hypoxic brain injuries. In some embodiments, the comorbidities comprise congenital abnormalities, metabolic disorders, organ failure, and/or meningitis. In some embodiments, the neonate is eligible for a hypothermia therapy (HT). In some embodiments, the neonate who qualifies for an HT completes the HT. In some embodiments, the neonate who qualifies for an HT does not receive or complete the HT. In some embodiments, the neonate does not qualify for a hypothermia therapy (HT) and does not receive the HT. In some embodiments, the neonate has an Apgar score of equal or less than 5 at 10 min after birth and continue to need resuscitation at 10 minutes. In some embodiments, the neonate has a blood pH lower than 7.00 or a base deficit of greater or equal to 16.0 mmol/L, indicated by an umbilical arterial or venous blood sample prenatally, or by a venous, arterial, or capillary blood sample within 60 minutes of birth. In some embodiments, the neonate presents encephalopathy signs comprising lethargy, stupor, or coma, and/or hypotonia, abnormal reflexes, absent/week sucking, and seizures. Administration

Another aspect provides a pharmaceutical composition comprising hNSCs or progenitors thereof to be administered to a human subject in need thereof. The composition comprising hNSCs can be administered after the birth of the neonate with or without the administration of a hypothermia therapy (e.g., HT). In some embodiments, the hNSCs are administered within 6 hours of birth of a neonate, including prior to commencing a hypothermia therapy (e.g., HT). In some embodiments, the hNSCs are administered after 6 hours of birth of a neonate, including upon or after the completion of an HT. In some embodiments, the hNSCs are administered concurrently or sequentially with an HT.

The composition comprising hNSCs or progenitors thereof can be administered from 0.5 to 15 days after birth. In some embodiments, the composition comprising hNSCs can be administered 0.5 to 15 days after the birth of the neonate. In some embodiments, the composition comprising hNSCs can be administered at least 0.5, at least 1, at least 2, at least 3, at least 4, at least 5, at least 6, at least 7, at least 8, at least 9, at least 10, at least 11, at least 12, at least 13, at least 14, or at least 15 days after birth of a neonate, or a time point within a range defined by any of the preceding values. In some embodiments, the composition comprising hNSCs can be administered about 0.5, about 1, about 2, about 3, about 4, about 5, about 6, about 7, about 8, about 9, about 10, about 11, about 12, about 13, about 14, or about 15 days after the birth of a neonate. In some embodiments, the composition comprising hNSCs can be administered 0.5, 1, 2, 3, 4, 5, 6, 7, 8, 9, or 10 days after the birth of a neonate, or a time point within a range defined by any of the preceding values. In some embodiments, the composition comprising hNSCs can be administered about 2.5, about 3.0, or about 3.5 days after the birth of a neonate, or a time point within a range defined by any of the preceding values. In some embodiments, the composition comprising hNSCs can be administered about 2.5 days after the birth of a neonate. In some embodiments, the composition comprising hNSCs can be administered about 3 days after the birth of a neonate. In some embodiments, the composition comprising hNSCs can be administered 3 days after the birth of a neonate. In some embodiments, the composition comprising hNSCs can be administered 2.5, 3, or 3.5 days after the birth of a neonate, or a time point within a range defined by any of the preceding values. In some embodiments, the composition comprising hNSCs is administered to the subject from 0.5 days to about 15 days, from 0.5 days to about 12 days, from 0.5 days to about 10 days, from 0.5 days to about 9 days, from 0.5 days to about 8 days, from 0.5 days to about 7 days, from 0.5 days to about 6 days, from 0.5 days to about 5 days, from 0.5 days to about 4 days, from 0.5 days to about 3 days, from 0.5 days to about 2 days, or from 0.5 days to about 1 day after birth. In some embodiments, the composition comprising hNSCs is administered to the subject from 0.5 days to about 10 days, from 0.5 days to about 9 days, from 0.5 days to about 8 days, from 0.5 days to about 7 days, from 0.5 days to about 6 days, from 0.5 days to about 5 days, from 0.5 days to about 4 days, from 0.5 days to about 3 days, from 0.5 days to about 2 days, or from 0.5 days to about 1 day after birth of the neonate. In some embodiments, the composition comprising hNSCs is administered to the subject from 0.5 days to about 6 days, from 0.5 days to about 5 days, from 0.5 days to about 4 days, from 0.5 days to about 3 days, from 0.5 days to about 2 days, or from 0.5 days to about 1 day after birth. In some embodiments, the composition comprising hNSCs is administered to the subject from about 1 day to about 15 days, from about 1 day to about 12 days, from about 1 day to about 10 days, from about 1 day to about 9 days, from about 1 day to about 8 days, from about 1 day to about 7 days, from about 1 day to about 6 days, from about 1 day to about 5 days, from about 1 day to about 4 days, from about 1 day to about 3 days, or from about 1 day to about 2 days after birth. In some embodiments, the composition comprising hNSCs is administered to the subject from about 1 day to about 4 days after birth. In some embodiments, the composition comprising hNSCs is administered to the subject from about 2 days to about 4 days after birth. In some embodiments, the composition comprising hNSCs is administered to the subject from about 2 days to about 15 days, from about 2 days to about 12 days, from about 2 days to about 10 days, from about 2 day to about 9 days, from about 2 days to about 8 days, from about 2 days to about 7 days, from about 2 days to about 6 days, from about 2 days to about 5 days, from about 2 days to about 4 days, or from about 2 days to about 3 days after birth. In some embodiments, the composition comprising hNSCs is administered to the subject from about 3 days to about 15 days, from about 3 days to about 12 days, from about 3 days to about 10 days, from about 3 days to about 9 days, from about 3 days to about 8 days, from about 3 days to about 7 days, from about 3 days to about 6 days, from about 3 days to about 5 days, or from about 3 days to about 4 days after birth. In some embodiments, the composition comprising hNSCs is administered to the subject from about 4 days to about 15 days, from about 4 days to about 12 days, from about 4 days to about 10 days, from about 4 days to about 9 days, from about 4 days to about 8 days, from about 4 days to about 7 days, from about 4 days to about 6 days, or from about 4 days to about 5 days after birth. In some embodiments, the composition comprising hNSCs is administered to the subject from about 5 days to about 15 days, from about 5 days to about 12 days, from about 5 days to about 10 days, from about 5 days to about 9 days, from about 5 days to about 8 days, from about 5 days to about 7 days, or from about 5 days to about 6 days after birth of the neonate. In some embodiments, the composition comprising hNSCs is administered to the subject from about 6 days to about 15 days, from about 6 days to about 12 days, from about 6 days to about 10 days, from about 6 days to about 9 days, from about 6 days to about 8 days, or from about 6 days to about 7 days after birth of the neonate. In some embodiments, the composition comprising hNSCs is administered to the subject from about 7 days to about 15 days, from about 7 days to about 12 days, from about 7 days to about 10 days, from about 7 days to about 9 days, or from about 7 days to about 8 days after birth. In some embodiments, the composition comprising hNSCs is administered to the subject about 3 days after birth. In some embodiments, the composition comprising hNSCs is administered to the subject 3 days after birth. In some embodiments, the composition comprising hNSCs can be administered to the subject 2.5, 3, or 3.5 days after birth, or a time point within a range defined by any of the preceding values.

The composition comprising hNSCs or progenitors thereof can be administered from 0.5 to 15 days post hypoxic-ischemic brain injuries. In some embodiments, the composition comprising hNSCs can be administered at least 1, at least 2, at least 3, at least 4, at least 5, at least 6, at least 7, at least 8, at least 9, at least 10, at least 11, at least 12, at least 13, at least 14, or at least 15 days post hypoxic-ischemic brain injuries. In some embodiments, the composition comprising hNSCs can be administered at about 1, about 2, about 3, about 4, about 5, about 6, about 7, about 8, about 9, about 10, about 11, about 12, about 13, about 14, and about 15 days post hypoxic-ischemic brain injuries. In some embodiments, the composition comprising hNSCs is administered to the subject from the time of hypoxic-ischemic brain injuries to about 15 days, from the time of hypoxic-ischemic brain injuries to about 12 days, from the time of hypoxic-ischemic brain injuries to about 10 days, from the time of hypoxic-ischemic brain injuries to about 9 days, from the time of hypoxic-ischemic brain injuries to about 8 days, from the time of hypoxic-ischemic brain injuries to about 7 days, from the time of hypoxic-ischemic brain injuries to about 6 days, from the time of hypoxic-ischemic brain injuries to about 5 days, from the time of hypoxic-ischemic brain injuries to about 4 days, from the time of hypoxic-ischemic brain injuries to about 3 days, from the time of hypoxic-ischemic brain injuries to about 2 days, or from the time of hypoxic-ischemic brain injuries to about 1 day post hypoxic-ischemic brain injuries. In some embodiments, the composition comprising hNSCs is administered to the subject from about 1 day to about 15 days, from about 1 day to about 12 days, from about 1 day to about 10 days, from about 1 day to about 9 days, from about 1 day to about 8 days, from about 1 day to about 7 days, from about 1 day to about 6 days, from about 1 day to about 5 days, from about 1 day to about 4 days, from about 1 day to about 3 days, or from about 1 day to about 2 days post hypoxic-ischemic brain injuries. In some embodiments, the composition comprising hNSCs is administered to the subject from about 2 days to about 15 days, from about 2 days to about 12 days, from about 2 days to about 10 days, from about 2 day to about 9 days, from about 2 days to about 8 days, from about 2 days to about 7 days, from about 2 days to about 6 days, from about 2 days to about 5 days, from about 2 days to about 4 days, or from about 2 days to about 3 days post hypoxic-ischemic brain injuries. In some embodiments, the composition comprising hNSCs is administered to the subject from about 3 days to about 15 days, from about 3 days to about 12 days, from about 3 days to about 10 days, from about 3 days to about 9 days, from about 3 days to about 8 days, from about 3 days to about 7 days, from about 3 days to about 6 days, from about 3 days to about 5 days, or from about 3 days to about 4 days post hypoxic-ischemic brain injuries. In some embodiments, the composition comprising hNSCs is administered to the subject from about 4 days to about 15 days, from about 4 days to about 12 days, from about 4 days to about 10 days, from about 4 days to about 9 days, from about 4 days to about 8 days, from about 4 days to about 7 days, from about 4 days to about 6 days, or from about 4 days to about 5 days post hypoxic-ischemic brain injuries. In some embodiments, the composition comprising hNSCs is administered to the subject from about 5 days to about 15 days, from about 5 days to about 12 days, from about 5 days to about 10 days, from about 5 days to about 9 days, from about 5 days to about 8 days, from about 5 days to about 7 days, or from about 5 days to about 6 days post hypoxic-ischemic brain injuries. In some embodiments, the composition comprising hNSCs is administered to the subject from about 6 days to about 15 days, from about 6 days to about 12 days, from about 6 days to about 10 days, from about 6 days to about 9 days, from about 6 days to about 8 days, or from about 6 days to about 7 days post hypoxic-ischemic brain injuries. In some embodiments, the composition comprising hNSCs is administered to the subject from about 7 days to about 15 days, from about 7 days to about 12 days, from about 7 days to about 10 days, from about 7 days to about 9 days, or from about 7 days to about 8 days post hypoxic-ischemic brain injuries.

The composition comprising hNSCs or progenitors thereof can be administered from 0.5 to 15 days post a diagnosis of hypoxic-ischemic brain injuries. In some embodiments, the composition comprising hNSCs can be administered at the time of the diagnosis of the hypoxic-ischemic brain injuries. In some embodiments, the composition comprising hNSCs can be administered at least 1, at least 2, at least 3, at least 4, at least 5, at least 6, at least 7, at least 8, at least 9, at least 10, at least 11, at least 12, at least 13, at least 14, or at least 15 days post diagnosis of hypoxic-ischemic brain injuries. In some embodiments, the composition comprising hNSCs can be administered at about 1, about 2, about 3, about 4, about 5, about 6, about 7, about 8, about 9, about 10, about 11, about 12, about 13, about 14, and about 15 days post diagnosis of hypoxic-ischemic brain injuries. In some embodiments, the composition comprising hNSCs is administered to the subject from the time of the diagnosis of hypoxic-ischemic brain injuries to about 15 days, from the time of the diagnosis of hypoxic-ischemic brain injuries to about 12 days, from the time of the diagnosis of hypoxic-ischemic brain injuries to about 10 days, from the time of the diagnosis of hypoxic-ischemic brain injuries to about 9 days, from the time of the diagnosis of hypoxic-ischemic brain injuries to about 8 days, from the time of the diagnosis of hypoxic-ischemic brain injuries to about 7 days, from the time of the diagnosis of hypoxic-ischemic brain injuries to about 6 days, from the time of the diagnosis of hypoxic-ischemic brain injuries to about 5 days, from the time of the diagnosis of hypoxic-ischemic brain injuries to about 4 days, from the time of the diagnosis of hypoxic-ischemic brain injuries to about 3 days, from the time of the diagnosis of hypoxic-ischemic brain injuries to about 2 days, or from the time of the diagnosis of hypoxic-ischemic brain injuries to about 1 day post the diagnosis of hypoxic-ischemic brain injuries. In some embodiments, the composition comprising hNSCs is administered to the subject from about 1 day to about 15 days, from about 1 day to about 12 days, from about 1 day to about 10 days, from about 1 day to about 9 days, from about 1 day to about 8 days, from about 1 day to about 7 days, from about 1 day to about 6 days, from about 1 day to about 5 days, from about 1 day to about 4 days, from about 1 day to about 3 days, or from about 1 day to about 2 days post the diagnosis of hypoxic-ischemic brain injuries. In some embodiments, the composition comprising hNSCs is administered to the subject from about 2 days to about 15 days, from about 2 days to about 12 days, from about 2 days to about 10 days, from about 2 day to about 9 days, from about 2 days to about 8 days, from about 2 days to about 7 days, from about 2 days to about 6 days, from about 2 days to about 5 days, from about 2 days to about 4 days, or from about 2 days to about 3 days post the diagnosis of hypoxic-ischemic brain injuries. In some embodiments, the composition comprising hNSCs is administered to the subject from about 3 days to about 15 days, from about 3 days to about 12 days, from about 3 days to about 10 days, from about 3 days to about 9 days, from about 3 days to about 8 days, from about 3 days to about 7 days, from about 3 days to about 6 days, from about 3 days to about 5 days, or from about 3 days to about 4 days post the diagnosis of hypoxic-ischemic brain injuries. In some embodiments, the composition comprising hNSCs is administered to the subject from about 4 days to about 15 days, from about 4 days to about 12 days, from about 4 days to about 10 days, from about 4 day to about 9 days, from about 4 days to about 8 days, from about 4 days to about 7 days, from about 4 days to about 6 days, or from about 4 days to about 5 days post the diagnosis of hypoxic-ischemic brain injuries. In some embodiments, the composition comprising hNSCs is administered to the subject from about 5 days to about 15 days, from about 5 days to about 12 days, from about 5 days to about 10 days, from about 5 days to about 9 days, from about 5 days to about 8 days, from about 5 days to about 7 days, or from about 5 days to about 6 days post the diagnosis of hypoxic-ischemic brain injuries. In some embodiments, the composition comprising hNSCs is administered to the subject from about 6 days to about 15 days, from about 6 days to about 12 days, from about 6 days to about 10 days, from about 6 days to about 9 days, from about 6 days to about 8 days, or from about 6 days to about 7 days post the diagnosis of hypoxic-ischemic brain injuries. In some embodiments, the composition comprising hNSCs is administered to the subject from about 7 days to about 15 days, from about 7 days to about 12 days, from about 7 days to about 10 days, from about 7 days to about 9 days, or from about 7 days to about 8 days post the diagnosis of hypoxic-ischemic brain injuries.

The composition comprising hNSCs or progenitors thereof can be administered concurrently or subsequently with an HT treatment. In some embodiments, the comprising hNSCs is administered without an HT treatment.

In some embodiments, the composition comprising hNSCs or progenitors thereof is administered within 10 hours, 9 hours, 8 hours, 7 hours, 6 hours, 5 hours, 4 hours, 3 hours, 2 hours, or 1 hour after the birth of the subject. In some embodiments, the composition comprising hNSCs is administered within 6 hours after the birth of the subject.

In some embodiments, the composition comprising hNSCs or progenitors thereof is administered prior to the start of the HT treatment. In some embodiments, the composition comprising hNSCs is administered after the HT treatment is completed. In some embodiments, the composition comprising hNSCs is administered during the HT treatment. In some embodiments, the HT treatment starts immediately after the birth of the subject. In some embodiments, the HT treatment starts within 10 hours, 9 hours, 8 hours, 7 hours, 6 hours, 5 hours, 4 hours, 3 hours, 2 hours, or 1 hour after the birth of the subject. In some embodiments, the HT treatment starts within 6 hours after birth of the subject. In some embodiments, the HT treatment starts within 7 hours after the birth of the subject. In some embodiments, the HT treatment starts about 6 hours after the birth of the subject. In some embodiments, the HT treatment starts 6 hours after the birth of the subject. The HT treatment can last about 1 day, about 2 days, about 3 days, about 4 days, or about 5 days. In some embodiments, the HT treatment lasts about 3 days. In some embodiments, the HT is a whole-body HT. In some embodiments, the subject receives a full HT (i.e., a completed HT). In some embodiments, the subject receives a partial HT (i.e., an incomplete HT). In some embodiments, the composition comprising hNSCs is administered to the subject immediately after the HT treatment starts. In some embodiments, the composition comprising hNSCs is administered to the subject about 1 day, about 2 days, about 3 days, about 4 days, or about 5 days after the HT treatment starts. In some embodiments, the composition comprising hNSCs is administered to the subject immediately after the HT treatment ends. In some embodiments, the composition comprising hNSCs is administered to the subject about 1 day, about 2 days, about 3 days, about 4 days, or about 5 days after the HT treatment ends.

Another aspect provides methods of administering the compositions comprising or consisting of NSCs into a central nervous system (CNS) of a neonate. In some embodiments, the methods of administering comprise administering NSCs into a cerebral ventricle of a neonate. In some embodiments, the methods of administering comprise administering NSCs via intracerebral instillation through a fontanelle of a neonate. In some embodiments, the methods of administering the cells are compatible and/or suitable for administering a second agent into the CNS of a neonate. In some embodiments, the methods of administering the NSCs comprising administering a second therapeutic agent in combination with the NSCs, such as a second type of cells or an antimicrobial agent. In some embodiments, the second therapeutic agent is an antibiotic or an immunosuppressive agent. Exemplary antibiotics include mupirocin, defensin, gentamycin, geneticin, cefmenoxime, penicillin, streptomycin, xylitol, or other antibiotics to assist in protecting the subject who is receiving the NSCs or progenitors thereof. Exemplary immunosuppressive agents include cyclosporine A, tacrolimus, prednisolone, azathioprine, methylprednisolone, mycophenolate mofetil, or sirolimus. The immunosuppressive agents can also comprise genetically engineered cells. The second therapeutic agent can be combined with pre-, co-, or post-treatment with the NSCs or progenitor thereof. In some embodiments, the methods of administering the NSCs are used in combination with a second compatible procedure, such as removing/aspirating excess cerebrospinal fluid (CSF) to reduce intracranial pressure.

In some embodiments, the methods of administering the NSCs comprises a minimally invasive procedure, such as an anterior fontanelle tap (e.g., AF tap). In some embodiments, the methods of administering the NSCs comprises instillation of the NSCs to the cerebral ventricles via an AF tap. In some embodiments, the cerebral ventricles comprise a lateral ventricle. The NSCs provided herein can be administered using a catheter or a needle of appropriate caliber for administering the NSCs provided herein. In some embodiments, the NSCs provided herein are administered using a butterfly needle. In some embodiments, the NSCs provided herein are administered using a 21 or 23-gauge catheter or a needle. In some embodiments, the NSCs provided herein are administered using an 18 to 27-gauge catheter or needle. In some embodiments, the NSCs provided herein are administered using a 24-gauge catheter or needle. In some embodiments, the NSCs provided herein are administered using a 30 to 34-gauge catheter or a needle. The needle or catheter can be inserted into the anterior fontanelle of a subject the appropriate depth for administering the NSCs provided herein. In some embodiments, the needle or catheter can be inserted about 2 cm into the anterior fontanelle of a subject. In some embodiments, the inserting of a needle or catheter can be monitored and/or guided, such as by a cranial ultrasound. In some embodiments, the needle or catheter is inserted into the dilated lateral ventricle unilaterally via the fontanelle tap under direct ultrasound guidance. In some embodiments, the NSCs are injected into the lateral ventricle by a syringe connected to the catheter.

In some embodiments, the methods of administering the NSCs comprises inserting a needle into the non-bony anterior fontanelle (AF). The needle may be inserted into the AF to a depth of about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, or about 2.5 cm. The needle may be inserted into the AF to a depth of at least about 1.5 cm, at least about 1.6 cm, at least about 1.7 cm, at least about 1.8 cm, at least about 1.9 cm, at least about 2.0 cm, at least about 2.1 cm, at least about 2.2 cm, at least about 2.3 cm, at least about 2.4 cm, or at least about 2.5 cm. The needle may be inserted into the AF to a depth of at most about 1.5 cm, at most about 1.6 cm, at most about 1.7 cm, at most about 1.8 cm, at most about 1.9 cm, at most about 2.0 cm, at most about 2.1 cm, at most about 2.2 cm, at most about 2.3 cm, at most about 2.4 cm, or at most about 2.5 cm.

The administration of the NSCs provided herein can further comprise surgical procedures appropriate for the administration of the therapeutic agents into the central nervous system of a human subject. In some embodiments, the surgical procedures can be administering anesthesia, applying dressing, flushing administration tubing with buffer, adjusting volume of cerebrospinal fluid (CSF), adjusting cranial pressure (CP), or a combination thereof. In some embodiments, the methods of administering the NSCs comprise (i) inserting a needle or a catheter percutaneously into a cerebral lateral ventricle through an anterior fontanelle (AF) via an AF tap; (ii) aspirating a volume of cerebrospinal fluid (CSF) equivalent to that of the hNSCs to be administered from the catheter; (iii) instilling the hNSCs into the AF; (iv) flushing the catheter with buffer; (v) removing the catheter; (v) compressing the area of puncture and applying an aseptic dressing; and (vi) optionally repeating steps (i) to (v) on ipsilateral side. In some embodiments, the needle or the catheter is a 24-gauge needle or catheter. In some embodiments, the needle or the catheter is a 30 to 34-gauge needle or catheter. In some embodiments, the needle or the catheter is a 30 or 34-gauge needle or catheter. In some embodiments, the buffer used to flush the catheter is suitable for administering into the CNS, such as normal saline or artificial CSF. In some embodiments, the NSCs are administered to only one hemisphere of the brain. The methods of administering the NSCs can be performed with or without general anesthesia. In some embodiments, the minimally invasive procedure is performed without general anesthesia. In some embodiments, the minimally invasive procedure can be performed with or without local anesthesia. In some embodiments, the minimally invasive procedure is performed with local anesthesia. In some embodiments, the method further comprises transporting the hNSCs at wet-ice temperature (e.g., about 0° C.), optionally the hNSCs are in a vial or syringe.

In some embodiments, the methods of administering the NSCs can be performed within a short period of time, such as about 5 minutes, about 10 minutes, about 15 minutes, or about 20 minutes. The administration provided herein can be performed at a rate suitable for instillation of the cells provided herein. In some embodiments, the cells, composition, and formulation comprising hNSCs or progenitors thereof are administered at about 0.5 mL/minute, about 1 mL/minute, or about 1.5 mL/minute. In some embodiments, the methods of administering the NSCs is performed once. In some embodiments, the methods of administering the NSCs is performed more than once on one or more occasions. For example, the methods comprise administering the NSCs 2 times, 3 times, 4 times, 5 times, 6 times, 7 times, 8 times, 9 times, or 10 times during the course of treatment.

In some embodiments, the amount of suspension containing the NSCs injected is at most 1 mL, at most 2 mL, at most 3 mL, at most 4 mL, at most 5 mL, at most 6 mL, at most 7 mL, or at most 8 mL per ventricle. In some embodiments, the amount of suspension containing the NSCs injected is at least 0.1 mL, at least 0.2 mL, at least 0.3 mL, at least 0.4 mL, at least 0.5 mL, at least 0.6 mL, at least 0.7 mL, at least 0.8 mL, at least 0.9 mL, at least 1 mL, at least 2 mL, at least 3 mL, at least 4 mL, at least 5 mL, or at least 6 mL per ventricle. In some embodiments, the amount of suspension containing the NSCs injected is about 1 mL, about 1.5 mL, about 2 mL, about 2.5 mL, about 3 mL, about 3.5 mL, about 4 mL, about 4.5 mL, about 5 mL, about 5.5 mL, about 6 mL, about 6.5 mL, or about 7 mL per ventricle.

The administration of NSCs may not alter or effect the usual care and monitoring of a neonate being treated. In some embodiments, the methods of administering the NSCs can be performed at the same time of usual care and monitoring of a neonate being treated, such as a magnetic resonance imaging (MRI), EEG and serial neurological assessments. In some embodiments, the methods of administering the NSCs further comprise being guided and/or monitored in real-time, such as by a cranial ultrasound.

In some embodiments, the method further comprises administering a magnetic resonance imaging (MRI) diagnostic test, wherein the results of the MRI inform treatment parameters. In some embodiments, the method further comprises administering a magnetic resonance imaging (MRI) diagnostic test, wherein the results of the MRI inform treatment parameters. In some embodiments, the method further comprises analyzing the MRI, by a method disclosed in U.S. Pat. No. 8,965,089 B2 incorporated herein by reference in its entirety. In some embodiments, the MRI comprises one or more than one region of interest. In some embodiments, analyzing the MRI comprises: configuring at least one processor to perform the functions of: 1) providing the MRI comprising a set of actual image values; 2) rescaling the actual image values to produce corresponding rescaled image values and to produce a rescaled image from the rescaled image values; 3) deriving a histogram of the rescaled image values; 4) using the histogram to derive an adaptive segmentation threshold that can be used to split the rescaled image into two sub-images, a first sub-image with intensities at or below the adaptive segmentation threshold and a second sub-image with intensities above the adaptive segmentation threshold, or a first sub-image with intensities below the adaptive segmentation threshold and a second sub-image with intensities at or above the adaptive segmentation threshold; 5) using the adaptive segmentation threshold to recursively split the rescaled image to generate a Hierarchical Region Splitting Tree of sub(sub) images based on consistency of the rescaled image values of the rescaled image; 6) terminating the recursive splitting of the sub(sub) images using one or more than one predetermined criteria thereby completing the Hierarchical Region Splitting Tree; and 7) identifying one sub(sub) image in the terminated Hierarchical Region Splitting Tree which comprises the region of interest. In some embodiments, analyzing the MRI comprises:
a) providing the MRI comprising a set of actual image values;
b) rescaling the actual image values to produce corresponding rescaled image values and to produce a rescaled image from the rescaled image values;
c) deriving a histogram of the rescaled image values;
d) using the histogram to derive an adaptive segmentation threshold that can be used to split the rescaled image into two sub-images, a first sub-image with intensities at or below the adaptive segmentation threshold and a second sub-image with intensities above the adaptive segmentation threshold, or a first sub-image with intensities below the adaptive segmentation threshold and a second sub-image with intensities at or above the adaptive segmentation threshold, or a first sub-image with intensities below the adaptive segmentation threshold and a second sub-image with intensities above the adaptive segmentation threshold;
e) using the adaptive segmentation threshold to recursively split the rescaled image to generate a Hierarchical Region Splitting Tree of sub(sub) images based on consistency of the rescaled image values of the rescaled image;
f) terminating the recursive splitting of the sub(sub) images using one or more than one predetermined criteria thereby completing the Hierarchical Region Splitting Tree; and
g) identifying one sub(sub) image in the terminated Hierarchical Region Splitting Tree which comprises the region of interest;
the method further comprising performing a secondary rescaling of the rescaled image values of every rescaled sub(sub) image in the Hierarchical Region Splitting Tree back to the actual image values present in the MRI to create a secondary rescaled MRI, thereby producing a secondarily rescaled sub(sub) image comprising the region of interest;
where the rescaled image values fit in [0,255] unsigned 8-bit integer range;
where the predetermined criteria are selected from the group consisting of area threshold=50 pixels and (standard deviation threshold=10 rscVals (StdDevTh=10 rscVals) and kurtosis threshold=1.5);
where the region of interest is a representation of an abnormality in the living human tissue, and
where the method further comprises quantifying the abnormality in the living human tissue;
where the method further comprises performing a secondary rescaling of the rescaled image values in every rescaled sub(sub) image in the Hierarchical Region Splitting Tree back to the actual image values present in the MRI to create a secondary rescaled MRI, and determining an image value or a set of image values of actual image values in the MRI after the secondary rescaling, where the image value or a set of image values of actual image values determined identifies the abnormality represented in the MRI for the modality being used to generate the MRI; and where the method further comprises preparing a mask of the sub(sub) image containing the representation of the abnormality, and cleaning the mask to remove small outlier regions to generate a cleaned mask of the sub(sub) image containing the representation of the abnormality. In some embodiments, the one sub(sub) image in the terminated Hierarchical Region Splitting Tree comprising the region of interest is two-dimensional. In some embodiments, the one sub(sub) image in the terminated Hierarchical Region Splitting Tree comprising the region of interest is three-dimensional. In some embodiments, the method further comprising analyzing the MRI, wherein analyzing the MRI comprises: measuring of water content and of movement (diffusion) of water molecules within brain tissues. In some embodiments, the measuring of water content and of movement (diffusion) of water molecules within brain tissues provides a data set. In some embodiments, the method further comprises calculating average diffusion coefficient (ADC) maps using the data set. In some embodiments, the method further comprises determining an injury state of the human subject. In some embodiments, the injury state informs a treatment protocol. The MRI can be performed before, during, or after a treatment with HT.

### Dosing

The method provided herein comprises administering an effective amount of the NSCs provided herein (e.g., HFB2050 hNSCs) or progenitors thereof to treat perinatal HII. The effective amount of NSCs comprises any dose of NSCs (e.g., HFB2050 hNSCs) sufficient for treating perinatal HII. In some embodiments, the effective amount of NSCs can be 1 dose, 2 doses, 3 doses, 4 dose, 5 doses, 6 doses, 7 dose, 8 doses, 9 doses, or 10 dose of the cells and/or compositions provided herein.

A dose of cells comprising HFB2050 hNSCs administered to a subject can be based on the subject's body weight (e.g., number of cells per kg of body weight). In some embodiments, the body weight of a subject (e.g., neonate) can be the actual body weight, ideal body weight, and/or adjusted body weight of the subject. A dose of cells comprising HFB2050 hNSCs administered to a subject can be administered to one or more ventricles of the subject. In some embodiments, the dose of cells comprising HFB2050 hNSCs is administered to one ventricle of the subject. (e.g., number of cells per ventricle). In some embodiments, the dose of cells comprising HFB2050 hNSCs is measured by number of cells per kg of body weight of a subject provided herein per ventricle administered (e.g., number of cells/kg/ventricle).

The NSCs can be suspended at about 1×10⁷, about 2×10⁷, about 3×10⁷, about 4×10⁷, about 5×10⁷, about 6×10⁷, about 7×10⁷, about 8×10⁷, about 9×10⁷, about 1×10⁸ cells/mL in a buffer (e.g., normal saline). In some embodiments, the NSCs can be suspended at about 4×10⁷ cells/mL in a buffer. In some embodiments, the NSCs can be suspended at about 5×10⁷ cells/mL in a buffer. In some embodiments, the NSCs can be suspended from about 2×10⁷ to about 7×10⁷ cells/mL in a buffer. In some embodiments, the NSCs can be suspended from about 2.5×10⁷ to about 6.5×10⁷ cells/mL in a buffer. In some embodiments, the NSCs can be suspended from about 3×10⁷ to about 6×10⁷ cells/mL in a buffer. In some embodiments, the NSCs can be suspended from about 4×10⁷ to about 5×10⁷ cells/mL in a buffer.

A dose of NSCs (e.g., HFB2050 hNSCs) or progenitors thereof can comprise at least about 0.1×10⁷, at least about 0.5×10⁷, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸ cells per kg of subject's body weight per ventricle administered (e.g., cells/kg/ventricle).

A dose of NSCs (e.g., HFB2050 hNSCs) or progenitors thereof can comprise at most about 0.1×10⁷, at most about 0.5×10⁷, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸ cells per kg of subject's body weight per ventricle administered (e.g., cells/kg/ventricle).

A dose of NSCs (e.g., HFB2050 hNSCs) or progenitors thereof can be from about 0.1×10⁷ to about 1×10⁸, from about 0.1×10⁷ to about 9.5×10⁷, from about 0.1×10⁷ to about 9×10⁷, from about 0.1×10⁷ to about 8.5×10⁷, from about 0.1×10⁷ to about 8×10⁷, from about 0.1×10⁷ to about 7.5×10⁷, from about 0.1×10⁷ to about 7×10⁷, from about 0.1×10⁷ to about 6.5×10⁷, from about 0.1×10⁷ to about 6×10⁷, from about 0.1×10⁷ to about 5.5×10⁷, or from about 0.1×10⁷ to about 5×10⁷ cells/kg/ventricle. A dose of NSCs (e.g., HFB2050 hNSCs) can be from about 0.5×10⁷ to about 1×10⁸, from about 0.5×10⁷ to about 9.5×10⁷, from about 0.5×10⁷ to about 9×10⁷, from about 0.5×10⁷ to about 8.5×10⁷, from about 0.5×10⁷ to about 8×10⁷, from about 0.5×10′ to about 7.5×10⁷, from about 0.5×10 7 to about 7×10⁷, from about 0.5×10⁷ to about 6.5×10⁷, from about 0.5×10⁷ to about 6×10⁷, from about 0.5×10⁷ to about 5.5×10⁷, or from about 0.5×10⁷ to about 5×10⁷ cells/kg/ventricle. A dose of NSCs (e.g., HFB2050 hNSCs) can be from about 1×10⁷ to about 1×10⁸, from about 1×10⁷ to about 9.5×10⁷, from about 1×10⁷ to about 9×10⁷, from about 1×10⁷ to about 8.5×10⁷, from about 1×10⁷ to about 8×10⁷, from about 1×10⁷ to about 7.5×10⁷, from about 1×10⁷ to about 7×10⁷, from about 1×10⁷ to about 6.5×10⁷, from about 1×10⁷ to about 6×10⁷, from about 1×10⁷ to about 5.5×10⁷, or from about 1×10⁷ to about 5×10⁷ cells/kg/ventricle. A dose of NSCs (e.g., HFB2050 hNSCs) can be from about 1.5×10⁷ to about 1×10⁸, from about 1.5×10⁷ to about 9.5×10⁷, from about 1.5×10⁷ to about 9×10⁷, from about 1.5×10⁷ to about 8.5×10⁷, from about 1.5×10⁷ to about 8×10⁷, from about 1.5×10⁷ to about 7.5×10⁷, from about 1.5×10⁷ to about 7×10⁷, from about 1.5×10⁷ to about 6.5×10⁷, from about 1.5×10⁷ to about 6×10⁷, from about 1.5×10⁷ to about 5.5×10⁷, or from about 1.5×10⁷ to about 5×10⁷ cells/kg/ventricle. A dose of NSCs (e.g., HFB2050 hNSCs) can be from about 2×10⁷ to about 1×10⁸, from about 2×10⁷ to about 9.5×10⁷, from about 2×10⁷ to about 9×10⁷, from about 2×10⁷ to about 8.5×10⁷, from about 2×10⁷ to about 8×10⁷, from about 2×10⁷ to about 7.5×10⁷, from about 2×10⁷ to about 7×10⁷, from about 2×10⁷ to about 6.5×10⁷, from about 2×10⁷ to about 6×10⁷, from about 2×10⁷ to about 5.5×10⁷, or from about 2×10⁷ to about 5×10⁷ cells/kg/ventricle. A dose of NSCs (e.g., HFB2.5050 hNSCs) can be from about 2.5×10⁷ to about 1×10⁸, from about 2.5×10⁷ to about 9.5×10⁷, from about 2.5×10⁷ to about 9×10⁷, from about 2.5×10⁷ to about 8.5×10⁷, from about 2.5×10⁷ to about 8×10⁷, from about 2.5×10⁷ to about 7.5×10⁷, from about 2.5×10⁷ to about 7×10⁷, from about 2.5×10⁷ to about 6.5×10⁷, from about 2.5×10⁷ to about 6×10⁷, from about 2.5×10⁷ to about 5.5×10⁷, or from about 2.5×10⁷ to about 5×10⁷ cells/kg/ventricle. A dose of NSCs (e.g., HFB3050 hNSCs) can be from about 3×10⁷ to about 1×10⁸, from about 3×10⁷ to about 9.5×10⁷, from about 3×10⁷ to about 9×10⁷, from about 3×10⁷ to about 8.5×10⁷, from about 3×10⁷ to about 8×10⁷, from about 3×10⁷ to about 7.5×10⁷, from about 3×10⁷ to about 7×10⁷, from about 3×10⁷ to about 6.5×10⁷, from about 3×10⁷ to about 6×10⁷, from about 3×10⁷ to about 5.5×10⁷, or from about 3×10⁷ to about 5×10⁷ cells/kg/ventricle. A dose of NSCs (e.g., HFB3.5050 hNSCs) can be from about 3.5×10⁷ to about 1×10⁸, from about 3.5×10⁷ to about 9.5×10⁷, from about 3.5×10⁷ to about 9×10⁷, from about 3.5×10⁷ to about 8.5×10⁷, from about 3.5×10⁷ to about 8×10⁷, from about 3.5×10⁷ to about 7.5×10⁷, from about 3.5×10⁷ to about 7×10⁷, from about 3.5×10⁷ to about 6.5×10⁷, from about 3.5×10⁷ to about 6×10⁷, from about 3.5×10⁷ to about 5.5×10⁷, or from about 3.5×10⁷ to about 5×10⁷ cells/kg/ventricle. A dose of NSCs (e.g., HFB4050 hNSCs) can be from about 4×10⁷ to about 1×10⁸, from about 4×10⁷ to about 9.5×10⁷, from about 4×10⁷ to about 9×10⁷, from about 4×10⁷ to about 8.5×10⁷, from about 4×10⁷ to about 8×10⁷, from about 4×10⁷ to about 7.5×10⁷, from about 4×10⁷ to about 7×10⁷, from about 4×10⁷ to about 6.5×10⁷, from about 4×10⁷ to about 6×10⁷, from about 4×10⁷ to about 5.5×10⁷, or from about 4×10⁷ to about 5×10⁷ cells/kg/ventricle.

A dose of NSCs (e.g., HFB2050 hNSCs) or progenitors thereof can be from about 0.1×10⁷ to about 1×10⁸, from about 0.5×10⁷ to about 1×10⁸, from about 1.5×10⁷ to about 1×10⁸, from about 2×10⁷ to about 1×10⁸, from about 2.5×10⁷ to about 1×10⁸, from about 3×10⁷ to about 1×10⁸, from about 3.5×10⁷ to about 1×10⁸, from about 4×10⁷ to about 1×10⁸, from about 4.5×10⁷ to about 1×10⁸, from about 5×10⁷ to about 1×10⁸, from about 5.5×10⁷ to about 1×10⁸, or from about 6×10⁷ to about 1×10⁸ cells/kg/ventricle. A dose of NSCs (e.g., HFB2050 hNSCs) can be from about 0.1×10⁷ to about 9.5×10⁷, from about 0.5×10⁷ to about 9.5×10⁷, from about 1.5×10⁷ to about 9.5×10⁷, from about 2×10⁷ to about 9.5×10⁷, from about 2.5×10⁷ to about 9.5×10⁷, from about 3×10⁷ to about 9.5×10⁷, from about 3.5×10⁷ to about 9.5×10⁷, from about 4×10⁷ to about 9.5×10⁷, from about 4.5×10⁷ to about 9.5×10⁷, from about 5×10⁷ to about 9.5×10⁷, from about 5.5×10⁷ to about 9.5×10⁷, or from about 6×10⁷ to about 9.5×10⁷ cells/kg/ventricle. A dose of NSCs (e.g., HFB2050 hNSCs) can be from about 0.1×10⁷ to about 9×10⁷, from about 0.5×10⁷ to about 9×10⁷, from about 1.5×10⁷ to about 9×10⁷, from about 2×10⁷ to about 9×10⁷, from about 2.5×10⁷ to about 9×10⁷, from about 3×10⁷ to about 9×10⁷, from about 3.5×10⁷ to about 9×10⁷, from about 4×10⁷ to about 9×10⁷, from about 4.5×10⁷ to about 9×10⁷, from about 5×10⁷ to about 9×10⁷, from about 5.5×10⁷ to about 9×10⁷, or from about 6×10⁷ to about 9×10⁷ cells/kg/ventricle. A dose of NSCs (e.g., HFB2050 hNSCs) can be from about 0.1×10⁷ to about 8.5×10⁷, from about 0.5×10⁷ to about 8.5×10⁷, from about 1.5×10⁷ to about 8.5×10⁷, from about 2×10⁷ to about 8.5×10⁷, from about 2.5×10⁷ to about 8.5×10⁷, from about 3×10⁷ to about 8.5×10⁷, from about 3.5×10⁷ to about 8.5×10⁷, from about 4×10⁷ to about 8.5×10⁷, from about 4.5×10⁷ to about 8.5×10⁷, from about 5×10⁷ to about 8.5×10⁷, from about 5.5×10⁷ to about 8.5×10⁷, or from about 6×10⁷ to about 8.5×10⁷ cells/kg/ventricle. A dose of NSCs (e.g., HFB2050 hNSCs) can be from about 0.1×10⁷ to about 8×10⁷, from about 0.5×10⁷ to about 8×10⁷, from about 1.5×10⁷ to about 8×10⁷, from about 2×10⁷ to about 8×10⁷, from about 2.5×10⁷ to about 8×10⁷, from about 3×10⁷ to about 8×10⁷, from about 3.5×10⁷ to about 8×10⁷, from about 4×10⁷ to about 8×10⁷, from about 4.5×10⁷ to about 8×10⁷, from about 5×10⁷ to about 8×10⁷, from about 5.5×10⁷ to about 8×10⁷, or from about 6×10⁷ to about 8×10⁷ cells/kg/ventricle. A dose of NSCs (e.g., HFB2050 hNSCs) can be from about 0.1×10⁷ to about 7.5×10⁷, from about 0.5×10⁷ to about 7.5×10⁷, from about 1.5×10⁷ to about 7.5×10⁷, from about 2×10⁷ to about 7.5×10⁷, from about 2.5×10⁷ to about 7.5×10⁷, from about 3×10⁷ to about 7.5×10⁷, from about 3.5×10⁷ to about 7.5×10⁷, from about 4×10⁷ to about 7.5×10⁷, from about 4.5×10⁷ to about 7.5×10⁷, from about 5×10⁷ to about 7.5×10⁷, from about 5.5×10⁷ to about 7.5×10⁷, or from about 6×10⁷ to about 7.5×10⁷ cells/kg/ventricle. A dose of NSCs (e.g., HFB2050 hNSCs) can be from about 0.1×10⁷ to about 7×10⁷, from about 0.5×10⁷ to about 7×10⁷, from about 1.5×10⁷ to about 7×10⁷, from about 2×10⁷ to about 7×10⁷, from about 2.5×10⁷ to about 7×10⁷, from about 3×10⁷ to about 7×10⁷, from about 3.5×10⁷ to about 7×10⁷, from about 4×10⁷ to about 7×10⁷, from about 4.5×10⁷ to about 7×10⁷, from about 5×10⁷ to about 7×10⁷, from about 5.5×10⁷ to about 7×10⁷, or from about 6×10⁷ to about 7×10⁷ cells/kg/ventricle. A dose of NSCs (e.g., HFB2050 hNSCs) can be from about 0.5×10⁷ to about 5×10⁷, from about 1×10⁷ to about 5×10⁷, from about 1.5×10⁷ to about 5×10⁷, from about 2×10⁷ to about 5×10⁷, from about 2.5×10⁷ to about 5×10⁷, from about 3×10⁷ to about 5×10⁷, from about 3.5×10⁷ to about 5×10⁷, from about 4×10⁷ to about 5×10⁷, or from about 4.5×10⁷ to about 5×10⁷ cells/kg/ventricle. A dose of NSCs (e.g., HFB2050 hNSCs) can be from about 0.5×10⁷ to about 4.5×10⁷, from about 1×10⁷ to about 4.5×10⁷, from about 1.5×10⁷ to about 4.5×10⁷, from about 2×10⁷ to about 4.5×10⁷, from about 2.5×10⁷ to about 4.5×10⁷, from about 3×10⁷ to about 4.5×10⁷, from about 3.5×10⁷ to about 4.5×10⁷, or from about 4×10⁷ to about 4.5×10⁷ cells/kg/ventricle. A dose of NSCs (e.g., HFB2050 hNSCs) can be from about 0.5×10⁷ to about 4×10⁷, from about 1×10⁷ to about 4×10⁷, from about 1.5×10⁷ to about 4×10⁷, from about 2×10⁷ to about 4×10⁷, from about 2.5×10⁷ to about 4×10⁷, from about 3×10⁷ to about 4×10⁷, or from about 3.5×10⁷ to about 4×10⁷ cells/kg/ventricle. A dose of NSCs (e.g., HFB2050 hNSCs) can be from about 0.5×10⁷ to about 3.5×10⁷, from about 1×10⁷ to about 3.5×10⁷, from about 1.5×10⁷ to about 3.5×10⁷, from about 2×10⁷ to about 3.5×10⁷, from about 2.5×10⁷ to about 3.5×10⁷, or from about 3×10⁷ to about 3.5×10⁷ cells/kg/ventricle. A dose of NSCs (e.g., HFB2050 hNSCs) can be from about 0.5×10⁷ to about 3×10⁷, from about 1×10⁷ to about 3×10⁷, from about 1.5×10⁷ to about 3×10⁷, from about 2×10⁷ to about 3×10⁷, or from about 2.5×10⁷ to about 3×10⁷ cells/kg/ventricle. A dose of NSCs (e.g., HFB2050 hNSCs) can be from about 0.5×10⁷ to about 2.5×10⁷, from about 1×10⁷ to about 2.5×10⁷, from about 1.5×10⁷ to about 2.5×10⁷, or from about 2×10⁷ to about 2.5×10⁷ cells/kg/ventricle. A dose of NSCs (e.g., HFB2050 hNSCs) can be from about 0.5×10⁷ to about 2×10⁷, from about 1×10⁷ to about 2×10⁷, or from about 1.5×10⁷ to about 2×10⁷ cells/kg/ventricle.

A dose of NSCs (e.g., HFB2050 hNSCs) or progenitors thereof can be from about 0.5×10⁷ to about 5×10⁷, from about 0.5×10⁷ to about 4.5×10⁷, from about 0.5×10⁷ to about 4×10⁷, from about 0.5×10⁷ to about 3.5×10⁷, from about 0.5×10⁷ to about 3×10⁷, from about 0.5×10⁷ to about 2.5×10⁷, or from about 0.5×10⁷ to about 2×10⁷ cells/kg/ventricle. A dose of NSCs (e.g., HFB2050 hNSCs) can be from about 1×10⁷ to about 5×10⁷, from about 1×10⁷ to about 4.5×10⁷, from about 1×10⁷ to about 4×10⁷, from about 1×10⁷ to about 3.5×10⁷, from about 1×10⁷ to about 3×10⁷, from about 1×10⁷ to about 2.5×10⁷, or from about 1×10⁷ to about 2×10⁷ cells/kg/ventricle. A dose of NSCs (e.g., HFB2050 hNSCs) can be from 1.5×10⁷ to about 5×10⁷, from about 1.5×10⁷ to about 4.5×10⁷, from about 1.5×10⁷ to about 4×10⁷, from about 1.5×10⁷ to about 3.5×10⁷, from about 1.5×10⁷ to about 3×10⁷, from about 1.5×10⁷ to about 2.5×10⁷, or from about 1.5×10⁷ to about 2×10⁷ cells/kg/ventricle. A dose of NSCs (e.g., HFB2050 hNSCs) can be from 2×10⁷ to about 5×10⁷, from about 2×10⁷ to about 4.5×10⁷, about 2×10⁷ to about 4×10⁷, from about 2×10⁷ to about 3.5×10⁷, from about 2×10⁷ to about 3×10⁷, or from about 2×10⁷ to about 2.5×10⁷ cells/kg/ventricle. A dose of NSCs (e.g., HFB2050 hNSCs) can be from 2.5×10⁷ to about 5×10⁷, from about 2.5×10⁷ to about 4.5×10⁷, from about 2.5×10⁷ to about 4×10⁷, from about 2.5×10⁷ to about 3.5×10⁷, or from about 2.5×10⁷ to about 3×10⁷ cells/kg/ventricle.

A dose of NSCs (e.g., HFB2050 hNSCs) or progenitors thereof can be in at least 0.1 mL, at least 0.2 mL, at least 0.3 mL, at least 0.4 mL, at least 0.5 mL, at least 0.6 mL, at least 0.7 mL, at least 0.8 mL, at least 0.9 mL, at least 1.0 mL, at least 1.1 mL, at least 1.2 mL, at least 1.3 mL, at least 1.4 mL, at least 1.5 mL, at least 1.6 mL, at least 1.7 mL, at least 1.8 mL, at least 1.9 mL, or at least 2.0 mL.

A dose of NSCs (e.g., HFB2050 hNSCs) or progenitors thereof can be in at most 0.1 mL, at most 0.2 mL, at most 0.3 mL, at most 0.4 mL, at most 0.5 mL, at most 0.6 mL, at most 0.7 mL, at most 0.8 mL, at most 0.9 mL, at most 1.0 mL, at most 1.1 mL, at most 1.2 mL, at most 1.3 mL, at most 1.4 mL, at most 1.5 mL, at most 1.6 mL, at most 1.7 mL, at most 1.8 mL, at most 1.9 mL, or at most 2.0 mL.

A dose of NSCs (e.g., HFB2050 hNSCs) or progenitors thereof can be in about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, or about 2.0 mL.

The effective amount of NSCs can comprise any dose of NSCs (e.g., HFB2050 hNSCs) or progenitors thereof sufficient for treating perinatal HII. For example, the effective amount comprises 2 doses, 3 doses, 4 doses, 5 doses, 6 doses, 7 doses, 8 doses, 9 doses, or 10 doses of NSCs during the course of treatment.

### Method of Preparation

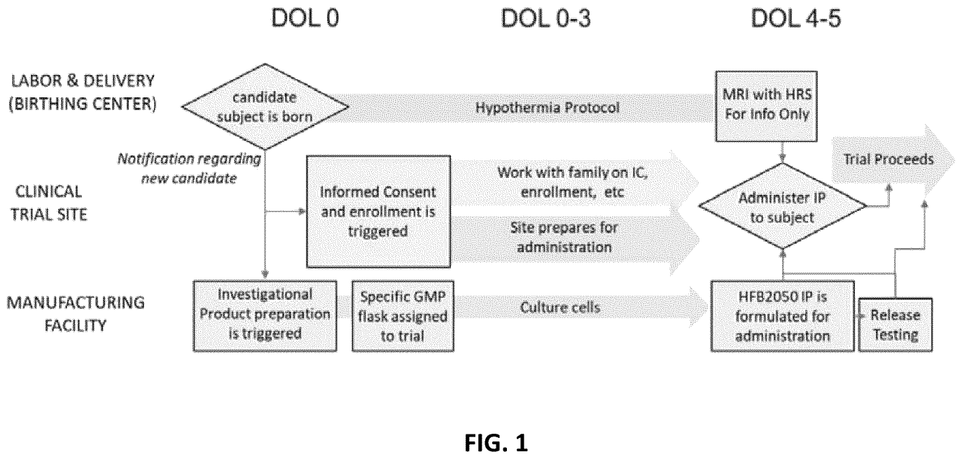

Another aspect provided herein comprises a method of preparing a patient-ready pharmaceutical composition comprising hNSCs or progenitors thereof for intracerebral administration to a subject. The hNSCs (e.g., HFB2050 hNSCs) can present the characteristics described herein, such as a doubling time of about 6 days and a sigmoidal growth curve (FIG. 3). Detailed protocols of the method of preparation can be determined based on birth of a baby with perinatal HII, as described in the process flow diagram in FIG. 1. Because HII is an emergent acute condition and there is limited time for preparing the pharmaceutical composition provided herein, multiple cultures of hNSCs or progenitors thereof can be maintained in parallel at all times to ensure a late log-phase growth condition. In some embodiments, the method of preparing the pharmaceutical composition comprises obtaining the hNSCs or progenitors thereof in a late log-phase growth condition. The hNSCs or progenitors thereof in a late log-phase growth condition can be obtained from hNSCs originated from one or more containers of hNSCs growing in parallel and/or from one or more frozen stocks. In some embodiments, the hNSCs are growing in parallel in cultures, staggered in their seeding, such that at any time a culture is in a late-log phase growth condition (e.g., staggering culture). In some embodiments, frozen stocks are cultured at a time interval such that at any time point there is a culture of the hNSCs that is in a late log-phase growth condition (e.g., staggering culture).

In some embodiments, the method of preparing can comprise (i) obtaining a first frozen stock of human neural stem cells (hNSCs) or progenitors thereof comprising (a) thawing the hNSCs or progenitors thereof; (b) culturing the hNSCs or progenitors thereof; (c) passaging the hNSCs or progenitors thereof; and the method further comprising (ii) obtaining a second frozen stock of the hNSCs or progenitors thereof, and repeating steps (a)-(c) at a time point that is different from the first frozen stock; and the method further comprising (iii) at the time of preparing the patient-ready pharmaceutical composition, collecting the hNSCs or progenitors thereof that are in a late log-phase growth condition. The method of preparing a patient-ready pharmaceutical composition can further comprise obtaining a third, a fourth, a fifth, a sixth, a seventh, a ninth, a tenth, an eleventh, or a twelfth frozen stock of the hNSCs or progenitors thereof in step (ii), wherein each of the frozen stocks is cultured at a different time point from each other. In some embodiments, each of the frozen stocks is seeded with the same time interval. The time interval can be about 3 hours, about 6 hours, about 12 hours, about 18 hours, about 24 hours, about 36 hours, about 2 days, about 3 days, about 4 days, about 5 days. For example, the first frozen stock is seeded on Day 0, the second frozen stock is seeded on Day 1, and the third frozen stock is seeded on Day 2. In some embodiments, each of the frozen stocks is seeded with different time interval. For example, the first frozen stock is seeded on Day 0, the second frozen stock is seeded on Day 1, and the third frozen stock is seeded on Day 3.

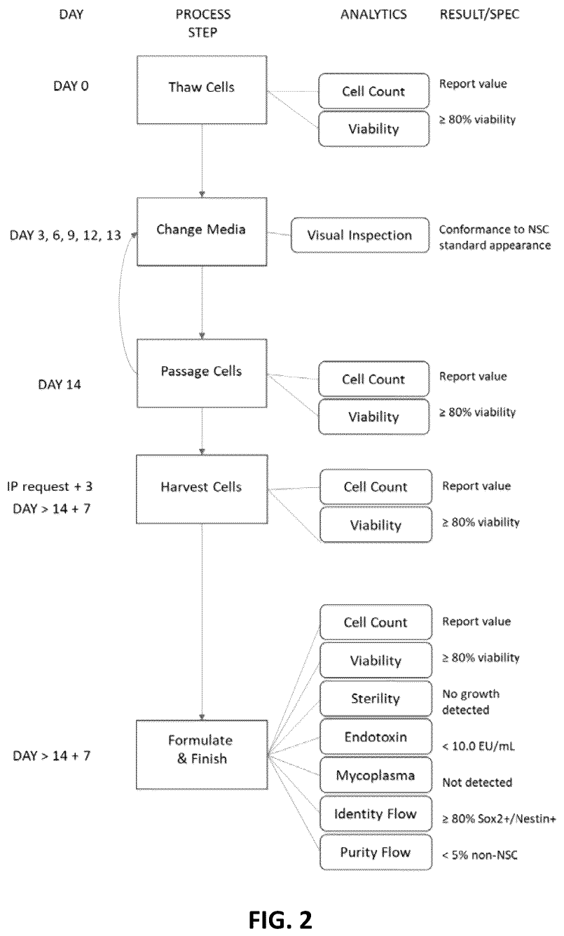

The method of preparing the compositions provided herein comprises thawing the cells comprising hNSCs or progenitors thereof, culturing the cells, changing media, passing the cells, harvest the cells, and formulating the composition. The method further comprises performing analytical tests to ensure the quality and characteristics of the compositions comprising the hNSCs provided herein, such as cell count, viability, visual inspection, sterility, endotoxin level, purity test, or a combination thereof. An exemplary process of the method of preparing the composition is illustrated in FIG. 2 and detailed in Table 1. In some embodiments, hNSCs are passaged about every 10 days, about every 11 days, about every 12 days, about every 13 days, about every 14 days, about every 15 days, about every 16 days, about every 17 days, about every 18 days, about every 19 days, or about every 20 days. In some embodiments, cells can be dissociated before passaging, such as by enzymatically dissociation with accutase. Cell can be plated onto coated surfaces, such as in a monolayer onto T75 flasks coated with human recombinant laminin.

The hNSCs or progenitors thereof can be cultured with multiple initial seeding numbers of cells. In some embodiments, the hNSCs or progenitors thereof can be cultured with the initial seeding number from about 300,000 to about 900,000 cells, from about 400,000 to about 800,000 cells, or from about 500,000 to about 700,000 cells. In some embodiments, the initial seeding number is about 350,000, about 400,000, about 450,000, about 500,000, about 550,000, about 600,000, about 650,000, about 700,000, about 750,000, about 800,000, or about 850,000 cells. In some embodiments, the initial seeding number is about 550,000 cells. In some embodiments, the initial seeding number is about 650,000 cells. In some embodiments, the initial seeding number is about 750,000 cells.

The methods of preparing the compositions provided herein comprise utilizing aseptic techniques in cell culture, comprising using a biosafety cabinet, sterilizing work area, using sterile reagents and media, sterile handling, and using personal protective equipment. In some embodiments, the compositions provided herein are prepared aseptically without a sterilizing procedure, such as filtration, microfiltration, heating, or autoclaving. In some embodiments, the compositions provided herein are prepared without the use of an anti-microbial agent. The method of preparing the compositions provided herein further comprises resuspending the cells comprising hNSCs in a buffer at various concentrations appropriate for the administration of the compositions provided herein. In some embodiments, The NSCs can be suspended at about 1×10⁷, about 2×10⁷, about 3×10⁷, about 4×10⁷, about 5×10⁷, about 6×10⁷, about 7×10⁷, about 8×10⁷, about 9×10⁷, about 1×10⁸ cells/mL in a buffer (e.g., normal saline). In some embodiments, the NSCs can be suspended at about 4×10⁷ cells/mL in a buffer. In some embodiments, the NSCs can be suspended at about 5×10⁷ cells/mL in a buffer. In some embodiments, the NSCs can be suspended from about 2×10⁷ to about 7×10⁷ cells/mL in a buffer. In some embodiments, the NSCs can be suspended from about 2.5×10⁷ to about 6.5×10⁷ cells/mL in a buffer. In some embodiments, the NSCs can be suspended from about 3×10⁷ to about 6×10⁷ cells/mL in a buffer. In some embodiments, the NSCs can be suspended from about 4×10⁷ to about 5×10⁷ cells/mL in a buffer. In some embodiments, the hNSCs or progenitors thereof are stored in a 1 mL volume container comprising the hNSCs at the concentration described herein in a buffer. In some embodiments, the buffer is normal saline. The method provided herein can comprise making one or more aliquots of the compositions provided herein for a single treatment. In some embodiments, the method comprises making 2 aliquots of the compositions provided herein for instilling into 2 ventricles of a subject.

### Pharmaceutical Composition

Pharmaceutical compositions or formulations comprising hNSCs (e.g., HFB2050 hNSCs) or progenitors thereof, of the described compositions and for use in any of the described methods can be prepared according to conventional techniques well known in the pharmaceutical industry and described in the published literature. In some embodiments, the hNSCs or progenitors thereof are in a late log-phase growth condition. In some embodiments, a pharmaceutical composition comprising hNSCs can further comprise a pharmaceutically acceptable excipient, a diluent, a buffer, or a carrier. In some cases, the pharmaceutical composition is formulated for any suitable administration method for a subject with perinatal HII, such as parenteral administration. In some cases, the pharmaceutical composition is formulated for any suitable administration method for a subject with perinatal HII, such as administration to the central nervous system. In some embodiments, the buffer is normal saline. In some embodiments, the buffer is artificial CSF. As used herein, the phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrastemal injection and infusion. In some embodiments, the intrathecal administration is an AF tap provided herein. In some embodiments, the compositions provided herein is formulated for intrathecal administration.

Pharmaceutical compositions or formulations comprising hNSCs (e.g., HFB2050 hNSCs) or progenitors thereof can have a defined level of purity for hNSCs (e.g., HFB2050 hNSCs) cells. In some embodiments, the composition comprising the HFB2050 hNSCs can comprise at least 5%, at least 10%, at least 15%, at least 20%, at least 25%, at least 30%, at least 35%, at least 40%, at least 45%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more HFB2050 hNSCs as a percentage of total cells, or HFB2050 hNSCs as a percentage of total cells within a range defined by any of the preceding values. The compositions or formulations provided herein can comprise cells derived from hNSCs, such as differentiated neurons, astrocytes, and oligodendrocytes.

Pharmaceutical compositions or formulations comprising hNSCs (e.g., HFB2050 hNSCs) or progenitors thereof can have a defined level of viability for hNSCs (e.g., HFB2050 hNSCs) cells. In some embodiments, the composition comprising the HFB2050 hNSCs can comprise at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99.5%, or more viable HFB2050 hNSCs as a percentage of total cells, or HFB2050 hNSCs as a percentage of total cells within a range defined by any of the preceding values.

The pharmaceutical compositions or formulations comprising hNSCs (e.g., HFB2050 hNSCs) or progenitors thereof are free of contaminants, pathogens, or endotoxins. A pathogen provided herein comprises Mycoplasma pulmonis, human cytomegalovirus (hCMV), Hepatitis A virus, Hepatitis B virus, Hepatitis C virus, human immunodeficiency virus 1 (HIV-1), or human immunodeficiency virus 2 (HIV-2). The compositions comprising hNSCs (e.g., HFB2050 hNSCs) or progenitors thereof are aseptic.

The pharmaceutical compositions or formulations comprising hNSCs or progenitors thereof (e.g., HFB2050 hNSCs) comprises any concentrations of the cells provided herein. The NSCs can be suspended at about 1×10⁷, about 2×10⁷, about 3×10⁷, about 4×10⁷, about 5×10⁷, about 6×10⁷, about 7×10⁷, about 8×10⁷, about 9×10⁷, about 1×10⁸ cells/ml in a buffer (e.g., normal saline). In some embodiments, the NSCs can be suspended at about 4×10⁷ cells/mL in a buffer. In some embodiments, the NSCs can be suspended at about 5×10⁷ cells/mL in a buffer. In some embodiments, the NSCs can be suspended from about 2×10⁷ to about 7×10⁷ cells/mL in a buffer. In some embodiments, the NSCs can be suspended from about 2.5×10⁷ to about 6.5×10⁷ cells/mL in a buffer. In some embodiments, the NSCs can be suspended from about 3×10⁷ to about 6×10⁷ cells/mL in a buffer. In some embodiments, the NSCs can be suspended from about 4×10⁷ to about 5×10⁷ cells/mL in a buffer. In some embodiments, the hNSCs or progenitors thereof are stored in a 1 mL volume container comprising the hNSCs at the concentration described herein in a buffer. In some embodiments, the buffer is normal saline.

In some embodiments, the pharmaceutical composition is a patient-ready pharmaceutical composition for intracerebral administration to a subject. In some embodiments, the patient-ready pharmaceutical composition is prepared by any of the methods disclosed herein.

The pharmaceutical compositions or formulations comprising hNSCs or progenitors thereof (e.g., HFB2050 hNSCs) can comprise a second therapeutic agent, an excipient, or a combination thereof. In some embodiments, the second therapeutic agent is an antibiotic or an immunosuppressive agent. Exemplary antibiotics include mupirocin, defensin, gentamycin, geneticin, cefmenoxime, penicillin, streptomycin, xylitol, or other antibiotics to assist in protecting the subject who is receiving the NSCs or progenitors thereof. Exemplary immunosuppressive agents include cyclosporine A, tacrolimus, prednisolone, azathioprine, methylprednisolone, mycophenolate mofetil, or sirolimus. The immunosuppressive agents can also comprise genetically engineered cells.

The pharmaceutical composition can comprise the HSCs and progenitor thereof described herein suspended in a buffer. The buffer can be normal saline (NS) or artificial CSF.

In embodiments, the compositions are formulated as suspensions in aqueous, non-aqueous or mixed media. Aqueous suspensions can further contain substances that increase the viscosity of the suspension including, for example, sodium carboxymethylcellulose, sorbitol and/or dextran. The suspension can also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein can comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions.

### Kits

Another aspect provides a kit that comprises the pharmaceutical composition and/or formulations comprising the cells (e.g., HFB2050 hNSCs) or progenitors thereof provided herein. In some embodiments, the hNSCs or progenitors thereof are in a late log-phase growth condition. The kit can further comprise an apparatus for administering the compositions provided herein, such as appropriate tubing for intrathecal administration. In some embodiments, the tubing for intrathecal administration can be a hollow needle or catheter. In some embodiments, the needle or catheter has a size from about 20 to about 30 gauges, from about 22 to about 28 gauges, or from about 24 to about 26 gauges. In some embodiments, the needle or catheter has a size of about 23, about 24, about 25, about 26, about 27, or about 28 gauges. In some embodiments, the needle or catheter has a size of about 29, about 30, about 31, about 32, about 33, about 34, or about 35 gauges. In some embodiments, the needle or catheter has a size of about 30 to 34 gauges. In some embodiments, the needle or catheter has a size of about 30 gauges. In some embodiments, the needle or catheter has a size of about 31 gauges. In some embodiments, the needle or catheter has a size of about 32 gauges. In some embodiments, the needle or catheter has a size of about 33 gauges. In some embodiments, the needle or catheter has a size of about 34 gauges. In some embodiments, the hollow needle is a butterfly needle.

Some embodiments further provide a kit comprising a second therapeutic agent. In some embodiments, the second therapeutic agent comprises a small molecule drug, a protein, a nucleic acid, a virus, or a combination there of.

In any herein-disclosed method, the method further comprises providing instructions for use (IFU), the IFU including instructions for administering the cell populations to the patient. In some cases, the IFU also include instructions for administering one or more pharmaceutical agents or compositions to the patient. In some embodiments, the user instruction directs a user to deliver the hNSCs to a patient via anterior fontanelle tap by using the tubing.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art can alternatively be used.

Provided herein are methods of treating perinatal hypoxic-ischemic injuries in a human subject comprising administering an effective amount of human neural stem cells (hNSCs) comprising an HFB2050 cell or a progenitor thereof. The following examples demonstrates characteristics of the exemplary cells, potential mechanism of action of the hNSC treatments, exemplary administration of hNSCs comprising patient selection, indication, dosing regimen, routes, and assessment of clinical outcome.

### Example 1: Preparation of hNSCs and Pharmaceutical Composition

HFB2050 is comprised of hNSCs which were isolated from the forebrain ventricular zone (VZ) of a single anonymous human female fetus cadaver aged 13 weeks gestation via techniques that are now known to those skilled in the art. HFB2050 hNSCs remained stable after scores of passages. HFB2050 hNSCs were tested as karyotypically normal female and have an expression profile consistent with other human fetal forebrain-derived hNSCs characterized by primary ventricular zone cells. No evidence of transformed, neoplastic, or pluripotent cells was observed in the HFB2050 line. Drug Substance Process: Staggering Culture

Because HII is an emergent acute condition, and the manufacturing team will only have 2-5 days to prepare the Investigational Product (i.e., pharmaceutical composition comprising hNSCs) (when the patient has been rewarmed over the course of 1 day following 72 hours of therapeutic hypothermia [IT]) once a request is received from the clinical site, the cells need to be ready “on-demand”. Thus, at most one flask of HFB2050 human neural stem cells (hNSCs) needs to be in cGMP culture and ready to be administered in 2-5 days at all times. Cells of various passage numbers can be administered clinically, and non-clinical studies will be performed with cells at low and high passage numbers to ensure safety and efficacy throughout and beyond the pre-established acceptable range. The anticipated coordination of events is shown in FIG. 1. While the asphyxiated neonate is classified as having Sarnat Stage 2 or Stage 3 HII, is started on the 1-IT protocol, and is enrolled (following parental consent) to receive hNSCs following rewarming 3 days later, there is time to prepare the investigational product with full phase-appropriate QC/QA of the hNSCs. This strategy requires that multiple flasks of hNSCs, staggered in their seeding, are growing in parallel, such that at most one is robustly populated, in late log-phase growth at all times. FIG. 1 illustrates a cascade of events triggered by the birth of a baby with neonatal HII leading to investigational product (IP) administration on fourth or fifth day of life (DOL 4-5). A baby is a candidate only if at Sarnat Stage 2 or Stage 3 HIE, and if he/she will undergo the hypothermia protocol. In parallel, over the next ˜4 days, the clinical site will perform activities including site preparation and informed consent, and the cells will be processed at the manufacturing facility such that all elements will be ready when the baby completes the hypothermia protocol and is ready for Investigational Product (IP; HFB2050) administration. Babies who fit criteria but whose families elect not to receive IP can be consented as standard-of-care control subjects.

Cells will be harvested with Accutase, rinsed 3 times, counted, and resuspended in NS at a concentration of 5×10⁷ cells/mL (to target a cell dosage of between 1×10⁷ and 5×10⁷ cells/ventricle in a volume of ˜1 mL or less); 2 sterile syringes will be provided to each POC, 1 for each of the 2 cerebral ventricles, ready for instillation by the neurosurgeon who will have been handed the sterile syringes by a gowned and gloved member of the Manufacturing Team (Table 10 and Example 13 for dose rationale and calculation).

As illustrated in FIG. 2, cells are passaged every 14 days by enzymatic dissociation with accutase and plated onto T75 flasks coated with human recombinant laminin and maintained in a monolayer. In order to be ready with Investigational Product at all times, multiple staggered rolling cultures will be maintained under cGMP at all times. In this way, at most one culture will be ready in late log-phase growth, which is optimal in terms of cell health, cell numbers, and engraftability at all times. Continuous seed trains will be terminated after 8 passages and new cultures will be started regularly from frozen vials of Working Cell Bank (WCB) in order to have optimal cells ready to formulate as Drug Product for administration at all times. When cells of low passage (<18) are in late log-phase growth, which is optimal in terms of cell health, cell numbers, and engraftability, they will be cryostored in vials at patient ventricle-appropriate cell doses. The cryostored supply of hNSCs will be constantly replenished by having five T75 flasks continuously maintained; after 20 passages, new cultures will be started regularly from frozen vials of WCB in order to have optimal cells ready to formulate as IP for administration at all times. Reliable stability, safety, and efficacy of the cells will be demonstrated beyond 18 passages to ensure all cultures within and beyond this 18-passage window yield usable cryovials of cells with the intended quality attributes characteristic of the IP. A compositional biochemical read-out will be an enzyme-linked immunoassay (ELISA) for the presence of glial cell line-derived neurotrophic factor (GDNF) and brain-derived neurotrophic factor (BDNF). A functional read-out will be the ability of the cells juxtaposed with mouse spinal cord slice to promote the outgrowth of murine ventral horn motor neurons (20). Full secretome analysis to provide a proteomic profile will be performed as part of IND-enabling studies and will be performed whenever any new vial from the WCB is thawed and used to start a new cohort of cultures. Reliable stability, safety, and efficacy of the cells will be demonstrated beyond 12 passages to ensure all cultures within and beyond this 8-passage window and available for administration will have the intended quality attributes characteristic of the investigational product.

HFB2050 drug substance will be further processed as per Tables 1-3 (WCB Processes). Stability program will ensure maintenance of drug substance attributes at most four passages beyond Passage 8, as a margin of safety. Table 1 further describes an exemplary protocol for processing the HFB2050 drug substance.

TABLE 1
Drug Substance Process

| | | | REAGENTS, | |
|---|---|---|---|---|
| | | | MATERIALS and | DEVELOPMENT |
| STEP | TIME | DESCRIPTION | EQUIPMENT | NOTES |
| 1 | Day 0 | Thaw Cells | 1 vial WCB ~10⁶ | Gently shake vial in |
| | | | HFB2050 cells; | water bath as it thaws |
| | | | Water bath | to achieve homogeneity |
| | | | | of solution. |
| 2 | Day 0 | Plate Cells | In HFB2050 Complete | Place thawed cells |
| | | | Media to dilute | directly into a T25 |
| | | | out DMSO (20:1); | flask containing 10 |
| | | | T25 plastic flask | mL complete medium; |
| | | | | gently swirl the flask |
| | | | | the intermix the cells |
| | | | | evening in the medium |
| | | | | but do not triturate |
| | | | | or overly manipulate |
| | | | | the cells because |
| | | | | they are fragile coming |
| | | | | out of freeze. Do not |
| | | | | move the flask after |
| | | | | a few gentle swirls |
| | | | | until the cells have |
| | | | | settled at most 16-24 |
| | | | | hrs after plating. |
| 3 | D 3-D 14 | Change Media | HFB2050 Complete | Visually inspect |
| | Every 3 days | (Completely | Media | Aspirate 10 mL spent |
| | | change medium | | media and add 10 mL |
| | | with 1st medium | | fresh media without |
| | | change post-thaw. | | rinsing or jostling |
| | | Subsequently | | of the cells. |
| | | remove Y2 the | | |
| | | medium and | | |
| | | replace with Y2 | | |
| | | fresh medium) | | |
| 4 | Day 14 | Passage Cells | HFB2050 | Dissociate cells |
| | | | Complete Media | and prepare as |
| | | | 15-mL propylene | described below in |
| | | | tubes TC75 | Step 5. Plate 1:10 of |
| | | | plastic flask; | cells onto fresh |
| | | | accutase | TC75; At this stage, |
| | | | | with the 1st post- |
| | | | | thaw passage, cells |
| | | | | can be moved from a |
| | | | | T25 flask to a T75 |
| | | | | flasks for additional |
| | | | | expansion |
| 5 | Day 15 | Continue to passage | HFB2050.SBP | Warm up HFB2050.SBP |
| | | cells (Repeat Steps #1- | Complete Media; | Media in 37° C. bath. |
| | | #4) OR freeze vials of | Accutase; | Aliquot 3 mL DPBS |
| | | cells if at Passage 8. | Neurobasal | into 15 mL conical |
| | | | Medium. | tube; Prepare |
| | | | | Freezing Media (50% |
| | | | | FBS, 10% DMSO, 40% |
| | | | | Complete HFB2030 |
| | | | | Media). |
| | | | | Dissociate cells in |
| | | | | flask using 1 mL |
| | | | | Accutase for 90 |
| | | | | seconds at 37° C. |
| | | | | Add 1 mL Neurobasal |
| | | | | Medium to flask |
| | | | | and triturate 3 |
| | | | | times to create a |
| | | | | cell suspension. |
| | | | | Transfer mixture |
| | | | | to conical tube |
| | | | | with 3 mL DPBS. |
| | | | | Wash flask with 1 |
| | | | | mL Neurobasal |
| | | | | Medium and |
| | | | | triturate 3 times. |
| | | | | Combine into conical |
| | | | | tube and gently |
| | | | | mix cell suspension. |
| | | | | Centrifuge cell |
| | | | | suspension at 0.2 |
| | | | | rcf for 4 minutes. |
| | | | | Aspirate supernatant. |
| | | | | Gently mix 0.4 mL |
| | | | | Complete Media |
| | | | | with cell pellet |
| | | | | intended for freeze. |
| | | | | Gently add |
| | | | | Freezing Media to |
| | | | | cell suspension |
| | | | | (final number of |
| | | | | cells should be |
| | | | | that appropriate |
| | | | | for 1 ventricle |
| | | | | of a generic 3.5 kg |
| | | | | full-term newborn |
| | | | | (3.5 × 10⁷ |
| | | | | cells). Transfer |
| | | | | 1 mL solution to |
| | | | | cryogenic tube |
| | | | | which is then |
| | | | | transferred to a |
| | | | | controlled-rate |
| | | | | freezing container |
| | | | | which cools at rate |
| | | | | of 1° C./minute). |
| | | | | Place in −80° C. |
| | | | | freezer for 24 |
| | | | | hours. Then |
| | | | | transfer to liquid |
| | | | | nitrogen storage |
| | | | | container |
| 6 | Upon being | Thaw patient-ready | HBSS; NS; 15 | Remove vials from |
| | called to | vial into 32 mm wells | mL propylene | liquid nitrogen |
| | prepare hNSCs | to recover from cryo- | tubes TC75 | cryotank and |
| | for a baby | stress before transport | plastic flask; | place on dry ice. |
| | placed on HT | and transplantation | Accutase. | Then thaw vial in |
| | who will | | | 37° C. bath |
| | receive cells 3 | | | quickly until |
| | days later. | | | small piece of ice |
| | | | | remains floating |
| | | | | at top of liquid. |
| | | | | Spray exterior of |
| | | | | vial with 70% |
| | | | | ethanol and place |
| | | | | in sterile hood. |
| | | | | Pre-add 5 ml |
| | | | | HFB2050.SBP |
| | | | | Complete Media |
| | | | | to each well. |
| | | | | Gently aspirate |
| | | | | the full content |
| | | | | of hNSCs from a |
| | | | | thawed vial |
| | | | | (rinse the interior |
| | | | | vial with 1 ml |
| | | | | HFB2050.SBP |
| | | | | Media to capture |
| | | | | every cell) and |
| | | | | transfer that full |
| | | | | contents of the |
| | | | | vial in a |
| | | | | dropwise manner |
| | | | | to these pre-filled |
| | | | | wells. Return |
| | | | | wells to incubator. |
| | | | | Allow to grow for |
| | | | | 3 days |
| 7 | Day of hNSC | Prepare hNSCs for | Cells will be | To harvest the |
| | transplantation. | delivery | suspended in NS | cells to create a |
| | | | and aspirate into 2 | cell suspension |
| | | | (1 for each | of an appropriate |
| | | | ventricle) sterile 3 | patient ventricular |
| | | | mL syringes at | dose in an |
| | | | concentration of | administration |
| | | | 5 × 104 cells/μl | syringe, aspirate |
| | | | and a volume based | media from the |
| | | | on baby's weight | well, add 3 ml |
| | | | and guided by | Accutase, |
| | | | parameters in | incubate for 1 |
| | | | Table 10. | minute at 37° C., |
| | | | | stop the reaction |
| | | | | with 3 mL fresh |
| | | | | medium, transfer |
| | | | | cells to a tube |
| | | | | with 6 mL HBSS, |
| | | | | rinse with 3 mL |
| | | | | medium, add to |
| | | | | tube, spin and |
| | | | | wash 1-2 times |
| | | | | with HBSS and |
| | | | | resuspend in |
| | | | | NS to achieve a |
| | | | | concentration of |
| | | | | 5 × 104 cells/μl |
| | | | | in a volume and |
| | | | | cell dose- |
| | | | | appropriate for |
| | | | | administration at |
| | | | | a per-kg-per- |
| | | | | ventricle-dose as |
| | | | | listed in Table |
| | | | | 13. Syringes will |
| | | | | be sealed in a |
| | | | | sterile package |
| | | | | and transported |
| | | | | to the POC at |
| | | | | 4° C., and, with |
| | | | | manufacturer |
| | | | | team member |
| | | | | gowned and |
| | | | | gloved, handed |
| | | | | sterilely to the |
| | | | | neurosurgeon, |
| | | | | one syringe at a |
| | | | | time, for |
| | | | | administration |
| | | | | via the anterior |
| | | | | fontanelles. |
| As | As needed for | Harvest Cells by | HBSS; Normal | Visually inspect |
| needed | admin in trial | enzymatic | Saline (NS); 15- | Aspirate media, |
| | | dissociation with | mL propylene | add 3 mL (to T75 |
| | | accutase and | tubes TC75 plastic | flask) accutase, |
| | | resuspension in | flask; accutase | incubate for 1 |
| | | complete growth | | min at 37° C., stop |
| | | medium | | the reaction with |
| | | | | 3 mL fresh |
| | | | | medium, transfer |
| | | | | cells to a tube |
| | | | | with 6 mL HBSS, |
| | | | | rinse with 3 mL |
| | | | | medium, add to |
| | | | | tube, spin and |
| | | | | wash 1-2x with |
| | | | | HBSS. |
| | | | | Resuspend in NS. |
| As | For | Formulate for | Cells will be | |
| needed | trial | Delivery | suspended in | |
| | | | normal saline | |
| | | | (NS) in sterile | |
| | | | falcon tubes on | |
| | | | wet ice | |

As the process transitions from research to clinical-grade production, reagents are assessed, and clinically compatible versions incorporated, as shown in Table 2. Stage-appropriate risk assessments are performed for all reagents and sources. Bridging and comparability studies are performed in vitro/in vivo as needed.

TABLE 2
Reagent List Currently used in Manufacture of HFB2050

| Item | Manufacturer | Supplier # |
|---|---|---|
| Neurobasal Medium | Gibco | 21103-049 |
| Normocin | Invivogen | ant-nr-2 |
| Heparin | Sigma-Aldrich | H3149 |
| LIF | EMD Millipore | LIF1050 |
| bFGF | Rand D Systems | 4114-TC-01M |
| Accutase | Millipore | SCR005 |
| Glutamax | Gibco | 35050-061 |
| Penicillin/Streptomycin | Gibco | 15140122 |
| B27 without vitamin A | Gibco | 12587-010 |
| Fetal Bovine Serum | Sigma-Aldrich | F9423 |
| Dimethyl sulfoxide | ATCC | 67-68-5 |

### Drug Product Manufacture and Quality Control

As the final drug product, cells comprising HFB2050 are harvested with accutase, rinsed 3×, counted, and resuspended in normal saline (NS) at a concentration of 5×10⁷ cells/mL (to target a cell dosage of between 1×10⁷ and 5× cells/ventricle in a volume of 1 mL or less); 2 such aliquots will be provided to each point-of-care, one for each of the 2 cerebral ventricles, ready for instillation.

The following assays are performed during the manufacture of HFB2050 hNSCs: cell count and viability; sterility (14 day; report post-administration); endotoxin; Mycoplasma; identity by flow cytometry; and purity by flow cytometry. Some of these assays are performed in-process of making the compositions comprising HFB205 as provided herein, listed as “in process” in Table 3. And some of these assays will be performed to release the cells for administration to study subjects, listed as “release” in Table 3.

TABLE 3
Analytics and Quality Control/Release Criteria - Drug Substance and Drug Product

| Assay | Step(s) | Analytical Procedure | Specification |
|---|---|---|---|
| Cell Count | In Process; | Hemocytometer | Report value: |
| | release | | Concentration for passage |
| | | | or dosing in formulation |
| Cell Viability | Thaw | Trypan blue | 2 80% viability |
| | Passage; | exclusion | |
| | release | | |
| Identity as NSCs | In-process; | Flow Cytometry | 2 80% Nestin+ |
| | release | | |
| Lack of Differentiation | In-process; | Flow Cytometry | <5% each Tuj1+, |
| | release | | GFAP+, or Olig2+ |
| Proliferative at time of | In-process; | Immunostaining or | >50% BrdU incorporation |
| grafting | release | flow cytometry | or >50% Ki67+ |
| Sterility | Release | USP<71> 14-day | No growth detected |
| | Testing | sterility assay | |
| Endotoxin | Release | USP<85> | ≤10.0 EU/mL |
| | Testing | Quantitative | |
| | | Chromogenic | |
| | | Limulus | |
| | | Amebocyte Lysate | |
| | | (LAL) | |
| Mycoplasma | Release | USP<63> | None detected |
| | Testing | | |
| Karyotype | Release | G-banding | 25/25 cells have normal |
| | Testing | | eukaryotic 46XX profile |
| Adventitious Agents | Release | Various | Negative for Hepatitis A, |
| | Testing | | B, C and HIV 1 and 2. |
| Optional: “Snapshot” of | In Process; | ELISA | Either of those below with |
| secretome for some | Release Testing | | caveat that remains unclear |
| cardinal secreted | | | which if any of these |
| neurotrophic agents | | | agents are mediating |
| (GDNF, BDNF, NT-3, | | | neuroprotection: |
| NT-4, or NGF) | | | BDNF 0.3-0.9 ng/mL/day |
| | | | GDNF 0.1-0.7 ng/mL/day |
| | | | NT-3 0.1-0.7 ng/mL/day |
| | | | NT-4 0.1-0.3 ng/mL/day |
| | | | NGF 0.1-0.5 ng/mL/day |

Master Cell Banks (MCBs) and Working Cell Banks (WCBs) will be manufactured as described in FIG. 2 and using essentially the same processes as used to manufacture HFB2050 Drug Substance, described in the Process Table (Tables 1-3) and the Process Flow Diagram (FIG. 2). To support future non-clinical studies and to ensure scalability for the clinical trial, a Research Cell Bank (RCB) of the HFB2050 hNSCs will be prepared based on the process flow in FIG. 2. The same cell culture processes as used in generating the RCB and the hNSCs studied in the earlier proof-of-concept (POC) experiments, including those listed in Table 7. Approximately 150-200 vials of MCB will be generated under cGMP conditions. The MCB process will be characterized and qualified to use in the production of approximately three WCBs of HFB2050 hNSCs (each WCB approximately 100-150 vials). The WCBs will be further qualified to generate drug substance for pre-clinical and clinical testing. Based on the cells' doubling time of ˜5 days, it was calculated that the three WCBs will be sufficient for pre-clinical and clinical material needs, including assay development, process development, and overages/retains. Outputs from the qualification of the WCB include procedures for cell banking processes (FIG. 2), reagents and material specifications (Table 2) and completion of characterization, purity, and potency tests (Table 3).

To ensure sufficient cells for both clinical and non-clinical use, multiple cells banks comprising HFB2050 can be prepared and maintained at the same time in the same fashion. Table 4 lists tests that were performed in characterizing an exemplary research cell bank comprising HFB2050 and experimental results.

TABLE 4
Analytics and Quality Control of MCB and WCB

| Test | Result |
|---|---|
| Growth Kinetics Profile | Approximately 7-fold increase from the initial seeding |
| (safety) | number (650,000 cells) at Day 21; standard sigmoidal |
| | growth curve (normal growth) |
| Contact Inhibition (safety) | Statistically significant reduction of dividing hNSCs |
| | towards confluence. Cells become contact-inhibited by |
| | 2-3 weeks post-seeding upon reaching confluence. |
| Expression of Pluripotency | Negative for pluripotency markers Oct-4, NANOG, |
| Markers by Flow | TRA1-81, TRA1-61, and SSEA4 (absence of |
| Cytometry or | contaminating pluripotent cells) |
| Immunocytochemistry | |
| (purity, safety) | |
| Multipotency and Self- | Differentiation of hNSCs into 3 neural lineage neurons, |
| Renewal (identity, potency, | astrocytes, and oligodendrocytes as well as new hNSCs, |
| safety) | and maintain a similar profile (with <20% variability) |
| | multiple passages later which is indicative of self-renewal |
| Genetic Stability (Karyotype) | Normal female karyotype and demonstrated stability over |
| (safety) | multiple passages. |
| Testing for Adventitious | Negative for mycoplasma, Hepatitis A, B, C and HIV 1 |
| Agents (safety) | and 2. |
| Cell Viability Post-cryopreservation | ≥88% viability of HFB2050 hNSCs following |
| (safety, potency) | cryopreservation and re-thawing |

### Example 2: Characterization of hNSCs

As an early assessment of the safety and other important properties of the HFB2050 hNSCs, the following tests evaluating growth kinetics, viability, contact inhibition, pluripotency, multipotency, self-renewal, karyotype and adventitious agents were completed on the HFB2050 hNSC Research Bank. The results of the testing are described in this section below and summarized in Table 5 and Table 6.

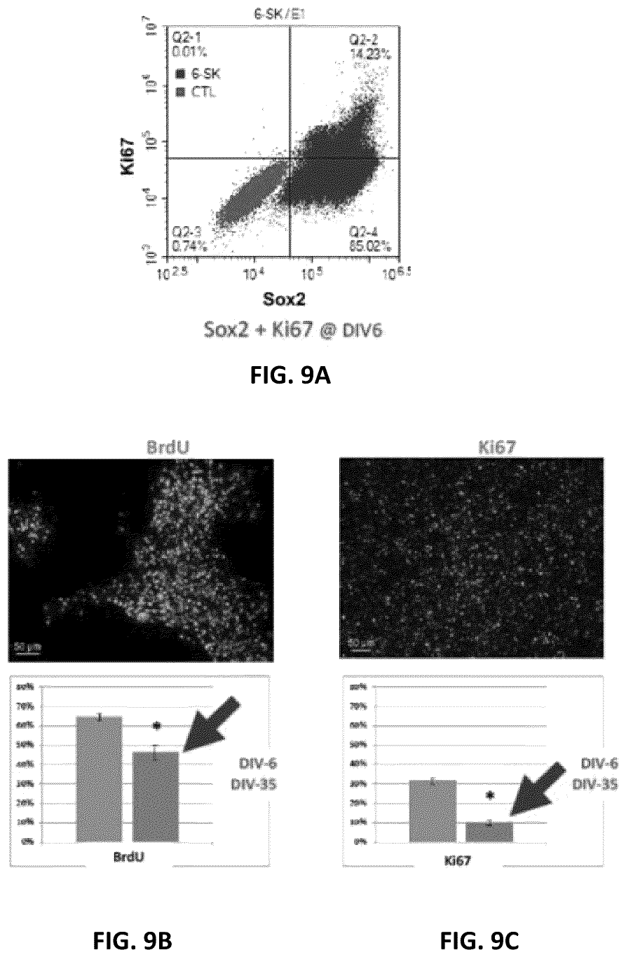

TABLE 5
Tests Performed on HFB2050 Research Bank

| Test | Result |
|---|---|
| Growth Kinetics Profile | Almost 7-fold increase from the initial seeding |
| (safety) | density (650,000 cells) by Day 21 (FIG. 3) |
| Cell Viability Post-Cryopreservation | ≥80% viability of HFB2050 hNSCs following |
| (Safety, potency) | cryopreservation and re-thawing (FIGS. 13-14) |
| Contact Inhibition (safety) | Statistically significant reduction of dividing |
| | hNSCs towards confluence. Cells become contact- |
| | inhibited by 2-3 weeks post-seeding upon reaching |
| | confluence. (FIGS. 9-11) |
| Expression of Pluripotency Markers | Negative for pluripotency markers Oct-4, |
| (Purity, safety) | NANOG, TRA1-81, TRA1-61, and SSEA4 |
| | (FIG. 12) |
| Multipotency and Self-Renewal | Differentiation of hNSCs into 3 neural lineages: |
| (identity, potency, safety) | neurons, astrocytes, and oligodendrocytes as well |
| | as new hNSCs, and maintain a similar profile |
| | (with <20% variability) multiple passages later |
| | which is indicative of self-renewal (FIGS. 6-8) |
| Genetic Stability (Karyotype) | Normal female karyotype and demonstrated stability |
| (safety) | over multiple passages. (FIG. 13) |
| Testing for Adventitious Agents | Negative for mycoplasma, Hepatitis A, B, C and HIV |
| (safety) | 1 and 2. |
| Maintenance of release criteria at | ≥88% viability, ≥80% Nestin immunopositivity. |
| point-of-care following “dry run” | ≥30% Ki67+ immunoreactivity, sterility (no growth |
| transport from point-of- | on bacterial cultures). Also, extrusion of the hNSCs |
| manufacture/presentation | through an administration needle does not change |
| | viability of the cells or the ability to continue |
| | growing. |

### Growth Kinetics

HFB2050 hNSCs were plated at a density of 650,000/well in 6-well chambers with 3 wells dedicated to each time point. Sample collection began on Day 3 after plating and assessments continued every other day (Days 5, 7, 9 . . . 21). Each of the 3 wells was sampled twice at a 1:1 dilution of cell suspension with trypan blue (to determine viability). Each sample of cell suspension was then counted twice on a hemocytometer to generate 12 distinct values used to estimate the cell population of a given time point. The media was changed at Day 14 following the maintenance procedure. During the normal expansion protocol, cells are typically left unperturbed in the container for 4 days after passage to ensure adequate adhesion.

The first sampling time point (Day 3 post-passage) represents the normal and well-recognized recovery (lag) phase of the hNSCs immediately after enzymatic dissociation, explaining the well-recognized sigmoidal shape of the typical growth curve—an initial, slight transient downward inflection of the growth curve followed by progressive growth after ˜5 days during which the cells recover, re-enter their ˜3-5 day cell cycle and begin dividing again after having been passaged. The initial decrease in cell number at Day 3 is the normal attrition from handling during passage and/or loss of end-differentiated cells and is quickly compensated for by Day 5. As depicted in FIGS. 3A-3B, the HFB2050 hNSCs exhibit an almost 7-fold increase from the initial seeding density (650,000 cells) by Day 21. HFB2050 hNSCs in monolayers showed healthy growth and proliferation as depicted in FIG. 3A. Representative phase microscopic images of hNSC proliferation and expansion in FIG. 3A showed different confluency levels of HFB2050s, along with an example of the optimal density at which the cells are passaged. As depicted in FIG. 3B, a standard sigmoidal growth curve was observed, and the growth kinetics demonstrate doubling time of about 6 days over a 21-day time course.

### Multipotency and Self-Renewal

Cytometry and/or immunostaining was used to assess the ability of hNSCs to differentiate into neurons, oligodendrocytes, and astrocytes (multipotency) as well as into new hNSCs at Passage 1 and to maintain a similar profile (with <20% variability) multiple passages later which is indicative of self-renewal. Shortly after passaging and prior to differentiation, as depicted in FIG. 4A, there is a predominance (≥90%) of expression of the classic hNSC markers Sox2 and Nestin, the former representing a somewhat earlier developmental stage than the latter and giving rise to the latter. These immunocytochemical data were corroborated by flow cytometry; high expression of Sox2 (≥90%) was confirmed using 2 different antibodies, as depicted in FIG. 4B. Both immunocytochemistry and cytometry confirmed dual expression of Sox2 and Nestin in ˜99% of HFB2050 hNSCs shortly after passaging, as depicted in FIG. 5A and FIG. 5B respectively. These undifferentiated Sox2+ and/or Nestin+ cells are the basis of self-renewal and, indeed, as shown in FIGS. 8-11, persist from passage-to-passage as a pool that is constantly renewed and never depleted. Developmentally, SOX2 is a slightly more immature marker than Nestin. It is thought that SOX2+ cells give rise to Nestin+ cells. At early stages, however, both markers are used to recognize an hNSC. SOX2 was determined as the most reliable hNSC marker. SOX2+ cells typically divide whereas Nestin+ cells can exit the cell cycle and remain as quiescent (or very slowly cycling) undifferentiated cells both in vitro and in vivo.

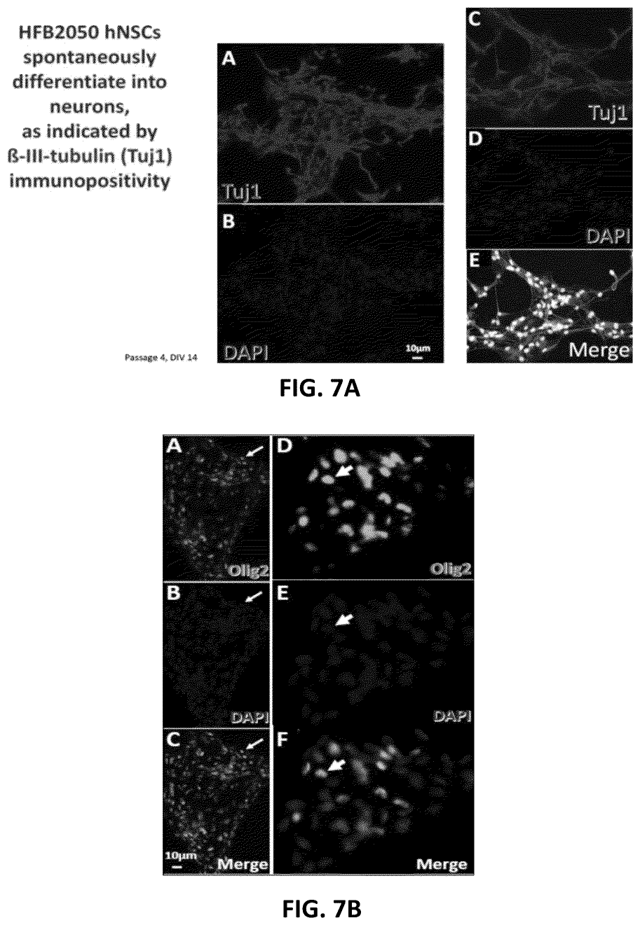

When permitted to become confluent, contact-inhibited, and exit the cell cycle, the cells differentiate spontaneously (as would be expected clinically, in a recipient brain) into all 3 cardinal neural cell types. (FIGS. 6-8) Specifically, they give rise to astrocytes expressing astrocyte marker GFAP and EATT1, the predominant neural cell type in vivo, as depicted in FIGS. 6A-6C. As depicted in FIG. 7A and FIG. 7B, the hNSCs also give rise to neurons and oligodendrocytes respectively, the latter being the most populous and final type known to emerge in the differentiation process.

Taken together, these data confirm that HFB2050 hNSCs are capable of differentiating into the three cardinal neural lineages (neurons, astrocytes, oligodendrocytes) as well as into new and/or residual undifferentiated hNSCs (the basis of self-renewal). This baseline profile, as presented in FIG. 8A, is essentially unaffected by the initial seeding density of the hNSCs (i.e., high vs. low confluence), shown in FIG. 8B. Functional proof of self-renewal is provided by the fact that early passages and later passages retain the same differentiation profile, including the generation of new hNSCs (100% Sox2+ cells upon initial plating and prior to differentiation) to insure persistence of the line (FIG. 8C).

### Contact Inhibition

Cells were incubated in the presence of BrdU for 24-72 hours at various time points over a 5-week period between initial seeding and re-passaging. The long incubation time in BrdU at each time point allowed maximum BrdU incorporation and wide capture of any dividing cells, even those that are slowly dividing at any given time point yet avoided toxicity from excessively long BrdU exposure. BrdU incorporation studies were complemented and corroborated by immunostaining for Ki67, an independent marker of cell proliferation. These experiments showed that HFB2050 hNSCs routinely experience contact inhibition, have a statistically significant fewer number of dividing cells (e.g., BrdU+ and/or Ki67+) as they become confluent (i.e., a large number of dividing cells just after passaging and re-seeding) but have a much-diminished number of dividing cells just prior to being re-passaged at confluence. (FIGS. 9-11)

Microscopically, the hNSCs become contact-inhibited by 2-3 weeks post-seeding upon reaching confluence. The cells do not become heaped upon each other or lift off the plate, but rather remain healthy with non-pyknotic DAPI nuclear profiles. They express differentiated neural markers as profiled in FIGS. 6-8.

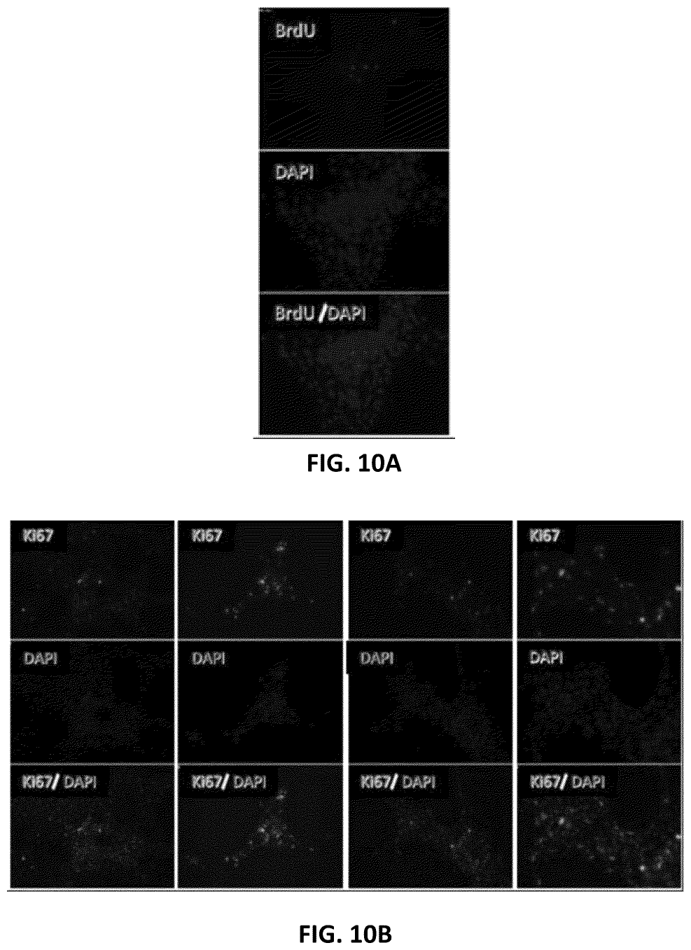

As shown in FIGS. 9A-9C, HFB2050 hNSCs have large populations of cells that incorporate BrdU and stain for Ki67 at early timepoints post-plating (i.e., dividing), but many fewer as they become contact-inhibited. FIG. 9A illustrates that on DIV6, shortly after passaging, most cells (99%) are undifferentiated early hNSCs, i.e., Sox2, and, of those, many are proliferative (Ki67+). After 35 days in vitro, when the cultures become confluent, the percentage of BrdU+ cells fall from 65% at DIV 6 to ˜40% (FIG. 9B) and the percentage of Ki67+ cells (analyzed by flow cytometry) fall from 35% to just 10% (FIG. 9C). Equally significant, however, is the distribution of those proliferative cells at the 2 different time points: at DIV 6, BrdU+ cells are distributed uniformly throughout the colony (FIG. 9B, upper panel), but, by DIV35, as per FIG. 10A, they are absent from the leading-edge (which is contact-inhibited and comprised of differentiated cells), but rather are in the central core where undifferentiated hNSCs (some slowly cycling) reside.

In summary, this analysis is consistent with the proliferative behavior of the hNSCs becoming contact-inhibited: after 35 days in vitro, the hNSC culture contains dramatically fewer BrdU+ and/or Ki67+ cells than cultures analyzed 6 days after post-seeding, concomitant with the appearance of progeny that have differentiated into the 3 cardinal neural cell types as well as new (or residual) hNSCs.

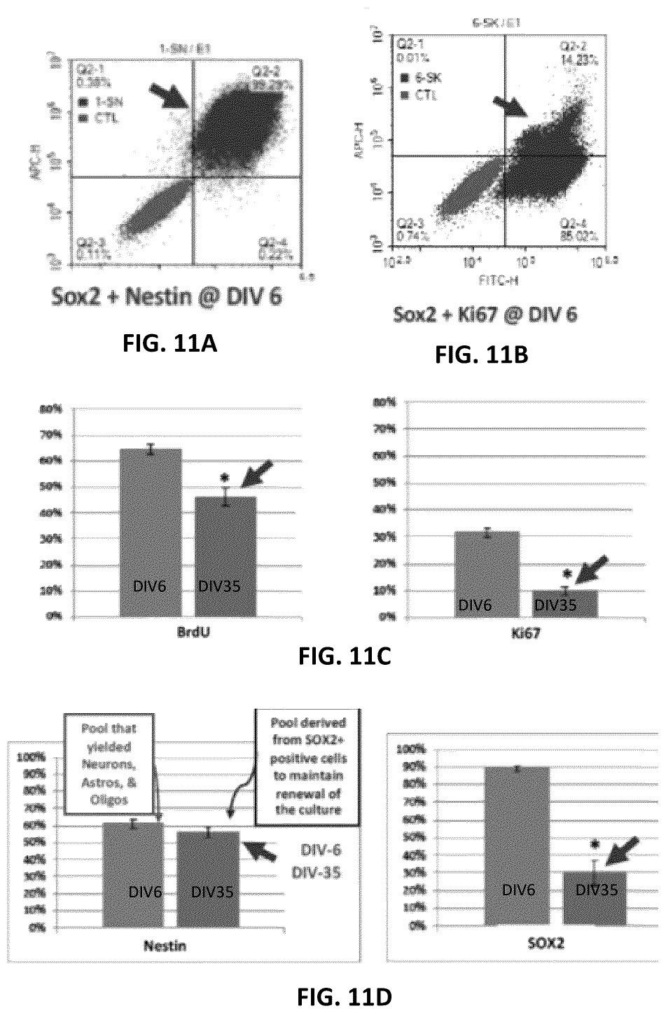

The analysis also confirmed the operation of normal neural stem cell dynamics for both contact inhibition and self-renewal for the HFB2050s, as dissected in FIGS. 11A-11D. Briefly, Sox2+ hNSCs give rise to Nestin+ hNSCs, as shown in FIGS. 11A-11B. Both are abundant and proliferative after passaging (DIV6). With time, and as the culture becomes contact-inhibited, the number of proliferative (BrdU+/Ki67+) hNSCs falls (FIG. 11C), as does the number of Sox2+ early hNSCs (FIG. 11D). The subpopulation of quiescent (non- or minimally-dividing/slowly cycling) Nestin+ late hNSCs in the center of the culture (which derived from the Sox2+ hNSCs) remains constant as a reservoir for self-renewal, poised for the next split/passage. The original Nestin+ cells in the passage gave rise, with time in culture, to differentiated progeny, but Sox2 replenished that pool and the Sox2+ subpopulation consequently diminished since proliferation had largely been suppressed by contact inhibition. Upon the next passage, however, Sox2+ numbers will rebound with the re-onset of proliferation. This is evidenced in self-renewal activity (FIG. 8C) wherein the Sox2+ subpopulation re-attains 99% prevalence immediately upon passaging and remains the same in that regard from passage-to-passage, whether an early passage or a later passage.

### Markers for Pluripotency

Flow cytometry was used to rule out the presence of any pluripotency markers. Flow analysis confirmed that HFB2050 hNSCs are negative for pluripotency markers Oct4, NANOG, TRA1-81, TRA1-60, and SSEA4, as depicted in FIG. 12. No hNSCs expressed these pluripotency markers while they did express the classical hNSC markers Sox2 and Nestin, as illustrated in FIGS. 4-5.

### Karyotype

The HFB2050 hNSCs were karyotyped using G-banding. Recent testing again confirms a normal female karyotype, as illustrated in FIG. 13. These results demonstrate karyotype stability, which has not changed since June 2011 when Passage 8 HFB 2050s from a different vial were also shown to have a normal female karyotype.

Testing for Adventitious Agents Multiple samples from different passages of the HFB2050 hNSC WCB were screened for adventitious agents. Results were negative for Mycoplasma pulmonis, human cytomegalovirus (hCMV), Hepatitis A, B and C viruses, and human immunodeficiency virus 1 and 2 (HIV 1 and 2), as listed in Table 6.

TABLE 6
Results from PCR Testing for Adventitious Agents

| | HFB2050 | HFB2050 | HFB2050 |
|---|---|---|---|
| Test | Sample 1 | Sample 2 | Sample 3 |
| HCMV | Negative | Negative | Negative |
| Hepatitis A | Negative | Negative | Negative |
| Hepatitis B | Negative | Negative | Negative |
| Hepatitis C | Negative | Negative | Negative |
| HIV 1 | Negative | Negative | Negative |
| HIV 2 | Negative | Negative | Negative |
| Mycoplasma sp. | Negative | Negative | Negative |

### Stability Following Cryopreservation and Thawing

On average, ≥80% viability is preserved following cryopreservation and thawing of HFB2050 hNSCs. Cell counts from 14 independent samples ranging from 1-3 million cells-per-vial were frozen; upon thawing, ≥80% viable cells on average were recovered (FIG. 14A) and could be further maintained and passaged, achieving the same growth kinetics as in FIG. 3, and the same differentiation profile as baseline in FIG. 14B. Multipotency and self-renewal were unaffected by cryopreservation. As shown in FIG. 14B, there no changes imposed by freezing and thawing on the markers for multipotency and self-renewal previously established as the HFB2050 hNSC profile.

### Successful “Dry Run Stability Studies of SOP

Successful “Dry Run Stability Studies” of the Standard Operating Procedure (SOP) for Sending Human Neural Stem Cells (at Patient-Ready Concentrations) from Point-of-Production/Preparation to Point-of-Care Without Loss of Key Attributes and Release Criteria (Viability, Nestin Positivity, Sterility, Proliferative Capacity).

A series of pilot studies of the clinical trial logistics have been performed and have confirmed that hNSCs coming out of, and recovering from, cryopreservation and being transported at 4° C. to a designated POC (UCSD neonatal intensive care unit (NICU) as a cell suspension at a patient-ready concentration does not compromise cell viability or the proportion of Nestin+ cells.

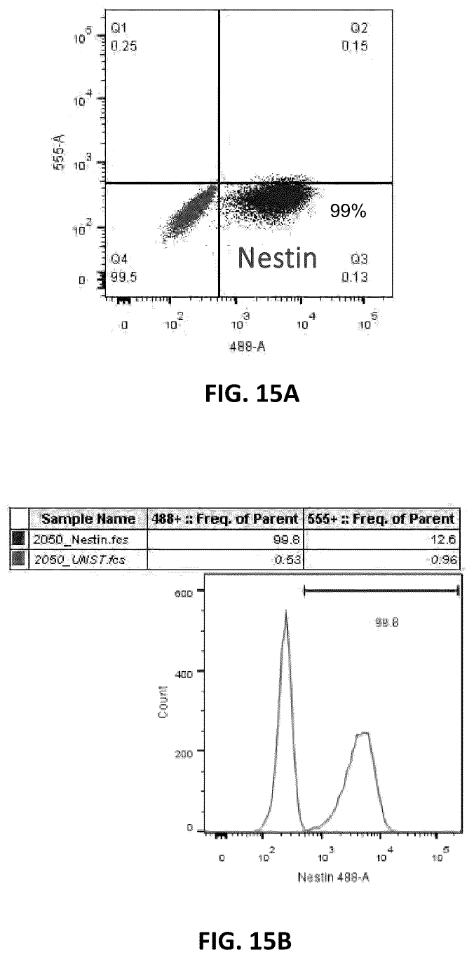

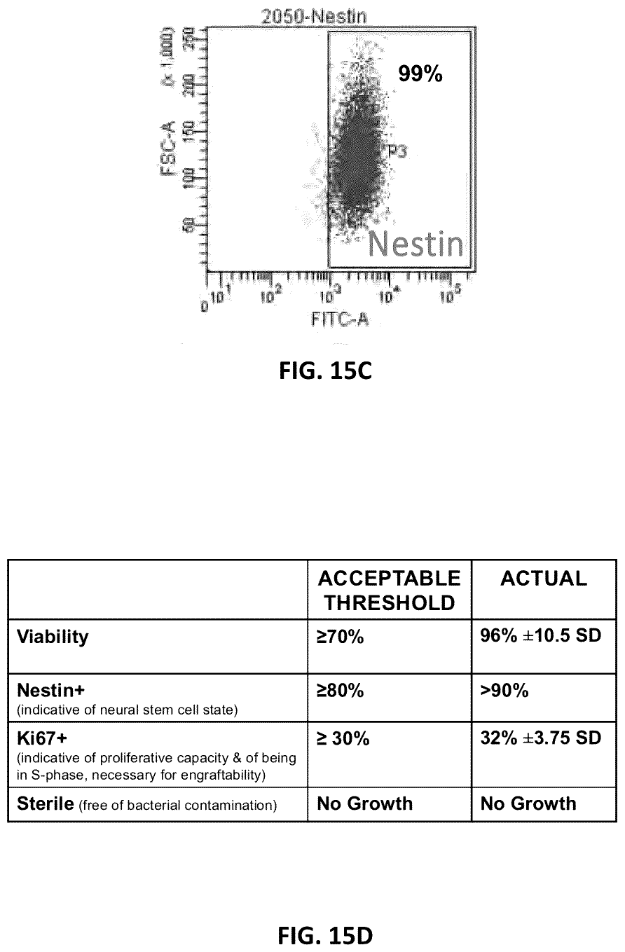

Vials of HFB2050.SBP hNSCs, cryopreserved in liquid nitrogen, were thawed, and allowed to recover from “cryo-stress” for 72 hours in growth/maintenance medium at 37° C. in a 5% air humidified incubator. Following this recovery period, cells were dissociated using Accutase, their viability was quantified using trypan blue exclusion, and they were resuspended at a patient-ready concentration of 50,000 cells/μL in an Eppendorf tube that would contain the typical dose for the cerebral ventricle of a full-term human newborn. Next, the cells were driven for ˜45 minutes on wet ice to simulate transport from point-of-production/preparation to POC (procedure room in the NICU). Several key criteria were used to assess the quality of the HFB2050.SBP hNSCs after thaw to ensure that the Standard Operating Procedure (SOP) met the release criteria: >80% cell viability upon thaw (determined by trypan blue exclusion), retention of >80% Nestin immunopositivity (indicative of a neural stem cell state), at least 30% Ki67+ immunoreactivity (indicative of proliferative capacity and of being in S-phase, necessary for engraftability), sterility (free of any bacterial contamination). Ten samples from the WCB were thawed. Cell viability was 96%±10.5 standard deviation (SD). Flow cytometry confirmed that the hNSCs maintained >90% Nestin positivity following transportation (FIG. 15A-D) (that sample in that figure actually showed 99% Nestin positivity). To determine Ki67 activity, HFB2050.SBP hNSCs were plated following the simulated transportation and stained after 6 days in vitro. The hNSCs maintained 32%±3.75 SD Ki67 positivity. No bacteria grew upon culturing.

Taken together, these data suggest that the aseptic SOP for thawing hNSCs, preparing them for a given patient at the desired concentration in the cell production facility, and transporting them to the POC for administration by the neurosurgeon to the recipient patient in the procedure room adjoining the NICU does not alter the release criteria which ensured a sterile, viable, engraftable donor hNSC population.

### Example 3: In Vitro Studies Showing Neuroprotection by hNSCs

While the precise mechanism of action (MOA) is unknown, it is believed that the hNSCs offer neuroprotection against acute/sub-acute perinatal HII by rescuing the salvageable penumbra through various mechanisms. The key anticipated hNSC-mediated mechanisms for this indication are paracrine (“the Chaperone Effect”). Proof of concept studies for this indication were designed in support of this MOA, that is, rescuing the penumbra. Other possible MOAs include circuit repair, neo-circuit creation, and remyelination. The HFB2050 intervention is administered along with the current standard-of-care (SOC) for HII which is immediate institution (within 6 hours of birth) of whole-body hypothermia (HT). HT is only marginally effective in Sarnat clinically moderate (Stage 2) HII and ineffective in Samat clinically severe (Stage 3) HII. Although HT's actual MOA is unknown, it is believed that it invokes different but complementary neuroprotective actions to those employed by hNSCs. Administration of hNSCs following HT is additive or even synergistic in minimizing cerebral damage from HII. Furthermore, there is a need for more effective and later stage (for neonates who miss the 6-hour window for HT), and more broadly applicable adjunctive treatments against perinatal HII that more elegantly target its multiple injurious processes, most of which are not addressed by HT, e.g., inflammatory, apoptotic, necrotic, excitotoxic, oxidative, vascular and demyelinative causes.

In vitro studies have shown 100% nestin+(for grafting) with the ability to differentiate into electrophysiologically-active neurons, myelinating oligodendrocytes, and astrocytes that scavenge toxins and free radicals. Studies have also shown the cells produce a range of cytokines, including those with trophic and neuroprotective actions (e.g., GDNF, BDNF, NGF, NT-3), pro-angiogenic (e.g., VEGF), and anti-inflammatory (e.g., IL-6, NO, PGE2) and studies have demonstrated the formation of a gap junction with endangered host cells that enable the passage of homeostatic molecules following cell-cell contact. hNSCs also form an extracellular matrix (Studies 1-5 in Table 7). In vivo studies have demonstrated cell engraftment, migration, and differentiation into region-appropriate electrophysiologically-active neurons, and cytokine- or myelin-elaborating glia. Inhibition of scarring and scavenging ROS (induced in vitro by H₂O₂) and glutamate (Studies 6-13 in Table 7) has also been observed.

TABLE 7
Completed Proof-of-Concept Studies on NSCs

| | | Article and | | |
|---|---|---|---|---|
| | Study Type, Objectives, | Manufacturing | | |
| # | and Reference | Process Used | Model and # of Animals | Key Outcomes |
| 1 | Establish behavior of | Therapeutic candidate | Murine [symbol] model [symbol] | NSCs [symbol] (from as far as [symbol] |
| | [symbol] in [symbol] brain: | analogue ( [symbol] | P7 mouse [symbol] - [symbol] | hemisphere) and integrate into [symbol] most robustly at |
| | Administration of | NSCs [mNSCs]) | (200,000 cells/animal) | 3 d [symbol] . |
| | [symbol] NSCs (mNSCs) | | n = 100 mice. | Differentiation into all 3 lineages and new NSCs in right |
| | at various time points & | | No immunosuppression. | [symbol] . No tumors, excessive inflammation, cell overgrowth, |
| | locations following [symbol] | | Follow-up for 1-3 mos. | deformation. |
| | to establish NSC | | | Extensive crosstalk between NSCs and host. Help reconstitute |
| | behavior concurrent with | | | [symbol] promote [symbol] of region by |
| | [symbol] . | | | host, promote host [symbol] outgrowth; [symbol] inflammation |
| | | | | and scarring; promote differentiation of host NSCs into |
| | | | | greater number of [symbol] . |
| | | | | NSCs are induced to upregulate genes mediating migration, |
| | | | | proliferation, differentiation, and [symbol] |
| | | | | No evidence of immunorejection. |
| | | | | No adverse effects |
| 2 | Efficacy & Toxicity | Therapeutic candidate | P7 mouse pups - RVM | [symbol] mediated by expression of [symbol] by host [symbol] |
| | Determine molecular | analogue (mNSCs) | (200,000 cells/animal) | and reactive [symbol] which engages CXCR4 receptor on NSCs |
| | mechanisms | plus Therapeutic | n = 50 mice | (Both [symbol] and human). |
| | mediating [symbol] of | candidate ( [symbol] 2050 | No immunosuppression | NSCs, when they engage pathologic niche [symbol] expression |
| | [symbol] & human | hNSCs) (research | Follow-up for 1-3 mos. | of [symbol] . |
| | NSCs to [symbol] in vivo | grade) | | No evidence of [symbol] |
| | | | | No adverse effects |
| 3 | Efficacy & Toxicity | Therapeutic candidate | P10 rat pups - RVM | [symbol] validity of MRI with HRS in [symbol] both with and |
| | Prove validity of [symbol] | ( [symbol] 2050 hNSCs) | (500,000 cells/animal) 20 | without hNSCs as well as ability to track [symbol] -labeled hNSCs |
| | [symbol] in [symbol] rats both | (research grade but | males, 25 females, 3 [symbol] | long-term (at least 58 [symbol] post-implant in [symbol] rat). MRI |
| | with and without hNSCs | [symbol] compatible) | [symbol] groups. | [symbol] correlated with histology and “rat pup severity score” |
| | as well as ability to track | | N = 29 for moderate, [symbol] | (RPSS). |
| | [symbol] -labeled hNSCs | | labeling, | hNSCs preferentially migrate to and integrate within areas of |
| | long term. Show similar | | n = 19 labeled, | moderate and severe [symbol] (influenced, in part, by [symbol] of |
| | | | n = 26 unlabeled. | repair-associated gene products such as HSP27 which is most |
| | | | | [symbol] in [symbol] regions where cells still salvageable and self-repair |
| | | | | mechanisms triggered (i.e., the penumbra); |
| | MRI [symbol] apply to | | No immunosuppression | HSP27 also [symbol] integrated donor hNSCs. Regions of [symbol] |
| | human [symbol] . | | Follow-up for 3-4 | HSP27 coincide with those determined by MRI to be [symbol] , |
| | | | months (for hNSCs) and | likely allowing for patient selection with high sensitivity and |
| | | | for [symbol] wks. For mNSCs, | specificity. HSP27 [symbol] most dramatically when penumbra, based |
| | | | (a therapeutic candidate | on [symbol] is most prominent and salvageable, which also |
| | | | analogue). | correlates with where hNSCs are found to have migrated most |
| | | | | robustly and with the greatest degree of [symbol] salvage |
| | | | | and functional recovery. HSP27 and donor hNSCs co-localize |
| | | | | consistently in penumbra. Although stably integrated hNSCs |
| | | | | can differentiate into ( [symbol] ) neurons, their principal action is |
| | | | | [symbol] neurons in salvaging the penumbra. Gender was not a |
| | | | | variable. |
| | | | | No evidence of immunorejection. |
| | | | | [symbol] works in human [symbol] . MRS also applicable in |
| | | | | rats and humans. |
| | | | | No evidence of rejection. |
| | | | | No adverse effects. |
| 4 | Efficacy & Toxicity | Therapeutic candidate | NSCs exposed in vitro to | Proliferation, migration, and protein synthesis suppressed by HT |
| | (abstract) | ( [symbol] 2050 hNSCs) | [symbol] assessed for viability, | starting at <3 d of HT; hence would not want donor hNSCs in that |
| | Determine whether HT | (research grade but | proliferation, migration, | HT environment. |
| | alters hNSC activity & | cGMP compatible) | metabolism. | Future [symbol] Given that we had previously published that 3 d |
| | whether an [symbol] | | n = 15/group in triplicate | post- [symbol] was actually the optimal time for therapeutic NSC |
| | time window for | | (biological and technical | implantation, and that SOC therapeutic HT is terminated at |
| | coadministration of | | replicates) | 72 h post- [symbol] should be possible to coordinate timing of both |
| | [symbol] can be | | | interventions - hNSC after HT and [symbol] . |
| | ascertained. | | | |
| 5 | Efficacy & Toxicity | Therapeutic candidate | All work in [symbol] (in | Interactions between hNSCs and TNF-alpha stimulated |
| | Interaction between | ( [symbol] 2050 hNSCs) | triplicate); to understand | human [symbol] (simulating an inflamed [symbol] ) are |
| | hNSCs & inflamed | (Research grade but | some [symbol] | [symbol] -a2, a6, and [symbol] , but not a4, [symbol] , or CXCR4-SDF1a- |
| | [symbol] mediated by | cGMP compatible) | underlying hNSCs | suggesting not only that the mechanisms mediating hNSC |
| | integrins | | [symbol] | [symbol] via the vasculature differ from the mechanisms mediating |
| | | | | [symbol] through [symbol] , but also that each step invokes a |
| | | | | distinct pathway mediating a specialized function in the hNSC |
| | | | | homing cascade. |
| | | | | [symbol] Selective use of integrin subgroups to mediate |
| | | | | homing of hNSCs may also be used to ensure that cells within the |
| | | | | systemic circulation are delivered to the pathological region of a |
| | | | | given organ to the exclusion of other, perhaps undesired, organs. |
| 6 | Efficacy & Toxicity | Therapeutic candidate | mNSCs and hNSCs grafted | When hNSCs were pre-differentiated towards glial lineage before |
| | Utility of hNSCs to be | analogue (mNSCs) | into newborn and juvenile | [symbol] , these subpopulations were eliminated. About 40% of |
| | resistant to [symbol] | [symbol] Therapeutic | [symbol] mice, assessed for | undifferentiated hNSCs (including O2A precursors) survived, |
| | toxicity and to produce | candidate (HFB 2050 | differentiation, resistance | however, and were resistant to [symbol] , even at levels found |
| | GalC when confronted | hNSCs) (research | to [symbol] , & GalC | in Krabbe's disease. These precursors would probably be the |
| | with cells from the | grade but cGMP | production (n = 28). | basis for later differentiation of the grafted hNSCs into |
| | [symbol] mouse | compatible) | Follow-up for 3.3 months. | [symbol] |
| | model of Krabbe Disease | | | No immunorejection. |
| | ( [symbol] cell | | | No adverse outcomes |
| | leukodystrophy) | | | |
| 7 | Efficacy & Toxicity | Therapeutic candidate | P0 SD pups implanted with | Implanting [symbol] mice with hNSCs improved life |
| | Utility of hNSCs in a | (HFB 2050 hNSCs) | hNSCs and evaluated for | expectancy by [symbol] compared to controls. |
| | [symbol] mouse model | (Research grade but | [symbol] performance at 13- | Implanted mice also demonstrated improved motor performance, |
| | of Sandhoff Disease (SD) | cGMP compatible) | 17 weeks of age (n = [symbol] ) | decreased [symbol] activation, as well as increased [symbol] |
| | | | and survival (n = 37) | [symbol] levels and decreased ganglioside storage. |
| | | | Follow-up for 5 months | |
| 8 | Efficacy & Toxicity | Therapeutic candidate | hNSCs HFB2050 grafted | Assessment 4 & 7-8 months post-op: hNSCs survived, migrated, |
| | Utility of hNSCs in | (HFB2050 hNSCs) | into the [symbol] and | and led to improved behavior. hNSCs, when grafted both |
| | MPTP- [symbol] African | (research grade but | [symbol] of | into [symbol] and SN exhibited migration along the lesioned |
| | Green Monkey Model of | cGMP compatible) [symbol] | MPTP-lesioned adult | [symbol] pathway. Only [symbol] hNSCs differentiated into TH+ |
| | Parkinson's Disease (PD) | cells prepared for | African Green Monkeys. | & DAT+ cells and [symbol] reacted in a “cell-replacing” way to the |
| | | packaging & long | Assessment of hNSC | deficits in the environment. |
| | | distance travel to | survival, differentiation, | A larger number of undifferentiated hNSCs or hNSCs that |
| | | point-of-care 3-4 | [symbol] , and | assumed a [symbol] factors like GDNF for |
| | | days later | behavioral improvement as | their rescue and re-establishment of homeostasis. Host [symbol] |
| | | | well as rescue of host | neurons re- [symbol] normal morphometric parameters. |
| | | | neurons. [symbol] with | TH+ host neurons were preserved, and biochemically measurable |
| | | | sham-operated [symbol] & | DA was restored [symbol] aggregates were decreased. |
| | | | MPTP-lesioned controls. | No immunorejection. |
| | | | Follow-up for 7 months. | No adverse outcomes. |
| 9 | Efficacy & Toxicity | Therapeutic candidate | To determine whether | [symbol] with hNSC [symbol] , GDNF delivered to the |
| | Utility of hNSCs in | (HFB 050 hNSCs) | further manipulation of | [symbol] vector and the fate of grafted cells was |
| | MPTP-lesioned African | (research grade but | the microenvironment by | assessed after 11 mos. Donor-derived cells remained |
| | Green Monkey Model of | cGMP compatible) [symbol] | Overexpression of [symbol] | predominantly within midbrain at injection site and sprouted |
| | Parkinson's Disease (PD) | cells prepared for | in the host could enhance | [symbol] NF+ fibers that [symbol] the |
| | | packaging & long- | DA differentiation of | striatum in parallel with TH+ fibers from the host SN but did |
| | | distance travel to | hNSCs injected into the | not mature into DA neurons. |
| | | point-of-care 3-4 | SN and elicit growth of | Suggested that hNSCs can generate neurons that project long |
| | | days later. | their [symbol] to the GDNF - | fibers in the [symbol] brain. However, in the absence of |
| | | | expressing target. Follow- | region-specific signals and despite GDNF overexpression, |
| | | | up at 11 mos. | hNSCs did not differentiate into mature DA neurons in large |
| | | | N = 10 | numbers. |
| | | | | The adult primate brain, however, retains [symbol] guidance |
| | | | | cues. No immunorejection. |
| | | | | No adverse effects. |
| 10 | Efficacy & Toxicity | Therapeutic candidate | Adult rats at 2 and 4 wks. | Implantation of hNSCs into hemi- [symbol] mid-cervical |
| | hNSCs can rescue | (HFB [symbol] hNSCs) | Of age (100,000 cells/ | spinal cords demonstrated gap junctional coupling between |
| | endangered host rodent | (research grade but | injection; 3 injection | stem cells and host tissue. |
| | cells by forming gap | cGMP compatible) | [symbol] ) | By 2 weeks post-implantation hNSCs exhibit [symbol] |
| | junctions with them | | n = 3/per condition | 43 [symbol] and plaques. |
| | (cell-cell contact) | | No immunosuppression | At 8 weeks post-implantation, [symbol] tracing |
| | | | (n = 6; 3 [symbol] at 2 wks. | with [symbol] indicates hNSC |
| | | | post-Tx; [symbol] followed for | [symbol] into functional host circuitry. |
| | | | 2 mos. | Rescue of motor function and respiratory improvement in |
| | | | | [symbol] adult rats is directly [symbol] when gap junction |
| | | | | formation between donor and host cells are allowed to |
| | | | | establish. Immunochemical staining for gap-junctions |
| | | | | [symbol] is absent without engraftment of hNSCs. |
| 11 | Efficacy & Toxicity | Therapeutic candidate | [symbol] transgenic | Insertion of hNSCs into the lumbar spinal cord of ALS mice |
| | hNSCs can rescue motor | (HFB [symbol] hNSCs) | adult mouse model of | into a region known to [symbol] with [symbol] movement had |
| | neurons in the [symbol] | (research grade but | [symbol] . | better [symbol] function and weight-loading abilities |
| | mouse model of ALS | cGMP compatible). | N = 10 for [symbol] | when compared to non-injected ALS mice. |
| | | Cells prepared for | experimental group | hNSCs migrated from the central canal to the [symbol] |
| | | packaging & long- | (n = 100). | Although a small amount of hNSCs differentiated into immature |
| | | distance travel to | Immunosuppressed. | neurons [symbol] , it was the GDNF-expressing [symbol] |
| | | point-of-care 3-4 days | Follow-up for [symbol] . | that conferred protection of the host [symbol] . |
| | | later. | | The [symbol] fate of the hNSCs in vivo was [symbol] |
| | | | | [symbol] |
| | | | | undifferentiated [symbol] NPCs. |
| | | | | No immunorejection. |
| | | | | No adverse effects |
| 12 | Efficacy & Toxicity | Therapeutic candidate | [symbol] of migration [symbol] | Engrafted hNSCs migrated [symbol] to the volume |
| | Early beneficial effects | (HFB [symbol] hNSCs) | differentiation & | was reduced relative to [symbol] controls. Behavioral deficits also |
| | of grafted [symbol] | (research grade but | [symbol] effects of | improved. Such rapid improvement suggested anti-inflammatory |
| | behavior in a mouse | [symbol] compatible) | hNSCs [symbol] 4-7 mos. | action of the hNSCs rather than cell replacement. This was |
| | model of stroke | | Postimplant Mice with | supported by observed reduction of [symbol] activation, reduced |
| | | | [symbol] MCA [symbol] | production of pro-inflammatory factors and [symbol] damage. |
| | | | grafted with hNSCs into | |
| | | | [symbol] hippocampus | |
| | | | 24 h post-injury. | |
| | | | Assessment of effects on | |
| | | | inflammation & behavior. | |
| | | | N = 18 + 2 sham groups. | |
| | | | Followed for [symbol] month. | |
| 13 | Efficacy & Toxicity | Therapeutic candidate | Undifferentiated hNSCs | No adverse outcomes. No immunorejection. Improvement in |
| | Utility of hNSCs in | (HFB [symbol] hNSCs) | implanted into [symbol] | behavior and DA levels in the [symbol] not as good as differentiating |
| | [symbol] African | (research grade but | & SN of MPTP-lesioned | hNSCs into DA [symbol] . |
| | [symbol] Monkey Model of | [symbol] compatible). | [symbol] bilaterally [symbol] | |
| | Parkinson's Disease | Cells prepared for | monkey [symbol] for [symbol] | |
| | | packaging and long | year & 4 monkeys | |
| | | distance travel to | followed for [symbol] years. | |
| | | point-of-care 3-4 | | |
| | | days later. | | |
| 14 | Biodistribution, safety, | [symbol] cell line named | Undifferentiated hNSCs | Procedure & hNSCs safe. [symbol] procedure, including [symbol] of |
| | & [symbol] of hNSCs injected | [symbol] (reported in Ref. | injected via [symbol] | mother, took only 30 min per animal. Mother well. Fetuses |
| | into the ventricles of a | [symbol] similar to [symbol] , not | guidance through the | developed normally with no premature labor or physiological |
| | normal developing [symbol] | identical to, [symbol] | pregnant mother's | aberrations. Donor-derived cells [symbol] with endogenous |
| | [symbol] monkey ( [symbol] | [symbol] prepared in the | abdominal cavity, through | monkey ventricular [symbol] ( [symbol] ) NSCs & participated [symbol] in [symbol] |
| | [symbol] ) (Ref. 1) | | the uterine wall, [symbol] into a | of the monkey brain [symbol] a chimeric human-monkey cortex |
| | | same way from the | fetal ventricle: [symbol] | differentiating into [symbol] |
| | | same type of source | cells/kg/vent = [symbol] | NSCs as per normal development. No tumors, deformations, cell |
| | | | cells/vent in a volume of | overgrowth, bleeding. Immunosuppression likely not needed |
| | | | 0.9-1.3 mL injected over 2 | because the [symbol] & number of the cells in the [symbol] fetus |
| | | | mins via an [symbol] gauge | was identical to those in the 2 [symbol] fetuses. There was |
| | | | spinal needle attached to a | no T-cell infiltration or [symbol] or [symbol] scarring evident. |
| | | | 10-mL [symbol] density of | |
| | | | cells = [symbol] | |
| | | | Fetuses (weighing [symbol] | |
| | | | kg, [symbol] of [symbol] wk | |
| | | | human fetus) [symbol] | |
| | | | one month post-implant | |
| | | | at term, [symbol] prior to [symbol] | |
| | | | Two of the mothers | |
| | | | received cyclosporine | |
| | | | [symbol] the | |
| | | | [symbol] mother did not | |

[symbol]

indicates data missing or illegible when filed

### Example 4: In Vivo Animal Model Studies Showing Neuroprotection by hNSCs

Additional pilot pre-clinical studies in Rice Vanucci Model (RVM) rats are performed to further characterize the optimal dose, route of administration (ipsilateral, contralateral, or both ventricles), and timing of cell administration relative to HT. Biological activity objectives are consistent with clinical endpoints: demonstrate a decrease in size of the HII lesion (as assessed by MRI and histology) based on a decrease in salvageable penumbra, no increase in necrotic core, and no parenchymal loss as well as an improvement in cognitive and motor tasks (see Studies 1 and 3 in Table 8).

The pilot dose ranging study are performed in 10-day old (d/o) Wistar rats having HII-induced by unilateral carotid ligation followed by normothermic (NT) exposure for 90′ to 8% FiO2 (i.e., RVM). This rodent model is validated on the basis of literature for the studied condition. For HT, pups are cooled (33° C., 6 hours—the equivalent of cooling 72 hours in human neonates) in a temperature-controlled chamber, within 6 hours of experimental HII induction. hNSCs in a dissociated single cell suspension (at 5×10⁴ cells/μL NS; 10 μL delivers approximately 2×10⁷/kg/ventricle) administered by a Hamilton syringe as previously described into the cerebral ventricle contralateral to the HII. It is known that NSCs possess an intrinsic pathotropism that impels them to migrate to injured regions even over long distances, including to the opposite side of the brain; furthermore, the lesioned area itself, at this time post-HII, is inhospitable to the cells, making direct implantation a less favored approach (although contralateral vs. ipsilateral instillation will be compared). Timing of administration in relation to lesioning and HT vary per experimental group. No immunosuppression is used. MRI is used for real-time monitoring: T2WI and DWI is used to generate a Hierarchical Region Splitting (HRS) image (which maps and identifies core vs. penumbra within the lesion) as well as to track SPIO-loaded hNSCs and monitor the evolution of tissue injury at early (1-3d) and chronic (90d) post-HII time points. Behavior is assessed at most 90 days post-injury. After behavioral assessments are completed, these adult rats are sacrificed for histological and molecular analysis. Table 8 summarizes the non-clinical pilot study plan.

In addition to the planned pilot studies, two IND-enabling studies are anticipated: (1) a GLP safety/toxicity/tumorigenicity study in rats, and (2) an acute feasibility/biodistribution/safety study in neonatal sheep. A brief summary overview of the plans is provided here for context.

(1) The GLP safety/toxicity/tumorigenicity/biodistribution study in rats is intended to demonstrate the short-term and long-term safety, as well as examine tumorigenicity and biodistribution of HFB2050 manufactured under phase-appropriate controls, and delivered at several dose multiples higher than the planned highest clinical dose on a per kg basis. This study has this type of design:
Subjects: 120 neonatal rats (˜10 days of age; ˜25 g); 60 male, 60 female
Route of Administration: Intracerebroventricular bilateral
Dose groups: 10/sex/group: vehicle, low dose (1×10⁶ cells), high dose (5×10⁶ cells)
Endpoints: in-life and prior to sacrifice—cageside observations; body weight; temperature; CBC, hematology; upon sacrifice—necropsy; organ weight; histopathology; PCR for human Alu; IHC for human nuclear antigen.
Terminal timepoints: 3-month and 9-month.

(2) The acute feasibility/biodistribution/safety study in hypoxic neonatal sheep is intended to test and ensure the product handling and administration system, including delivery instruments and protocol; as well this study will ensure the acute safety of the procedure and acute biodistribution of the cells, in an animal of similar size to a human infant. The study has this type of design:
Subjects: 12 neonatal hypoxic sheep (˜3.5 kg); ideally 6 male, 6 female
Route of Administration: Intracerebroventricular bilateral
Dose groups: 3/sex/group: vehicle, high dose (2.5×10⁸ cells)
Endpoints: in-life and prior to sacrifice—cageside observations; body weight; temperature; CBC, hematology; upon sacrifice—necropsy; organ weight; histology; PCR for human Alu; IHC for human nuclear antigen and Ki67.
Terminal timepoints: 24-hours and 72-hours.

TABLE 8
Pilot Non-Clinical Studies

| # | Objective | Aniaial, Sex, Number | Assesments | | |
|---|---|---|---|---|---|
| 15 | Assess effects of | RVM Rat Pups (Wistar) | Baseline measurements are established for [symbol] followed by 6-24 hours | | |
| | hypothermia [symbol] | HT Treatment Group [symbol] | of HT alone at Day 2 post- [symbol] vs. Day 90 post- [symbol] (compared to | | |
| | Quantify baseline | n = 8 male, n = 8 female (minimum | untreated RVM controls) for the following readouts [symbol] | | |
| | impact of whole | of 15 aiming for equal sex). | i. | [symbol] infact volume (total lesion volume [TLV]; core vs. | |
| | body hypothermia | Untreated Control Group [symbol] | | [symbol] ) via MRI | |
| | (HT) in rat pups | N = [symbol] male, n = [symbol] female (minimum | ii. | Histology | |
| | subjected to | of 10 aiming for equal sex). | | a. | Total lesion volume (TLV) measured by |
| | experimental | | | | triphenyltetrazolium chloride (TTC) staining on small [symbol] |
| | hypoxic-ischemic | | | | of RVM rat pups (within 72 hrs. of lesion and not be |
| | brain injury ( [symbol] ) | | | | followed longitudinally) comparable to TLV by MRI within |
| | via the Rice- [symbol] | | | | 50% tolerance. |
| | Model (RVM) | | | b. | Toxicity markers (e.g., ROS, caspase 3, TUNEL staining) |
| | | | | | are established |

| | |
|---|---|
| iii. | Baseline measurements for behavior are established in the same |
| | model at Day 90 (compared to untreated RVM controls) in at |
| | least one measure of the 2 categories below [symbol] |

| | |
|---|---|
| a. | Locomotor testing to characterize [symbol] |
| | coordination/balance abilities (e.g., accelerating rotarod, |
| | general open-field activity, [symbol] tests, cylinder-paw |
| | preference, [symbol] -walk, and/or rotarod) |
| b. | Cognitive-behavioral testing (e.g., modified [symbol] maze, |
| | [symbol] Water Maze [symbol] radial arm water maze, novel object |
| | place recognition, and/or passive avoidance) |

| 16 | Test ROAs [symbol] | RVM Rat Pups (Wistar) | 1. | Comparing ipsilateral ventricle vs. [symbol] ventricle of |
|---|---|---|---|---|
| | Combined study | Treatment Group [symbol] hNSCs administered | | treatment and controls. |
| | to assess proposed | after rat pups [symbol] (following | 2. | A statistically significant improvement is demonstrated with MRI |
| | route-of- | completion of HT; represent 2-3 days | | assessment (e.g., decreased [symbol] volume), at least one behavior |
| | administration | post- [symbol] ) at a dose of [symbol] | | modality, and/or histology when HFB2050 hNSCs are administered |
| | (ROA) and HFB2050 | hNSCs/ventricle (minimum n = [symbol] /ROA) | | in conjuction with HT via at least one of the tested ROA as |
| | hNSC stability using | | | compared to using HT alone. |
| | unlabeled cells | Control Groups [symbol] | 3. | Stability studies with unlabeled HFB2050 hNSCs are completed with |
| | from WCB | Either PBS vehicle alone or acellular | | no statistically significant changes in [symbol] criteria. Cells should |
| | | [symbol] -depleted medium | | remain ≥70% viable after simulated shipping at 4° C. or −20° C. |
| | | conditioned by [symbol] hNSCs after | | |
| | | RVM animals [symbol] after HT | | |
| | | (minimum n = [symbol] /ROA) | | |
| | | HT alone in RVM animals (n = 10) | | |
| | | Untreated RVM animals (n = 10) | | |
| | | PBS or acellular [symbol] -depleted | | |
| | | conditioned medium alone | | |
| | | administered to RVM animals | | |
| | | without HT (minimum n = 10 [symbol] | | |
| | | each ventricle) | | |
| 17 | Assess dose and | RVM Rats | Comparing timing of WCB administration between treatment and | |
| | timing of cell | Test Groups [symbol] | control groups. Optimal timing of hNSC administration in relation to | |
| | administration. | Group 1 [symbol] RVM rats receive | HT is selected by the statistically significant timing that achieves the | |
| | Combined Study [symbol] | [symbol] hNSCs within 24 h post- | greatest improvment in MRI measurement, at least one behavior | |
| | Part A | HT (once [symbol] , which is equal | modality, and/or histology relative to the HT alone control group. | |
| | Determine optimal | to 2-3 d post- [symbol] ) with optimal ROA | | |
| | timing of HFB2050 | (minimum n = 15) at standard dose | | |
| | administration in | [symbol] hNSCs/ventricle | | |
| | relation to HT | RVM rats receiving [symbol] hNSCs | | |
| | using unlabeled | at variable times in relation to HT [symbol] | | |
| | cells from WCB | Group 2 [symbol] hNSCs at same time as | | |
| | | institution of HT (minimum n = 15) | | |
| | | Group 3 [symbol] 5 days post-HT, 7 days | | |
| | | post- [symbol] (minimum n = 15) | | |
| | | Control Groups: | | |
| | | Conditioned medium in place of | | |
| | | hNSCs timed with Standard Group 1 | | |
| | | (n = 15) | | |
| | | Conditioned medium in place of | | |
| | | hNSCs timed with Group 2 (n = 7) | | |
| | | Conditioned medium in place of | | |
| | | hNSCs timed with Group 3 (n = 7) | | |
| 18 | Part B | Comparing dose of HFB2050 hNSC | 1. | Optimum dose of HFB2050 hNSC per ventricle is selected (i.e., |
| | Determine optimal | administration by adding the following | | 1x, 2x, 4x SD) |
| | dose of HFB2050 | experimental conditions: | 2. | A statistically significant improvement in at least 2 of the following |
| | administration & | Group 1 (see Part A) Standard Dose | | 3 catagories: (1) MRI (infarct reduction); (2) behavior (at least one |
| | assess safety | (SD) [symbol] 2.5 × 10⁵/ventricle | | modality); (3) histology relative to the HT-alone control group when |
| | using unlabeled | Group 4: 2 × SD/ventricle (minimum | | using the optimized conditions for hNSCs administration: ROA, |
| | cells from WCB | n = 15) | | timing, dose. |
| | | Group 5: 4 × SD/ventricle (minimum | 3. | HFB2050 derivatives are <2% in any non-neural organ as |
| | | n = 15) | | determined by Alu [symbol] and/or human-specific |
| | | | | immunohistochemistry at 90 days post-HII |
| | | | 4. | Safety under optimized conditions is established based on following |
| | | | | criteria: |

No intra- or extra-cranial malignant tumors that are derived from
hNSCs
<10% cell overgrowth, deformation, or inappropriate cell types
compared to total human cell composition.
<10% deterioration in neurologic/mental status (including loss of
motor/sensory skills, state-of-alertness, responsiveness,
respiratory control, new asymmetries, no new-onset or worsening
of seizures) compared to HT control group.
<10% pain/discomfort compared to HT control group
<10% systemic toxicity compared to HT control group
<10% exce|ss vascularity, compromised blood-brain barrier,
hemorrhage compared to HT control group.
<10% loss of parenchyma or worsening
ventriculomegaly compared to HT control group.
<10% increased intracranial pressure (ICP) compared to
HT control group
<10% sepsis of experimental animals compared to HT
control group
<10% increased cerebral scarring compared to HT control
group <10% increased mortality/morbidity compared to HT
control group (other than perioperative or from institution of the
RVM itself)

[symbol]

indicates data missing or illegible when filed

### Example 5: Synergistic Effect of hNSCs and HT for Treating HII

Presently, there is no impactful treatment for perinatal HII based on its pathophysiology. There is a need for treatments that can be initiated at any time after birth and directed against multiple downstream pathophysiological mechanisms, most of which cannot be addressed by HT (Table 9) to improve outcomes. Limitations of the standard of care (SOC) treatment for perinatal HII (e.g., HT) include the following: 1) HT is ineffective in clinically “severe” cases (Sarnat Stage 3) and only marginally effective in ˜10% of clinically “moderate” cases (Sarnat Stage 2); 2) HT believed to be neuroprotective by slowing metabolic demand and preventing re-perfusion injury, but does not address the multiple other pathogenic mechanisms that contribute to development of CP (inflammation, apoptosis, necrosis, excitotoxicity, oxidative damage, mitochondrial dysfunction, vascular disruption and rupture of the blood-brain barrier, demyelination, etc.); 3) There is no therapy at all for asphyxiated infants who are unable to receive HT within 6 hours of birth; and 4) There is no therapy for asphyxiated infants who do not respond to HT.

Table 9 lists the mechanisms of treating perinatal HII using hNSCs and HT, and how they can be additive or synergistic.

TABLE 9
Potentially Synergistic Mechanisms of Neuroprotection: NSCS Vs Hypothermia
Table 9. Potentially Additive/Synergistic Mechanisms of Neuroprotection: NSCS Vs Hypothermia

| | |
|---|---|
| Neural Stem Cells | Hypothermia |

Possible Similar mechanisms

| | |
|---|---|
| Reduces inflammation and tissue scarring | Decreases pro-inflammatory cytokines |
| Scavenging reactive oxygen species (ROS) | Reduces Nitric Oxide and ROS production |
| Promotion of endogenous neurite outgrowth | Blocks protein postsynaptic densitites formation |
| Neuroprotection | Reduces apoptotic injury |

Possible Dissimilar mechanisms

| | |
|---|---|
| Pro-angiogenesis | Reduces cerebral metabolism and blood flow |
| Trophic support | Prevents reperfusion injury |
| Mobilization of endogenous NSCs | Suppress NMDA receptor phosphorylation |
| Replacement of interneurons and glial support | Reduces expression of DNA glycosylases |

Route and Technique of Administration of Intracranial hNSCs

hNSCs comprising HFB2050 described herein can be administered much later than HT, perhaps up to 7-10 days post-injury or they can be administered soon after a course of HT on around day 4 post birth. The preparation and follow-up post-administration, and the actual administration itself do not alter the usual care and monitoring of these patients (including MRI, EEG, and serial neurological assessments). Administration of the hNSCs is minimally-invasive—via a single injection via a very narrow-gauge needle percutaneously through the anterior fontanelle into the cerebral ventricles. hNSCs within the ventricles circumvent blood-brain barrier (BBB) restrictions to migrate seamlessly into the cerebral parenchyma.

hNSCs comprising HFB2050 present long-distance pathotropism, hence, when they enter the parenchyma, they preferentially home to the ischemic lesion. The administration process can be imaged in real-time. The cells are non-tumorigenic, amenable to repeated administration, and do not provoke immunorejection in newborns. hNSCs comprising HFB2050 can integrate stably in host parenchyma as constituents from all fundamental neural lineages and exhibit extensive paracrine effects.

Serial MRI with Hierarchical Region Splitting (HRS) can be used for monitoring the impact of hNSC treatment combined with HT and can be integrated into routine care (HRS is a post-hoc mathematical manipulation of digital data already routinely obtained on all HII infants that received HT (including T2WI, DWI, SWI). MRI with HRS can monitor noninvasively evolution of HII and the advent of any adverse reactions (tumor formation, deformation, cell overgrowth, ventriculomegaly). Magnetic Resonance Spectroscopy (MRS) can be obtained during the same imaging session, providing data on metabolic impact of the hNSCs (e.g., looking for resolution of any abnormal NAA peaks and ratios, indicative of neuronal injury or repair).

Ultimately, MRI can have prognostic value as well as aid in rational patient stratification/selection. The present clinical staging of patients is based solely on neurological exam within the first 6 hours of life and on systemic non-CNS biochemical measures of acidemia. There is no direct objective measure of the CNS. MRI with HRS may allow clinical staging based on direct assessment of the state and extent of CNS asphyxia injury.

Instillation of hNSCs into the cerebral lateral ventricles (LVs) will be performed using a minimally invasive route and device (catheter) often used in neonatology to instill intrathecal antibiotics, to remove excess cerebrospinal fluid (CSF), or to reduce intracranial pressure (ICP). Because the sutures in a newborn are not yet closed, entrance into the LVs can be achieved percutaneously without a need for skin incisions or craniotomies using a simple 24-gauge IV catheter inserted only 2 cm into the anterior fontanelle (AF). Similarly, there is no need for general anesthesia (sedation and/or swaddling is sufficient as well as local topical analgesia to the insertion site). The procedure can be accomplished relatively quickly: cells are instilled into each LV over a 2-5-minute period. The entire procedure is completed within 10-15 minutes.

The desired dose of hNSCs (see Table 10) are suspended in normal saline (NS) at a concentration of 5×10⁴ cells/μL. The suspension is infused intraventricularly, via an anterior fontanelle (AF) tap, using a 24-gauge medicut catheter guided by cranial ultrasound. The administration protocol comprises the following steps:
The AF skin area is prepared and draped for the aseptic procedure.
The ultrasound probe is covered with a sterile sheath.
A 24-gauge medicut needle is inserted percutaneously into each LV via the AF tap, with entrance of the needle tip into the center of the ventricle directly visualized by the ultrasound; the distance inserted is usually 1-2 cm.
After withdrawing the needle stylet, a volume of cerebrospinal fluid (CSF) equivalent to that of the cell suspension to be instilled is gently aspirated through the indwelling catheter in order maintain a stable ICP.
After obtaining CSF, a syringe containing the cells is connected to the catheter, and the cells are slowly infused into the LV by gentle intermittent pressure on the syringe plunger at a rate of ˜1 mL/min.
The catheter is then flushed with NS (˜0.5 mL) and left in place for 1 minute. >The catheter is then removed
The site of the needle puncture is manually compressed for 5 minutes, and an aseptic dressing is applied.
Repeat for the second ventricle

### Dose and Dose-Escalation

In order to calculate an appropriate starting dose for the human studies, the established safe and effective pre-clinical doses in mice, rats, and non-human primates have been extrapolated to human subjects based on weight, as detailed in Table 10.

In the planned GLP safety/toxicity studies, the dose escalation studies are performed as follows: starting at 2×10⁷ cells/kg/ventricle, to match the dose found to be effective as well as safe in asphyxiated rat pups which has not shown adverse effects in research studies. A dose escalation (5×) to 1×10⁸ cells/kg/ventricle to well exceed the safe and effective dose in asphyxiated rat pups is then implemented. In the proposed neonatal sheep study, will further establish, bracket, and exceed cell dosages found to be safe and effective in fetal bonnet monkeys as well as address the planned high dose in human subjects. Dose escalations beyond 1.5×10⁷/kg/ventricle (currently planned as sheep low dose; human high dose) in human newborns will be informed by the studies in fetal sheep, which approximate the size (both of body and brain) of human newborns. Preclinical data suggest that the ideal density of hNSCs in a NS suspension is 5×10⁴/μL, indicating that a volume of 1 mL can be instilled per ventricle to achieve 1×10⁷ cells/kg/ventricle.

TABLE 10
Approximate Dose Comparison Across Species - Previous and Planned Studies

| | | Animal | Approx. | Total | Approx. | | | | | |
|---|---|---|---|---|---|---|---|---|---|---|
| | | weight | cell | dose | dose/kg/ | Approx. | Cells/ | Approx. | | |
| Study* | Animal | (g) | dose/vent | (bilat) | vent | dose/kg | l · tL | Vol/vent | | notes |
| 7 | Mouse pup | 5 | 1 × 10⁵ | 2 × 10⁵ | 2 × 10⁷ | 4 × 10⁷ | 5 × 10⁴ | 2 | l · tL | safe & |
| | P0 LSD | | | | | | | | | effective |
| 14 | Fetal | 400 | 2 × 10⁷ | 2 × 10⁷ | 5 × 10⁸ | 5 × 10⁸ | 2 × 10⁴ | 1 | mL | safe |
| | Bonnet | | | (one vent) | | | | | | |
| | monkey | | | | | | | | | |
| | (Macaca | | | | | | | | | |
| | radiata) | | | | | | | | | |
| 17 | Rat pup | 25 | 5 × 10⁵ | 1 × 10⁶ | 2 × 10⁷ | 4 × 10⁷ | 5 × 10⁴ | 10 | l · tL | safe & |
| | P10 HII | | | | | | | | | effective |
| Low dose | Rat pup | 25 | 5 × 10⁵ | 1 × 10⁶ | 2 × 10⁷ | 4 × 10⁷ | 5 × 10⁴ | 10 | l · tL | TBD |
| GLP | P10 | | | | | | | | | |
| planned | | | | | | | | | | |
| High dose | Rat pup | 25 | 2.5 × 10⁶ | 5 × 10⁶ | 1 × 10⁸ | 2 × 10⁸ | 5 × 10⁴ | 50 | l · tL | TBD |
| GLP | P10 | | | | | | | | | |
| planned | | | | | | | | | | |
| Low dose | Neonatal | 3500 | 3.5 × 10⁷ | 7 × 10⁷ | 1.0 × 10⁷ | 2 × 10⁷ | 5 × 10⁴ | 0.7 | mL | TBD |
| planned | sheep HII | | | | | | | | | |
| High dose | Neonatal | 3500 | 1.05 × 10⁸ | 2.1 × 10⁸ | 3 × 10⁷ | 6 × 10⁷ | 5 × 10⁴ | 2.1 | mL | TBD |
| planned | sheep HII | | | | | | | | | |
| (3×) | | | | | | | | | | |
| Low dose | Human | 3500 | 3.5 × 10⁷ | 7 × 10⁷ | 1 × 10⁷ | 2 × 10⁷ | 5 × 10⁴ | 0.7 | mL | TBD |
| planned | clinical | | | | | | | | | |
| | trial | | | | | | | | | |
| High dose | Human | 3500 | 7.0 × 10⁷ | 1.4 × 10⁸ | 2 × 10⁸ | 4 × 10⁸ | 5 × 10⁴ | 1.4 | mL | Dose found |

| | | |
|---|---|---|
| planned | clinical | to be safe |
| (2×) | trial | & effective |
| | | preclinically |

(*as denoted in Table 7 and Table 8)

### Study Design

This is a multicenter FIH Phase 1/2a trial testing the hypothesis that intracerebroventricular (ICV) implantation of HFB2050 hNSCs following HT (SOC) will improve outcomes when compared to SOC alone. Non-clinical studies suggest complementary and possibly additive/synergistic neuroprotective effects from these two modalities. To evaluate safety of the HFB2050 hNSCs in this treatment population. Evaluators will be blinded to treatment status.

Term HII newborns who meet criteria for and receive SOC (3-days of therapeutic hypothermia; HT) will be randomized to receive one of the following:
Non-Interventional: Continued follow-up (symptomatic care; n=20)
Investigational Treatment: hNSC implantation on Day of Life (DOL) 4-5, administered via a 24-gauge catheter inserted percutaneously through the anterior fontanel into each of the two lateral cerebral ventricles, with each ventricle receiving 5×10⁶ cells/kg/ventricle in 0.35 mL of normal saline (NS; Cohort 1; n=6) or 1.5×10⁷ cells/kg/ventricle in 1 mL NS (Cohort 2; n=6)

Additional control groups will be comprised of:
Neonates who would qualify clinically for, but do not get, HT (e.g., delay-to-treatment [>6 hours of age]; lack of parental consent); will be enrolled for symptomatic care and follow-up only.
Neonates who receive HT but parents do not give consent for hNSC implantation; will be enrolled for symptomatic care and follow-up only.

All subjects will undergo MRI to document severity (DOL 4, upon rewarming) and to quantify “Penumbra: Core Ratios” (via HRS); while these data will be recorded for future correlations with outcome, they will not be used at this point for inclusion or exclusion criteria.

Primary endpoint: Safety comparable to controls, which includes the following measures.
No intra- or extra-cranial tumors, cell overgrowth, deformation, or inappropriate cell types
No deterioration in neurologic/mental status (including loss of motor/sensory skills, developmental milestones, sensorium, state-of-alertness, responsiveness, or respiratory control); no new asymmetries, new-onset or worsening of seizures, or loss of milestones
No pain/discomfort
No systemic toxicity
No excess vascularity, compromised blood-brain barrier, hemorrhage
No loss of parenchyma or worsening ventriculomegaly
No increased intracranial pressure (ICP)
No infection
No inflammatory or immune reaction
No increased scarring
No increased mortality/morbidity

### Secondary Endpoints:

Improved outcome by addition of hNSCs to HT
   Neuro exam (including seizure history) at 18 mos.
   Behavior (including developmental milestones and Bayley Scales of Infant development on follow-up) at 18 mos.
   MRI at 9 and 18 mos: Resolution or reduction in volume of penumbra; No increase in core volume; No loss of parenchyma; No enlargement of ventricles (relevant to the given patient's pre-treatment condition and to untreated age-matched asphyxiated patients); No worsening of other CNS structures
Retrospective assessment of the predictive value of the proposed MRI-based HII severity scale:
   a. For prognosticating outcome
   b. For selecting candidates for receiving neuroprotective hNSCs
   c. For ruling out or ruling in adverse outcomes from the hNSC implantation

Study population: Full term HII newborns with Sarnat clinical scores of 2 or 3 and meet criteria for and receive 3d hypothermia (HT) (SOC)

### Inclusion Criteria

Neonates of at most 37 weeks gestation (full-term or post-term):
Sarnat Stage 2-3 HII
No other anomalies or dysmorphologies
Qualify for HT: Apgar ≤5 at 10 minutes; Continued need for resuscitation at 10 minutes; pH <7.00 or base deficit?16.0 mmol/L: In umbilical arterial or venous blood sample prenatally or in a venous, arterial, or capillary blood sample within 60 minutes of birth
Encephalopathy: Lethargy, stupor, or coma; At most one of the following: Hypotonia, abnormal reflexes (including oculomotor or pupillary), absent or weak suck, and seizures.

A neonate is excluded if he/she fits any one of the following Exclusion Criteria:
Preterm neonates
Sarnat Stage 0 or 1 HII
Pre-existing anomalies, dysmorphologies, genetic conditions
Pre-existing intracranial bleeds, masses, hydrocephalus
Do not qualify for HT: 1) Do not qualify based on biochemical or clinical criteria; 2) >6 hrs. post-event; 3) Pre-term; 4) No informed consent for HT
Point-of-care is unable to obtain or administer hNSCs
Point-of-care unable to perform MRI and/or bedside EEG, assess infants for HT, administer HT, perform neurological follow-up on infants until discharge
No informed consent for hNSC implantation

### Investigational Product

HFB2050: Freshly dissociated hNSCs suspended as single cells in NS (48 hrs after last passage) at a concentration of 5×10⁴ cells/μL, maintained at 4° C. before administration.

### Dose Form and Routes of Administration (ROA)

Minimally invasive aseptic percutaneous instillation of a dissociated hNSC suspension in NS via a 5-cm 24-gauge needle through the anterior fontanelle into each of the two lateral cerebral ventricles.
Insertion guided by cranial ultrasound
Two distinct needle passes—one into each ventricle

### Dose-Escalation:

1×10⁷/kg/ventricle administered as a sufficient volume of a 5×10⁴ cell/μL suspension of NS=1st dose level for Cohort 1
2×10⁷/kg/ventricle in 1 mL NS=2× dose escalation (2nd dose level)
May consider progressing to 3.5×10⁷ cells/kg/ventricle (to match cell dosage found to be safe and effective in fetal monkeys, but dose escalations >2.0×10⁷/kg/ventricle in human newborns will depend upon non-clinical studies yet to be performed in fetal sheep, which approximate the size (both of body and brain) of human newborns.

Pre-clinical studies comparing various cell densities, doses, and ROAs (intra-lesional, ipsilateral ventricle, contralateral ventricle, intravascular) suggest the above to be optimal.

### Example 6: In Vivo Preclinical Studies Relevant to the Use of HFB2050.SBP hNSCs in Perinatal HIT

### Background

The over-riding premise of these preclinical studies has been that the neuroprotection conferred by human neural stem cell (hNSC) transplantation for perinatal HII (also termed “asphyxia”) is more efficacious than (and/or could enhance the efficacy of) present SOC, which is hypothermia (HT) (i.e., lowering the total body temperature of an asphyxiated encephalopathic full-term baby to 33 to 34° C. for 3 days starting within 6 hours of birth). HT is currently the only approved clinical treatment of perinatal hypoxic-ischemic encephalopathy (HIE) in newborns. Despite the fact that the clinical results from HT are variable (for example, HT is not effective, and may be inimical, in low-and-middle income countries), and HT provides only a marginal improvement in a subset of moderately-injured (based on clinical staging) newborn brains (Sarnat Stage 2) who receive this treatment within the first 6 hours of life, there is currently no better approved treatment method; all neuroprotective drugs have fail in clinical trial.

The unmet medical need here, of course, includes better neuroprotection for a greater number of even more severely injured babies, including those who are unable to obtain HT within 6 hours of birth (often because of health care disparity in society) or do not qualify for HT or would benefit from greater neuroprotection than HT's mechanism-of-action (MOA) can confer. To quote a recent review, “Although therapeutic hypothermia has improved outcomes, there is still considerable morbidity and mortality that results from HIE in neonates . . . . Combination therapies with single or multiple adjunctive agents . . . should be further studied to augment neuroprotection from cooling.”

Early studies gave hope for neuronal circuitry replacement (FIGS. 28A-B), but the work as disclosed herein with hNSCs (FIGS. 19-29) is convincing to (a) hNSC-derived neurons were not restoring a sufficient population of functional circuitry and (b) such restoration was actually not necessary nor was even responsible for the improvement being observed in rodent models of acute/sub-acute perinatal HII (the major cause of CP); the actual MOA of the NSCs was impressive neuroprotection (mediated by the abundant donor-derived non-neuronal population) that was preserving endangered host neural circuitry. Furthermore, data suggesting that, HT, which is SOC, can unfortunately impair hNSC function (donor hNSCs and likely endogenous hNSCs—do not “like” being cold for long) (FIG. 35) and is not sufficiently neuroprotective (FIGS. 31-35), administering hNSCs at the conclusion of HT (when the animal/patient has been rewarmed) can enable their successful co-administration (and potential synergy between the 2 modalities) (FIGS. 34A-B).

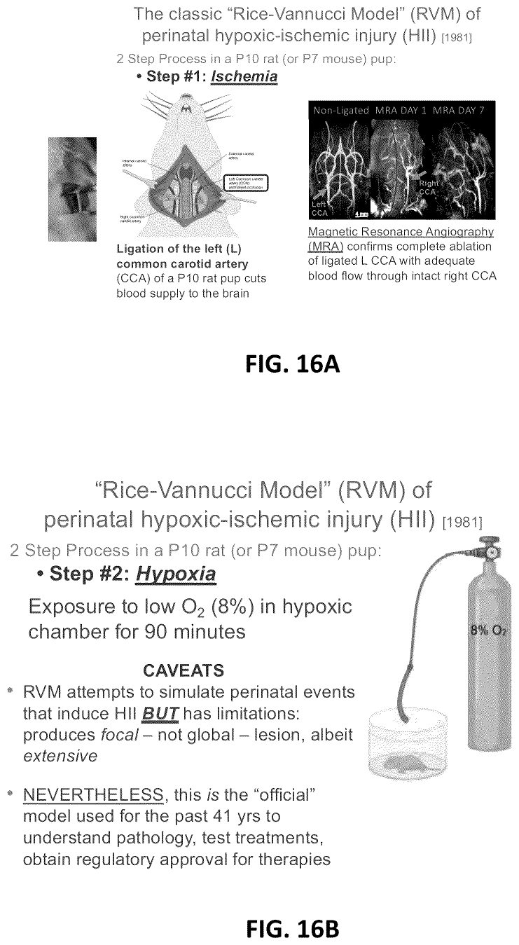

Neonatal HII causes rapid irreversible necrotic injury in the ischemic “core” with an outward radiating gradient of potentially reversible injury and/or salvageable tissue in the “penumbra” (cell death here typically results from delayed, slower apoptosis and other mechanisms of secondary injury). Preclinical work has been done primarily in the FDA-accepted classic 40-year-old Rice-Vannucci Model (RVM) in which HII is induced in the left hemisphere of a 10-day-old rat pup (or 7-day-old mouse pup)—equivalent to a full-term human neonate—by permanently ligating the left common carotid artery (CCA) followed by a 90 minute exposure to a normothermic (37° C.) hypoxic (8% FiO2) environment (FIGS. 16A-B, 19, 25). The injuries occurring in the RVM affect the cerebral cortex, the deep gray matter (caudoputamen and thalamus), the subcortical and periventricular white matter, and the hippocampus. An HII-induced lesion is detectable by using T2-weighted imaging (T2WI) MRI as early as 1- to 2-day post-HII (FIG. 19). Therefore, as early as that timepoint post-HII, animals can be classified based on the rat pup severity score (RPSS), a number (on a scale of 0 to 4) determined by a measurement of the total HII brain lesion volume delineated by T2WI. The RPSS, which has been well-established to reflect accurately the histological and behavioral hallmarks of HII, enables stratification of experimental groups into “mild” (0.25-1.49), “moderate” (1.5-2.49), and “severe” (≥2.5) HII (FIGS. 27A-D, 28A-D).

An automated objective computational imaging analysis method has been devised (Hierarchical Region Splitting [HRS]), which combines T2WI with diffusion weighted imaging (DWI) to reliably (comparable to histology) delineate not only injured (vs. normal) brain parenchyma, but also, within the HII lesion, distinguish and quantify salvageable penumbra from irreversibly-injured necrotic core following HII (based on diffusion-perfusion mismatch) in both rodent (FIGS. 20-24, 26A-C, 29) and human neonates (FIGS. 21A-B, 23). This additional granularity of being able to sub-categorize the composition of the HII lesion into “necrotic core” (pseudocolored red) vs. “penumbra” (pseudocolored blue) allows for a more refined classification (FIGS. 24A-B): a lesion of a given size composed primarily of necrotic core is designated as “severe” (core>penumbra); one in which a measurable penumbra constitutes more of the lesion than does the core, is designated as “moderate” (penumbra>core). A lesion with little or no core and all penumbra, if detectable at all, is designated as “mild”. In other words, the RPSS taken together with the HRS relates MRI to the histology and behavior of an untreated HII rat pup. This MRI-based lesion-based classification system is not the same as the routine Samat clinical staging system used at the bedside by neonatologists categorizing human neonates in the NICU based on their Apgar scores, neurologic exams, and systemic blood values of pH; no imaging, electroencephalogram (EEG), or assays of other markers are presently involved in present-day routine clinical decision-making or practice.

Quantitative analysis of T2WI at 1 to 2 days post-HII (1 to 2 days before hNSC implantation) reveals that the RPSS correlates with the lesion size (r=0.76, p<0.002). As an example (FIG. 25), for moderately injured rat pups, with an average brain volume of 641.9 mm³, the average total lesion is 19.2% of total brain volume (TBV) and the RPSS averages 2.4. The total lesion consists of a small core (6.5% of TBV, 33.9% of the lesion) surrounded by a larger penumbra (12.7% of TBV, 66.1% of lesion). These imaging tools have been complemented by Magnetic Resonance Spectroscopy (MRS) which allows a real-time, longitudinal, noninvasive biochemical/metabolic correlate.

It has been demonstrated that this type of lesion does have functional and behavior consequences in rat pups, not simply abnormalities found on imaging or histology (FIG. 25). In other words, significant neural networks have been disrupted.

New data shows that NSCs, including those appropriate for clinical use (Table 7) introduced into the lateral cerebral ventricles (LVs) of the RVM rodent model of HII home to parenchymal areas of HII (FIGS. 18A-B, 19, 27A-D, 28A-D), an intrinsic pathotropism, based in large part, on chemoattraction to inflammatory cytokines, which impels them to (a) migrate to injured regions even over long distances, including to the opposite side of the brain (Table 7; FIGS. 18A-B, 19, 27A-D, 28A-D, 30A-B), (b) often alter their differentiation fate to help reconstitute the elements of the damaged region, but, most importantly, (c) recruit host neural and non-neural self-repair elements and (d) salvage the penumbra (via multiple mechanisms) leading to histological, MRI, and behavioral “rescue” (e.g., FIGS. 19, 26-30, 32-34, 37-40). These therapeutic mechanisms include providing trophic, neuroprotective, and extracellular matrix support, promoting host neurite outgrowth and angiogenesis, mobilizing endogenous NSCs, direct anti-inflammatory and anti-scarring actions, inhibiting gliosis, scavenging free radicals and excitotoxins, restoring host neuronal energetics and lysosomal function, providing inter-neuronal connections and glial support, etc. (Table 9).

No immunosuppression has been used in these studies and no immunosuppression is planned for the proposed clinical trials. The hNSCs lack MHC class 2 and are not immunogenic, at least not in the newborn brain.

The use of MRI was pioneered to monitor and document these actions non-invasively and longitudinally in real-time, tracing the fate of superparamagnetic iron oxide (SPIO)-pre-labeled hNSCs as early as 1 to 2 days post-transplant and for at least 58 weeks in vivo, with no adverse effects on the recipient's neural and general somatic development. That system has also been used to quantify migrational speed and NSC proliferation/survival (FIG. 19). Note that SPIOs, although FDA-approved, will not be used to label the hNSCs in the proposed clinical trials; they have only been used for preclinical proof-of-concept studies to track the hNSCs and have even been excluded for the latest Pre-IND-enabling studies.

Although implanted on the side contralateral to the infarct, by 90 days post-HII, the majority of donor-derived cells in the moderately and severely injured brains are located in or surrounding the lesion (both by MRI and histological criteria) (FIGS. 18A-B, 19, 26-28, 30A-B). Although about a third of these donor-derived cells expressed the cell division marker PCNA (“proliferating cell nuclear antigen”), suggesting they were initially proliferative post-transplant, the hNSCs become quiescent (after 1-2 cell divisions) and never cause tumors, overgrowth, or deformation of normal structures. Speaking to safety, no NSC-recipient animal has ever been worse than saline-treated controls.

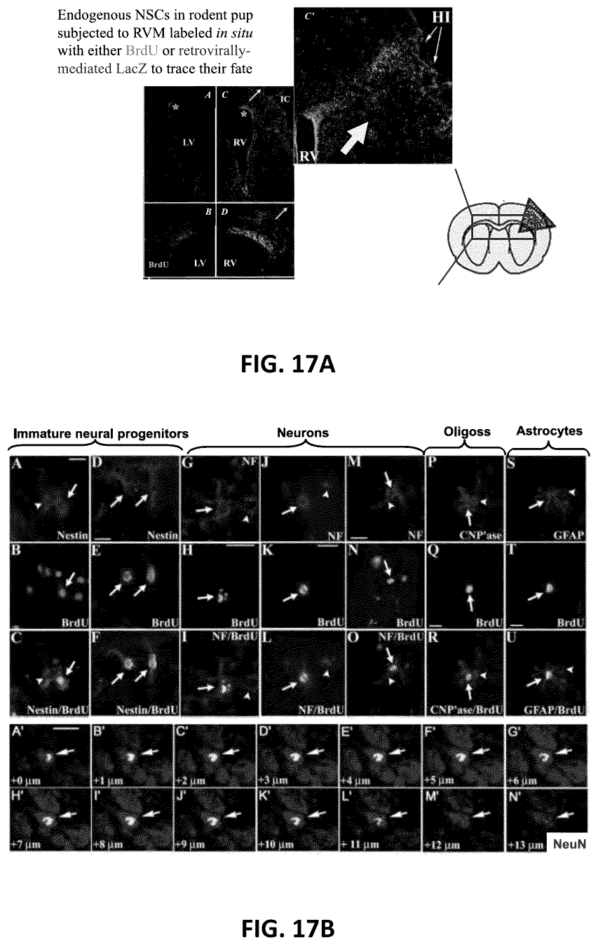

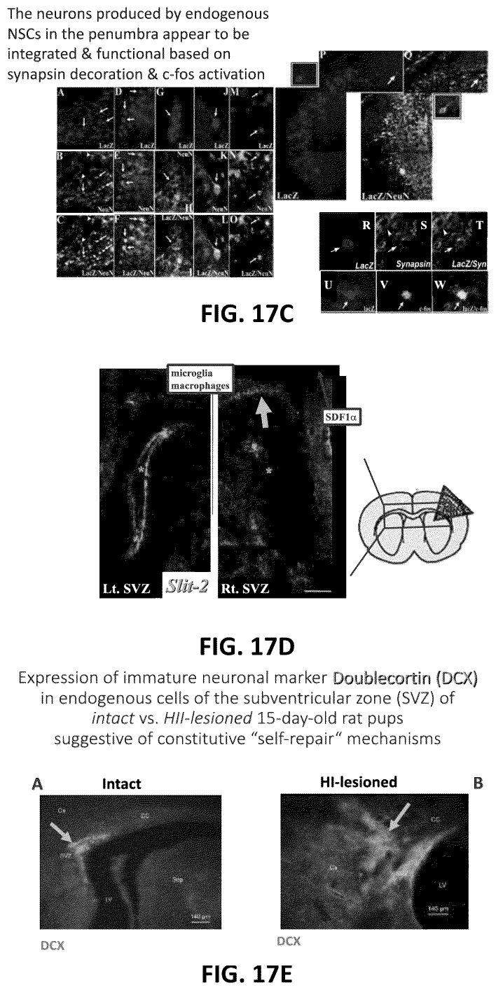

Important to the rationale of this proposed clinical trial is the recognition that this NSC-based approach simply augments in a safe and minimally-invasive manner a natural, “self-repair” mechanism constitutively induced in the newborn mammalian brain by HII, emanating from the SVZ (the germinal zone for postnatal NSCs) (FIGS. 17A-E). Indeed, it is the only proposed therapy (likely in the history of perinatal HIE translational research) that attempts to harness and exploit one of the brain's own intrinsic, innate homeostasis-maintaining programs. Also, unlike most treatments that target one pathogenic process, a cell-based therapy such as this invokes (by its very nature and via its teleological role) multiple mechanisms-of-action targeting simultaneously many of the pathogenic processes triggered during acute and subacute HII (including necrotic and apoptotic cell death, excitotoxicity and oxidative stress, vascular and extracellular matrix disruption, inflammation and scarring, energy failure, loss of trophic support, axotomy, demyelination, etc.) Complex neurological conditions require multimodal therapeutic approaches—and a cell-based approach using a cell native to the brain (a pivotal component of Nature's own homeostatic mechanisms) (Table 11) and employing its normal migratory routes (Table 12) is not only the right cell, but likely the only cell programmed to do so. As published before, there is a danger of putting something in the brain that does not belong there.

TABLE 11
Comparing Various Types of Cells for Rescuing the
Penumbra in a Perinatal Hypoxic-Ischemic Lesion

NEURAL STEM CELLS (NSCs)
Native to brain and integral constitutive components of organogenic and homeostatic processes
Intrinsic properties designed to restore homeostasis (including neuroprotection, detoxification,
trophic support, and cell replacement) in the brain.
Endogenous NSCs programmed to migrate from the SVZ to the region of
injury to attempt to “repair”
Essential property includes pathotropism (homing to pathology for purposes of restoring
homeostasis).
Established migratory routes
Intermix with endogenous NSCs
Integrate and differentiate following transplantation
Proliferation controlled by brain environment
Off-the-shelf reagent, patient-ready, already quality controlled
Homogenous
No lot-to-lot variability
Defined by neural function as well as by markers
30 years of safety and efficacy in preclinical models and safety in clinical trials
True NSCs not capable of forming tumors
MESENCHYMAL STROMAL CELLS (MSCS) (FROM ANY SOURCE)
MSCs are not native to the brain and do not have the intrinsic, constitutive neural-specific
homeostatic, self-repair molecular programs that true neural cells have
No natural migratory route for MSCs to reach the penumbra (whether administration is
intraventricularly, intranasally, intravascular, intraparenchymal).
Migration and homing of MSCs not as great as NSCs (which are “programmed” to normally
migrate from the SVZ to regions of pathology)
Don't integrate
Short-lived (unclear whether they will persist long enough in a healthy state to produce the
neuroprotective factors necessary)
Will continue to proliferate in response to inflammatory cytokines within brain (because not
equipped to respond to neural-specific cues)
No evidence that BM-derived MSCs are drawn from the vascular space or the bone marrow to
the injury site and do anything therapeutic
Hard to “quality control”
Heterogenous
Poorly characterized other than markers
Risk of extensive lot-to-lot variability since must be prepared anew for each patient, even
if allogenic
Time-consuming preparation (must be on a case-to-case basis) to reach release criteria for
safety and efficacy within the 3-4 day window time period necessary to rescue the penumbra
Risk that hematopoietic-derivatives that enter a sub-acute ischemic lesion will yield toxic
inflammatory cells.
UMBILICAL CORD BLOOD
Not native to the nervous system - therefore same limitations as MSCs above
Hard to quality control because must be prepared fresh for each patient within 3-4 days
producing lot-to-lot variability with regard to efficacy, safety, purity
Will not pass through intact BBB
Those that pass through a breach in the BBB do not persist long enough to be therapeutic, do
not integrate, and do not concentrate within the penumbra

TABLE 12
Comparing Various Routes of Administration for an Allogenic
Cell-Based Neuroprotection of the Penumbra of a Perinatal
Hypoxic-Ischemic Injury (HII) Lesion

INTRAVENTRICULAR ROUTE:
Only route that augments the normal self-repair mechanism of the newborn mammalian
brain in response HIE; places hNSCs in a natural NSC germinal zone (SVZ)
Ventricles are residua of the neural tube, the basis for CNS development
Well-dissociated, immature, hNSCs in G1 cell cycle phase, do not remain in the
ventricles, but rather intermingle with host hNSCs in the SVZ and then participate in,
while augmenting, natural constitutive self-repair processes --homing with endogenous
NSCs to the infarct (as endogenous NSCs are induced to do by HII (see FIG. 16).
Will replace any endogenous NSCs that were killed or impaired by the HII
No need to target the infarct (which often is large and difficult to visualize or access)
Inherent biology of NSCs includes pathotropism, but specifically tailored to nervous
system pathophysiology
Like intrathecal administration except places cells closer to region-in-need of
neuroprotection hence optimizing homing
Route efficiently and unequivocally ensures thatadequate threshold ratio of donor-to-host
cells are achieved in the penumbra to exert a bioactive impact on host cells (i.e., a
paracrine effect) (as defined in Ref. 18)
Minimally-invasive
30-34 gauge needle
No craniotomy - needle inserted percutaneously through open anterior fontanelle
No general anesthesia or systemic analgesia (topical anesthetic, swaddling ± mild
sedation)
No post-op recovery period
No separation of baby from mother
No need to withhold feeds
Can be performed at bedside or in procedure room adjoining NICU (not need for OR)
Impossible to “miss” ventricles and enter parenchyma, especially under ultrasonic
guidance and with evidence of free backflow of CSF before injecting cells
Procedure is performed only once for each ventricle (not repeated) (Ref. 89)
Route of administration has been safely used for stem cells in previous clinical trials in
both newborns (for IVH) (Ref. 28) and older children (Batten's Disease) (Ref. 79)
[NCT00337636, ClinicalTrials.gov]
Insertion of small gauge needle into ventricles of newborns via the open anterior
fontanelle performed by generations of neonatologists (e.g., to instill antibiotics for
ventriculitis, withdraw CSF when hydrocephalus)
Routine minimally-invasive neurosurgical procedure (see §13.2.3)
Administration needle much thinner than reservoirs or shunts routinely inserted for
CSF drainage or intraventricular endoscopy (Ref. 80)
Smaller than needles used for DRIFT procedure in preterm babies for PHH (Refs. 90-91)
Not chronically indwelling
Brief period of administration (<30 secs).
Extensive preclinical data presented here demonstrates safety and efficacy.
No evidence of parenchymal damage or inflammation or even evidence of a needle track
[see FIG. 51]
Never hydrocephalus, obstruction to CSF flow, increased intracranial pressure, distortion
of normal cytoarchitecture
Adult animal injected with saline as a pup looks identical to an untouched intact adult
animal at region of insertion
Risk benefit ratio favors this “one-and-done” injection with an ultrathin needle for this
heretofore untreatable extensively cerebral disruptive condition
Most efficacious way to get a sufficient number of therapeutic cells quickly to region in
need of neuroprotection
Safe in the hands of a competent pediatric neurosurgeon
INTRAVENOUS:
Cells typically lodge in spleen, liver, and lung as first pass filters; very few enter brain
Cells do not normally pass through the blood-brain barrier (BBB)
Those that do make it through a breached BBB are not in sufficient numbers to exert a
paracrine effect
INTRACAROTID:
Risk of obstruction to vascular perfusion of region, exacerbating ischemia
INTRANASAL:
Is being administered by a non-natural route that does not ensure delivery of cells under
normal or injury conditions
No natural neuroanatomical route for cells to migrate seamlessly to the penumbra
No nasal-neocortical migratory route
Only endogenous neurons known to migrate developmentally from the nasal epithelium
to the brain are specialized GnHR-1+ neurons, but they migrate only to the anterior brain.
Not possible to achieve the critical threshold number of hNSCs needed into the lesion to
mediate a therapeutic paracrine effect.
Difficult to be administer to babies on constant distending airway pressure (CPAP) with
nasal prongs

### Data

Transplanted Human-Derived Neural Stem Cells (hNSCs) Alone are Very Neuroprotective

The initial standard procedures pursued in the rodents was based on 25 years of experience. 2.5×10⁵ hNSCs were slowly infused in 5 μl of phosphate buffered saline (PBS) through a 34-gauge Hamilton syringe into the rostral horn of the lateral cerebral ventricle (coordinates: 0.4 mm caudal to bregma, 1.5 mm lateral from the midline, 3.5 mm ventral to the pial surface) of P12-13 rat pups, 2 to 3 days after they have induced cerebral HII via RVM (at P10). The passage of this very fine needle into the ventricles and the injection procedure causes no damage on its own, as assessed in intact normal rat pups (FIG. 41). No immunosuppression is used and yet there is no evidence of an intracerebral or systemic immune or inflammatory reaction against the cells (even in this xenograft setting), no monocyte or T-cell infiltration, no microgliosis or astrogliosis, no evidence of cell rejection. In order to know exactly the severity of the lesion on the day of transplantation, just prior to grafting, an MRI is performed (T2WI, T2 maps, and, if longer follow-up with HRS analysis is necessary, also DWI).

The natural history of an RVM HII lesion in a rat pup is illustrated in FIGS. 22A-B. Using HRS, one can see that a lesion characterized predominantly by a salvageable penumbra (therefore, “moderate”) evolves into predominantly irretrievable core as the cells in the penumbra die over the course of ˜2 weeks (therefore, evolving into a “severe” classification) unless there is a neuroprotective intervention, ideally beginning at 3 days post-HII. That period is a “window of opportunity for rescue” (FIGS. 19, 26-46) and has indicated it can be exploited by hNSCs to impede or even reverse the spatiotemporal evolution of the lesion and return the parenchyma (and, critically, the fibres de passage coursing through the penumbra (FIG. 48) to normal.

By way of example, in the experimental series illustrated in FIGS. 25-28, at 3 months post-perinatal HII (i.e., at adulthood), the average total lesion volume of pups that received hNSCs was smaller than in saline-treated pups (p<0.04); lesion volumes decreased by ˜67%. Importantly, the decrease in lesion size was solely attributable to decreased penumbral volumes (tissue that was injured but salvageable) (p<0.000001). Penumbral tissue that cannot be rescued continues to cascade toward death by necrosis (FIG. 25). The penumbra was almost completely gone in the hNSC group (0.5% of TBV; 6.7% of the remaining lesion) but remained prominent in the saline group (4.4% of TBV; 49.4% of remaining lesion). This outcome was shown repeatedly (˜200 animals have been so treated to date) (FIGS. 19, 26-28, 42, 46). In the saline-treated group, 19.2% of the penumbra evolved into “core” (i.e., progressed to cell death). In contrast, for the hNSC-treated group, only 8.2% of the original “penumbra” converted to “core”, and 87.8% converted to “normal” brain tissue (i.e., presumably “rescued” from death) (FIG. 25). Severely infarcted brains, characterized almost entirely by unreclaimable core and minimal penumbra, were unimproved by hNSCs; lesion sizes remained essentially unchanged.

The rescue by hNSCs noted at 1-month post-HII appears to be permanent, remaining stable into adulthood, to at least 3 months post-lesion/post-transplantation, the time at which the study was terminated, and the animals sacrificed for analysis.

Neither spontaneous lesion recovery nor neuroprotection by vehicle alone or by hNSC-conditioned medium alone was ever observed (the latter control ruling out a role for simply neurotrophic factors or exosomes being secreted into the medium and mediating the neuroprotection).

Regions of severe injury (defined as a necrotic core where host cells have already died) do not disappear. If the HII volume is >30% of the TBV and is comprised principally of core red regions) with little or no penumbra (blue regions), then the grafted hNSCs have little substate upon which to exert their neuroprotective actions and have no impact. However, most lesions in the rats and live births in human babies do have a penumbra.

### Molecular Signature

Investigation of whether the regions of the brain that appeared most impacted by the hNSCs had a molecular signature supportive of the hNSCs' neuroprotective MOA was conducted, for which an appropriate MRI signal might be a clinically-useful predictive biomarker. A previously published extensive gene expression profile of the impact of the acutely-injured rodent brain on NSCs following experimental HII. Compared to the response to uninjured or chronically-injured brain, in response to acute HII, there was an upregulation of genes that mediate migration, proliferation, differentiation, and anti-apoptosis—a very metabolically and molecularly active region indicative of “regenerative processes”. Heat shock protein-27 (HSP27) is recognized as a surrogate marker for that constellation of molecular processes; indeed, HSP27 is an established sensitive marker for the ischemic penumbra. The team observed that, in the brain regions most highly-populated by donor hNSCs (FIGS. 27A-D), as well as those most robustly rescued by the hNSCs, host cells in the region showed increased HSP27 expression following HII (FIGS. 27 A-C). HSP27 expression was more prevalent in “moderately” injured brains compared to “severely” or “mildly” injured brains (FIGS. 28 A, B). This observation is consistent with the presence of a “penumbra” (greater than “core”) as the defining characteristic of moderate HII, and the therapeutic target of hNSCs. Moreover, penumbral HSP27 expression positively correlated with the RPSS, with maximum HSP27 expression at a moderate RPSS values (FIGS. 28 A, B). Further linking hNSC's MOA with rescue of salvageable brain, they found HSP27 expression also to be upregulated in the engrafted donor hNSCs themselves in the penumbra, though not in hNSCs prior to transplantation or those found in the necrotic core (FIG. 28 D). Although HSP27 co-localizing with the human-specific marker Stem-101 (identifying hNSCs) could be observed in all 3 post-HII severity designations, the number of HSP27+ hNSCs was greatest in the penumbra of moderately injured animals (FIGS. 28 A, B).

With regard to the upregulation of HSP27 in areas that are likely “hNSC's therapeutic target”, 3 processes are likely ongoing in concert: (1) HSP27+ endogenous cells are producing an injury-related chemoattractant that draws hNSC migration and engraftment to where there is HSP27 expression, and the reclaimable penumbra it defines (a pathotropism described nearly 20 years ago); (2) engrafted hNSCs induce the further expression of HSP27 in target cells based on the “cocktail” of “reparative” cytokines secreted by the hNSCs (many of which have been previously described); (3) the acutely-injured milieu induces a molecular “repair program” within the donor hNSCs, marked by their own upregulation of HSP27.

As affirmed by both MRI (FIGS. 19 and 27A) and histology (FIGS. 27B, C), hNSC migration from its injection site to the injury area in the contralateral hemisphere positively correlated with severity of the HII (FIGS. 27A-C) and the extent of hNSC engraftment positively correlated with RPSS (FIG. 27B). “Moderate” and “severe” HII brains were characterized by the presence of more engrafted hNSCs compared to the “mild” HII brain (both as an absolute count and as a proportion of all cells in the area). While there was a difference in hNSC engraftment between the “mild” and “moderate” HII brains, and between the “mild” and “severe” HII brains, maximum engraftment was reached at “moderate” severity (and was sustained even with greater severity) (FIG. 27C). Only the “moderate” HII brains had a salvageable penumbra (i.e., the most HSP27+ cells); the number of hNSCs present was, indeed, sufficient to reduce lesion size and, as reported below, lead to functional improvement. Because severe lesions are defined as having a predominance of unsalvageable core and little HSP27-expression, no greater number of hNSCs were drawn there than were present in moderate HII lesions. In other words, the strong correlation between hNSC concentration and RPSS (severity) appears to reach a saturation point at a “moderate” RPSS and moderate tissue injury before plateauing at the point where a penumbra is undetectable and the lesioned area can no longer be deemed “moderate”, but rather “severe” (FIG. 27B). It is impressive that, while the number of hNSCs in the severe brains did not increase, it also did not decrease. That maximum number of hNSCs together with a larger penumbra-to-core ratio identifies best “responders” to the hNSC intervention (FIGS. 27B, 28A, B).

It becomes important to note that simply the presence of hNSCs does not produce a therapeutic outcome. Both components of efficacy must be present: (a) sufficient numbers of reparative cells, and (b) a region that can be repaired, reinforcing the utility of a mechanistically-based patient selection/prognostic/diagnostic tool for efficacy, HRS. (However, for the proposed clinical trial, HRS will not be used for patient selection, although HRS data [simply a post-hoc mathematical algorithm performed on the digital MRI data that will already be accumulated as SOC] will be collected for retrospective analysis and clinical correlation).

### Differentiation Fate

As to the differentiation fate of the engrafted hNSCs, ˜5% (±1%) came to co-express the mature neuronal marker neuronal nuclear protein (NeuN). Although this co-localization between NeuN and human-specific markers could be observed in all 3 ischemic injury conditions (FIG. 27D), none of these NeuN+ cells made functional connections in the way mouse NSCs (mNSCs) may have. Indeed, it is believed that the MOA of hNSC-mediated recovery in these models is not the replacement of destroyed neural networks (into which these NeuN+ cells would need to integrate) but rather the preservation of pre-existing, though endangered, neural networks likely coursing through or located within the penumbra (“fibers de passage”). It is more plausible that the HSP27+ milieu pushes the molecular, though not the functional, differentiation of these multipotent undifferentiated hNSCs toward a neuronal expression profile, an action well-established for HSP27. It is the more numerous non-neuronal progeny of the hNSCs, reported by us and others to produce neuroprotective and homeostasis-promoting cytokines that are responsible for salvaging the penumbra: quiescent undifferentiated hNSCs (Nestin+, 54%), astrocytes (GFAP+, 39%), and oligodendrocytes (CNPase+, 2%). The presence of donor-derived NeuN+ cells supports that hNSC-derived cells remain stably engrafted and long-lasting throughout the lifetime of the animal.

In view of the relatively quick neuroprotective effects seen following transplantation hNSCs (FIGS. 33A-B), it is likely that part of the molecular mechanism of therapeutic action of the cells is a quick distribution of cells that produce anti-oxidative, anti-excitotoxic, anti-inflammatory, scavenger, and neurotrophic factors, dissemination of which is abetted by both the migratory nature of the hNSCs and the flow of the cerebrospinal fluid (CSF). However, the explanation cannot be that simple because cell-free hNSC-conditioned medium alone cannot replicate this effect; in fact, conditioned medium alone conferred no neuroprotection; the hNSCs themselves are critical and are providing additional neuroprotective support through additional mechanisms. This speculation is consistent with the extension of astrocytic GFAP+ processes by the donor cells through the ventricular walls (FIG. 45), suggesting that these cells might be also exert beneficial influence locally in the affected parenchyma of the penumbra through cell-cell contact with endangered host cells located there. For example, HII can induce an overproduction in astrocytes of IL-33, (a novel member of the IL-1 family of cytokines), and the activation of IL-33/ST2 signaling in the ischemic brain influences astrocytic behavior which, in turn, affords protection to ischemic neurons in a GDNF-dependent manner. Therefore, astrocytic derivatives of the grafted hNSCs may be augmenting or re-establishing such function. The donor cells may also be change the fate of endogenous astrocytes to return to a fetal, trophic state rather than cascading towards an injurious reactive one (e.g., FIGS. 46, 47A-B).

Induction by HII of Host Self-Repair Mechanisms Mediated by Endogenous NSCs (A Process which Exogenous hNSCs Simply Mimic and Augment)

It is important to note that the minimally-invasive instillation of hNSCs (in suspension) into the cerebral ventricles (percutaneously through the open anterior fontanelles), enabling them to integrate into the SVZ and intermix seamlessly with endogenous NSCs, is simply augmenting a constitutive developmental, self-repair, homeostasis-promoting process that HII invokes in mammalian brains wherein endogenous NSCs in the SVZ migrate to the ischemic lesion in an “attempt” to limit damage and to repopulate the infarcted cerebrum (including cortex) with a normal array of neural constituents. Data for this self-repair “program” is provided in FIGS. 17A-D. This process—in which endogenous NSCs re-enter the cell cycle (e.g., incorporate BrdU) (FIG. 17A) and are “pushed” and/or drawn to the infarct (FIG. 17D) where they give rise to replacement neural constituents (FIGS. 17-B, C, E)—is probably not sufficient for greater than mild injuries. Therefore, in the past, that process has been augmented with additional exogenous NSCs, employing the same route-to-the lesion and encountering the same inductive cues. In FIGS. 18A-B, they illustrate their used of mouse NSCs. In the remaining figures of this proposal (e.g., FIGS. 26-30, 32-35, 37-40, 42-45) they employed human-derived NSCs (hNSCs) in the same manner. And, the proposed clinical trial will employ these same hNSCs in a similar manner, optimized for route-of-administration, dose, and timing in relation to HT, as detailed in the remainder of this package.

In addition to augmenting the number of NSCs in the SVZ, the exogenous hNSCs also appear to have a direct influence on the host NSCs in terms of enhancing their own “regeneration-inducing” actions. For example, although HII alone will induce host brain cells to proliferate (as measured by their incorporation of BrdU pulsed daily for the first 3 days post-HII (Postnatal Day [PD]) (FIGS. 17A, B), in the presence of engrafted hNSCs, there is even greater proliferation (although the hNSCs themselves rarely incorporate the BrdU [i.e., do not themselves divide in vivo]); in fact, these quiescent hNSCs were often enveloped by dividing host cells. The hNSCs also induce in the host an upregulation (beyond that seen following injury alone (FIG. 17-E)) of cell types that are pivotal for self-repair: e.g., doublecortin (DCX)-expressing cells (DCX is a microtubule-associated protein that marks migratory neural progenitor cells or neuroblasts destined to become young migratory neurons) and TuJ1-expressing (Tuj1 is a marker of β-III-tubulin which is associated specifically with young neurons). In fact, it is likely that these cells have differentiated from the large proportion of BrdU+ host cells induced by HII and augmented by the hNSCs.

Exogenous hNSCs may also increase, within host NSCs, expression of the “regenerative biomarker” HSP27 in ischemic areas, particularly the penumbra (FIGS. 18A-B).

The hNSCs, while proliferative in the dish (FIGS. 9A-C, 10A-B) quickly exit the cell cycle in the brain, become contact-inhibited (as they do in the dish (FIGS. 11A-D)), and begin to differentiate (FIGS. 5-8, 14B). So far, using a human-specific antibody against GFAP (STEM123), which detects only hNSC-derived cells, the donor-cell-derived astrocytes were found extending their processes away from the ventricles and into the host's brain parenchyma (FIG. 45). Donor-derived cell expression of DCX and TuJ1 was present, as well, particularly in hNSCs that had integrated into the host ependyma or parenchyma. Similarly, donor-derived hNSCs, when encountering the penumbra, have their own HSP27 expression “turned on” (FIG. 28D).

Route of Administration (ROA): HNSCs Instilled into the Ipsilateral or Contralateral Ventricle are Equally Safe and Effective

As indicated above, under “Background”, it was easy to rule out routes of administration (ROA) other than into the lateral cerebral ventricles percutaneously (Table 12): cells administered intravenously lodge principally in lung, spleen, and liver, and cells administered via the carotid, intranasally, or intrathecally (i.e., into the spinal CSF space) simply do not deliver a sufficient number of cells to come close to reaching the threshold ratio of donor:host (optimally 1:10 and nor more dilute than 1:140) to achieve a paracrine effect. Likely these poor strategies for enabling donor NSCs to intermix with host hNSCs is attributable to the fact that routes other than via the SVZ are not natural routes-of-migration and repair in the developing and immature brain mammalian brain. The intraventricular route has been used safely, effectively, and preferentially in many preclinical and clinical studies addressing brain abnormalities or diseases.

To determine whether the best ROA of the hNSCs into the ventricles was ipsilateral versus contralateral to the HII, another series of experiments were performed. They infused 17 rat pups with hNSCs into the cerebral ventricle contralateral to the HII and 13 rat pups with hNSCs into the ventricle ipsilateral to the HII and followed them until 3 months-of-age (adulthood). During that period, these animals and the behavior of their lesions were monitored in MRI sessions 5 hours prior to transplantation and on post-transplantation days (PTrD) 3, 30, and 90. Controls included untreated animals and lesioned animals injected with Hanks Balanced Salt Solution (HBSS) or hNSC-conditioned medium. FIG. 30A illustrates the robust shift towards mild (no necrotic core) lesions (blue sections) in the grafted animals over time (middle and bottom rows) in comparison to the non-grafted or vehicle-grafted controls (top row). In the latter animals, severe lesions persist (red sectors), moderate lesions (orange sectors) evolved into severe ones (red sectors), and mild lesions (blue sectors) progressed into moderate lesions (orange sectors). By comparison, the neuroprotective effect of the grafted hNSCs lead to robust amelioration, reflected in the reversion of moderate lesions (orange sectors) into mild lesions (blue sectors) via rescue of the penumbra, the predominance of which defines a moderate lesion categorization by MRI criteria. This recovery, in the case of grafted animals, was often quite rapid, was stable by 1-month post-transplant, and persisted until at least 3 months postlesion/post-transplant (i.e., adulthood) with no progression or regression (as seen in untreated brains).

The MRIs with HRS in FIG. 29 capture the dynamics of the rescue. They show the brain of a representative PTrD 90 rat that received hNSCs into the cerebral ventricle contralateral to the lesioned hemisphere. Starting with a lesion of a moderate degree of severity, one can see complete resolution of the penumbra (blue region) and no increase (indeed, a seeming decrease) in the core (red region). (A similar dynamic can be seen in FIG. 38 which strikingly reverses the natural history of an HII lesion wherein the penumbra evolves into core [FIGS. 22A-B]; rather with interposition of neuroprotection via the hNSCs, penumbra reverts to normal.)

The presence of the hNSCs leads in all mild and moderate cases to a significant reduction of the lesion—and certainly little-to-no progression of severity—whether they are grafted contralateral or ipsilateral to the lesioned site. Few if any moderate lesions progress into severe lesions, as they do (quite dramatically) in the absence of hNSC implantation (red regions in top row at PLD90 in FIG. 30A).

The analysis above, based on HRS, suggests that ipsilateral administration perhaps salvages a bit more of the “moderate” regions, improving them to “mild” (i.e., clinically insignificant), and prevents a bit more of those “moderate” regions from becoming “severe”. Analysis of the same animals in FIG. 30B, based on hemispheric volume (rather than clinical/MRI severity classification, but required histology) gives a slight advantage to contralateral administration. (Clinically, severity classification based on MRI and exam will be what is actually used in living babies). Therefore, on balance there seems to be little difference between the two ROA based on efficacy (reversing severity or suppressing progression of severity of the HII lesions), safety, and ease of injection. This conclusion that an ipsilateral vs. contralateral ROA yields essentially equally beneficial results supports clinical trial in which hNSCs will be injected into both ventricles. First, it puts to rest concern that immediate direct contact of the hNSCs with the “toxic milieu” of an infarcted area is inimical to their health and function; they can still effectively exert their neuroprotective action. Second, unlike in the RVM animal model, actual perinatally-asphyxiated babies usually have bilateral, even global, cerebral lesions; it is important to know that the hNSCs can home to lesions whether ipsilateral or contralateral to the site of implantation and provide neuroprotective relief Because of this clinical reality, in the actual clinical trial, hNSCs will be injected bi-ventricularly.

Not only was there no spontaneous lesion recovery (an absence of intervention allows “moderate” HII to progress to “severe” and “mild” HII to worsen to “moderate”), but the absence of any neuroprotective effect after injections of medium conditioned by the hNSCs rules out a role for neurotrophic factors or exosomes simply being secreted acutely by the hNSCs as mediators of the rescue. Presence of the hNSCs themselves appears required because they likely mediate their therapeutic actions via multiple mechanisms, including impacting endogenous host reparative responses (as discussed previously). Importantly, the hNSCs play this neuroprotective role even in the hostile milieu of the lesioned hemisphere, suggesting that they are somewhat resistant to such insults, and that they themselves may play a role in neutralizing or scavenging these toxins.

### No Adverse Effects

No adverse effects were ever observed in >300 animals. There was never evidence of ventriculomegaly from hydrocephalus or obstruction to CSF flow. There was never cell overgrowth, deformation of the host cytoarchitecture, tumors, neoplasms, mass, or pressure effects. A needle track was not obvious at adulthood nor was there any scarring or host cell death (FIG. 41). No host- or donor-derived non-neural cells were observed or any cells or structures that were inappropriate to the brain. In addition, the animals did not encounter any systemic adverse symptoms: no pain or discomfort, no increased mortality/morbidity attributable to the hNSCs. There was no sepsis, ventriculitis, or dermatitis. No hNSCs were observed in any organs outside the CNS. (FIGS. 44A-B). Although the hNSCs represent a xenograft in the rat pup brain and although immunosuppression is not used, there was never evidence—either as early as 3 days post-transplant or 3 months post-transplant—of host monocytic, macrophage, or T-cell invasion that might indicate an immunogenic reaction or immunorejection. Immunosuppressive drugs are not planned to be used in the babies during clinical trials, and the lack of an obvious immunologic reaction in these animals despite donor-host species incompatibility, suggests that such an approach is valid. hNSCs lack major histocompatibility complex [MHC] class II and hence are not likely to be immunogenic, certainly not in the newborn brain.

The Optimal Timing of Human Derived Neural Stem Cells Administration in Relation to HT to Maximize Efficacy is 3 Days Post-HII which Coincides with the Completion of HT in Human Neonates

The temporal “window of opportunity” is an important factor influencing the outcome of neurotransplantation. A fresh lesion represents a hostile, cytotoxic environment where inflammatory factors, ROS, edema, excitotoxicity, and dying cells create an unfavorable milieu for grafted human hNSCs, compromising their own survival. On the other hand, an older brain lesion is already characterized by permanent cell loss, glial scarring, vascular disruption, aberrant connectivity, and a microenvironment which lacks the “plasticity signals” donor hNSCs must “sample” to react in a reparative fashion; furthermore, the milieu lacks cells that are still receptive to such rescue and/or ameliorative actions. The situation that persists in these 2 polar extremes explains the 2 negative outcomes reported here below—grafting too soon (including at the initiation of HT) (FIGS. 34A, 35, 36A-C) or grafting too late (1 week following rewarming following HT) (FIG. 34B).

Grafting hNSCs Contemporaneously with Initiation of HT (“Immediate Grafting”) is Less Effective than Waiting for the “Patient” to be Rewarmed

PD10 rat pups (experimentally asphyxiated via the RVM) received an implantation of hNSCs into both lateral cerebral ventricles (5 μL/ventricle; 50,000 cells/μL [HBSS]) at the same time as instituting HT 4 hours post-insult (emulating the actual clinical condition). (Cooling for 4 hours in a rat pup emulates 3 days in a human full-term newborn.) After the period of HT, the pup was rewarmed for 15 minutes, and returned to the dam. On Post-Transplantation Day (PTD) 5 (“PTD 5”) and 30 (“PTD 30”) (FIGS. 34A, B), MRI evaluation was performed to characterize the HII lesion—including applying routine HRS post-hoc analysis to distinguish “penumbra” from “core” within the infarct and, hence, to be able to grade severity of the injury based on MRI. Although the logistics of doing immediate grafting precluded health care provider being able to perform an immediate pre-grafting MRI, the RVM protocols are so consistent and reproducible that it can reliably be known what the spectrum of lesion types and their prevalence would be (FIGS. 34A, A): “mild” (blue) (16%), “moderate” (orange) (72%), and “severe” (red) (12%). Interesting, MRI on PTD 5 (FIGS. 34A, B, Left Pie Chart), initially suggested a beneficial effect from immediate grafting: a disappearance of “severe” lesions and reduction of “moderate” lesions to 20%, with the majority being minor lesions (80%). However, the beneficial effect of immediate grafting was not stable or sustainable, as seen 1 month later (PTD 30) (FIGS. 34A, B, Right Pie Chart): “mild” lesions reverted back to “moderate” ones, and “moderate” lesions progressed to becoming “severe” ones [FIG. 35]. This was never observed in rats grafted during “optimal window” of 3 days after rewarming (which coincided with 3 days post-HII), where lesion reduction remained stable for up to 3 months or longer. The efficacy of HT alone, as reported above, was minor if present at all (FIGS. 31, 32A-B). Similarly, conditioned medium or vehicle alone, as reported above, was also non-impactful.

The findings above in vivo are consistent with other experiments the team performed that indicated that hNSC behavior and function are compromised or impaired by prolonged exposure to the temperatures used for HT (FIG. 35). Both proliferation and migration appear to be reduced by HT (p=0.006 and p<0.0001, respectively); migration, of course, is critical. Metabolism, as assessed by protein synthesis, after an initial bump (likely stress proteins), is suppressed in a somewhat protracted manner (p=0.0004). (n=15/group; each assay performed in triplicate biological and technical replicates). Most of these changes start at <3 days of HT and become quite significant by completion of 3 days. Hence one would not want the donor hNSCs in that HT environment. Given the conclusion that 3 days post-HII is the optimal time for therapeutic hNSC transplantation, and that SOC therapeutic HT is terminated at 72 hours post-HII, it should be possible to coordinate the timing of both interventions in the clinical trial—hNSC administration after HT and rewarming.

Grafting Human Neural Stem Cells 1 Week Following a Course of HT (“Delayed Grafting”) is Ineffective in Altering the Fate of an HII Lesion—in Stark Contrast to the Efficacy Reported Above when Implantation was Performed 3 Days Post-Rewarming/Post-HII

Asphyxiated PD10 rat pups (via RVM) received HT starting 4 hours post-HII (emulating the ≤6-hour window used clinically). After being rewarmed, the pups were returned to the dam. One week later (post-HT Day 7 and post-lesion day (PLD) 7, the lesions were evaluated by MRI with HRS, and the animals were grafted with hNSCs into both lateral cerebral ventricles as described above (5 μL/ventricle; 50,000 cells/μL HBSS).

In this “delayed grafting” paradigm, MRI could be performed on PLD 6, just 12 hours prior to grafting, enabling identification of the spectrum of lesion severity in experimental animals pre-intervention (FIG. 34B, Left Pie Chart; “PLD 6”). Here, it was found, as expected, all the lesion severity types: 20% “severe” (red), 20% “moderate” (orange), 60% “mild” (blue). By this time, 6 days after the insult, the lesions had already started to adopt their final appearance. Delayed bilateral hNSC grafting of these animals (on PLD 7-1-week post-rewarming and post-lesion) did not appear to have any beneficial effects on the resolution of lesion severity or forestalling disease severity progression. On PTD 3 (FIG. 34B, Right Pie Chart), a time point where, in rats grafted 3 days post-HII, a beneficial impact from hNSCs reflected in lesion reduction can be seen, the “delayed grafting” animals showed exactly the same lesion spectrum as before transplantation. To reiterate, delaying the transplantation of hNSCs to 1-week post-HII and post-HT compromised their neuroprotective actions; HT did widen the “window-of-opportunity” to that extent.

In summary, it has been determined the optimal time for transplanting hNSCs in relation to the occurrence of the perinatal HII and to the initiation and conclusion of therapeutic HT is 3 days post-HII and post-rewarming following HT. These observations also ruled out the speculation that HT might significantly broaden the window post-HII during which hNSCs might be implanted. However, 3 days post-HII for effective hNSC transplantation gives plenty of time to treat babies that miss the 6-hour window for HT, including in low- to middle-income countries (given the SOP for preparation and administration of the hNSCs).

### Hypothermia (HT) Alone, as Presently Administered as Standard of Care, is not as Neuroprotective as Human Derived Neural Stem Cells—Does not Sufficiently Rescue the Penumbra or Prevent Evolution of the HII Lesion

As alluded to above, while the above insights were being obtained, a new challenge emerged: immediate (within 6 hours of birth) institution of whole-body HT (33-34° C.) for 3 days—a neuroprotective strategy that is only marginally effective in “moderate” (Sarnat Stage 2) HII and ineffective in “severe” (Sarnat Stage 3) HII (clinical, not pathophysiological or MRI designations)—had become SOC since 2012 in all NICUs. HT has reduced mortality, but it remains unclear whether it reduces morbidity or the prevalence of neurological abnormalities in the survivors. HT also comes with a series of potential side-effects: pulmonary hypertension (causing hypoxia and hypotension); coagulopathy (causing bleeding, including intracranial bleeding); the need for continuous opiate infusions for sedation and to combat pain/discomfort, which inevitably leads to opiate withdrawal syndrome at the end of the procedure; maternal touch and breastfeeding are precluded; and usually babies are not fed enterally in order not to put a metabatic demand on a gut where blood flow is likely compromised from the cold (nutrition is provided intravenously). Such low temperatures may impair (the permanence is unknown) fundamental neural circuits that are cold-sensitive, and likely inhibits the babies cerebral self-repair mechanisms (FIGS. 18A-B); hNSCs “do not like being cold” for 72 hours (FIGS. 36A-C). Furthermore, the recent HELIX clinical trial showed HT to be ineffective (and even inimical) when used in middle- and low-income countries.

A critical part of the study entailed determining the actual efficacy of HT as a neuroprotective strategy using state-of-the-art parameters for quantifying rescue (including using MRI criteria reflect the molecular milieu of the lesion and is predictive of recovery). Interestingly, this type of analysis of HT has never actually been done in the literature. Therefore, the studies reported here, (which are critical for providing the threshold against which hNSC transplantation), are also new to the field, and are quite revealing and sobering.

To mimic the actual clinical situation in the RVM model of perinatal HIE, rats were cooled 4 hours after their left CCA ligation and exposure to 8% O₂ (actually, clinically, an even longer intervening period between HII and HT is permitted—up to 6 hours).

As can be seen in FIG. 31, the entire lesion, and most particularly the unsalvageable core, grows with HT alone over the course of the first month post-lesion. This figure quantifies the contribution of core (hashed) histograms) and penumbral (gray histograms) volumes to the entire lesion (black histograms) after HT in mild-moderate lesions on PLD 2 and 32. While HT alone may provide some initial, transient benefit in slightly reducing the severity of the HI lesion, the lesion volume actually grows because the core volume increases. The lesion characterization resembles that of an HI lesion with no intervention at all.

Given these observations, it was concluded that HT, as presently administered as SOC (and as clinically feasible to administer), is not capable of preventing an asphyxiated baby's brain from suffering from continued oxidative stress, inflammation, and apoptosis.

Synergistic Beneficial Impact on Perinatal Hypoxic-Ischemic Cerebral Injury of Supplementing HT with Human Neural Stem Cell Infusions into Both Ventricles

Although HT's actual MOA is unknown, no clinical trial can be performed that is not coordinated with HT. The proposed clinical trial must be piggy-backed upon SOC (i.e., HT) until equipoise has been established allowing a test of hNSC alone vs. HT alone. Yet it remained uncertain whether HT would antagonize the actions of hNSCs or, more optimistically, complement/synergize with them (given that each modality likely invokes different neuroprotective actions (Table 9)), and, if the latter, what should be the timing of hNSC administration in relation to HT to minimize antagonism or maximize benefits? Surprisingly, up until this report, HT alone or in combination with hNSCs for perinatal HII has never been evaluated rigorously, including by MRI or MRS (specifically, T2WI, DWI, and susceptibility weighted imaging (SWI) which enables one to perform HRS). An unsatisfactory attempt at looking at the effect of HT on MSCs has been reported, but, MSCs should never be injected into the brain, particularly in an inflammatory milieu. The tools proved herein (complemented by histological assessment and correlation) have allowed answers these multiple questions.

In these studies, the combined HT with hNSC transplantation, affords synergistic effects of the 2 modalities.

While FIGS. 32A-B illustrates that SOC HT never attained the level of neuroprotection achieved by hNSCs alone, it does demonstrate convincingly that adding hNSCs to HT rendered the tissue and hemispheric volumes of HII rats statistically indistinguishable from those of intact normal controls by adulthood (PD 90).

Those experiments were performed as follows: On PLD 2, after checking of the lesion status by MRI, rat pups exposed to HT were grafted into the cerebral ventricle contralateral to the HII with hNSCs (2.5×10⁵ cells); the lesion status was followed in vivo in subsequent MRI sessions. Volumetric data were obtained from MRI-based HRS analyses and expressed as percentages of the volume of the entire brain.

The gross histology of brains (FIG. 32A) and quantification of the brain volumes (FIG. 32B) are shown. Both sets of data graphically demonstrate the impact at adulthood (PD90) of HT ±hNSCs on tissue and hemispheric size following acute treatment of experimental unilateral HI cerebral injury (HII) (via the RVM) in PD10 rats. The conclusion is that hNSCs, when added to HT renders the brain volumes of HII rats statistically indistinguishable from those of intact normal controls. In FIG. 32A, one can see that HI did not forestall extensive damage to the ipsilateral neocortex and hippocampus. In brains injected with hNSCs, (as seen in representative sections from the HI+Tr and HI+HT+Tr groups) such extensive tissue damage at PD90 was never observed. In FIG. 32B, one can see brain tissue preservation in injured animals cooled on PLD 0 and subsequently receiving hNSCs on PLD 2 (stippled bar HI+HT+Tr); their brain volumes were statistically indistinguishable from those of intact controls. There is the suggestion that HT together with hNSCs act synergistically. These results using grafts contralateral to the lesion were also confirmed using ipsilateral hNSC grafts, with volumetric measurements of the lesioned hemisphere.

The fact that the lesion size following HT alone resembles that of an HI lesion with no intervention at all and that HII lesion reduction is achieved only when HT is followed by hNSC transplantation is shown quite graphically not only in the histology in FIG. 32A, but also in the MRIs shown in FIGS. 33A-B.

In FIGS. 33A-B, rat pups were subjected to left common carotid occlusion and exposure to 8% O₂ at PD10 (The “RVM”). After 4 hours at 36.5° C., they were cooled for 4 hours. At PLD 2, after checking the lesion status by MRI (T2WI), the rat pups were grafted into the cerebral ventricle contralateral to the injury with hNSCs (2.5×10⁵ cells). Coronal MRI images from corresponding brain levels of [A] a representative grafted or [B] a representative “hNSC-conditioned medium”-grafted control animal on PLD 2, 5, and 32, and on PTrD 3 and PTrD 30, respectively are shown. While at PLD 2, ˜5 hours before hNSC transplantation, the T2WIs in both rats show comparably “moderate” HII, by as soon as 1 week after hNSC transfer, the grafted pup shows lesion improvement (i.e., resolution of T2 signal intensity [white]) compared to the control in [B]. This progressive amelioration continues until one month after grafting; the lesion resolves into a very “mild” (clinically insignificant) form, while the hNSC-conditioned medium-alone-grafted control continues to develop a “severe” lesion (excessive T2 signal intensity [white]) with large porencephalic cysts taking up large portions of the neocortex and hippocampus of the lesioned hemisphere.

Because medium conditioned by the hNSCs alone had no therapeutic effect, this experiment rules out a role being played by simply growth factors or exosomes being acutely secreted by the hNSCs as the therapeutic agent; the hNSCs themselves are the mediators of therapeutic action by likely multi-modal mechanisms (FIG. 46).

Data examining the impact of HT on hNSCs in vitro suggest that timing is critical (FIG. 35). Both proliferation and migration appear to be reduced by HT (p=0.006 and p<0.0001, respectively); migration, of course, is critical. Metabolism, as assessed by protein synthesis, after an initial bump (stress proteins), is suppressed in a somewhat protracted manner (p=0.0004). Most of these changes start at <3 days of HT and become quite significant by completion of 3 days. Hence one would not want the donor hNSCs in that HT environment.

Given that it is shown here that administering hNSCs 3 days post-HII and following rewarming was actually the optimal time for therapeutic NSC transplantation (FIGS. 34A-B), and that, clinically, as per SOC, HT is terminated 72 hours post-HII, it should be possible to coordinate the timing of both interventions: hNSC administration after HT and rewarming. Indeed, these data reaffirmed a window of-optimal efficacy previously established for murine NSCs (30). HT did not appear to broaden the window during which hNSCs can be administered effectively.

### Human Neural Stem Cells Normalize Both Motor and Cognitive Behavior in a Perinatally-Asphyxiated Rat Pup Tested at Adulthood Whose Lesion Volume was Also Improved

The clinical relevance of preserving penumbral tissue—and the multiple local and global neural network circuits that pass through it and may be contained within it (FIGS. 47A-B)—has been reflected in the preservation or recovery of complex function (FIG. 42). hNSC recipients performed better on open field testing compared to saline recipients during multiple 3-minute periods over a 30-minute trial (p<0.006) at 90 days post-HII (FIG. 42—open field exploration). Spatial water maze testing revealed deficits in spatial memory for the saline-treated HII group, but not for the hNSC-treated HII group (p<0.05) (FIG. 42—spatial memory). Among the hNSC-treated rats, a smaller penumbra volume correlated with better working spatial memory (r=0.69, p<0.040.

Similarly, another cognitive behavior, the “modified novel object recognition (NOR)” test, confirmed that HII in PD10 Wistar rats abolished the ability of a rat to evince normal self-protective responses because they are unable to encode memories and/or retrieve encoded memories that distinguished a new, potentially life-threatening object from a familiar, safe object. hNSC grafts (vs. saline alone) which reduced the size of a “moderate” lesion to “mild” or “absent” by rescuing the penumbra of asphyxiated rat pups (i.e., “moderate” being defined by rats with a penumbra>core) improved performance in this “cognition and memory” test to levels statistically indistinguishable from normal (FIG. 43). The NOR test is informative on a number of levels. Generally, rodents approach objects and investigate them physically by touching, sniffing, and rearing up on them; spending just enough time to gather data as to whether the object is familiar (i.e., not anxiety-provoking) or novel (and potentially life-threatening and to be avoided). Like many mammals, rats use their vibrissae (whiskers) to be aware of the environment. Because they are nocturnal, rats rely on nonvisual stimuli as much as on visual ones by using a sophisticated array of sensory receptors (the whisker pad) carrying information from the snout along the trigeminal nerve to the somatosensory cortex. There, the cellular representation of the whisker pad, the barrel field, takes up almost half of the somatosensory (SI) area of cortical layer IV, which speaks to the importance of this somatosensory pathway for the rat. Therefore, this short-term memory test using novelty-based recognition is also a visual test and a test of vibrissal function and sensitivity. Since experimental animals can suffer injuries in several cerebral areas, including the somatosensory cortex and the hippocampus, the NOR test was chosen to evaluate their cognitive and memory deficits as well as somatosensory function.

The NOR TEST was performed on 3-month-old animals that had been lesioned, cooled, and transplanted with hNSCs 3 days post-HII. Again. an animal with a significant cerebral lesion is incapable of distinguishing strange from familiar objects and spends too much time with an unfamiliar (potentially dangerous) object. Hence, the less time spent with an unfamiliar object, the more normal the rat's behavior. When treatment led to a mild or lesion-free state (which can be achieved as per FIGS. 39, 40), the rats displayed normal avoidance of—i.e., spent less time with—an unfamiliar, novel, threatening object [FIG. 43, Left Histogram]. However, in animals with an untreated “moderate-severe” lesion, the rats spent a large amount of time around the unfamiliar object as if unable to recall that they had no memory engram for that object or to retrieve such an engram [FIG. 43, Right Histogram]; this is an abnormal cognitive/somatosensory behavior.

In other tests of motor function, a greater number of hNSCs engrafted in the injured

(left) hemisphere correlated with improved rotarod performance (r=0.67, p<0.05). Again, no hNSC-recipient animal was worse than a saline-treated rat or a conditioned medium-treated rat, suggesting that there were no adverse effects from the transplant.

In the rat, P14, P30, P50, P90 are roughly equivalent, respectively, to the human toddler (1 to 2 years old), the human pre-pubescent school-age child (8 to 9 years old), the human post-pubescent adolescent (18 years old), and the human young adult (20 to 30 years old) in terms of brain development. A number of studies using the RVM have assessed relatively short-term outcomes (up to ˜PD21), but few have examined long-term outcomes. The demonstrated ability to perform long-term follow-up at later stages is very important because evidence is emerging that the encephalopathic process following HII may not be solely acute and then static, but rather might continue to develop and worsen over time. Therefore, studies of safety and efficacy of therapeutic interventions should follow the full progression of injury and functional deficits as has been done here. The experimental animals are followed up to at least PD90. The use of hNSCs has consistently arrested the progression of HII and typically promotes reversion.

### Dose-Related Actions and Effects of Human Neural Stem Cells

To the extent that different doses of cells in the various non-clinical studies have been explored a “no observed effect level (NOEL)” for hNSC efficacy against HII has been documented. However, because it is believed the mechanism of neuroprotective action of the hNSCs is at least in part due to the secretion of diffusible neurotrophic molecules, an extrapolation from pre-clinical studies in which the mechanism-of-action (MOA) of the donor NSCs in a mouse model of a lysosomal storage disease (LSD) was providing a sufficient amount of exogenous lysosomal enzyme to restore normal metabolism via cross-correction is possible. This ratio of donor cells-to-mutant (non-enzyme producing) host cells is summarized in Table 10. The optimal ratio of donor:host cells was 1:10 which conferred 28% of normal enzyme levels to that region; the ratio could not be <1:140. If a dose of NSCs was administered at 2×10⁷ cells/kg/ventricle yielding 1×10⁵ NSCs/ventricle (2×10⁵ cells total per animal), then enzyme levels of 5% normal could be achieved cerebrum-wide, with anything >2% of normal being therapeutic for restoring normal metabolism. While similar studies are more difficult to do for HII because read-outs of single cytokines or enzymes are not informative and have not been validated by the field, and, hence have not been done, as Table 10 indicates, the dose reported to be effective in HII (17), was similar: 2×10⁷ cells/kg/ventricle.

Dose-Escalation Studies Showed the Optimal Number and Volume of hNSCs to be Administered.

hNSC dose escalation experiments were performed under the same conditions as described previously and FIGS. 26-30, 32A-B, 33A-B, where a “standard dose (SD)” was used, i.e., 250,000 cells in 5 μL (i.e., 50,000 cells/μL) of vehicle (PBS or HBSS, similar to normal saline [NS] as will be used in the trial) (“standard volume”) instilled slowly (over 1 minute) into only one ventricle (either contralateral or ipsilateral to the HI lesion). Administration of hNSCs was performed following HT (mimicking standard-of-care [SOC]).

The actual newborns in the clinical trial will receive biventricular injections because clinical perinatal HII is bilateral compared to experimental perinatal HII generated by the Rice-Vannucci Model (RVM). The data suggests that transplants contralateral vs. ipsilateral to the HII lesion are equally effective (given the pathotropism of the hNSC) [FIGS. 27A-D]. Therefore, the rat pup studies in which hNSCs were administered into both cerebral ventricles constituted a test of the “2×SD” condition. MRI analysis suggested that these “2×SD” animals improved equal to, but no better than, those receiving hNSCs instilled into a single ventricle at SD. With the double dose, however, there were no adverse effects: no hydrocephalus, CSF obstruction, ventriculomegaly, cell overgrowth, increased intracranial pressure, tumors/masses, displacement of normal cytoarchitectural structures, edema.

Therefore, additional hNSC dose escalation experiments were performed using the following scheme:

3 groups of RVM/HT Wistar rat pups were operated and cooled on PD10 and grafted on PD12-13, the time (2-3 days post-HII) previously established as the optimal post-HII grafting window [FIGS. 32-39]. hNSCs doses were increased using bilateral intraventricular injections as follows [FIG. 37A]:
1. 50,000 cell/μl in 2×10 μl volumes (4×SD in 2× standard volume)
2. 100,000 cell/μl in 2×5 μl volumes (4×SD with double the cell concentration in a standard volume)
3. 100,000 cell/μl in 2×10 μl volumes (8×SD with double cell concentration and standard volume)

This experimental design was employed to investigate not only the effects of increased total dose of grafted hNSCs, but also whether the manner in which that increased dose was achieved impacted outcome e.g., higher cell concentration (which could impact the cells' survival, “clumpiness”, injectability) vs. greater volume (which could increase intraventricular pressure).

As can be seen in FIGS. 37B, 38-40, a 4-8× escalation in the number of hNSCs grafted—which included both an increased concentration and an increased volume into both ventricles—led to a significant improvement in clinical category from “moderate” and “severe” to “mild/no lesion”. Severity categories are defined by size and composition (penumbral volume vs. core volume) of the lesion.

Because improvement in such MRI-based “severity categories” will be required to quantify objectively cerebral parenchymal improvement longitudinally in real-time in actual living babies during the clinical trial, it is worthwhile repeating how these categories have been defined preclinically (showing how easily they can be extrapolated from animals to humans). When the HRS algorithm is applied to raw MRI data (T2WI and DWI), not only can the lesion size be quantified, but also the proportion of the lesion that is salvageable “penumbra” vs. unsalvageable necrotic “core” can also be distinguished and quantified (FIG. 20). These relative proportions are used to define the category of lesion severity: “mild”, “moderate”, or “severe” based both on “total lesion volume” in relation to “total brain volume (TBV)” as well as the volume of penumbra compared to core, recognizing that a total lesion volume can get smaller only if the penumbra gets smaller; core will almost never get smaller. See FIGS. 24A-B for definition in volume of the categories. Examples are shown in FIGS. 38-40.

See the dose-response curve in FIG. 37B. At an increased dose, all “moderate” lesions resolved into “mild” lesions (i.e., lesion volume <2.9% of TBV) or, in the case of pretransplant “mild-moderate” lesions, into lesion-free brains based on MRI criteria. FIG. 39, top provide a more graphic demonstration of the data shown in this curve. Shown are MRIs from a representative animal with an initial “high-moderate” HII lesion on PD12-13 (prior to transplantation) [FIG. 39, top left] that appears lesion-free on Post-Transplant Day 30 (PTD30) [FIG. 39, top right]. FIG. 39 (bottom two pie graphs) illustrate that such dramatic recovery upon increasing the hNSC dose was typical: FIG. 39, bottom shows that, after HII lesioning and immediate HT of PD10 pups but before grafting on PD12-13, the expected spectrum of lesion severities was present in a routine population of experimental animals. However, following administration of donor hNSCs at 8×SD (2×10⁶ hNSCs/animal) [FIG. 39, bottom right], there was complete disappearance of animals with moderate lesions (meaning the penumbra had resolved) and even the appearance of some lesion-free animals (9%) (based on MRI/HRS and histological data, including of PTD 90 rats). Such resolution was evident as early as 1 month post-transplant and was sustained throughout the lifetime of the animal. A similar total normalization of the penumbra is seen in FIG. 40.

It bears reinforcing the importance of these dose-response studies. After injections of hNSCs at SD (both unilateral or bilaterally) following HT, even though outcomes at PTD90 were very much improved (dramatically better than HT alone), there were always some residual “moderate” lesions [e.g., FIG. 30A]. While reduced in prevalence, “moderate” category lesions did not completely disappear but remained, sometimes improving into “mild type” lesions (based on MRI grading). However, with increased cell dose, moderate lesions (i.e., those with a salvageable penumbra) seemed to disappear entirely in a substantial proportion of animals, persisting into adulthood.

FIG. 38 illustrates the normalization (disappearance) of the penumbra (and perhaps even a decrease in core) when all variables have been optimized and an SOP is followed for the administration of hNSCs that employs best dose, ROA, and timing following HII and HT in rat pups asphyxiated using the RVM. Strikingly a reversal of the natural history of an HII lesion was seen wherein the penumbra, without a neuroprotective intervention evolves into core [FIGS. 22A-B].

No Adverse Effects Noted from an Increased hNSC Dose

Even at an escalated dose of hNSCs (up to 8×SD=2×10⁶ cells/animal) at 3 months post-transplant, i.e., adulthood) and an increased volume, no adverse effects have been observed (based on histology, imaging, and neurological and physical examination): no signs of CSF obstruction (i.e., no hydrocephalus, ventriculomegaly, excessive edema); no intra- or extra-cranial malignant tumors derived from the hNSCs; no cell overgrowth, deformation, or inappropriate cell types; no deterioration in neurologic status (including loss of motor/sensory skills, state-of-alertness, responsiveness, respiratory control, new asymmetries, no new-onset or worsening of seizures) compared to the HT control group; no extra pain or discomfort compared to HT control group; no systemic toxicity compared to HT control group; no sepsis, ventriculitis, or dermatitis at the puncture site; no excess vascularity, compromised blood-brain barrier, or hemorrhage compared to HT control group; no greater loss of parenchyma or worsening ventriculomegaly compared to HT control group; no increased intracranial pressure (ICP) compared to HT control group; no increased cerebral scarring compared to the HT control group; no increased mortality/morbidity compared to the HT control group. Of course, where lesions persisted post-grafting, the cytoarchitecture of that region was disturbed (though never worse than an untreated or HT-alone treated control) and never resulting in any compromise of the animal's function.

Importantly, no signs of an increased inflammatory reaction were observed despite not having used immunosuppressive agents; no signs of microgliosis, astrogliosis, or invasion of macrophages or T-cells were present on histology. No evidence of rejection of the hNSCs was present. This is encouraging for the clinical trial in actual patients where also no immunosuppressive agents will be used. The risk of graft rejection and inflammation will be even less in human babies given that hNSC HFB2050.SBP represents a human allograft rather than xenografts as was the case in the preclinical work. As previously indicated, these hNSCs lack MHC class 2 on their surface.

The NOAEL (“no observed adverse effect level”) for this study was determined to be the highest dose tested.

Therefore, an increased dose—up to 8×SD (2×10⁶ cells)—increases efficacy without any observable safety concerns clinically.

The histology of all experimental animals was also improved compared to controls, 3 months after grafting. A benign residual population of quiescent donor-derived cells remained at or near the lesion site. Immunohistochemical analysis of grafted brains using human-specific antibodies to the hNSC marker Nestin and to human astrocytes (hGFAP) indicated that, as in a study using hNSCs in a fetal monkey, that the grafted hNSCs segregated, even in the HII lesion, into 2 sub-populations: the first remained undifferentiated but quiescent (i.e., hNestin+) but likely continuing to exert humoral homeostatic and neuroprotective support; the 2nd had differentiated heavily into hGFAP+ astrocytes that also appeared to interact with host cells (through diffusible factors, direct cell-cell contact and intercellular signaling, and possibly via exosomes (FIG. 45).

This histological picture has helped give rise to a model of the molecular and cellular mechanism by which the donor hNSCs may be salvaging the penumbra (FIGS. 46, 47A-B) and, importantly, the fibres de passage coursing through the penumbra that are critical components of global neural networks (undergirding complex behavioral and cognitive functions [e.g., FIGS. 42, 43]) which would otherwise be disrupted were the penumbra to die and these neural projections axotomized.

The highest dose of hNSCs tested was 5×10⁷ cells/kg/ventricle in fetal monkeys, with no adverse effects and normal cerebral development. In the planned GLP safety/toxicity studies, the dose escalation studies will be done as follows: starting at 1×10⁷ cells/kg/ventricle, the highest dose explored in a clinical study performed by another team instilling mesenchymal stem cells (MSCs) into the ventricles of human newborns and found to be safe. A dose escalation (2×) to 2×10⁷ cells/kg/ventricle to match that found to be effective as well as safe in rat pups will be done. Progressing incrementally to 5×10⁷ cells/kg/ventricle (to match the cell dosage found to be safe and effective in fetal bonnet monkeys) will then be considered. Dose escalations beyond 2×10⁷ kg/ventricle will not be performed in human newborns until preclinical studies have been performed in fetal sheep (one of the proposed IND-enabling studies), which approximate the size (both of body and brain) of human newborns.

The data suggests that the ideal density of hNSCs in a NS suspension is 4-5×10⁴ μL, indicating that a volume of 1 mL will be instilled per ventricle for 1×10⁷ cells/kg/ventricle and a volume of 2 mL will be instilled per ventricle for a dose of 2×10⁷ cells/kg/ventricle. Volumes as high as 7 mL/ventricle can be well-tolerated by babies if an equal volume of CSF is removed in order to maintain a stable intracranial pressure (ICP).

a One-Time Intraventricular Injection Via an Ultrathin Needle Causes No Mechanical or Inflammatory Injuries in the Overlying Cortex Through which the Needle Passes

To determine whether a single intraventricular injection via an ultrathin needle, as will be used in the clinical trial (e.g., 30-34 gauge Hamilton syringe), will cause any mechanical or inflammatory injury to the overlying cortex through which the needle must pass, the following experiment (FIG. 41) was performed: Intact, normal PD12 Wistar rats received stereotactic-guided bilateral intraventricular injections of hNSC culture medium (10 μL per site) into the intact brain and then were sacrificed on PD90. The coordinates (in mm) were as follows: from Bregma −0.4, medio-lateral 1.5, dorso-ventral 3.5). For this experiment, even a larger gauge needle was used than is planned for the clinical trials to push the threshold (24 gauge Hamilton syringe). FIG. 41 (top) shows a coronal, hematoxylin-stained histological section from the PD90 rat brain with the injection sites marked by arrows. The brains of such animals (n=5) were examined for needle-induced damage to the cortical parenchyma. Neither mechanical nor inflammatory injuries were detected.

Biodistribution Studies Show that hNSCs Administered into the Cerebral Ventricles do not Infiltrate Organs Outside of the CNS

Although grafted into the ventricular system of the brain, an important question is whether the donor hNSCs will migrate or be transported to other organs of the recipient. This possibility was tested 3 months after transplantation using a highly sensitive qPCR assay developed for specific Alu-based quantification of human cells among rodent cells. Alu sequences are primate-specific ˜300 nucleotide-long interspersed elements present in >1 million copies in the human genome. These characteristics make them ideal targets for the qPCR detection of human cells among the cells of other species. Using a primer and probe set carefully designed to avoid possible cross-reactions with the rodent genome, allowed, in principle, 1 human cell among 100 million rodent cells to be detected.

The following non-neural tissues from the transplanted rats: thymus, lung, liver, and spleen were tested. The organs were quickly removed from freshly sacrificed adult rats (that had been perfused with PBS) and were fixed overnight at 4° C. in 4% paraformaldehyde (PFA). The tissue was mechanically crushed and digested with proteinase K, the DNA isolated using a commercial kit (Zymo Research), and its purity and concentration determined using a NanoDrop spectrophotometer (Thermo Fisher Scientific). DNA from cultured hNSCs was used as a positive control for the human Alu signal.

Alu-qPCR was performed with a custom-made primer/probe set (Integrated DNA Technologies). A total volume of 20 μL contained 10 μL TaqMan Universal Master Mix II (no UNG); 1 μL custom TaqMan gene expression assay (giving a final 900 nM of forward and reverse primers and 250 nM of a fluorescent, FAM-labeled TaqMan MGB probe); and 100 ng (9 μl) DNA. (Except for the DNA, all solutions were ordered from Thermo Fisher Scientific). The PCR cycling conditions were 1 cycle of 95° C. for 5 min., followed by 45 cycles of 95° C. for 15 sec, 56° C. for 30 sec, and 72° C. for 30 sec. For controls, reactions without a DNA template (NTCs) were used as well as reactions specific for the housekeeping gene glyceraldehyde 3-phosphate dehydrogenase (GAPDH). To confirm the sensitivity of the detection of human Alu sequences, the DNA from hNSCs was also titrated and qPCR reactions with template concentrations from 100 ng to 0.1 pg run. The results of both experiments are presented in FIGS. 44A-B.

While the DNA from the hNSCs used for grafting showed a clear signal from amplified human Alu sequences, all non-neural tissues were, as expected, negative [FIG. 44A, red lines]; the weak signal starting beyond 40 cycles coincided with the background signal of the NTC. On the other hand, amplification of the control rat-specific GAPDH sequence was clearly visible in the non-neural tissues while no signal was detectable in reactions using the DNA of hNSCs [FIG. 44A, blue lines]. This result demonstrated a clear absence of any human (i.e., grafted) cells in non-neural tissues and indicated that the donor stem cells remained confined to the neural compartment of the rat brain. FIG. 44B shows that the PCR reactions were sensitive enough to amplify Alu sequences from even 0.1 pg of template. Since a mammalian cell contains (on average) 26 pg of DNA, the reactions were 260× more sensitive and would, therefore, have easily picked up any isolated human cells in the rat tissues.

From these qPCR results with rats on Post-Transplantation Day 90 (adulthood), one can say with confidence that the hNSCs instilled into the ventricles of rat pup brains at PD10 were neither transported into nor did they infiltrate other organs. This, in fact, confirms the data from previous studies with the hNSCs in rodents and non-human primates (Table 7).

Summary with an Emphasis on Mechanisms of Therapeutic Action

In summary, hNSCs—which preferentially migrate to and integrate within areas of moderate and severe HII (influenced, in part, by upregulation of repair-associated gene products such as HSP27 which are most robust in host regions where cells are still salvageable and self-repair mechanisms are triggered, i.e., the penumbra)—have their greatest impact not on a necrotic core where host cells have already died, but rather where there is penumbra that can “rescued”. (In the absence of a neuroprotective intervention, the penumbra will convert to core as the cells within that region succumb to secondary death processes [FIG. 46]). These treatable rats—and patients—can be selected using MRI data routinely obtained as standard-of-care (SOC) of all babies that receive therapeutic HT if those data are then analyzed post-hoc via an HRS algorithm (which is protocolized and quite easy and acceptable to all points-of-care) [FIG. 46]. Emulating in human newborns the intraventricular instillation of hNSCs that was done in the rodents is actually a minimally invasive procedure; it can be performed non-surgically in the NICU itself or in the adjoining procedure/operating room that all NICUs have by simply aseptically inserting percutaneously an ultrathin needle (e.g., 30-34 gauge) under ultrasonic (or even landmark) guidance through the open anterior fontanel. Enabling NSCs direct access in this way to the periventricular germinal zones, into which they integrate seamlessly and intermix with endogenous NSCs, simply augments a natural homeostasis-preserving, self-repair mechanism invoked by the mammalian brain in response to HII.

Clinically, if the penumbra is allowed to progress to a necrotic core (i.e., transitioning from “moderate” to “severe” HII), the outcome is worse (FIG. 46). Data are compelling that hNSCs applied at a strategic time (3 days post-HII) and location (biventricularly) can prevent that transition alone and even more completely when administered following HT. HT alone, however, does not have the ability to rescue the penumbra or shrink the lesion size. The ability to use MRI to distinguish between irretrievable core parenchyma and HSP27-expressing salvageable penumbra will facilitate patient selection, timing of the initial injury, timing for the intervention, and possibly even prognosis.

The mechanism of therapeutic action of the hNSCs is multimodal. A likely process is schematized in FIGS. 47A-B. In the region surrounding the core of immediate cell death due to necrosis, i.e., the penumbra, resident astrocytes are induced by HII to become “reactive”. Initially, reactive astrocytes are supportive; however, with time they transition into a type of reactive astrocyte that attempts to “clean out” the area and contain the region with scar tissue and extracellular matrix. The exogenous hNSCs suppress the transition of local astrocytes into reactive astrocytes but change their fate instead into more fetal neurotrophic astrocytes. Similarly, the donor exogenous hNSCs themselves are fetal, producing not only supportive molecules but also inhibiting inflammation and scavenging ROS and excitotoxins. Salvaging the penumbra has such a profound positive impact on the animal's (and presumably baby's) function not so much because of what is contained within that region but rather because of the multiple complex neural circuitry which passes through it (fibres de passage); disruption of these global neural networks that connect all parts of the brain can be catastrophic. hNSCs prevent axotomy (and/or forestall demyelination if such fibers are already myelinated).

### Summary and Conclusion

Hypothermia (HT) (as employed under present SOC) and hNSCs can act synergistically to neuroprotect the perinatally-asphyxiated brain. Not only is this approach feasible and safe, but potentially obligatory to ameliorate damage in perinatally-asphyxiated babies.

Absence of an intervention following HII allows moderate lesions to progress to severe lesions, and mild lesions to worsen to moderate lesions. Contrary to the classical view of perinatal asphyxia, acute HII lesions are not static, but rather continue to progress for at least another 1 to 3 months if not neuroprotected.

In the RVM experimental model, the outcome from HT alone (administered in a manner that emulates SOC), is significantly worse than that achieved with hNSC transplantation alone (the latter leading to a complete resolution of penumbra by adulthood when the proper dose, ROA, and timing are employed, as elucidated by the non-clinical work in this proposal).

While HT may provide some initial, transient benefit in slightly reducing the severity of the HI lesions, the lesion volume, and its severity (as represented by significantly increased core volume) actually grows over the course of a month as if there had been no intervention at all.

HII lesion reduction is achieved by HT (in these non-clinical studies) only when it is followed by hNSC transplantation.

Improvements following hNSC transplantation become evident as early as several days after implantation, stabilize by 1-month post-treatment, and persist for at least 3 months (adulthood for a rat, and the pre-determined termination point of these pre IND-enabling studies). Not only was there no progression of severity (as seen in untreated brains), but brain volumes in HI animals that are cooled in a manner emulating SOC and subsequently receive hNSCs are statistically indistinguishable from those of intact normal controls, suggesting that hNSCs and HT act synergistically.

While HT cannot achieve lesion reduction if delayed, hNSC administration alone can be delayed; in fact, in these non-clinical experiments, hNSCs were not administered until 3 days post-lesioning (a window previously determined to be the optimal window-of-opportunity for protection/repair.

In lesioned rats receiving hNSCs even without prior cooling, an HI injury was virtually absent at adulthood, making hNSC grafting a potential neuroprotective alternative for human babies who miss their 6-hour window for HT as per SOC or, in low- and middle-income countries where HT proved ineffective (HELIX trial).

Lesion reduction was similar whether the hNSC grafts were administered contralateral or ipsilateral to the lesion hNSC.

That administration of hNSCs in the cerebral ventricle contralateral vs. ipsilateral to the lesion appeared equally efficacious and safe, bodes well for the biventricular administration of hNSCs in the clinical trial because HI lesions in human newborns under true clinical conditions are usually bilateral (asymmetric) or even global. Bilateral administration, therefore, will increase the likelihood of clinical improvement.

Medium conditioned by the hNSCs alone had no therapeutic effect, ruling out factors or exosomes acutely secreted by the hNSCs as being the therapeutic agent; the hNSCs themselves are key mediators of the therapeutic action (by likely multi-modal mechanisms). Indeed, the hNSCs that integrate into the host parenchyma engage in an ongoing “dialogue” with the host.

The hNSCs induce in the host an upregulation—beyond that seen following injury alone—of cell proliferation and neural cell types that are pivotal for self-repair: DCX (which represent migratory neuroblasts destined to become neurons) and TuJ1-expressing cells (which represent young neurons).

The donor hNSCs themselves, which rarely proliferate in vivo, often differentiate into astrocytes that extend their processes into the host brain parenchyma. Donor-derived cell expression of DCX and TuJ1 was present as well, particularly in hNSCs that had integrated into the host ependyma or parenchyma.

HII constitutively but transiently induces a molecular milieu supportive of restoring homeostasis—a biomarker for which is HSP-27, most prevalent in the penumbra. hNSCs home to regions of HSP-27 expression, enhance that expression, and come also to express HSP-27 themselves.

There was never evidence of ventriculomegaly from hydrocephalus or obstruction to CSF flow. There was never deformation of the host cytoarchitecture, tumors, neoplasms, mass or pressure effects or any suggestion of uncontrolled donor or host cell proliferation. No host- or donor-derived non-neural cells were observed nor any cells or structures that are inappropriate to the brain. In addition, the animals never evinced any systemic adverse symptoms: no pain or discomfort, no increased mortality/morbidity attributable to the hNSCs or to administration of the hNSCs

Although the hNSCs represent a xenograft in the rat pup brain and although immunosuppression was not used, there was never any evidence (either as early as 3 days post-transplant or 3 months post-transplant) of host monocytic, macrophage, or T-cell invasion that might indicate an immunogenic reaction or immunorejection. Immunosuppressive drugs are not planned to be used in the babies during the clinical trial, and the lack of an obvious immunologic reaction in these animals despite donor-host species incompatibility, suggests that such an approach is valid (hNSCs lack MHC class II making them minimally immunogenic in the newborn brain).

hNSCs normalized cognitive and motor behaviors in perinatally-asphyxiated rat pups tested at adulthood, particularly if the lesion volume was also reduced; lesion reduction resulted almost exclusively from normalization of the penumbra, not diminution of the core.

In summary, using the MRI-based HRS assessment tool illustrated in FIGS. 20-24, 26-34, 38-40 (complemented by histological, molecular, and behavioral correlation), the synergistic actions of hNSCs when combined with HT for neuroprotection have been evaluated in the FDA-accepted RVM (FIGS. 32, 33, 38-40) and biventricular administration of 1×10⁷ cells/kg/ventricle 3 days post-HII and following rewarming is likely the optimal protocol for hNSC administration.

The data suggests hNSCs will address many of injurious processes not addressed by HT (e.g., inflammatory, apoptotic, necrotic, excitotoxic, oxidative, vascular, demyelinative, etc.) and hence improve outcome. These actions work synergistically with HT which provides neuroprotection by different yet complementary/additive mechanisms (Table 9). The data suggests that MRI/HRS will be an effective “biomarker” for assessing treatments and outcomes for perinatal HII clinically in babies. Indeed, quantifying the “penumbra:core” ratio following HII (FIG. 46), has given Regenerative Medicine its 1st “biomarker” for selecting patients who will be responsive to cell-based therapy based on its MOA, while sparing others that intervention (allowing for them the efficient institution of alternative pathology-specific therapies). Rescuing penumbra (with both its intrinsic neural networks and fibers de passage contributing to other networks brain-wide) is a critical and achievable target.

### Example 7: Product Description

HFB2050.SBP is comprised of hNSCs which were isolated in 2000 from the forebrain VZ of a single anonymous human female fetus cadaver aged 13 weeks gestation. Although the starting primary culture of dissociated cells isolated from the VZ was plated as a monolayer in medium containing fetal bovine serum (FBS) (10%) and horse serum (HS) (5%) in 2000 (none of which came from regions where prion disease is endemic), the cells were subsequently propagated, starting with their first passage, and hNSCs were isolated with mitogens alone (epidermal growth factor [EGF]+fibroblast growth factor 2 [FGF2]+leukemia inhibitory factor [LIF]) under defined, serum-free, xeno-free conditions as a monolayer on recombinant human laminin. This well-characterized hNSC line has been studied for over 23 years and has been tested extensively in rodent and NHP normal healthy and disease models of glioblastoma (GBM), spinal cord injury (SCI), stroke, Parkinson's disease PD, amyotrophic lateral sclerosis (ALS), and other neurodegenerative conditions such as Krabbe and Sandhoff diseases.

The hNSCs are conceptually, functionally, and transcriptomically comparable to those used in clinical trials for neuronal ceroid lipofuscinosis, ALS and SCI.

Comparable hNSCs have been studied in three US trials for GBM which were derived in the same procedure from the same cohort of fetuses as those described here by the same investigators involved in generating HFB2050.SBP. The only fundamental difference between the hNSCs in the GBM trials (called HB1.F3.CD) and HFB2050.SBP is that the primary cells, obtained from a similar fetal VZ, were genetically modified to express v-myc and cytosine deaminase (CD) prior to human testing, to enhance their self-renewal and GBM killing properties, respectively. The early acute safety of HB1.F3.CD in patients in these trials suggests that analogous fetal-derived hNSC such as HFB2050.SBP may also be safe to use therapeutically in HII patients The HFB2050.SBP hNSCs have remained stable after scores of passages over the years, have been repeatedly tested as karyotypically normal female, and have an expression profile consistent with other human fetal forebrain-derived hNSCs characterized by primary VZ cells. No evidence of transformed, neoplastic, or pluripotent cells has ever been observed in the HFB2050.SBP line.

### Example 8: Indication, Dosage, and Route of Administration

HFB2050.SBP hNSCs will be studied for the treatment of Sarnat clinical Stage 2 (moderate) to 3 (severe) perinatal HII in full-term neonates who qualify for therapeutic hypothermia (HT) and will be administered to newborns after the completion of their 3 days of HT treatment.

HFB2050.SBP hNSCs suspended at 5×10⁷ cells/mL in normal saline (NS) are administered once in a minimally-invasive aseptic intracerebral instillation through the open non-bony anterior fontanelle (AF) into each of the lateral ventricles (LVs) using a 5-cm 30-34-gauge needle attached to a Hamilton syringe inserted percutaneously with simple local topical analgesia at the puncture site. This the manner in which ventricular taps are performed at the bedside in newborns when cerebral spinal fluid [CSF] is aspirated or substances [e.g., antibiotics] are infused, typically using even larger gauge needles. Needle insertion and tip placement, to be performed by the team's pediatric neurosurgeon, is guided by cranial ultrasound. The proposed clinical doses of approximately 1×10⁷ and 3×10⁷ cells/kg (Table 10) in up to 1 mL suspension per ventricle have been assessed and verified in research and pivotal safety and efficacy studies in which the hNSCs have been shown to outperform HT alone and synergistic with HT when used after that standard-of-care (SOC) course (Table 13). Progressive rescue of the penumbra has been documented by use of hierarchical region splitting (HRS), a mathematical algorithm applied post-hoc to routine T2-weighted and diffusion weighted imaging data obtained under magnetic resonance imaging (MRI).

The route of administration (ROA), administration technique, and dose are comparable to those used in a recent clinical test of stem cell injections into the cerebral ventricles of pre-term human infants, as well as by extrapolation by weight from doses found to be safe and effective in the classic perinatal asphyxia rat pup model, the Rice-Vannucci Model (RVM) (Table 13), as well as safe and efficacious in fetal monkeys.

TABLE 13
Nonclinical Studies Performed

| # | Objective | Animal, Sex, Number | Results |
|---|---|---|---|
| 1 | Assess efficacy & | RVM P10 rat pup model of perinatal [symbol] | See FIGS. 26-28, 32, 33, 42 and text. |
| | safety of | n = 106 | Experimental Paradigm (FIG. 19) [symbol] |
| | HFB2050.SBP | 10 μl dissociated single cell suspension | A 10 μl dissociated single cell suspension of 5 × 10 [symbol] hNSCs infused at |
| | hNSCs alone | of 5 × 105 hNSCs infused at a rate of | a rate of 0.5 μl/min over 10 min. |
| | (research grade | 0.5 μl/minutes over 10 minutes. | No immunosuppression. |
| | but cGMP | No immunosuppression. | Follow-up for 1-3 months (histology, MRI, behavior). |
| | compatible) in | Follow-up for 1-3 months. | Control: vehicle alone (PBS, HBSS, or NS) or medium [symbol] by |
| | rescuing the | | hNSCs. |
| | penumbra when | | hNSCs alone dramatically neuroprotective |
| | instilled into one | | “Moderate” injury converted to “mild” injury FIG. 30A. |
| | of the cerebral | | Total lesion volume decreased by −67%. |
| | ventricles of rat | | Decrease in size solely [symbol] to decreased [symbol] volumes |
| | pups subjected to | | (p < 0.000001) (not achieved by saline or conditioned medium |
| | experimental [symbol] | | controls). |
| | via RVM & | | Penumbra was almost completely gone in hNSC group FIG. 29 |
| | analyzed at | | (0.5% of TBV; 6.7% of the remaining lesion) but remained prominent |
| | adulthood. | | in the saline group (4.4% of TBV; 49.4% of remaining lesion). |
| | (Note: these data | | Penumbral tissue that cannot be rescued continues to cascade toward death by |
| | were published in | | necrosis FIG. 26 |
| | the course of | | In saline treated group. of lesion categorized as “penumbra”, 19.2% |
| | pursuing Pre-IND | | converted to “core”; 46% converted to MRI- classified “normal” |
| | enabling studies & | | tissue. But, in hNSC-treated groups, only 8.2% of the “penumbra” |
| | constitute a pivotal | | converted to “core”; 87.8% converted to “normal” FIG. 26 |
| | pre-clinical study) | | TBV of hNSCs-treated animals = 90% of intact TBV |
| | | | FIG. 30 [symbol] |
| | | | Severely infarcted brains, characterized principally by unreclaimable core and |
| | | | minimal penumbra, unimproved by hNSCs; lesion sizes unchanged FIG. 26 |
| | | | Histological improvement to close to intact animals FIG. 32A |
| | | | Behavioral improvement to that of intact animals FIG. 42 |
| | | | See FIG. 46 for summary of this pivotal pre-clinical study: |
| | | | hNSCs improve lesional, motor, and/or cognitive outcomes when there is an |
| | | | MRI-measurable penumbra that can be forestalled from evolving into |
| | | | [symbol] the core never improves. Unlike the core, a penumbra is |
| | | | characterized by a molecular profile associated with salvageability. Also |
| | | | [symbol] was the [symbol] of a clinically [symbol] |
| | | | MRI algorithm. [symbol] which can subdivide & quantify an [symbol] into |
| | | | salvageable [symbol] vs. unreclaimable necrotic cores [symbol] & hence provides a |
| | | | rigorous, [symbol] , prospective, noninvasive “biomarker” for identifying |
| | | | [symbol] bearing a molecular profile indicative of responsiveness to [symbol] |
| | | | [symbol] mechanism of action. This tool was used in the [symbol] |
| | | | studies described below. |
| 2 | Test ROAs: | RVM Rat Pups (Wistar). | See FIG. [symbol] and text. |
| | Compare | [symbol] (n = 13) [symbol] | [symbol] and [symbol] administration are equally [symbol] and safe in |
| | [symbol] of | (n = 17). | rescuing the penumbra and suppressing progression of lesion severity and size |
| | hNSCs into the | | (sometimes reversing severity). |
| | cerebral ventricle | | Medium conditioned by the hNSCs does not have any efficacy. |
| | [symbol] vs. | | That an [symbol] vs. [symbol] ROA yields essentially [symbol] beneficial |
| | [symbol] to | | results [symbol] well for success in the clinical trial in which hNSCs will be injected |
| | the [symbol] lesion. | | into both ventricles. Unlike in the RVM animal model, actual asphyxiated |
| | | | neonates usually have bilateral - even global - cerebral lesons; it is important to |
| | | | know that the hNSCs can [symbol] to lesions whether [symbol] or [symbol] to |
| | | | the site of implantation and provide neuroprotective relief. |
| 3 | Assess effects of | RVM Rat Pups (Wistar). | See FIGS. [symbol] and text. |
| | hypothermia | HT Treatment Group. | Hypothermia alone does not rescue the penumbra or prevent lesion severity from |
| | (HT) alone: | n = 8-14 (equal sex distribution). | worsening and lesion volume from growing. |
| | Quantify baseline | [symbol] Control Group: | |
| | impact of | N = 8-14 (equal sex distribution). | |
| | whole- body HT | | |
| | [symbol] pups | | |
| | subjected to | | |
| | experimental [symbol] | | |
| | via the RVM. | | |
| 4 | Assess potential | RVM Rat Pups (Wistar). | See FIGS. [symbol] and text. |
| | synergy between | HT Treatment Group: | HT and hNSCs are neuroprotective by different and complementary actions |
| | HT & hNSC | n = 8-14 (equal sex distribution). | (Table 9) |
| | transplantation. | [symbol] Control Group: | Since HT alone shows minimal to no ability to rescue the penumbra or prevent |
| | | N = 8-14 (equal sex distribution). | conversion of penumbra to core FIG. 25 [symbol] while hNSCs are significantly |
| | | | neuroprotective, that HT + hNSCs yields brain volumes indistinguishable from |
| | | | [symbol] (FIG. 32B). |
| | | | Indicates that the 2 modalities are synergistic (not simply additive). |
| 5 | Determine | RVM Rat Pups (Wistar). | See FIG. 34 [symbol] and text. |
| | optimal timing of | “Immediate grafting” vs. “Delayed | Optimal time for grafting hNSCs in relation to occurrence of [symbol] and |
| | hNSC | grafting” in relation to HT. | administration of HT is 3 days-post-lesion which would also coincide with the |
| | administration in | n = 8-14 per group (equal sex | [symbol] of HT in [symbol] human neonates. |
| | relation to HT. | distribution). | MRI/HRS criteria used; results [symbol] administration 2-3 days |
| | | hNSCs administered into both ventricles, | post- [symbol] and post-HT (compared to FIG. 30). |
| | | at standard dose 2.5 × 10 [symbol] | Grafting [symbol] with [symbol] of HT (“Immediate”) less |
| | | hNSCs/ventricle | effective than waiting for the rat pup to be [symbol] ; progression not |
| | | Control [symbol] Vehicle alone and Medium | [symbol] . |
| | | conditioned by hNSCs (n = 8-14). | Grafting [symbol] week following course of HT (“Delayed”) ineffective in |
| | | | [symbol] of [symbol] lesion; window of opportunity not broadened post- |
| | | | lesion. |
| 6 | Determine | Comparing dose of [symbol] hNSC | Optimum dose of HFB2050.SBP hNSC per ventricle is 4 × SD (FIG. 47). |
| | optimal dose of | administration by adding the following | Statistically significant improvement in ( [symbol] ) MRI (lesion reduction); (2) behavior; |
| | [symbol] | experimental conditions [symbol] | (3) histology relative to the HT alone control group when using the optimized |
| | hNSCs | Group 1 SD: 2.5 × 10 [symbol] / [symbol] ventricle | conditions for hNSCs administration: ROA (biventricular), timing (3 d post- [symbol] ). |
| | administration & | (n = 50). | dose (8 × SD = 2 × 10 [symbol] /animal). |
| | assess safety | Group 2: 2 × SD by administering | Safety under optimized conditions: |
| | | hNSCs to both ventricles (n = 15) | HFBP2050.SBP derivatives not present in any non-neural organ |
| | | Group 3: 4 × SD/ [symbol] (by | immunohistochemistry at 90 days post- [symbol] ). |
| | | [symbol] dose to each [symbol] ) | No intra- or extra-cranial malignant tumors derived from hNSCs. |
| | | (represents 8 × SD/animal) | No cell overgrowth, deformation, or inappropriate cell types compared |
| | | (n = 12) | to total human cell composition. |
| | | | No deterioration in neurologic/mental status (including loss of |
| | | | motor/sensory skills, state-of-alertness, responsiveness [symbol] respiratory |
| | | | control [symbol] new asymmetries, no new-onset or worsening of seizures) |
| | | | compared to HT control group. |
| | | | No loss of [symbol] or worsening [symbol] compared to |
| | | | HT control group. |
| | | | No pain/ [symbol] |
| | | | No systemic toxicity |
| | | | No excess vascularity, [symbol] blood-brain barrier, hemorrhage |
| | | | compared to HT control group. |
| | | | No increased intracranial pressure compared to HT control group |
| | | | No sepsis |
| | | | No ventriculitis or dermatitis (infectious or inflammatory) |
| | | | No increased cerebral scarring compared to HT control group. |
| | | | No increased mortality/morbidity compared to HT control group. |
| | | | No HFB2050.SBP derivatives in any non-neural organ as determined by [symbol] PCR |
| | | | at 90 days post- [symbol] . |

[symbol]

indicates data missing or illegible when filed

### Example 9. Rationale for Use of Human Neural Stem Cells: They are Effective in Hypoxic-Ischemic Brain Injury Based on Several Mechanisms

Direct neuroprotection and neurotrophic support via diffusible factors, gap junctions, exosomes (e.g., providing cytokines as GDNF, BDNF, NT-3, NT-4, NGF, Nurturrin).
Scavenging ROS and excitoxins.
Reducing inflammation and scarring.
Promoting angiogenesis.
Repairing the blood-brain barrier.
Mobilizing endogenous NSCs.
Promoting endogenous neurite outgrowth.
Replacing interneurons.
Providing glial support, including astrocytes and oligodendrocytes (including myelinating oligodendrocytes).
Providing extracellular matrix.
Altering the niche.
Restoring normal metabolism to injured host neural cells.
Forestalling axotomy
Inducing neural self-repair, known to occur in the injured immature newborn brain.

Many therapeutic strategies that have been considered for perinatal HII and could be regarded as “competing” approaches; all suffer in comparison to hNSC-mediated treatments. The reasoning behind this statement is not solely based on the empirical evidence but also on the rationale behind treating a neurological disorder that has multiple pathophysiological processes such as HII: any therapy that targets simply one pathogenic process is inevitably going to fail. Only a “cell” possesses the multi-modal actions (mediated by complex intracellular sensing and response molecular pathways) required to target these multiple pathogenic processes constitutively and simultaneously. In addition, the cell used should be a one that is native to the CNS and has, as its natural biological repertoire, the performance of these “tasks” normally in fulfillment of its developmentally-specified teleological biological imperative: to participate in organogenesis and to maintain homeostasis in the organ of interest (FIG. 18).

### Example 10. Feasibility of Serial MRI with Hierarchical Region Splitting for Monitoring the Impact of hNSC Treatment Combined with HT

Can be integrated into routine care (HRS is a post-hoc mathematical manipulation of digital data already routinely obtained on all HII infants that received HT, including T2WI, DWI, SWI).
Can monitor noninvasively evolution of HII.
May help pinpoint when an HI event took place during the perinatal period. It is always challenging for a clinician to know precisely when an asphyxia episode took place in the perinatal period because, with a 6 hour window, there is no time to do an MRI before HT. One tends to surmise based on the neurological exam: for example, if there is increased tone rather than decreased tone, then it is likely the cortical injury occurred weeks before birth, perhaps supported by a report from the mother of decreased fetal movement at that time. However, using HRS can provide some additional objective data. For example, if, on the 4th day after birth MRI, all of the lesion is Core, then, the insult likely took place more than 2 weeks before delivery because every ischemic lesion has some component of a penumbra which transitions to Core over the course of 2-3 weeks (e.g., See FIG. 29).
Can monitor advent of any adverse reactions (tumor formation, deformation, cell overgrowth, ventriculomegaly).
Magnetic Resonance Spectroscopy is obtained during the same imaging session, providing data on metabolic impact of the hNSCs (e.g., looking for resolution of any abnormal N-acetylaspartate (NAA) peaks and ratios, indicative of neuronal injury or repair).
Ultimately, MRI may have prognostic value as well as aid in rational patient stratification/selection
   The present clinical staging of patients is based solely on neurological exam within the first 6 hours of life and on systemic non-CNS biochemical measures of acidemia. There is no direct objective measure of the CNS. Magnetic resonance imaging (MRI) with HRS may allow clinical staging based on direct assessment of the state and extent of CNS asphyxia injury

### Example 11 Route and Techniques of Administration

### Overview

Instillation of hNSCs into the cerebral LVs is performed by a health care provider using a minimally invasive route and device (an ultrathin 30-34-gauge needle), an approach often used in neonatology (performed by neonatologists, with even bigger needles) to instill intrathecal antibiotics, to remove excess CSF, or reduce ICP.
Because the sutures in a newborn are not yet closed, entrance into the LVs can be achieved percutaneously without a need for skin incisions craniotomies using a simple 30-34-gauge needle inserted only 2 cm into the non-bony anterior fontanelle (AF), which places the needle tip into the center of the ventricle (as will be directly visualized by the ultrasound). The puncture in the skin overlying the AF is pinhole size, rapidly closes on its own, and requires no sutures or steristrips, simply a small ≤1 cm diameter circular adhesive gauze bandage.
Insertion of the needle is similar to the way in which a spinal tap is performed in a newborn's lumbar region, but with a much smaller gauge needle.
Routine bedside cranial ultrasound will be used to guide catheter placement following percutaneous insertion, though, historically, this procedure (“ventricular taps”) has been performed by generations of neonatologists simply by using cranial landmarks.
No need for general anesthesia (mild sedation and/or simply swaddling is sufficient as well as local topical analgesia to the insertion site).
The procedure can be accomplished relatively quickly: cells are instilled into each LV over a 60 second period. The entire procedure, as detailed below, is completed within 30 minutes—from NICU bed to procedure room and back to NICU bed.
The desired dose of hNSCs (see Table 10) are suspended in NS at a concentration of 5×10⁴ cells/μL.
Sterile, pre-packaged, appropriately-dosed (based on Table 10), patient-specific, administration-ready syringes is prepared by the manufacturing team for each baby and a trained, gowned-and-gloved manufacturing team member will accompany the administration syringes to the procedure room, open the container using aseptic technique, perform one gentle round of trituration, and hand the sterile syringes (one at a time) to the neurosurgeon when needed for injection.
   a. Three syringes are prepared and sterilely packaged and sealed: one for each of the two ventricles and a back-up syringe should something happen to one of those two syringes. If the 3rd syringe is not needed, it will be used to analyze the hNSCs actually administered and cryopreserved as an “archive” of what was administered to that particular patient for subsequent analysis and clinical correlation with positive or negative outcome.
   b. All syringes are maintained at 4° C. until used and will be continuously gently agitated to prevent clumping or settling of the cells out of suspension.
To maintain blinding, patients who have randomized to receive no hNSCs will be wheeled into the procedure room and a small adhesive bandage, similar to those babies who have received hNSCs, will be applied to the scalp overlying each AF at a position approximating where a needle would have been inserted had hNSCs been instilled; however no actual needle insertion or the instillation of any substance will be performed.

### Detailed Procedure

Preoperatively, a complete blood count and serum electrolyte analysis is used to rule out any potential for infection or coagulopathy. The patients will also have received their post-hypothermia [HT] magnetic resonance imaging (MRI) (as per standard-of-care [SOC]). Any patients with pre-existing hemorrhages will be excluded from receiving hNSCs.
All babies who receive HT already have vascular access through which fluid and glucose (typically total parenteral nutrition [TPN]) is administered and through which medications are given. These will be continued.
The baby will be made NPO (nothing-per-mouth) 4 hours before the procedure (with all fluids provided as TPN).
SOC calls for all babies to have continuous cardiac, respiratory, oxygen saturation, and EEG monitoring at all times. These will be continued throughout the procedure (although any EEG leads near the insertion site will be removed during the procedure).
Sedation, if necessary, beyond swaddling, is provided by the NICU care team.
The patient is then transported on a warming table to the clean procedure room adjoining the NICU by the neonatologist, anesthesiologist, and NICU team. The patient is then placed onto the operating table.
Respiratory support, if needed (many babies do not), is provided by the NICU team (including neonatal respiratory therapist in conjunction with a pediatric anesthesiologist) at this time and throughout the procedure.
The operating table is turned 180°. The patient will remain supine, and the head placed on a donut. The head is stabilized utilizing paper tape which is placed on the patient's forehead and then attached to the operative table on either side for stability.
The entry sites is defined using a surgical marker. Specifically, the entry sites are marked at the intersection of the mid-pupillary line and coronal suture. Bilateral sites are marked. If the patient has any hair over the anterior fontanelle, it will be shaved prior to transport to the procedure room (a procedure often done in neonatology to gain access to scalp veins). The insertion site is prepped with Betadine that is quickly removed with alcohol to prevent iodine absorption. The area is also “draped” with a small transparent barrier in the usual surgical fashion.
Trans-fontanelle Ultrasound (H8041VW Venue Go R3 Focus Package, H45041DL 3SC-RS Phased Array Probe, and H40442LM 9L-RS Transducer) is then utilized to evaluate the entirety of the ventricular system to rule out the presence of pre-existing blood products or clot. The ultrasound probe is covered with a sterile sheath.
Under sterile ultrasonic guidance, an ultrathin 30-34 gauge 2.5 cm (BD Precision Glide) needle is placed into the anterior aspect of the ipsilateral lateral ventricle at an angle that is perpendicular to the surround skull. Upon ultrasound verification that the tip is positioned in the LV, 1.5 mL of cerebrospinal fluid (CSF) is aspirated into a 3 mL Hamilton syringe over a 60 second period. If CSF does not flow freely back into a well-positioned patent syringe, the procedure will be terminated, and the patient returned to the NICU. If CSF can be freely aspirated, then the syringe will be removed, and the needle left in place at this time. A preloaded sterile Hamilton Syringe containing 1 mL of the hNSCs in suspension will be attached to the 30-34 gauge needle. 1 mL of this fluid is administered over a 60 second period by gentle intermittent pressure on the syringe plunger (i.e., the standard rate for cell infusions is 1 mL/min).
The patient will continue to be monitored to verify an absence of any changes in vital signs. Once verified, the empty syringe is removed and the prior syringe containing the CSF is re-attached to the hub of the 30-34 gauge needle and 0.5-0.1 mL (of the 1.5 mL initially removed, based on the baby's dose cohort) replaced as a slow push over a 60 second period. This 0.5 mL CSF, added to the 1.0 mL cellular suspension, will allow the patient to remain “volume neutral” intraventricularly. The patient will continue to be monitored to verify stable vital signs. Once the above procedures have been completed, the 30-34 gauge needle and attached syringe will be left in place for 1 minute and then removed. The needle will be disposed of in a sharps container and the syringe submitted to pathology for evaluation of the remaining 1 mL of CSF.
Using the ultrasound, the continued absence of blood in the ventricle or along the insertion tract is verified.
The site of the needle puncture is gently manually compressed for 5 minutes, and an aseptic dressing is applied (a 1 cm circular adhesive gauze bandage over the pinhole-sized puncture site on the scalp, as one would do after a lumbar puncture or ventricular tap). Sutures and steristrips are not necessary. Although the puncture site usually closes spontaneously within hour, the bandage will be kept in place overnight. Once there is no evidence of CSF leakage (rarely, if ever, occurs because the skin covers the tiny puncture almost immediately), the bandage will be removed.
The contralateral side is similarly approached as described above. Again, under sterile ultrasonic guidance, a 30-34-gauge needle is placed into the anterior aspect of the LV. Upon the ultrasonic verification that the tip is in the center of the LV, 1.5 mL of CSF is aspirated into a syringe. The syringe is removed, and the needle left in place. A new syringe containing 1 mL of the hNSC suspension is attached to the 30-34-gauge needle. This suspension (1 mL) will be administered over 60 secs. The patient will continue to be monitored to verify stable vital signs. Once verified, the empty syringe will be removed and the prior syringe containing the CSF re-attached to the hub of the 30-34-gauge needle and 0.5-0.1 mL (of the initial 1.5 mL removed, based on baby's dose cohort) administered as a slow push over 60 secs., ensuring neutral intraventricular volume. The patient will continue to be monitored to verify stable vital signs. Once completed, the 30-34 gauge needle and attached syringe is removed. The needle is disposed of in a sharps container and the syringe submitted to pathology for evaluation of the remaining 1 ml of CSF.
Again, using the ultrasound, the absence of blood within the ventricle or along the insertion tract is verified.
An adhesive gauze bandage as described above is applied to the pinhole-sized needle puncture site on the scalp.
Drapes and paper tape is removed from the patient.
The patient is then placed back on a warming table with monitors still in place and transported back to their spot in the NICU.

### Example 12 Starting Human Neural Stem Cells Dose

To calculate an appropriate starting dose for the human studies, the established safe and effective preclinical doses in mice, rats, and NHPs have been extrapolated to human subjects based on weight, as detailed in Table 10. The dose of MSCs reported in a previous clinical trial to have been safely instilled in the cerebral ventricles of human preterm newborns (a similar patient population as in the present planned study) is also considered. While the therapeutic modality is different (enzyme replacement compared to supplying a neurotrophic agent), the optimal ratio of healthy donor cells to genetically deficient host cells to be 1:10, which conferred 28% of normal enzyme levels to a cerebral region. The ratio could not be less than 1:140 and still achieve a therapeutic enzyme level and restoration of metabolic function. When a dose of NSCs was administered at 2×10⁷ cells/kg/ventricle (i.e., an absolute dose of 1×10⁵ NSCs per ventricle or 2×10⁵ cells total NSCs per animal), then enzyme levels of 5% of normal could be achieved cerebrum wide, with any level >2% of normal being sufficient to restore normal metabolism, and hence deemed therapeutic. As Table 10 indicates, a dose of hNSCs demonstrated to be effective in HII (FIGS. 26-30, 32-35) is similar: 1×10⁷ cells/kg/ventricle. The dose can be escalated in rat pups to up to 2×10⁶ cells/rat pup (=8.0×10⁷/kg [pup weight]=4.0×10⁷/kg/ventricle) and eliminate most penumbral damage [FIGS. 37B, 39]. The dose-escalation studies shown in FIG. 37, yielded the excellent outcomes shown in FIGS. 38-40. The dose of at least 2.5×10⁵ cells/pup injected into 1 ventricle (=1.0×10⁷ cells/kg [pup weight]=1.0×10⁷/kg/ventricle) was termed “Standard Dose (SD)”, and was employed in the initial experiments illustrated in FIGS. 26-30, 32-35 (all of which involved injection into only 1 lateral ventricle). The first dose escalation step was to inject both ventricles of the same pup with a standard dose (SD) of hNSCs, meaning such recipients received, in total, a double dose of cells (e.g., 2×SD). From that point on, all animals received biventricular injections. The next cohort of pups received 2×SD per ventricle, meaning 4×SD per pup. The next cohort had a further doubling of the previous dose per ventricle, meaning 4×SD per ventricle or 8×SD per pup. These sequential dose escalations—and their impact on reducing the severity classification of the pups—are shown in FIG. 37B. By 4×SD per pup (divided between the 2 lateral ventricles), and definitely by 8×SD per pup (divided between the 2 ventricles), the penumbra in most animals was entirely normalized, meaning the category of “moderately injured animals” had fallen to 0. (Animals whose lesion was characterized predominantly by unsalvageable core, making them “severe”, remained essentially unchanged, although even the number of animals classified as having a “severe” injury seemed to improve/decrease.

In no preclinical HII model, including rat pups, were adverse effects observed at high cell doses, and therefore a NOAEL was not identified. The highest hNSC dose tested to date in any animal was 5.0×10⁷ cells/kg/ventricle in fetal bonnet monkeys with no observed adverse effects and normal cerebral development.

In the GLP safety/toxicity studies for IND-enabling studies in rat pups, a dose escalation starting at 4.0×10⁷ cells/kg/ventricle (i.e., 8×SD per rat pup) is performed, the dose found most effective yet safe in asphyxiated pups (FIG. 37B). From that point a escalate up to 5-times that dose (to 2×10⁸ cells/kg/ventricle) is implemented, exceeding the safe and effective dose established for asphyxiated rat pups (FIG. 37B) in order to establish a NOAEL, a ceiling never to exceed. The neonatal sheep logistics study, will further establish, bracket, and exceed doses found to be safe and effective in fetal bonnet monkeys defining the “high dose” in human subjects.

Because the doses found to be efficacious and safe in rat pups exceed the highest dose explored to date in the above-referenced clinical trial in which MSCs (a larger diameter cell than an NSC) were instilled into the ventricles of human newborns (and nevertheless found to be safe), escalations beyond that published dose of 1.0×10⁷/kg/ventricle in a human newborn trial (planned as “low dose” in sheep [Table 10]) will be informed by dose escalation in the fetal sheep, which approximate the body and brain size of human newborns. However, even this more conservative approach should be therapeutically impactful in human babies: note that a simple doubling of the previously-established safe cell dose, as is planned in the clinical trial (Table 10), constitutes 2.0×10⁷/kg/ventricle which, by the rat pup criteria equals 4×SD per animal, and still was sufficient for eliminating the penumbra (i.e., normalizing rat pups with a “moderately severe” injury (FIG. 37B).

The preclinical data suggests that the ideal density of hNSCs in a NS suspension is 5×10⁴ μL, indicating that a volume of 1-3 mL may be instilled per ventricle to achieve 1.5-4.0×10⁷ cells/kg/ventricle (the cell dose to be determined by the dose escalation studies). As suggested in previously published clinical trials instilling cells into the ventricles of human newborns, volumes as high as 7 mE/ventricle may be well-tolerated by human neonates if an equal volume of CSF is removed to maintain a stable volume and ICP.

### Example 13: Clinical Study Plan

### Overview

Term HII newborns who meet criteria for and receive SOC (3-days of hypothermia) are randomized to receive one of the following:
1. Non-Interventional: Continued follow-up (symptomatic care; n=20) (While these patients will not undergo a sham procedure, to preserve blinding, they will be wheeled into the procedure room and receive an adhesive bandage on the scalp overlying where a needle would have been inserted percutaneously)
2. Investigational Treatment: hNSC implantation on DOL 4-5, administered via a 30-34-gauge catheter inserted percutaneously through the open anterior fontanel into each of the 2 lateral cerebral ventricles, with each ventricle receiving 1.0×10⁷ cells/kg/ventricle in 0.7 mL of normal saline (NS; Cohort 1; n=6) or 2.0×10⁷ cells/kg/ventricle in 1.4 mL NS (Cohort 2; n=6)

Additional control groups will be comprised of:
Neonates who would qualify clinically for, but do not get, HT (e.g., delay-to-treatment [>6 hours of age]; lack of parental consent): will be enrolled for symptomatic care and follow-up only.
Neonates who receive HT but parents do not give consent for hNSC implantation: will be enrolled for symptomatic care and follow-up only

All subjects will undergo MRI, as per routine for all HII babies in a NICU, to document severity (DOL 4, upon rewarming) and to quantify “Penumbra:Core Ratios” (via HRS); while these data will be recorded for future correlations with outcome, they will not be used at this point for inclusion or exclusion criteria.

### Endpoints (Evaluators Blinded to Treatment Status)

### Primary

Safety.

### Secondary

Improved outcome by addition of hNSCs to HT.
   Neuro exam (including seizure history) at 12 and 18 months.
   Behavior (including developmental milestones and Bayley Scales of Infant development on follow-up) at 12 and 18 months.
   MRI at 12 months (reduction in penumbral volume? no increase in core volume? no ventriculomegaly? no significant loss in parenchymal volume?).
Prognostic value of the MRI-based HII severity scale for:
   Outcome.
   Selecting candidates that will be responsive to hNSC treatment.

This is a multicenter FIH Phase 1b/2a trial examining intracerebroventricular implantation of neuroprotective HFB2050.SBP hNSCs following HT (SOC) to improve outcomes when compared to SOC alone. Nonclinical studies suggest complementary and possibly additive/synergistic neuroprotective effects from these 2 modalities. To evaluate safety (primarily) and efficacy (secondarily) of the HFB2050.SBP hNSCs in this treatment population Population

Full-term HII newborns with Sarnat clinical scores of 2 or 3 and meet criteria for and receive 3 days hypothermia (SOC), and have no other dysgenetic or dysmorphic features Investigational Product

HFB2050.SBP: Freshly dissociated hNSCs suspended as single cells in NS at a concentration of 5×10⁴ cells/μL, maintained at 4° C. before administration.

### Dose Form and ROA

Minimally invasive aseptic percutaneous instillation of a dissociated hNSC suspension in NS via a 5-cm 30-34-gauge needle through the open anterior fontanelle into each of the 2 lateral cerebral ventricles.

Insertion guided by cranial ultrasound.

Two distinct needle passes—one into each ventricle.

### Dose Escalation:

1×10⁷/kg/ventricle administered as a sufficient volume of a 5×10⁴ cell/μL suspension of NS=1st dose level for Cohort 1.
2×10⁷/kg/ventricle in 1.4 mL NS=2× dose escalation (2nd dose level).
May consider progressing to 3.5×10⁷ cells/kg/ventricle (to match cell dosage found to be safe and effective in fetal monkeys), but dose escalations >2.0×10⁷ kg/ventricle in human newborns will depend upon nonclinical studies performed in fetal sheep, which approximate the size (both of body and brain) of human newborns.

Preclinical studies comparing various cell densities, doses, and ROAs (intra-lesional, ipsilateral ventricle, contralateral ventricle, intravascular) suggest the above to be optimal.

### Inclusion Criteria

Neonates ≥37 weeks gestation (full-term or post-term):
Sarnat Stage 2-3 HII.
No other anomalies or dysmorphologies.
Qualify for HT:
   Apgar ≤5 at 10 minutes.
   Continued need for resuscitation at 10 minutes.
   pH <7.00 or base deficit ≥16.0 mmol/L.
   In umbilical arterial or venous blood sample prenatally or in a venous, arterial, or capillary blood sample within 60 minutes of birth.
   Encephalopathy:
      Lethargy, stupor, or coma.
      At least one of the following:
      Hypotonia.
      Abnormal reflexes (including oculomotor or pupillary).
      Absent or weak suck.
      Seizures.

### Exclusion Criteria

Preterm neonates.
Sarnat Stage 0 or 1 HII.
Pre-existing anomalies, dysmorphologies, dysgenic features, genetic conditions.
Pre-existing intracranial bleeds, masses, hydrocephalus.
Do not qualify for HT:
   Do not qualify based on biochemical or clinical criteria.
   >6 hours post-birth (or known inciting event).
   Preterm.
   No informed consent for HT.
Point-of-care is unable to obtain or administer hNSCs.
Point-of-care unable to perform MRI and/or bedside EEG, assess infants for HT, administer HT, perform neurological follow-up on infants until discharge.
No informed consent for hNSC implantation.

### Endpoints and Outcomes

Evaluators blinded to newborn's treatment status.
Primary
   Safety comparable to controls.
Secondary
   Improved outcome by addition of hNSCs to HT regimen:
      Neuro exam (including seizure history) at 12 and 18 months.
      Behavior (including developmental milestones and Bayley Scales of Infant development on f/u) at 12 and 18 months.
      MRI improvement at 12 months.
      Resolution or reduction in volume of penumbra.
      No increase in core volume.
      No loss of parenchyma.
      No enlargement of ventricles (relevant to the given patient's pre-treatment condition and to untreated age-matched asphyxiated patients).
      No worsening of other CNS structures.
   Retrospective assessment of the predictive value of the proposed MRI-based HII severity scale:
      For prognosticating outcome.
      For selecting candidates for receiving neuroprotective hNSCs.
      For ruling out or ruling in adverse outcomes from the hNSC implantation.
Safety
   No intra- or extra-cranial tumors, cell overgrowth, deformation, or inappropriate cell types.
   No deterioration in neurologic/mental status (including loss of motor/sensory skills, developmental milestones, sensorium, state-of-alertness, responsiveness, or respiratory control); no new asymmetries, new-onset or worsening of seizures, or loss of milestones.
   No pain/discomfort.
   No systemic toxicity.
   No excess vascularity, compromised blood-brain barrier, hemorrhage.
   No loss of parenchyma or worsening ventriculomegaly.
   No increased intracranial pressure.
   No infection.
   No inflammatory or immune reaction.
   No increased scarring.
   No increased mortality/morbidity

### Trial Sites

Given the prevalence of perinatal HII and the need for statistical power, the Phase 1a/2b clinical trial will be multi-institutional enlisting up to four Level 3 or Level 4 NICUs in the Southern California area, within a 45-minute drive from UCSD's GMP manufacturing site (which will prepare patient-ready sterile syringes for administration by health care provider. Qualifying investigational sites will be NICUs with a multidisciplinary team of neonatologists, child neurologists, neuroradiologists, neurosurgeons, child development and infant follow-up pediatricians. Participating trial sites will be experienced in identifying and classifying potentially asphyxiated newborns by clinical and biochemical criteria, designating whether they are potential candidates for HT, administering HT (HT has become standard practice in all birthing units, nurseries, and NICUs in the US), monitoring cardiovascular-respiratory and EEG status of babies through the procedure, and providing multidisciplinary, neurological, neurodevelopment, and imaging follow-up to at least 2 years of age. In all cases, the health care provider will travel to the site to perform the administration procedure.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure can be employed in practicing the present disclosure. It is intended that the following claims define the scope of the present disclosure and that methods and structures within the scope of these claims and their equivalents be covered thereby.

1. A method of treating perinatal hypoxic-ischemic injuries (HII) in a human subject, comprising administering to the human subject an effective amount of human neural stem cells (hNSCs) or progenitors thereof comprising an HFB2050 cell or a progenitor thereof, thereby treating the perinatal hypoxic-ischemic injuries in a human subject.

2. The method of claim 1, further comprising providing a hypothermia therapy to the human subject.

3. A method of treating perinatal hypoxic-ischemic injuries (HII) in a human subject comprising administering to the human subject an effective amount of human neural stem cells (hNSCs) or progenitors thereof, wherein the human subject has received a hypothermia therapy.

4. The method of claim 3, wherein the hNSCs or progenitors thereof comprise an HFB2050 cell.

5. The method of claim 3, wherein the hNSCs or progenitors thereof are genetically modified.

6. The method of claim 5, wherein the hNSCs or progenitors thereof comprises a transgene.

7. The method of claim 6, wherein the transgene comprises SOX2 or Nestin.

8. The method of claim 3, wherein the hNSCs or progenitors thereof comprise an HSC-derived stable cell line.

9. The method of claim 3, wherein the method does not comprise immunosuppression.

10. The method of claim 3, wherein the hNSCs or progenitors thereof comprise a characteristic of
(i) able to differentiate into three cardinal neural cell types in a stable ratio after prolonged passaging,
(ii) self-renewal,
(iii) possess normal growth kinetics,
(iv) able to be cryopreserved and retain normal characteristics upon thaw and return to culture, or
any combination thereof.

11. The method of claim 3, wherein the hNSCs or progenitors thereof are able to integrate into periventricular germinal zone in the human subject.

12. The method of claim 3, wherein the human subject is a neonate.

13. The method of claim 12, wherein the neonate is about 3.5 kg.

14. The method of claim 12, wherein the neonate is full-term.

15. The method of claim 3, comprising administering the hNSCs or progenitors thereof to the human subject no more than 6 hours or no more than three (3) days after birth or post injury of the human subject.

16. The method of claim 3, comprising administering the hNSCs or progenitors thereof to the human subject 6 hours after birth of the human subject.

17. The method of claim 3, comprising administering the hNSCs or progenitors thereof to the human subject at about 3 days after birth or post injury of the human subject.

18. The method of claim 3, comprising administering the hNSCs or progenitors thereof to the human subject at about 2 days after birth or post injury of the human subject.

19. The method of claim 3, wherein the administration of the hNSCs or progenitors thereof and the hypothermia therapy occur concurrently or sequentially.

20. The method of claim 3, wherein the administration of the hNSCs or progenitors thereof occurs prior to the hypothermia therapy.
